(19) United States
(12) Patent Application Publication
(10) Pub. No.: US 2024/0344041 A1
(43) Pub. Date: Oct. 17, 2024

(54) ARTIFICIAL PROTEIN TO RESTORE SYNAPTIC FUNCTION

(71) Applicant: PRESIDENT AND FELLOWS OF HARVARD COLLEGE, Cambridge, MA (US)

(72) Inventors: Pascal S. KAESER, Cambridge, MA (US); Chao TAN, Cambridge, MA (US); Shan Shan WANG, Cambridge, MA (US)

(73) Assignee: PRESIDENT AND FELLOWS OF HARVARD COLLEGE, Cambridge, MA (US)

(21) Appl. No.: 18/517,417

(22) Filed: Nov. 22, 2023

Related U.S. Application Data
(60) Provisional application No. 63/427,309, filed on Nov. 22, 2022.

(51) Int. Cl. C12N 9/14; A61K 38/00; A61P 25/28; C07K 14/705

(52) U.S. Cl. CPC C12N 9/14; A61P 25/28; C07K 14/705; C12Y 306/05; A61K 38/00

(57) ABSTRACT

Technology described herein relates to a fusion protein comprising a zinc-finger domain (ZNF) of Regulating Synaptic Membrane Exocytosis Protein (RIMS); and a Ca_{V}β Ca2+ channel subunit. Compositions comprising the fusion protein and method of treatment utilizing the fusion protein are also provided herein.

### CROSS-REFERENCE TO RELATED APPLICATIONS

This application claims benefit under 35 U.S.C. § 119(c) to U.S. Provisional Application No. 63/427,309, filed Nov. 22, 2022, the contents of which are incorporated herein by reference in their entirety.

### GOVERNMENT SUPPORT

This invention was made with government support under MH113349 and NS083898 awarded by the National Institute of Health (NIH). The government has certain rights in the invention.

### SEQUENCE LISTING

The instant application contains a Sequence Listing which has been submitted electronically in XML format and is hereby incorporated by reference in its entirety. Said XML copy, created on Jul. 9, 2024, is named 002806-000103USPL_SL.xml and is 99,773 bytes in size.

### TECHNICAL FIELD

The technology described herein relates to fusion proteins and methods of use to restore and enhance transmitter secretion, e.g., neurotransmitter or hormone secretion.

### BACKGROUND

Active zones are molecular machines attached to the presynaptic plasma membrane that control neurotransmitter release through synaptic vesicle docking and priming, and through coupling of these vesicles to Ca²⁺ entry. The complexity of active zone machinery has made it challenging to determine which mechanisms drive these roles in release. Active zones are composed of families of scaffolding proteins including RIM, ELKS, Munc13, RIM-BP, Bassoon/Piccolo and Liprin-α. Each of these proteins is encoded by multiple genes and the individual proteins are large, ranging from 125 to 420 kDa, forming complex protein networks. Mechanisms for their assembly are not well understood. Better understanding of their assembly could help control the central functions of the active zone, namely the generation of releasable vesicles and the positioning of these vesicles close the Ca²⁺ channels for rapid fusion-triggering, which could enhance or repair transmitter secretion leading to improvements of synaptic or endocrine functions.

The active zone is important for transmitter secretion, including synaptic signaling, and many brain disorders are associated with mutations in active zone proteins or defective active zone function. With a better understanding of the active zone machinery and their function, transmitter secretion can be controlled to restore efficacy and spatiotemporal precision of neurotransmitter or hormonal release. Thus, there is a need in the art for molecules that enhance or repair transmitter secretion thereby improving synaptic and endocrine function by interacting with or reconstructing functions of the active zone.

### SUMMARY

The technology described herein is directed to a fusion protein for enhancing or repairing transmitter secretion, e.g., synaptic or hormonal secretion, thereby improving endocrine or synaptic function. Accordingly, one aspect described herein provides a fusion protein comprising a) a zinc-finger domain (ZNF) of Regulating Synaptic Membrane Exocytosis Protein (RIMS); and b) a Ca_{V}β Ca²⁺ channel subunit.

In one embodiment of any aspect provided herein, the RIMS is Regulating Synaptic Membrane Exocytosis Protein 1 (RIMS1) or Regulating Synaptic Membrane Exocytosis Protein 2 (RIMS2).

In one embodiment of any aspect provided herein, the Ca_{V}β Ca²⁺ channel subunit is Ca_{V}β1, Ca_{V}β2, Ca_{V}β3, or Ca_{V}β4.

In one embodiment of any aspect provided herein, the ZNF comprises a sequence selected from SEQ ID NOs: 1-4 and 38-41.

In one embodiment of any aspect provided herein, the wherein the Ca_{V}β Ca²⁺ channel subunit comprises SEQ ID NOs: 5-8 and 42-47.

Another aspect described herein provides a synthetic nucleic acid encoding any of the fusion proteins described herein.

Another aspect described herein provides an expression cassette comprising any of the synthetic nucleic acids described herein.

Another aspect described herein provides a vector encoding any of the fusion proteins described herein.

Another aspect described herein provides a vector comprising any of the expression cassettes described herein.

In one embodiment of any aspect provided herein, the vector is a DNA or RNA nucleic acid vector.

In one embodiment of any aspect provided herein, the vector further comprises a promoter that is operatively linked to the nucleic acid described herein.

In one embodiment of any aspect provided herein, the promoter is a constitutive promoter.

In one embodiment of any aspect provided herein, the promoter is a nervous tissue-specific promoter.

In one embodiment of any aspect provided herein, the vector is a viral vector. In one embodiment of any aspect provided herein, the viral vector is selected from of the group consisting of: an adeno associated virus (AAV), adenovirus, lentivirus vector, and a herpes simplex virus (HSV).

In one embodiment of any aspect provided herein, the viral vector is a recombinant AAV (rAAV).

Another aspect described herein provides a cell expressing any of the fusion proteins described herein, any of the synthetic nucleic acids described herein, any of the expression cassettes described herein, or any of the vectors described herein.

In one embodiment of any aspect provided herein, the cell is a neuronal cell. In one embodiment of any aspect provided herein, the cell is a non-neuronal cell. In one embodiment of any aspect provided herein, the cell is a hormone secreting cell.

Another aspect described herein provides a pharmaceutical composition comprising any of the fusion proteins described herein, any of the synthetic nucleic acids described herein, any of the expression cassettes described herein, any of the vectors described herein or any of the cells described herein.

In one embodiment of any aspect provided herein, the formulation of the pharmaceutical composition is selected from the group consisting of: direct injection or infusion into the central nervous system (CNS); formulation as a solution comprising a carrier protein; formulation as a nanoparticle; formulation as a liposome; formulation as a nucleic acid; formulation as a CNS-tropic viral vector; formulation with or linkage to an agent that is endogenously transported across the BBB; formulation with or linkage to a cell penetrating peptide (CPP); formulation with or linkage to a BBB-shuttle; and formulation with or linkage to an agent that increases permeability of the BBB.

In one embodiment of any aspect provided herein, the pharmaceutical composition is formulated for delivery across the blood-brain barrier (BBB).

In one embodiment of any aspect provided herein, the pharmaceutical composition is formulated for delivery to the brain.

Another aspect described herein provides a method of repairing or enhancing synaptic function in a subject, the method comprising administering to a subject in need thereof an effective amount any of the fusion proteins described herein, any of the synthetic nucleic acids described herein, any of the expression cassettes described herein, any of the vectors described herein or any of the cells described herein.

Another aspect described herein provides a method of treating a neurological disorder in a subject, the method comprising administering to a subject in need thereof an effective amount of any of the fusion proteins described herein, any of the synthetic nucleic acids described herein, any of the expression cassettes described herein, any of the vectors described herein or any of the cells described herein.

In one embodiment of any aspect provided herein, the administration is performed intracranially, epidurally, intrathecally, intraparenchymally, intraventricularly, or subarachnoidly.

In one embodiment of any aspect provided herein, the fusion protein, synthetic nucleic acid, expression cassette, vector, cell, or pharmaceutical composition is administered in a formulation that crosses the blood-brain barrier.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1M depicts that RIM restores active zone structure after knockout of RIM and ELKS. (FIG. 1A) Schematic of HA-tagged ELKS2αB and RIM1α that were expressed in cKO^{R+E} neurons using lentiviruses. H: HA tag, CC: coiled-coil region, Zn: zinc finger domain, PDZ: PDZ domain, C₂A and C₂B: C₂ domains, P: proline rich (PxxP−) motif. (FIGS. 1B-1C) Sample STED images (FIG. 1B) and quantification (FIG. 1C) of side-view synapses in cultured hippocampal neurons after conditional knockout of RIM and ELKS (cKO^{R+E}), stained for RIM1 (STED), PSD-95 (STED), and Synaptophysin (confocal) in control^{R+E} and cKO^{R+E} synapses, and in cKO^{R+E} synapses after lentiviral re-expression of ELKS2αB or RIM1α. Peak position and levels (FIG. 1C) were analyzed in line profiles (600 nm×200 nm, grey shaded area in FIG. 1B) positioned perpendicular to the center of PSD-95 ‘bars’ and aligned to the PSD-95 peak, dotted lines mark control (grey) and cKO^{R+E} (black) levels for comparison, n=60 synapses/3 independent cultures per condition. (FIGS. 1D-1M) Same as (FIGS. 1B-1C), but stained for ELKS2 (FIGS. 1D-1E), Munc13-1 (FIGS. 1F-1G), Bassoon (FIGS. 1H-1I), RIM-BP2 (FIGS. 1J-1K) or Ca_{V}2.1 (FIGS. 1L-1M), n=60/3 per condition. PSD-95 levels were assessed for all experiments as in C and outcomes were similar, but are not shown for simplicity. Data are mean±SEM; **P<0.01, ***P<0.001 compared to cKO^{R+E} as determined by two-way ANOVA followed by Dunnett's multiple comparisons post-hoc tests. For STED analyses workflow, assessment of Liprin-α3, and assessment of rescue protein expression by Western blotting, see FIG. 9A-9G; for independent confocal microscopic experiments, see FIGS. 10A-10F.

FIGS. 2A-2N show RIM restores active zone functions after active zone disruption. (FIGS. 2A-2B) Sample images (FIG. 2A) and analyses (FIG. 2B) of synapses of high-pressure frozen neurons analyzed by electron microscopy. Quantification was performed on single sections, control^{R+E} n=96 synapses/2 independent cultures, cKO^{R+E} n=100/2, cKO^{R+E}+ELKS2αB n=100/2, and cKO^{R+E}+RIM1α n=98/2. (FIGS. 2C-2D) Sample traces (FIG. 2C) and quantification (FIG. 2D) of EPSCs evoked by focal electrical stimulation, control^{R+E} n=19 cells/3 independent cultures, cKO^{R+E} n=18/3, cKO^{R+E}+ELKS2αB, n=18/3, cKO^{R+E}+RIM1α n=19/3. (FIGS. 2E-2F) Sample traces (FIG. 2E) and quantification (FIG. 2F) of EPSCs triggered by hypertonic sucrose, the first 10 s of the EPSC were quantified to estimate the RRP, n=19/3 per condition. (FIGS. 2G-2H) Sample traces (FIG. 2G) and quantification (FIG. 2H) of EPSC paired pulse ratios to estimate p, n=16/3 per condition. (FIGS. 2I-2J) Sample traces (FIG. 2I) and quantification (FIG. 2J) of IPSCs evoked by focal electrical stimulation, control^{R+E} n=20/3, cKO^{R+E} n=21/3, cKO^{R+E}+ELKS2αB n=21/3, and cKO^{R+E}+RIM1α n=22/3. (FIGS. 2K-2L) Sample traces (FIG. 2K) and quantification (FIG. 2L) of IPSCs triggered by hypertonic sucrose, the first 10 s of the IPSC were quantified to estimate the RRP, n=19/3 per condition. (FIGS. 2M-2N) Sample traces (FIG. 2M) and quantification (FIG. 2N) of IPSC paired pulse ratios, n=15/3 per condition. Data are mean±SEM; **P<0.01, ***P<0.001 compared to cKO^{R+E} as determined by Brown-Forsythe ANOVA followed by Games-Howell's multiple comparisons post-hoc test (B, docked vesicles), Brown-Forsythe ANOVA followed by Dunnett's T3 multiple comparisons post-hoc test (D), Kruskal-Wallis followed by Dunn's multiple comparisons post-hoc tests (F, J and L), or by two-way ANOVA followed by Dunnett's multiple comparisons post-hoc tests (H and N). For analyses of EPSCs in low extracellular Ca²⁺ or with competitive NMDAR antagonists, see FIGS. 11A-11H.

FIGS. 3A-3F show RIM PDZ domains mediate RIM active zone localization while zinc fingers associate with synaptic vesicles. (FIGS. 3A-3B) Schematic of key presynaptic interactors of full-length RIM1α (FIG. 3A) and of RIM1 deletion mutants or individual domains (FIG. 3B) that were expressed in cKO^{R+E} neurons. (FIGS. 3C-3D) Sample STED images (FIG. 3C) and quantification (FIG. 3D) of side-view synapses stained for HA (STED), PSD-95 (STED), and Synaptophysin (confocal), dotted lines mark cKO^{R+E} (black) and cKO^{R+E}+RIM1α (blue) levels for comparison, n=60 synapses/3 independent cultures per condition. (FIGS. 3E-3F) Same as (FIGS. 3C-3D), but for individual domains, n=60/3 per condition. Data are mean±SEM; *P<0.05, ***P<0.001 compared to cKO^{R+E}+RIM1α as determined by two-way ANOVA followed by Dunnett's tests. For assessment of rescue expression by Western blotting, see FIGS. 12A and 12B.

FIGS. 4A-4M show RIM zinc fingers render undocked vesicles release-competent through Munc13 recruitment. (FIG. 4A) Schematic of rescue proteins. (FIGS. 4B-4C) Sample STED images (FIG. 4B) and quantification (FIG. 4C) of side-view synapses stained for Munc13-1 (STED), PSD-95 (STED), and Synaptophysin (confocal), dotted lines mark control^{R+E} (grey) and cKO^{R+E} (black) levels for comparison, n=60 synapses/3 independent cultures per condition. (FIGS. 4D-4E) Same as (FIGS. 4B-4C), but for Ca_{V}2.1, n=60/3 per condition. PSD-95 levels were assessed as in FIG. 4C and outcomes were similar, but are not shown for simplicity. (FIGS. 4F-4G) Sample electron microscopic images (FIG. 4F) and analyses (FIG. 4G) of synapses of high-pressure frozen neurons, control^{R+E} n=105 synapses/2 independent cultures, cKO^{R+E} n=110/2, cKO^{R+E}+RIM1-Zn n=105/2, and cKO^{R+E}+RIM1-ΔZn n=102/2. It was not possible to include RIM1α full-length rescue in all electron microscopic experiments due to the laborious nature of processing and analyses. As essential controls, we included control^{R+E} and cKO^{R+E} neurons in each electron microscopic experiment. (FIGS. 4J-4K) Sample traces (FIG. 4J) and quantification (FIG. 4K) of EPSCs triggered by hypertonic sucrose to estimate the RRP, n=17/3 per condition. (FIGS. 4L-4M) Sample traces (FIG. 4L) and quantification (M) of paired pulse ratios to estimate p, n=25/4 per condition. Data are mean±SEM; **P<0.01, ***P<0.001 compared to cKO^{R+E} as determined by two-way ANOVA followed by Dunnett's tests (FIGS. 4C, 4E and 4M), Brown-Forsythe ANOVA followed by Games-Howell's multiple comparisons post-hoc test (FIG. 4G, docked vesicles), or by Kruskal-Wallis analysis followed by Dunn's tests (FIGS. 4I and 4K). For recordings of IPSCs, see FIGS. 12C-12H.

FIGS. 5A-5K show a Ca_{V}β4-RIM1 zinc finger fusion-protein recruits priming machinery close to Ca²⁺ channels. (FIG. 5A) Schematic of rescue proteins. (FIGS. 5B-5C) Sample STED images (B) and quantification (C) of side-view synapses stained for HA to visualize rescue proteins (STED), PSD-95 (STED), and Synaptophysin (confocal), dotted lines mark cKO^{R+E} (black) levels for comparison, n=60 synapses/3 independent cultures per condition. (FIGS. 5D-5K) Same as (FIGS. 5B-5C), but stained for Munc13-1 (FIGS. 5D-5E), Ca_{V}2.1 (FIGS. 5F-5G), Bassoon (FIGS. 5H-5I), and RIM-BP2 (FIGS. 5J-5K), n=60/3 in each condition. PSD-95 levels were assessed for all experiments as in C and outcomes were similar, but are not shown for simplicity, dotted lines mark control (grey) and cKO^{R+E} (black) levels for comparison. Data are mean±SEM; ***P<0.001 compared to cKO^{R+E} as determined by two-way ANOVA followed by Dunnett's tests. For assessment of various rescue fusion-proteins, STED analyses of Ca_{V}β4, and expression levels of rescue proteins by Western blot, see FIGS. 13A-13H.

FIGS. 6A-6N show β4-Zn reconstitutes vesicle docking and release. (FIGS. 6A-6B) Sample electron microscopic images (FIG. 6A) and analyses (FIG. 6B) of synapses of high-pressure frozen neurons, control^{R+E} n=83 synapses/2 independent cultures, cKO^{R+E} n=85/2, cKO^{R+E}+RIM1-Zn n=84/2, cKO^{R+E}+Ca_{V}β4 n=83/2 and cKO^{R+E}+β4-Zn n=87/2. (FIGS. 6C-6D) Sample traces (C) and quantification (D) of EPSCs evoked by focal electrical stimulation, control^{R+E} n=17 cells/3 independent cultures, cKO^{R+E} n=17/3, cKO^{R+E}+RIM1-Zn n=18/3, cKO^{R+E}+Ca_{V}β4 n=18/3 and cKO^{R+E}+β4-Zn n=17/3. (FIGS. 6E-6F) Sample traces (FIG. 6E) and quantification (FIG. 6F) of the EPSC triggered by hypertonic sucrose, the first 10 s of the EPSC were quantified to estimate the RRP, n=17/3 per condition. (FIGS. 6G-6H) Sample traces (FIG. 6G) and quantification (FIG. 6H) of EPSC paired pulse ratios to estimate p, control^{R+E} n=16/3, cKO^{R+E} n=16/3, cKO^{R+E}+RIM1-Zn n=17/3, and cKO^{R+E}+Ca_{V}β4 n=16/3, cKO^{R+E}+β4-Zn n=17/3. (FIGS. 6I-6J) Sample traces (I) and quantification (J) of IPSCs evoked by focal electrical stimulation, control^{R+E} n=21/4, cKO^{R+E} n=21/4, cKO^{R+E}+RIM1-Zn n=24/4, cKO^{R+E}+Ca_{V}β4 n=22/4, and cKO^{R+E}+β4-Zn n=22/4. (FIGS. 6K-6L) Sample traces (FIG. 6K) and quantification (FIG. 6L) of IPSC triggered by hypertonic sucrose, the first 10 s of the IPSC were quantified to estimate the RRP, n=20/3 per condition. (FIGS. 6M-6N) Sample traces (FIG. 6M) and quantification (FIG. 6N) of IPSC paired pulse ratios, control^{R+E} n=20/4, cKO^{R+E} n=19/4, cKO^{R+E}+RIM1-Zn n=20/4, cKO^{R+E}+Ca_{V}β4 n=19/4, and cKO^{R+E}+β4-Zn n=20/4. Data are mean±SEM; *P<0.05, **P<0.01, ***P<0.001 compared to cKO^{R+E} as determined by Brown-Forsythe ANOVA followed by Games-Howell's multiple comparisons post-hoc test (FIG. 6B, docked vesicles), one-way ANOVA followed by Dunnett's multiple comparisons post-hoc tests (FIG. 6B, total vesicles), Kruskal-Wallis followed by Dunn's tests (FIGS. 6D, 6F, 6J and 6L) or by two-way ANOVA followed by Dunnett's tests (FIGS. 6H and 6N).

FIGS. 7A-7G show Binding of β4-Zn to Munc13 mediates presynaptic Munc13 recruitment. (FIG. 7A) Schematic of rescue proteins. (FIGS. 7B-7C) Sample STED images (FIG. 7B) and quantification (FIG. 7C) of side-view synapses stained for HA (STED), PSD-95 (STED), and Synaptophysin (confocal) in cKO^{R+E} synapses, and in cKO^{R+E} synapses after re-expression of β4-Zn or β4-Zn^{K144/6E}, dotted lines mark cKO^{R+E} levels for comparison, n=60 synapses/3 independent cultures per condition. (FIGS. 7D-7G) Same as (FIGS. 7B-7C), but stained for Munc13-1 (D, E) and Ca_{V}2.1 (F, G), n=60/3 in each condition. PSD-95 levels were assessed for all experiments as in C, but are not shown for simplicity, dotted lines mark control^{R+E} (grey) and cKO^{R+E} (black) levels for comparison. Data are mean±SEM; **P<0.01, ***P<0.001 compared to cKO^{R+E} as determined by two-way ANOVA followed by Dunnett's tests. For assessment of rescue protein expression by Western blotting, see FIG. 14A.

FIG. 8A-8K show binding of β4-Zn to Munc13 is essential for restoring vesicle docking and release. (FIGS. 8A-8B) Sample electron microscopic images (FIG. 8A) and analyses (FIG. 8B) of synapses of high-pressure frozen neurons, control^{R+E} n=100 synapses/2 independent cultures, cKO^{R+E} n=99/2, cKO^{R+E}+β4-Zn n=99/2 and cKO^{R+E}+β4-Zn^{K144/6E} n=99/2. (FIGS. 8C-8D) Sample traces (FIG. 8C) and quantification (FIG. 9D) of EPSCs evoked by focal electrical stimulation, n=16 cells/4 independent cultures per condition. (FIG. 8E-8F) Sample traces (FIG. 8E) and quantification (FIG. 8F) of the EPSC triggered by hypertonic sucrose, the first 10 s of the EPSC were quantified to estimate the RRP, n=17/3 per condition. (FIGS. 8G-8H) Sample traces (FIG. 8G) and quantification (FIG. 8H) of paired pulse ratios to estimate p, n=16/4 per condition. (FIGS. 8I-8K) Schematic shows the active zone, a protein network assembled through liquid-liquid phase separation, which controls extent, speed and spatial precision of synaptic vesicle release (FIG. 8I). Knockout of RIM and ELKS leads to loss of active zone scaffolds and of vesicle docking, and impaired release (FIG. 8J). Reconstitution of synaptic function can be achieved in the absence of the active zone scaffolding network. Step-wise reconstitution of active zone function was accomplished by reintroduction of the RIM zinc finger, which localizes to synaptic vesicles, co-recruits endogenous Munc13, and renders non-docked vesicles highly fusogenic (FIGS. 3 and 4). When fused onto the Ca²⁺ channel subunit Ca_{V}β4 (FIG. 8K, FIGS. 5-7), vesicle docking and rapid exocytosis are restored. This reconstitution approach bypasses the need for the active zone scaffolding network. Data are mean±SEM; ***P<0.001 compared to cKO^{R+E} as determined by Brown-Forsythe ANOVA followed by Games-Howell's multiple comparisons post-hoc test (FIG. 8B, docked vesicles), Kruskal-Wallis followed by Dunn's tests (FIGS. 8D and 8F) or by two-way ANOVA followed by Dunnett's tests (FIG. 8H). For IPSC β4-Zn^{K144/6E} rescue data, see FIGS. 14B-14G.

FIG. 9A-9G show workflow for STED analyses and assessment of protein expression and localization after rescue of active zone disruption, related to FIG. 1. (FIG. 9A) Workflow for STED side-view synapse analyses as described in (Held et al., 2020; Nyitrai et al., 2020). (FIG. 9B) Example STED images of a side-view synapse (the control^{R+E} images shown in FIG. 1B are reproduced here) stained for PSD-95 (STED), RIM1 (STED) and Synaptophysin (confocal). (FIG. 9C) Quantification of fluorescent signals of PSD-95 and RIM1 labeling of the synapse shown in B. (FIG. 9D) Summary data of a full experiment for PSD-95 and RIM1, these are the same data as the control^{R+E} data shown in FIG. 1C. (FIGS. 9E-9F) Sample STED images (E) and quantification (F) of side-view synapses stained for Liprin-α3 (STED), PSD-95 (STED), and Synaptophysin (confocal) of control^{R+E} and cKO neurons, and of cKO^{R+E} neurons after re-expression of ELKS2αB or RIM1α, dotted lines mark control^{R+E} (grey) and cKO^{R+E} (black) levels for comparison, n=60 synapses/3 independent cultures per condition. (FIG. 9G) Western blot for RIM1, ELKS2 and Synapsin in homogenates of control^{R+E} and cKO neurons, and of cKO^{R+E} neurons after re-expression of ELKS2αB or RIM1α. Rescue proteins were expressed at overall levels similar to or below wild type proteins. Data are mean±SEM; ***P<0.001 compared to cKO^{R+E} as determined by two-way ANOVA followed by Dunnett's multiple comparisons post-hoc tests.

FIG. 10A-10F show synaptic RIM1α levels determine levels of interacting active zone proteins, related to FIG. 1. (FIG. 10A) Sample confocal images (left) and quantification (right) of RIM1 levels in control^{R+E} and cKO^{R+E} synapses, and in cKO^{R+E} synapses expressing HA-tagged full-length RIM1α or ELKS1αB. Neurons were infected at DIV3 with lentiviruses expressing RIM1α under a synapsin promoter for lower expression (RIM1αlow), or under a ubiquitin promoter for higher expression (RIM1αhigh). Synaptophysin staining was used to define ROIs, and levels of RIM1 were quantified within those ROIs and normalized in each culture to levels in control^{R+E} neurons. n=30 images/3 cultures per condition. (FIGS. 10B-10F) Same as FIG. 10A, but for ELKS1 (FIG. 10B), Munc13-1 (FIG. 10C), Ca_{V}2.1 (FIG. 10D), RIM-BP2 (FIG. 10E), and Bassoon (FIG. 10F). Note that the levels of Munc13-1 (FIG. 10C), Ca_{V}2.1 (FIG. 10D), and RIM-BP2 (FIG. 10E) correlate well with levels of RIM (FIG. 10A). Furthermore, in contrast to rescue ELKS2αB which does not localize to cKO^{R+E} synapses (FIG. 1), rescue ELKS1aB is localized at least in part to synapses and recovers some synaptic Bassoon (FIG. 10F). This may be related to their distinct functions and differential localization, with ELKS2αB at the active zone and ELKS1αB broadly distributed throughout the nerve terminal (Nyitrai et al., 2020). n=30/3 each. Data are mean±SEM; **P<0.01, ***P<0.001 compared to cKO^{R+E}+RIM1αlow as determined by Brown-Forsythe ANOVA followed by Dunnett's T3 test (FIG. 10A, 10B, 10C and 10E), or by one-way ANOVA followed by Dunnett's tests (FIGS. 10D and 10F).

FIG. 11A-11H show Characterization of NMDAR-mediated synaptic transmission, related to FIG. 2. (FIGS. 11A-11B) Sample traces (FIG. 11A) and quantification (FIG. 11B) of NMDAR-mediated EPSCs evoked by focal electrical stimulation with 0.5 mM Ca²⁺ in the extracellular solution (instead of 1.5 mM used in all other experiments), n=18 cells/3 independent cultures per condition. (FIGS. 11C-11D) Sample traces (FIG. 11C) and quantification (FIG. 11D) of NMDAR-mediated paired pulse ratios induced with focal electrical stimulation in extracellular solution containing 0.5 mM Ca²⁺ (instead of 1.5 mM used in all other experiments). The relationship of paired pulse ratios between cKO^{R+E} and control^{R+E} was similar in 0.5 (FIG. 11D) or 1.5 mM (FIG. 2H) Ca²⁺, n=16/3 per condition. (FIGS. 11E-11F) Sample traces (FIG. 11E) and quantification (FIG. 11F) of NMDAR-mediated EPSCs evoked by focal electrical stimulation in 1.5 mM Ca²⁺ and in the presence of 20 μM of the low affinity NMDAR antagonist L-AP5 to prevent NMDAR saturation, n=16/3 per condition. (FIGS. 11G-11H) Sample traces (FIG. 11G) and quantification (FIG. 11H) of NMDAR-mediated paired pulse ratios induced with focal electrical stimulation in extracellular solution containing 1.5 mM Ca²⁺ and 20 μM L-AP5. Paired pulse ratios between cKO^{R+E} and control^{R+E} were similar with (FIG. 11H) or without (FIG. 2H) L-AP5, n=16/3 per condition. Overall, FIGS. 11A-11G indicate that NMDAR saturation does not confound the estimates of p in our standard conditions (1.5 mM Ca²⁺, no L-AP5). Data are mean±SEM; ***P<0.001 compared to control^{R+E} as determined by Mann-Whitney tests (FIGS. 11B and 11F) or two-way ANOVA followed by Dunnett's tests (FIGS. 11D and 11H).

FIGS. 12A-12H show RIM1 zinc finger re-expression restores the RRP at inhibitory cKO^{R+E} synapses, related to FIGS. 3 and 4. (FIGS. 12A-12B) Expression levels of rescue constructs in homogenates of cultured cKO^{R+E} neurons were assessed with anti-HA antibodies, and anti-β-actin antibodies were used as loading controls. Several RIM1-C₂A constructs were generated in addition to the constructs that are shown, but their expression could never be detected by Western blotting, and constructs were hence not further used for experimentation. (FIGS. 12C-12D) Sample traces (FIG. 12C) and quantification (FIG. 12D) of IPSCs evoked by focal electrical stimulation, control^{R+E} n=19 cells/5 independent cultures, cKO^{R+E} n=19/5, cKO^{R+E}+RIM1α n=19/5, cKO^{R+E}+RIM1-Zn n=18/5, and cKO^{R+E}+RIM1-ΔZn n=19/5. (FIGS. 12E-12F) Sample traces (FIG. 12E) and quantification (FIG. 12F) of IPSC triggered by hypertonic sucrose, the first 10 s of the IPSC were quantified to estimate the RRP, n=17/3 per condition. (FIGS. 12G-12H) Sample traces (FIG. 12G) and quantification (FIG. 12H) of IPSC paired pulse ratios, control^{R+E} n=17/5, cKO^{R+E} n=17/5, cKO^{R+E}+RIM1α n=18/5, cKO^{R+E}+RIM1-Zn n=17/5, and cKO^{R+E}+RIM1-ΔZn n=18/5. Data are mean±SEM; *P<0.05, **P<0.01, ***P<0.001 compared to cKO^{R+E} as determined by Kruskal-Wallis followed by Dunn's tests (FIGS. 12D and 12F) or two-way ANOVA followed by Dunnett's tests (FIG. 12H).

FIGS. 13A-13H shows the screening of fusion-proteins for restoring action potential-triggered release at cKO^{R+E} synapses, related to FIG. 5. (FIG. 13A) Schematic representation of candidate fusion-proteins of the RIM1 zinc finger domain to Ca_{V}β1b, Ca_{V}β3, Ca_{V}β4, Ca_{V}2.1 (N- or C-terminal fusions), ELKS2αB, Liprin-α3 or RIM1-PDZ. (FIGS. 13B-13C) Sample traces (FIG. 13B) and quantification of IPSC amplitudes (FIG. 13C) evoked by a focal stimulation, n=4-5 cells/1 culture per condition. (FIGS. 13D-13E) Sample traces (FIG. 13D) and quantification (FIG. 13E) of IPSC paired pulse ratios at 20 ms interstimulus intervals, n=4-5 cells/1 culture per condition. (FIGS. 13F-13G) Sample STED images (FIG. 13F) and quantification (FIG. 13G) of side-view synapses stained for Ca_{V}β4 (STED), PSD-95 (STED), and Synaptophysin (confocal) in wild type synapses, n=23 synapses/1 culture. (FIG. 13H) Expression levels of RIM1 and rescue constructs in homogenates of cultured control^{R+E} and cKO^{R+E} neurons were assessed with anti-RIM1 PDZ (top), anti-Ca_{V}β4 (middle) and anti-RIM1 zinc finger (bottom) antibodies, and anti-Synapsin antibodies were used as loading controls. Data shown as mean±SEM, no statistics were performed for (FIG. 13C) and (FIG. 13E) due to the limited number of observations. Based on the rescue activity of β4-Zn for evoked IPSC amplitudes and PPR, and the active zone-like localization of endogenous Ca_{V}β4, β4-Zn was chosen for full characterization.

FIGS. 14A-14G show binding of β4-Zn to Munc13 is essential for restoring release at inhibitory cKO^{R+E} synapses, related to FIGS. 7 and 8. (FIG. 14A) Expression levels of RIM1 and rescue constructs in homogenates of cultured control^{R+E} and cKO^{R+E} neurons were assessed with anti-RIM1 PDZ (top), anti-Ca_{V}β4 (middle) and anti-RIM1 zinc finger (bottom) antibodies, and anti-Synapsin antibodies were used as loading controls. (FIG. 14B-14C) Sample traces (FIG. 14B) and quantification (FIG. 14C) of IPSCs evoked by focal electrical stimulation, control^{R+E} n=18/3, cKO^{R+E} n=17/3, cKO^{R+E}+β4-Zn n=16/3, and cKO^{R+E}+β4-Zn β4-ZnK144/6E n=18/3. (FIGS. 14D-14E) Sample traces (FIG. 14D) and quantification (FIG. 14E) of IPSC triggered by hypertonic sucrose, the first 10 s of the IPSC were quantified to estimate the RRP, n=16/3 per condition. (FIGS. 14F-14G) Sample traces (F) and quantification (G) of IPSC paired pulse ratios, n=16/3 per condition. Data are mean±SEM; ***P<0.001 compared to cKO^{R+E} as determined by Kruskal-Wallis followed by Dunn's tests (FIGS. 14C and 14E) or by two-way ANOVA followed by Dunnett's tests (FIG. 14G).

### DETAILED DESCRIPTION

The technology described herein relates, in part, to enhancing or repairing synaptic function by facilitating secretion of neurotransmitters and hormones. The technology is directed to a fusion protein that can repair or enhance secretion. Another aspect of this technology is directed to a fusion protein for use in treating neurological disorder or secretory disorders, including endocrine diseases.

### Polypeptides

Various aspects described herein include a polypeptide comprising a first domain and a second domain, wherein the first domain comprises at least a zinc-finger domain (ZNF) of a Regulating Synaptic Membrane Exocytosis protein (RIM) and the second comprises a Ca_{V}β Ca²⁺ channel subunit Exemplary RIMS-ZNF domains and Ca2+ channel subunits are described herein below.

### Zinc-Finger Domain (ZNF Domain)

Various aspects herein relate to a nucleic acid sequence or polypeptide comprising a ZNF domain of a Regulating Synaptic Membrane Exocytosis protein (RIMS) protein. RIMS are multidomain proteins of the Ras superfamily of genes. RIM proteins contain a Rab binding domain (RabBD), a zinc-finger domain (ZNF), a PDZ domain, a C₂A domain, and a C₂B domain. ZNF domain. In vertebrates two RIM genes (RIMS1 and RIMS2) synthesize the five principal RIM isoforms from independent promoters (RIM1α, RIM1β, RIM2α, RIM2β, and RIM2γ). Two other RIM genes (RIMS3 and RIMS4) produce only γ-isoforms. These isoforms are further diversified by alternative splicing. RIM proteins regulate synaptic vesicle exocytosis and play a role in regulation of voltage-gated calcium channels during neurotransmitter and insulin release.

Methods and compositions described herein require a zinc finger from RIMS1. As used herein, the “Regulating Synaptic Membrane Exocytosis 1” refers to protein that is a RAS gene superfamily member that regulates synaptic vesicle exocytosis. The gene encoding this protein also plays a role in the regulation of voltage-gated calcium channels during neurotransmitter and insulin release. Mutations have suggested a role cognition and have been identified as the cause of cone-rod dystrophy type 7. Multiple transcript variants encoding different isoforms have been described for this gene. Sequences for RIMS1, also known as RIM; RIM1; CORD7; RAB3IP2, are known for a number of species, e.g., human RIMS1 (NCBI Gene ID: 22999) polypeptide (e.g., NCBI Ref Seq: NP_01161879.1) and mRNA (e.g., NCBI Ref Seq: NM_001168407.2). RIMS1 can refer to human RIMS1, including naturally occurring variants, molecules, and alleles thereof. The human nucleic sequence of SEQ ID NO: 3 comprises the nucleic sequence which encodes RIMS1. The human polypeptide sequence of SEQ ID NO: 1 comprises the polypeptide sequence of RIMS1.

Methods and compositions described herein require a zinc finger from RIMS2. As used herein, the “Regulating Synaptic Membrane Exocytosis 2” refers to a presynaptic protein that interacts with RAB3, a protein important for normal neurotransmitter release. The encoded protein can also bind several other synaptic proteins, including UNC-13 homolog B, ELKS/Rab6-interacting/CAST family member 1, and synaptotagmin 1. This protein is involved in synaptic membrane exocytosis. Polymorphisms in this gene have been associated with degenerative lumbar scoliosis. Sequences for RIMS2, also known as OBOE; RIM2; CRSDS; RAB3IP3, are known for a number of species, e.g., human RIMS1 (NCBI Gene ID: 9699) polypeptide (e.g., NCBI Ref Seq NP_001093587.1) and mRNA (e.g., NCBI Ref Seq NM_001100117.3). RIMS2 can refer to human RIMS2, including naturally occurring variants, molecules, and alleles thereof. The human nucleic sequence of SEQ ID NO: 4 comprises the nucleic sequence which encodes RIMS2. The human polypeptide sequence of SEQ ID NO: 2 comprises the polypeptide sequence of RIMS2.

Exemplary sequences for RIMS can be found in NCBI with Accession Numbers as listed: human RIMS1 (NP 055804.2); human RIMS2 (NP 001093587.1), mouse RIMS1 (XP 444500.2), mouse RIMS2 (NP 001243311.1) Rat RIMS1 (NP 001385524.1), Rat RIMS2 (NP 446397.1), Chimpanzee RIMS1 (XP 016811302.1), Chimpanzee RIMS2 (XP 009454035.1), Rhesus monkey RIMS1 (XP 014992172.2), Rhesus monkey RIMS 2 (XP 028706840.1), Chicken RIMS1 (XP 015140258.1), and Chicken RIMS2 (XP 040552327.1).

Exemplary sequences for human RIMS proteins can be found in NCBI with Accession Numbers as listed: human RIMS1 isoform 1 (NP 055804.2), human RIMS1 isoform 2 (NP 001161879.1), human RIMS1 isoform 3 (NP 001161880.1), human RIMS1 isoform 4 (NP 001161881.1), human RIMS2 isoform a (NP 001093587.1), human RIMS2 isoform b (NP 055492.3), humanRIMS2 isoform c (NP 001269810.1), and human RIMS2 isoform d (NP 001269811.1).

Exemplary sequences for human RIMS proteins can be found in NCBI with Accession Numbers as listed: human RIMS1 isoform 1 (NM 014989.7), human RIMS1 isoform 2 (NM 001168407.2), human RIMS1 isoform 3 (NM 001168408.2), human RIMS1 isoform 4 (NM 001168409.2), human RIMS2 isoform a (NM 001100117.3), human RIMS2 isoform b (NM 014677.5), human RIMS2 isoform c (NM 001282881.2), and human RIMS2 isoform d (NM 001282882.2).

In some embodiments, the ZNF domain is from a mammalian RIMS protein, such as a mouse or human RIMS. For example, the ZNF domain is from human RIMS1 or human RIMS2. In some preferred embodiments, the ZNF domain is from human RIMS1.

Accordingly, in some embodiments, the first domain of the fusion protein comprises a RIMS ZNF (i.e., RIMS1 or RIMS2) domain polypeptide, e.g., a RIMS ZNF domain that comprises an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to a wild-type sequence of said RIMS domain.

In one embodiment, the first domain of the fusion protein comprises a RIMS1 ZNF domain polypeptide, e.g., a RIMS ZNF domain that comprises an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to a wild-type sequence of said RIMS1 domain.

In one embodiment, the first domain of the fusion protein comprises a RIMS2 ZNF domain polypeptide, e.g., a RIMS ZNF domain that comprises an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to a wild-type sequence of said RIMS2 domain.

In some embodiments of any one of the aspects, the first domain of the fusion protein comprises an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to a ZNF domain of mammalian RIMS. For example, the first domain comprises an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to a human RIMS1 or RIMS2.

In some embodiments, the first domain comprises an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence selected from the SEQ ID NOs: 1, 2, 40, and 41.

SEQ ID NO: 1 describes the Human RIMS1 amino acid sequence (Isoform 1: NP_055804.2). In SEQ ID NO: 1 bold and underlined text denotes the ZNF domain.

(SEQ ID NO: 1)
mssavgprgp rpptvpppmq elpdlshlte eerniimavm drqkeeeeke eamlkcvvrd
makpaacktp rnaenqphqp sprlhqqfes ykeqvrkige earryqgehk ddaptcgich
ktkfadgcgh lcsycrtkfc arcggrvslr snnedkvvmw vcnlcrkqqe iltksgawff
gsgpqqtsqd gtlsdtatga gsevprekka rlqersrsqt plstaaassq daappsappd
rskgaepsqq algpeqkqas srsrseppre rkktpglseq ngkgalkser krvpktsaqp
vegaveerer kerresrrle kgrsqdypdt pekrdegkaa deekqrkeed yqtryrsdpn
larypvkppp eeqqmrmhar vsrarherrh sdvalprtea gaalpegkag krapaaaras
ppdspraysa ertaetrapg akqltnhspp aprhgpvpae apelkagepl rkqsrldpss
avlmrkakre kvetmlrnds lssdqsesvr psppkphrsk rggkkrqmsv ssseeegvst
peytscedve lesesvsekg dldyywldpa twhsretspi sshpvtwqps kegdrligrv
ilnkrttmpk dsgallglkv vggkmtdlgr lgafitkvkk gsladvvghl ragdevlewn
gkplpgatne evyniilesk sepqveiivs rpigdiprip esshppless sssfesqkme
rpsisvispt spgalkdapq vlpgqlsvkl wydkvghqli vnvlqatdlp arvdgrprnp
yvkmyflpdr sdkskrrtkt vkkilepkwn qtfvyshvhr rdfrermlei tvwdqprvqe
eeseflgeil ieletalldd ephwyklqth desslplpqp spfmprrhih gessskklqr
sqrisdsdis dyevddgigv vppvgyrssa resksttltv peqqrtthhr srsvsphrgn
dqgkprsrlp nvplqrslde ihptrrsrsp trhhdasrsp vdhrtrdvds qylseqdsel
lmlprakrgr saeclhttrh lvrhyktlpp kmpllqsssh wniyssilpa htktksvtrq
dislhhecfn stvlrftdei lvselqpfld rarsastncl rpdtslhspe rergrwspsl
drrrppspri qiqhaspend rhsrkserss iqkqtrkgta sdaervlptc lsrrghaapr
atdqpvirgk hparsrsseh ssirtlcsmh hlvpggsapp splltrmhrq rsptqsppad
tsfssrrgrq lpqvpvrsgs ieqaslvvee rtrqmkmkvh rfkqttgsgs sqeldreqys
kynihkdqyr scdnvsakss dsdvsdvsai srtssasrls stsfmseqse rprgrissft
pkmqgrrmgt sgrsimksts vsgemytleh ndgsqsdtav gtvgaggkkr rsslsakvva
ivsrrsrsts qlsqtesghk klkstiqrst etgmaaemrk mvrqpsrest dgsinsysse
gnlifpgvrl gadsqfsdfl dglgpaqlvg rqtlatpamg diqigmedkk gqlevevira
rsltqkpgsk stpapyvkvy llengaciak kktriarktl dplyqqslvf despqgkvlq
vivwgdygrm dhkcfmgvaq illeeldlss mvigwyklfp psslvdptlt pltrrasqss
lesstgppci rs

SEQ ID NO: 40 describes the Rat RIMS1 amino acid sequence (Isoform 1: NP_001385524.1).

MSSAVGPRGPRPPTVPPPMQELPDLSHLTEEERNIIMAVMDRQKEEEEKE
EAMLKCVVRDMAKPAACKTPRNAESQPHQPPLNIFRCVCVPRKPSSEEGG
PERDWRLHQQFESYKEQVRKIGEEARRYQGEHKDDAPTCGICHKTKFADG
CGHLCSYCRTKFCARCGGRVSLRSNNEDKVVMWVCNLCRKQQEILTKSGA
WFFGSGPQQPSQDGTLSDTATGAGSEVPREKKARLQERSRSQTPLSTAAV
SSQDTATPGAPLDRNKGAEPSQQALGPEQKQASRSRSEPPRERKKAPGLS
EQNGKGGQKSERKRVPKSVVQPGEGIADERERKERRETRRLEKGRSQDYS
DRPEKRDNGRVAEDQKQRKEEEYQTRYRSDPNLARYPVKAPPEEQQMRMH
ARVSRARHERRHSDVALPHTEAAAAAPAEATAGKRAPATARVSPPESPRA
RAPAAQPPTEHGPPPPRPAPGPAEPPEPRVPEPLRKQGRLDPGSAVLLRK
AKREKAESMLRNDSLSSDQSESVRPSPPKPHRPKRGGKRRQMSVSSSEEE
GVSTPEYTSCEDVELESESVSEKGDLDYYWLDPATWHSRETSPISSHPVT
WQPSKEGDRLIGRVILNKRTTMPKESGALLGLKVVGGKMTDLGRLGAFIT
KVKKGSLADVVGHLRAGDEVLEWNGKPLPGATNEEVYNIILESKSEPQVE
IIVSRPIGDIPRIPESSHPPLESSSSSFESQKMERPSISVISPTSPGALK
DAPQVLPGQLSVKLWYDKVGHQLIVNVLQATDLPPRVDGRPRNPYVKMYF
LPDRSDKSKRRTKTVKKLLEPKWNQTFVYSHVHRRDFRERMLEITVWDQP
RVQDEESEFLGEILIELETALLDDEPHWYKLQTHDESSLPLPQPSPFMPR
RHIHGESSSKKLQRSQRISDSDISDYEVDDGIGVVPPVGYRASARESKAT
TLTVPEQQRTTHHRSRSVSPHRGDDQGRPRSRLPNVPLQRSLDEIHPTRR
SRSPTRHHDASRSPADHRSRHVESQYSSEPDSELLMLPRAKRGRSAESLH
MTSELQPSLDRARSASTNCLRPDTSLHSPERERHSRKSERCSIQKQSRKG
TASDADRVLPPCLSRRGYATPRATDQPVVRGKHPTRSRSSEHSSVRTLCS
MHHLAPGGSAPPSPLLTRTHRQGSPTQSPPADTSFGSRRGRQLPQVPVRS
GSIEQASLVVEERTRQMKMKVHRFKQTTGSGSSQELDHEQYSKYNIHKDQ
YRSCDNASAKSSDSDVSDVSAISRASSTSRLSSTSFMSEQSERPRGRISS
FTPKMQGRRMGTSGRAIIKSTSVSGEIYTLERNDGSQSDTAVGTVGAGGK
KRRSSLSAKVVAIVSRRSRSTSQLSQTESGHKKLKSTIQRSTETGMAAEM
RKMVRQPSRESTDGSINSYSSEGNLIFPGVRVGPDSQFSDFLDGLGPAQL
VGRQTLATPAMGDIQIGMEDKKGQLEVEVIRARSLTQKPGSKSTPAPYVK
VYLLENGACIAKKKTRIARKTLDPLYQQSLVFDESPQGKVLQVIVWGDYG
RMDHKCFMGVAQILLEELDLSSMVIGWYKLFPPSSLVDPTLAPLTRRASQ
SSLESSSGPPCIRS

SEQ ID NO: 41 describes a modified RIMS1 amino acid sequence comprising the ZNF and RabBD.

MSSAVGPRGPRPPTVPPPMQELPDLSHLTEEERNIIMAVMDRQKEEEEKE
EAMLKCVVRDMAKPAACKTPRNAESQPHQPPLRLHQQFESYKEQVRKIGE
EARRYQGEHKDDAPTCGICHKTKFADGCGHLCSYCRTKFCARCGGRVSLR
SNNEDKVVMWVCNLCRKQQEILTKSGAWFFGSGPQQPSQD

SEQ ID NO: 2 describes the Human RIMS2 amino acid sequence (Isoform a: NP_001093587.1). In SEQ ID NO: 2, bold and underlined text denotes the ZNF domain.

(SEQ ID NO: 2)
msapvgprgr lapipaasqp plqpempdls hlteeerkii lavmdrqkke eekeqsvlkk
lhqqfemyke qvkkmgeesq qqqeqkgdap tcgichktkf adgcghncsy cqtkfcarcg
grvslrsnkv mwvcnlcrkq qeiltksgaw fynsgsntpq qpdqkvlrgl rneeapqekk
pklheqtqfq gpsgdlsvpa veksrshglt rqhsikngsg vkhhiasdia sdrkrspsvs
rdqnrrydqr eereeysqya tsdtamprsp sdyadrrsqh epqfyedsdh lsyrdsnrrs
hrhskeyivd dedvesrdey erqrreeeyq sryrsdpnla rypvkpqpye eqmrihaevs
rarherrhsd vslanadled srismlrmdr psrqrsiser raamenqrsy smertreaqg
pssyaqrttn hspptprrsp lpidrpdlrr tdslrkqhhl dpssavrktk rekmetmlrn
dslssdqses vrppppkphk skkggkmrqi slssseeela stpeytscdd veiesesvse
kgdmdynwld htswhsseas pmslhpvtwq pskdgdrlig rillnkrlkd gsvprdsgam
lglkvvggkm tesgrlcafi tkvkkgslad tvghlrpgde vlewngrllq gatfeevyni
ileskpepqv elvvsrpigd ipripdstha qlesssssfe sqkmdrpsis vtspmspgml
rdvpqflsgq lssqslsrrt tpfvprvqik lwfdkvghql ivtilgakdl psredgrprn
pyvkiyflpd rsdknkrrtk tvkktlepkw nqtfiyspvh rrefrermle itlwdqarvr
eeeseflgei lieletalld dephwyklqt hdvsslplph pspymprrql hgesptrrlq
rskrisdsev sdydcddgig vvsdyrhdgr dlqsstlsvp eqvmssnhcs psgsphrvdv
igrtrswsps vpppqsrnve qglrgtrtmt ghyntisrmd rhrvmddhys pdrdrdceaa
drqpyhrsrs teqrpllert ttrsrsterp dtnlmrsmps Imtgrsapps palsrshprt
gsvqtspsst pvagrrgrql pqlppkgtld rkaggkklrs tvqrstetgl avemrnwmtr
qasrestdgs mnsyssegnl ifpgvrlasd sqfsdfldgl gpaqlvgrqt latpamgdiq
vgmmdkkgql eveiirargl vvkpgsktlp apyvkvylld ngvciakkkt kvarktlepl
yqqllsfees pqgkvlqiiv wgdygrmdhk sfmgvaqill delelsnmvi gwfklfppss
lvdptlaplt rrasqssles stgpsysrs

In some embodiments, the first domain of the fusion protein comprises an amino acid sequence having at least 90%, 91%, 92%, 93%, 94%, 95%, 96%, 97%, 98% or 99% identity to an amino acid sequence selected from the group consisting of SEQ ID NOs: 1, 2, 40, and 41. In some embodiments, the first domain comprises an amino acid sequence having at least 95%, 96%, 97%, 98% or 99% identity to an amino acid sequence selected from the group consisting of SEQ ID NOs: 1, 2, 40, and 41. In some embodiments, the first domain comprises an amino acid sequence having at least 97%, 98% or 99% identity to an amino acid sequence selected from the group consisting of SEQ ID NOs: 1, 2, 40, and 41. In some embodiments, the first domain comprises an amino acid sequence having 100% identity to an amino acid sequence selected from the group consisting of SEQ ID NO: 1, 2, 40, and 41.

In some preferred embodiments, the first domain comprises the amino acid sequence of SEQ ID NO: 1.

In some preferred embodiments, the first domain comprises the amino acid sequence of SEQ ID NO: 41.

In some embodiments, the first domain of the fusion protein comprises an amino acid sequence having at least 90%, 91%, 92%, 93%, 94%, 95%, 96%, 97%, 98% or 99% identity to an amino acid sequence selected from the group consisting of the ZNF domain of SEQ ID NOs: 1, 2, 40, and 41. In some embodiments, the first domain comprises an amino acid sequence having at least 95%, 96%, 97%, 98% or 99% identity to an amino acid sequence selected from the group consisting of the ZNF domain of SEQ ID NOs: 1, 2, 40, and 41. In some embodiments, the first domain comprises an amino acid sequence having at least 97%, 98% or 99% identity to an amino acid sequence selected from the group consisting of the ZNF domain of SEQ ID NOs: 1, 2, 40, and 41. In some embodiments, the first domain comprises an amino acid sequence having 100% identity to an amino acid sequence selected from the group consisting of the ZNF domain of SEQ ID NOs: 1, 2, 40, and 41.

In some embodiments, the first domain of the fusion protein is encoded by a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a wild-type sequence encoding RIMS (i.e., RIMS1 or RIMS2).

In some embodiments of any one of the aspects, the first domain of the fusion protein is encoded by a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a ZNF domain of RIMS (i.e., RIMS1 or RIMS2). For example, the first domain is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98% or 99% identity to a human RIMS1 or RIMS2 nucleic acid sequence.

In some embodiments, the first domain comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to the sequence of SEQ ID NOs: 3, 4, 38, and 39.

SEQ ID NO: 3 describes the Human RIMS1 nucleic acid sequence (NM_014989.7).

(SEQ ID NO: 3)
aaattggagg cagcagagcc tgggagcagc ctccacggcg gcagcggccg ccccagtccc
agccccagcc ccagccccag ccccgccccc gccccgcgcc ccgccggaga cgcccggatc
ggcggagcct ggcgcgagcc ctcgccccct cgcctctccc gccgcgcctc cgcctgcccg
cccccgccgg ccgaggctgg gctgcgggag gcggccgggc ggccccgagc ttcgctaggg
cgaccaaaac aaaggcagca tccggggctg ggtggatgca aacaaccatg aaagactggg
ttctcgctct ccccggctct gctgctgctg ctgctgccgc cgccgccgct gctcctcctc
ctgccgccgc cgctagggct ccgctgtgag ggggaagcag gggcgcagct gctgggcgtg
catccgaaag gtgagagcca gagagcgagc agagggggcg ggcaggccac gaaaatgtcc
tcggccgtgg ggccccgcgg tcctcgccca cccacggtgc ctccccccat gcaagagctg
cccgacctga gccacctgac cgaagaggag aggaacatta tcatggcagt gatggaccgg
cagaaggaag aggaggaaaa agaagaagcc atgctcaagt gtgttgtcag ggacatggcg
aagcctgctg cctgcaaaac accaagaaat gctgaaaacc agccccacca accttcaccg
agattgcatc aacagtttga aagctataag gaacaagtga gaaaaatagg ggaagaagcg
cggcgttacc agggcgagca caaagacgat gctccgactt gtggaatctg tcataaaaca
aagtttgctg atgggtgcgg tcatctctgc tcctattgtc gcactaagtt ctgtgcgcgc
tgcggaggcc gcgtgtctct acggtcaaac aacgaggaca aagtggttat gtgggtatgc
aatttatgtc gaaagcaaca agaaatctta accaaatctg gggcatggtt ctttggaagt
ggccctcagc agacaagtca ggatggaacc ctgagtgata cagctacagg tgctggctct
gaggtaccaa gagaaaagaa agcacgactc caagagcgat cgcggtctca gacaccccta
agcacagcag ctgcctcctc ccaggatgct gctcctccca gcgcaccacc agacaggagc
aaaggggctg agccctcgca gcaagccttg gggcctgaac agaagcaggc ttcatccagg
tctagaagtg aacctcctag agagagaaag aagaccccag ggctttccga gcagaatggc
aaaggagccc tgaagagcga gcggaaacgc gtgccaaaga cctcagcgca gcccgtggag
ggggccgtcg aagaacggga gcgcaaagaa aggcgggaaa gccgaaggct tgagaaaggg
cgatcacagg attacccaga cacgccggaa aaacgggatg agggcaaagc ggcggatgag
gaaaagcaaa gaaaagagga ggattatcag accaggtacc gcagcgaccc gaacctagct
cggtacccgg tgaaaccgcc gcctgaggag cagcagatgc gcatgcacgc ccgggtgtcc
cgcgccaggc acgagcggcg ccacagcgac gtggcgctcc cgcgcaccga ggcgggcgcg
gcgctgccgg agggcaaggc cggcaaacgc gcgccggcgg cagccagggc ctcgccgccg
gactcgccgc gggcttactc ggctgagaga actgcggaga ccagggcgcc gggcgccaag
cagctaacga accacagccc gccggcgccc agacatgggc cggttcccgc agaagccccg
gagctcaaag cccaggagcc cctcaggaag cagagccgcc tggaccccag ctcggcggtc
ctcatgcgga aggccaagcg cgagaaggtg gagaccatgc tgcggaacga ctctttgagc
tcagaccagt ccgagtcggt gcggccgtcc ccgcccaagc cgcaccggtc caagagaggc
ggcaagaagc ggcagatgtc ggtgagcagc tctgaggagg agggcgtgtc gacgcccgag
tacaccagct gcgaggacgt ggagctggag agcgagagcg tcagcgagaa aggtgatttg
gattattact ggttggatcc tgccacgtgg cacagccggg agacatcacc tattagttcg
catcctgtaa cgtggcaacc atctaaagag ggggaccgat taattggacg tgttattctt
aacaagagaa caaccatgcc caaagactca ggtgcattgc tgggtctgaa agttgttgga
ggaaaaatga ctgacttagg acgacttggt gctttcatca ccaaagtaaa gaagggtagc
ctagcagatg tagttggaca cctaagagca ggggatgaag ttctagaatg gaatggtaaa
cccctgccgg gagctacaaa tgaagaagtt tacaacatta ttttagaatc aaaatcagaa
cctcaagttg aaattattgt ttcaaggcct attggtgaca ttccccggat tcctgagagc
tcccaccctc cactggagtc cagttcaagt tcctttgaat ctcagaagat ggaaaggcct
tccatttctg ttatttctcc aacaagtcct ggagctctaa aagatgcccc acaagtctta
ccagggcaac tttctgtgaa gttgtggtat gataaagtgg gacaccagct gattgtaaat
gttctgcaag caacagatct acctgctaga gtagatggac gtcctcgaaa tccctatgta
aaaatgtatt ttcttccaga tagaagtgat aaaagtaaaa ggaggaccaa aacagtaaag
aaaatactag aaccaaaatg gaatcaaact tttgtctatt cacatgtaca tcgtagagat
tttagagaac gaatgttaga aataactgtg tgggaccaac caagagtgca agaagaagaa
agtgaatttc ttggagagat cctcatagaa ttggagacag cgcttttaga tgatgaaccg
cattggtata aacttcagac acatgatgag tcttcactac ctctgcctca gccatcacct
ttcatgccaa ggcgacatat tcatggagaa agctctagca aaaagctaca aagatctcag
cgaatcagtg atagtgacat ctcagattat gaggttgatg atggtattgg cgtagttcct
ccagtaggct ataggtctag tgctagagaa agtaaatcta caacattaac tgtgccagaa
cagcaaagaa caactcatca ccgctcacgt tcagtatctc ctcatcgcgg caatgatcag
ggaaagccgc gttcacgttt accaaatgtg ccattacaga ggagtttaga tgaaattcat
ccaacaagaa ggtcacgttc tccaaccaga caccatgatg cctcccgaag tccagttgat
catagaacca gagatgtgga tagtcagtat ttatcagaac aagacagtga gcttcttatg
ctgcccagag caaaacgagg acgaagtgca gaatgcctac atactaccag acatcttgtt
aggcactata aaacattacc tcccaagatg cctttattac agagcagttc tcactggaat
atttacagct caattctgcc tgcacatact aagaccaaat cagtgactag acaggacatt
tcccttcatc atgaatgctt taactcaaca gtattgagat ttactgatga aatactggtt
agtgaactgc agccctttct tgacagggct aggagtgcta gtaccaactg cttgagacca
gatactagtt tgcattcacc agaacgagaa aggggtagat ggtccccctc cctagatagg
agacgacctc ctagtcccag gattcaaatc cagcatgcgt ctccggagaa tgacaggcac
tccagaaagt ctgaaagatc tagcatccaa aaacagacta ggaaaggcac tgcctctgat
gcagaaaggg ttctcccaac atgtctttct agaaggggac acgcagcccc aagagcaact
gatcagccag tcattagggg aaaacatcct gctcgctcaa ggtcgagtga gcactctagt
atcagaacac tgtgttctat gcaccacctt gtccctggag ggtcggcgcc accttctccg
cttctgacaa gaatgcaccg acagagaagt ccaacacaat ctcctccagc agacacatcg
ttcagcagtc gcaggggaag acagctccca caagtgccag tgagaagcgg cagtatagaa
caagcaagct tagtagtgga ggagcgaaca agacagatga aaatgaaagt gcatcgattt
aagcagacaa cagggtctgg ttctagtcaa gaacttgatc gcgagcaata ttccaagtat
aacatacata aagatcagta cagaagctgt gataacgtct ctgccaaatc atcagatagt
gatgtcagtg atgtttccgc catttcccga accagcagtg cctcacgcct cagcagcaca
agctttatgt cagagcaatc tgagcgcccc aggggtagaa tcagttcatt tacccccaaa
atgcaaggca gacggatggg gacttcagga agatccatca tgaagagcac cagtgtcagt
ggagagatgt acacactgga gcataatgac ggcagccagt cagacacagc tgtgggtaca
gttggagcag gtggaaagaa acggagatcc agccttagtg ccaaagtggt tgccatagtg
tctcgaagga gtagaagcac atcccagctt agtcaaacag agtcgggcca caaaaagtta
aaaagtacca tccagagaag cacagaaaca ggcatggcag ctgaaatgag aaagatggta
aggcagccga gccgagagtc tactgatggc agcatcaaca gttacagctc tgagggcaat
ttaatatttc ctggagtgcg actgggagct gacagtcaat tcagtgattt tcttgatgga
ttgggaccag cccagcttgt tggccgccaa acccttgcca cccctgcaat gggtgatata
caaataggaa tggaggacaa aaagggccaa ttagaagtgg aagtcattag agcacgaagc
ctcacacaaa agcctggttc caaatctaca cctgctccat atgtcaaagt atatcttttg
gaaaatgggg cctgtatagc caagaagaag acaagaattg cacgaaaaac ccttgatcct
ttgtatcagc agtctctggt ttttgatgaa agtccacagg gtaaagttct tcaggtgatt
gtctggggag actatggcag aatggaccac aaatgcttta tgggtgtggc tcagatcttg
ttggaagaac tcgacctgtc cagcatggtg atcggatggt acaaattgtt cccaccgtcc
tcactggtgg atcccacact cactcccctc acccggcggg cttcccagtc atctctggaa
agttcaactg ggcctccctg tattcgatca tagtgaactc ataccagagt cattccaata
aaactctact tttcaggata ataatctgaa ccagatattt catgatcgaa agcattgttg
gagacagaca atcaacttgt gttttgcctg tagtagtttt tcaataatat gtcccaattg
ttatttaaaa catggcttca tatgacagaa caaggcaatc tatcaaattt acaggaagaa
tcaacatgct ggtgagagtc actgatgctt ctaacaaata gaaaaagagg aaactttaaa
tccacgcata cacgtacaca cacacatgca cacacacaca caccaaattg aacaaactgg
aaactctcac tctgtgaaaa gttgtattct acacatttct gcacagacca caagcagtgt
tatttccctg atgtttgagt tgttttgttc ttttgtgtgt tttgtttatt tgtgtgttgt
ttgttggttt tcgtttcttg agtttttgtt ttgttttcac cacatctgtt atttccacta
gttttttttt ggctgtgtca tgacaggcct catgatgcta acagagattc ttcccttctt
ttttccaaaa gcaccaaaaa aaagggctga agtagtctct gtagcatctc cccaaagtgt
taaacagcta cataaggtga agccaaggaa gggtttcact agctttcatt ttataattat
ttaaagtctt atgtgtggat acccaagaga caaaaagtta tcttccaaaa taaataatca
cagaagtact tttttaatga agagcctatc tgtttgcatt ctagaaaatt aatttatggt
ttccattgtt ttgcatggaa gacttttaaa gaagtcaatc tgcaactaat gctggggact
aaaactaact gcataattgt actttgaaaa cactccttgt aatgcctttt tcttcctgat
attaaaaaaa tgctcatgtg tataaaccat actttgactt cccaaaaatg gtgactagga
atggatttga gcctaagaca ctttgagcta tgcccaggct tctcagccat acctcagtgt
agacacatcc atctgccctg gaaagagcat ggtatgagct gttcaatatt ccactggaaa
ttccagttga aatattctgc actaaactat tgtttttatt gaattttagt ttacatttct
ttgagattga tgcaagcaca aagcttgatt ttttaatgca aagtatctta ctttttgggg
ggaaaataaa caaaatgaac tttcaatttg cattttcatt tagtttctct tggccttgaa
tttcccttgt gacattctgt acatgtgcta caaactgcag tctctgcagg tgcattgata
tgttctccct ccttttatga gtcactccac ttttgtgatt acacaaatag agcagcatga
cttggaactg ttcagagctg catgcacatt ttgtaaaaaa aaaaaatttt aaaaagaaaa
gaaacaaaag gttatttaat aatgtatgtt agttccacat aggccagctt gtatgttgca
tgtacttgta cagtttgctt gttaattact ataccgaaat aaccaagaaa tctaagccat
ccacttttgt tatagacatt ttgcaggttt tagccagact cagtctctgg gtctgtggtg
aaatgtctat agatggactt tattttttac cctccggaaa acgtgatgta gtacggacca
aattccttct aataaatatt ttggtgtaga ttcctactgt ggggctgtaa cacatgtaga
cactgtgtac acactaggtt ttaattcata gacatactgc ctgcaccaag tgaattattt
attattattt acacatgcca gaattatctg cccatcattc cacagggcac agattgacca
ctgctcacca tgctgagtag gaggaactga agcattggtg cacttctact agattccgtt
ctgtcttagt ggccaacaat caactgatta taattgggta gtatctttct attttgacca
cttgtcttaa cactcccctg tgcttttaaa tgaaattcca attcatgtat gtaaacatgt
ttaataaaat ttacttttca tgtcagtcag tagccatatg tgttctatgt cttgccagat
ttccattgtg ggttctatgg tgcaaaatgg gagccaggcc atgggaaggg gaggctgggg
tttgttgtct gctgcgggca tttgacttga cttgtcactg cactatatta tgtcttaatc
gatggcccaa agggtacttt gcatcacata caagttttca cttcgtcagc ctgtctaagt
gagcacatga ctatacttct gaaaagtggc attattaaaa ataataaatc tcagtaagaa
tcaaatggaa ggttgttttc tgctgctgtt tgaatgtgct aataaaagag acaactctat
tgtc

SEQ ID NO: 38 describes the Rat RIMS1 nucleic acid sequence (NM_001398595.1)

atgtcctcggccgtggggccccgaggtcctcgcccacccacggtgcctccccctatgcaagaactgcccg
acctgagccacctgaccgaggaggagaggaacattatcatggcagtgatggaccggcagaaggaagagga
ggaaaaagaagaggccatgctcaagtgtgttgtcagggacatggcgaagcctgctgcctgcaaaacacca
agaaatgctgaaagccagccccatcaaccaccactgaacattttcagatgtgtctgtgttcccagaaagc
caagcagcgaagagggaggcccagaaagagactggagattgcatcaacagtttgaaagctacaaggagca
agtgagaaaaatcggagaggaagcgaggcgttaccagggcgagcacaaggatgatgccccgacgtgtgga
atctgtcataagacaaagtttgctgatggatgtggccatctctgctcctattgtcgcaccaagttctgtg
cacgctgcggaggccgtgtgtctctgcgatcgaacaatgaggacaaagtggttatgtgggtatgcaattt
atgtcgaaagcaacaagaaatcttaacgaaatctggagcgtggttctttggaagtggccctcagcagcct
agtcaagatgggactctgagtgacacggccacaggtgctggatctgaggtgccaagagaaaagaaagcaa
ggctccaagagcgatcaaggtctcagacgcccttgagtacagcagctgtctcttcccaagacactgctac
ccccggtgcaccgttggacaggaacaaaggggctgagccctcacagcaagccttgggtcctgaacagaag
caggcatcaagatcaagaagcgagccaccgagggaaaggaagaaggctccagggctttcagagcagaatg
gcaagggaggccagaagagcgagcgcaaacgtgtccccaagtctgtggtgcaacccggggaagggatcgc
ggatgagagggagaggaaagagaggcgggaaacccgcaggttggagaaagggcgctcccaggactactca
gaccggcctgagaaacgcgacaatggcagggtggcggaagaccagaagcagaggaaggaggaggagtacc
agactaggtaccgcagcgaccctaacctggctcgctacccggtgaaggcgccgccagaggagcagcagat
gcgcatgcacgcccgggtgtcccgagcgaggcacgagcggcgccacagcgacgtggcgctcccgcacacc
gaggcagctgccgccgcgccggctgaggccacggcgggcaagcgcgcgccggccaccgccagggtctctc
ccccggagtccccgcgcgcacgcgcgcccgccgcccagcctcccaccgagcacgggccaccgccgccgcg
gccagccccgggtcccgcagagccacccgagccgcgcgtccccgagccgctccgtaagcagggccgcctg
gacccgggctcggccgtgcttctgcgcaaggccaagcgcgagaaggcggagagcatgctgcggaacgact
cgctgagctccgatcagtccgagtccgtgcggccatccccgcccaagcctcaccggcccaagcggggagg
caagagacgtcagatgtcggtgagcagctcggaggaggagggcgtgtccacaccggagtacacgagctgc
gaggacgtggagctggagagcgagagcgtgagcgagaaaggtgacttggattactactggttggatcccg
ccacgtggcacagcagggaaacgtcgcctatcagttcgcatcctgtaacgtggcagccgtctaaagaggg
agatcgactaatcggccgtgttattcttaacaaaagaacaaccatgcccaaagaatcaggtgcattattg
ggtctgaaggtggttggaggaaaaatgacggacttagggcgccttggtgctttcatcaccaaagtaaaga
agggcagcctggcagacgtcgtcggacacctaagagcaggggacgaagtcctagagtggaatggtaaacc
cctgccgggagcaacaaacgaagaagtttacaacattatcttagaatcaaaatcagaacctcaagttgag
attattgtttcaaggcctattggtgacatccccaggatccctgagagttcccatcctcccctggagtcca
gttcaagttcctttgaatctcagaaaatggaaaggccttctatttctgttatttctccaaccagccctgg
agctctgaaagatgccccacaagtcttaccagggcaactctcagtgaagctatggtatgataaagtgggg
caccagctgattgtaaatgttctacaagcaacagatctaccccctagagtagatggccgtcccaggaatc
cctatgtaaaaatgtattttcttccagatagaagcgacaaaagtaaaaggagaaccaaaacagtaaagaa
acttctagagccaaaatggaaccagacatttgtctactcacacgtacatcgtagagattttcgagagcga
atgttagagattaccgtgtgggaccagccgagagtacaggacgaagagagtgaatttcttggagagatcc
tcatagagttggaaacagcgcttttagatgatgagccccattggtataaactccagacacatgacgaatc
ttcactacctctgcctcagccatcaccgttcatgcccaggcggcatattcatggagagagctccagcaaa
aagctacaaagatctcagcgaatcagtgatagtgacatctcagattatgaggttgatgatggtattggag
tagtgcctccagtgggttatagagctagtgctagagagagtaaagcaaccacgttaacagtgccagagca
acaaagaactacacatcaccgctcacgttccgtgtctcctcatcgcggcgatgatcagggaaggcctcgt
tcacgtttaccaaatgtgccattacagaggagcttagatgaaattcatccaacacgaaggtcacgttctc
caacccgacaccatgatgcctcccgaagcccggccgatcacagatccagacatgtggaaagtcaatattc
gtcagagccagacagtgagcttctcatgctgcccagagcaaaacgaggacgaagtgcagaaagcctacac
atgaccagtgaactgcagccctctcttgacagggctaggagtgctagtaccaactgcttgagaccagata
ctagtttgcattcaccagaacgagaaaggcactccagaaagtctgaaagatgtagcatccaaaaacagtc
taggaaaggcacagcctctgatgcagacagggttctcccaccatgcctttctagaaggggatacgcaacc
ccaagagcaaccgatcaaccggtcgttaggggaaagcatcccactcgttcacggtcgagcgagcactcta
gtgtcagaaccctgtgttctatgcaccaccttgcccccggagggtcggcgccaccttctccacttctgac
aagaacgcaccgacaaggaagcccaacccagtctcctccagcagacacatccttcggcagtcgccgtgga
agacagctcccacaggtgccagttcgaagcggcagtatagaacaagcaagcttagtagtggaggagcgaa
cgagacagatgaaaatgaaagttcaccgatttaagcagacaacagggtctgggtctagtcaagaacttga
ccacgagcaatactccaagtacaacatacataaagatcagtacagaagctgtgataacgcgtctgccaag
tcttcagatagtgatgtcagtgatgtgtccgccatttccagagccagcagtacctcacgcctcagcagca
caagctttatgtcagagcagtctgagcgccccaggggtaggatcagttcatttacccccaaaatgcaagg
cagacggatggggacttcaggaagagccatcatcaagagcaccagtgtaagtggagagatatatacactg
gaacgtaatgacggtagccagtcggacacggccgtgggtaccgtcggagccggtggaaagaaacgaagat
ccagcctgagcgccaaagtggtagccattgtgtctcgaagaagcaggagcacgtcacagctcagccagac
agagtcgggccacaagaagttgaaaagcaccatccagaggagtacggaaacaggaatggcagctgaaatg
cggaagatggtgagacagccgagccgggagtccacggatggcagcatcaacagttatagctcggaaggaa
acttgatatttcctggagttcgagtaggacccgacagtcagttcagtgatttccttgatgggttgggacc
agcgcagctcgttggccgtcagacgctcgccaccccggccatgggcgatatccaaatcgggatggaggat
aagaagggtcagttggaggttgaggttatcagagcccggagccttacacaaaaacctggttccaaatcta
cacccgctccctatgtgaaagtatatcttttggaaaatggagcctgtattgccaaaaagaagacaagaat
tgcacggaaaactctcgatcctttgtatcagcagtccctggtttttgatgaaagtccacagggtaaagtt
cttcaggtgattgtctggggtgactatggaagaatggaccacaaatgctttatgggtgtggctcaaatct
tgttggaagaacttgatctatccagcatggtgattggatggtataaattgttccctccgtcctcactggt
ggatcccactctcgctcccctgacccgccgggcttcccaatcatctctggaaagttcgtccgggcctccc
tgcatccggtca

SEQ ID NO: 39 describes a modified Rat RIMS1 nucleic acid sequence comprising the ZNF and flanking regions.

atgtcctcggccgtggggccccgaggtcctcgcccacccacggtgcctcc
ccctatgcaagaactgcccgacctgagccacctgaccgaggaggagagga
acattatcatggcagtgatggaccggcagaaggaagaggaggaaaaagaa
gaggccatgctcaagtgtgttgtcagggacatggcgaagcctgctgcctg
caaaacaccaagaaatgctgaaagccagccccatcaaccaccactgagat
tgcatcaacagtttgaaagctacaaggagcaagtgagaaaaatcggagag
gaagcgaggcgttaccagggcgagcacaaggatgatgccccgacgtgtgg
aatctgtcataagacaaagtttgctgatggatgtggccatctctgctcct
attgtcgcaccaagttctgtgcacgctgcggaggccgtgtgtctctgcga
tcgaacaatgaggacaaagtggttatgtgggtatgcaatttatgtcgaaa
gcaacaagaaatcttaacgaaatctggagcgtggttctttggaagtggcc
ctcagcagcctagtcaagat

SEQ ID NO: 4 describes the Human RIMS2 nucleic acid sequence (NM_001100117.3).

(SEQ ID NO: 4)
agttcccctt tcccttgaac cgctcacttc acagcccttc gcccccggga agaagaaaca
tttcccgaag cgcactcctc agccctcctt ccccacgcgc tcgccctccc ctccccctgc
ttttcttggg ggaggggggc tgtcgccttg gattgaaggc cattgatttg tatgtatttg
tcccagcgct ggaggctgcc ccagccgccg cgccggtgcc gccgctgcca gtggagttgc
ctccccgctt ccctagggtg gttcggctcc accaaacatg tcggctcctg tcgggccccg
gggccgcctg gctcccatcc cggcggcctc tcagccgcct ctgcagcccg agatgcctga
cctcagccac ctcacggagg aggagaggaa aatcatcctg gccgtcatgg ataggcagaa
gaaagaagag gagaaggagc agtccgtgct caaaaaactg catcagcagt ttgaaatgta
taaagagcag gtaaagaaga tgggagaaga atcacagcaa cagcaagaac agaagggtga
tgcgccaacc tgtggtatct gccacaaaac aaagtttgct gatggatgtg gccataactg
ttcatattgc caaacaaagt tctgtgctcg ttgtggaggt cgagtgtcat tacgctcaaa
caaggttatg tgggtatgta atttgtgccg aaaacaacaa gaaatcctca ctaaatcagg
agcatggttt tataatagtg gatctaatac accacagcaa cctgatcaaa aggttcttcg
agggctaaga aatgaggagg cacctcagga gaagaaacca aaactacatg agcagaccca
gttccaagga ccctcaggtg acttatctgt acctgcagtg gagaaaagtc gatctcatgg
gctcacaaga cagcattcta ttaaaaatgg gtcaggcgtg aagcatcaca ttgccagtga
catagcttca gacaggaaaa gaagcccatc tgtgtccaga gatcagaata gaagatacga
ccaaagggaa gaaagagagg aatattcaca gtatgctact tcggataccg caatgcctag
atctccatca gattatgctg ataggcgatc tcaacatgaa cctcagtttt atgaagactc
tgatcattta agttataggg actccaacag gagaagtcat aggcattcca aagaatatat
tgtagatgat gaggatgtgg aaagcagaga tgaatacgaa aggcaaagga gagaggaaga
gtaccagtca cgctaccgaa gtgatccgaa tttggcccgt tatccagtaa agccacaacc
ctatgaagaa caaatgcgga tccatgctga agtgtcccga gcacggcatg agagaaggca
tagtgatgtt tctttggcaa atgctgatct ggaagattcc aggatttcta tgctaaggat
ggatcgacca tcaaggcaaa gatctatatc agaacgtaga gctgccatgg aaaatcagcg
atcttattca atggaaagaa ctcgagaggc tcagggacca agttcttatg cacaaaggac
cacaaaccat agtcctccta cccccaggag gagtccacta cccatagata gaccagactt
gaggcgtact gactcactac ggaaacagca ccacttagat cctagctctg ctgtaagaaa
aacaaaacgg gaaaaaatgg aaacaatgtt aaggaatgat tctctcagtt cagaccagtc
agagtcagtg agacctccac caccaaagcc tcataaatca aagaaaggcg gtaaaatgcg
ccagatttcg ttgagcagtt cagaggagga attggcttcc acgcctgaat atacaagttg
tgatgatgtt gagattgaaa gtgagagtgt aagtgaaaaa ggagacatgg attacaactg
gttggatcat acgtcttggc atagcagtga ggcatcccca atgtctttgc accctgtaac
ctggcaacca tctaaagatg gagatcgttt aattggtcgc attttattaa ataagcgtct
aaaagatgga agtgtacctc gagattcagg agcaatgctt ggcttgaagg ttgtaggagg
aaagatgact gaatcaggtc ggctttgtgc atttattact aaagtaaaaa aaggaagttt
agctgatact gtaggacatc ttagaccagg tgatgaagta ttagaatgga atggaagact
actgcaagga gccacatttg aggaagtgta caacatcatt ctagaatcca aacctgaacc
acaagtagaa cttgtagttt caaggcctat tggagatata ccgcgaatac ctgatagcac
acatgcacaa ctggagtcca gttctagctc ctttgaatct caaaaaatgg atcgtccttc
tatttctgtt acctctccca tgagtcctgg aatgttgagg gatgtcccac agttcttatc
aggacaactt tcaagccaaa gccttagtag aagaacaacg ccttttgttc ctagggttca
gataaaacta tggtttgaca aggttggtca ccaattaata gttacaattt tgggagcaaa
agatctccct tccagggaag atgggaggcc aaggaatcct tatgttaaaa tttactttct
tccagacaga agtgataaaa acaagagaag aactaaaaca gtaaagaaaa cattggaacc
caaatggaac caaacattca tttattctcc agtccaccga agagaatttc gggaacgaat
gctagagatt accctttggg atcaagctcg tgttcgagag gaagaaagtg aattcttagg
cgagatttta attgaattag aaacagcatt attagatgat gagccacatt ggtacaaact
tcagacgcat gatgtctctt cattgccact tccccaccct tctccatata tgccacgaag
acagctccat ggagagagcc caacacggag gttgcaaagg tcaaagagaa taagtgatag
tgaagtctct gactatgact gtgatgatgg aattggtgta gtatcagatt atcgacatga
tggtcgagat cttcaaagct caacattatc agtgccagaa caagtaatgt catcaaacca
ctgttcacca tcagggtctc ctcatcgagt agatgttata ggaaggacta gatcatggtc
acccagtgtc cctcctccac aaagtcggaa tgtggaacag gggcttcgag ggacccgcac
tatgaccgga cattataata caattagccg aatggacaga catcgtgtca tggatgacca
ttattctcca gatagagaca gggattgtga agcagcagat agacagccat atcacagatc
cagatcaaca gaacaacggc ctctccttga gcggaccacc acccgctcca gatccactga
acgtcctgat acaaacctca tgaggtcgat gccttcatta atgactggaa gatctgcccc
tccttcacct gccttatcga ggtctcatcc tcgtactggg tctgtccaga caagcccatc
aagtactcca gtcgcaggac gaaggggccg acagcttcca cagcttccac caaagggaac
gttggataga aaagcaggag gtaaaaaact aaggagcact gtccaaagaa gtacagaaac
aggcctggcc gtggaaatga ggaactggat gactcgacag gcaagccgag agtctacaga
tggtagcatg aacagctaca gctcagaagg aaatctgatt ttccctggtg ttcgcttggc
ctctgatagc cagttcagtg atttcctgga tggccttggc cctgctcagc tagtgggacg
ccagactctg gcaacacctg caatgggtga cattcaggta ggaatgatgg acaaaaaggg
acagctggag gtagaaatca tccgggcccg tggccttgtt gtaaaaccag gttccaagac
actgccagca ccgtatgtaa aagtgtatct attagataac ggagtctgca tagccaaaaa
gaaaacaaaa gtggcaagaa aaacgctgga acccctttac cagcagctat tatctttcga
agagagtcca caaggaaaag ttttacagat catcgtctgg ggagattatg gccgcatgga
tcacaaatct tttatgggag tggcccagat acttttagat gaactagagc tatccaatat
ggtgatcgga tggttcaaac ttttcccacc ttcctcccta gtagatccaa ccttggcccc
tctgacaaga agagcttccc aatcatctct ggaaagttca actggacctt cttactctcg
ttcatagcag ctgtaaaaaa attgttgtca cagcaaccag cgttacaaaa aaaaaaaaaa
aaatcacagg ttgcaaaccc tggtaacact gcatgcttaa tgttgtgtct tctgagcctg
tttctaggga tacaaagcaa tcctgtgttc tcagaggaag ttgcacacat tgtgccctaa
agaaggccct caggtgaaag agcagagctg tgaagaacta tcagatttgg aattcaatga
cactcgagtt ctggtccaat ctgaagccat ggattaatct caaagaatca gtcagtttca
tgcaacagaa gcccttttca atggcacctt tatattttta tcattccttt ttcttcattt
atctaacccc aaagccctga tatgccacag aaatggagct atacagccat gaagcggtgt
tacaggtgag gagtgtaatc ctaggaagca tcaggtgaaa agcaggagac caaagaagtg
gtcaggaaca atcatcagcc ctcctctggg cgggaatcag agcagtcagt ccagcaggaa
gagtggcaga ctttgtagct ccatgggcac gtcaattact aatgctaaga tgtgttggac
tctgaaaaac aaaattctgt ggctacactg tactgaatga aattaaagaa actttttttg
catggacaca gattagctga atacttaaat tattttcttg gggctgcaac ttgcaaaaaa
aaaaaaaaga ataaaaatca gccattttca acaatttata ttatttttaa aaataaattt
cactagtgca tggttttaaa aaggagagag aatgcaacag ggtgatacaa agatacacca
tgtttattct ttaatcatag tctgtgtttt ggcagacatt acaaatggaa atactttcta
gaagatactt aaaattctct ttatgtgaca aataagtata atatattcaa tttatttcca
tgttaaatat acaaatctta tgaagttcaa tatgtgcaaa tttttcacat ctttctcctt
ctctcacttt acctcttctc cctcttttaa acttttcttt ctccctgcca gagtgaacct
tatactaaaa aattacaagt tttgatctga tcctctctca taccccatgt ttgattcaga
gctgtagatg cctctgaatt tgcgaatttc tcaagggaaa attaacttta agagctttct
ttatttcaag catgttgaaa aggattttgc aacatgactt gggagtacat taaagtaagt
cagcatgtat ttgacgaaga agatatttga acttttgcag tttattgtac agtgcatggt
aattttttca cctttaaaat tcagtttaca ggaaaattct aaaatcatgt tgccattgtg
atgtccaata aatttgtttt tagcaccagc attattcata caggggttaa agtattattt
gtagaaggtc ttaggttttg tttgtttttt aatcatttaa agcaatttct ttagccagtt
tccatttact atgtgaatag aagcactgct aaaaattggg aaccctgaaa cacagggctg
tttattaatt catttttctg tagtaaaatt caatttttca caaattatat ttctaaagaa
atatagtaaa cataaatttg caacaatttt aaagctccag tttttaggtg actcaaagaa
agtcattatg cctattaata gttatttgat gccatcacca aaagtctatg tgaaaatctc
ctaaagtcaa aacccctgcc tttggtttta cagacggtta ttaccattgg gtggagctgc
aaggtcaaat ttctcctaag ttcccctatt tagaggaaaa gtcactggtt attgtaataa
accacccatg gttctttatg tacattttga taacacatta ttatagcttg attttaattt
tttgcattaa tttttgaaat ccacatacat ctcatttgtt taaattaagg ccatgcacaa
atattttttt tagttcagtg ctgaccatta aaaactatca tgcttgatac ggtgcaaaag
ttaaaatgag tatcactaaa aatgccttct ttttatgtgg tgcaatatga aatacaccaa
gactgtgtct tgacattctg atggacccag gtaaagttgt taaaagaacg aataaaactt
tattaaaata atttagacac ctgtgtacca gcaacaattg atttaataga cctatagtgt
ctatactatc ccttagaata aaggtttatg attttcctga tactaagatg cagtcacata
atcttttgtg catattccta tacaaattat ttctaatttt aataagaagg acgtgactac
ggaatatttg tacatacttg tcattatgca gtatttattt aaaagttggt gttttttttt
aattttcaca tctgcacctc gacttgtggt ttagtcatgt aactagcact atgccagtga
ccgttgttgc cctgtacata gtatgtttga aaagtaaagg gaattccagt tgggaaaaaa
gggcagatta gtcctgtaat gaacaccaac taatgtaaat caaattcatt ctggtgatgg
tatttaacac tttaaataaa acattttctt tacaggcgtc tgcagtgctt tctctgactt
ttctccccac acagccctga gcctgctgca gctcattccc tgaactcatg tgtcatttaa
agaatgaaat caccgtctcc tacttctcga taacataagt ggactgctgg tcttagcagc
ggccctcagt agagcatttc tttaaaacgc caaaggattt ctgctcacac tatgaaaagg
tgctgttttt taaaaggttg ttattttgga ttgagtttct ttctgattaa atgactcagc
aactcacaga ttttttgagt gaaattttta atttagtcat ggccttcact gacagcatag
tcacaaatac tcaggcacag gctctgctag cccctgggtg aagatggcga aggcataact
ggctttatgc agcatatgtg tttctgctaa agtgtcagtt ttgctttgtg gggagtggag
ggtgtgtttt cgggatgggg agacgtggta acctgacatg taacaacctg tccggagact
agcttctacg tgtggatatg aatgggtgag aggatttctc catatccttc tggggcgatt
cctcaactgg gagaaggaaa ccctgcagag ttctcatggg agtctgcttc aggtttgaaa
tttaagagct agtttggatt catgtttagt aatcgaactg aaatctaagt ctagctgtct
ctctattctt ggaaacaacc atttcctcca tttccaaaga ctcaactcga gtccaattcc
ccctatctgt cccatatatt tttcctttat cccatatata ccccctactc tagtgaattg
tttctttgtt gttcattcct gttctttgtt gttcatatac attcctgtat atgaacaaca
ttttccttta tcccatatat accccctact ctagtgaatt gttttttgt tgttcattcc
tgtttctttg ttgttcatat acattcctgt tataaaaatt cccttccctt tcttatgtgc
cctctcctga aaagcccttc tacttttctc aataatgatc catgcgagtc ccttcttgca
actcccagct cacgaatgag ctctttcggc aactcctgac taaaccctaa caacatggct
gccattgatg ccaacacctt cactttccca gggaccccag atgccaaggc tccataggca
acaataaagg atatgatggt cctgtagtgg gtatgataga attaggcaag agatcaccaa
agctgtctgc ctactactga tgtaaacctt gacattctgt gcacgtaaaa atcatgtgct
caatgtgttt gtctcaactc cctcagctcg tgatgccctc agggttctgt gggcatttat
gcactgaaga aacaggagtt cacacatcca cctctggact gtgaaatgtg tattgagaaa
tactttgcaa gagagaattt ttttaagtga acaaacaaca agtctgtgcc acacacatct
tccatatgcc ctgactcagg tcacttaatc tccaggattt catttcctca cctggaaaat
atggagtttg aggtagattc tcatctatca ttaaatcaac actttaacta aaacgtaagc
tccttcaggg cagagaccgt atcttcagta tcaaaaacaa tgtttgacac atagctgctc
aataaacatc tgtccaatga a

In some embodiments, the first domain of the fusion protein is encoded by a nucleic acid sequence having at least 90%, 91%, 92%, 93%, 94%, 95%, 96%, 97%, 98% or 99% identity to a nucleic acid sequence selected from the group consisting of SEQ ID NOs: 3, 4, 38, and 39. In some embodiments, the first domain is encoded by a nucleic acid sequence having at least 95%, 96%, 97%, 98% or 99% identity to a nucleic acid sequence selected from the group consisting of SEQ ID NOs: 3, 4, 38, and 39. In some embodiments, the first domain is encoded by a nucleic acid sequence having at least 97%, 98% or 99% identity to a nucleic acid sequence selected from the group consisting of SEQ ID NOs: 3, 4, 38, and 39. In some embodiments, the first domain is encoded by a nucleic acid sequence having 100% identity to a nucleic acid sequence selected from the group consisting of SEQ ID NOs: 3, 4, 38, and 39.

In some preferred embodiments, the first domain is encoded by a nucleic acid of SEQ ID NO: 3

In some preferred embodiments, the first domain is encoded by a nucleic acid of SEQ ID NO: 39.

### Calcium Subunits

In various aspects, the fusion protein described herein comprises a subunit of Ca_{V} voltage-dependent calcium channels, e.g., Calcium Voltage-Gated Channel Auxiliary Subunit Beta 4 (Ca_{V}β4). Ca_{V} voltage-dependent calcium channels, e.g., Ca_{V}2.1, are calcium channels found mainly in the brain that are composed of multiple subunits, including alpha-1, beta, alpha-2/delta, and gamma subunits. The α1 subunit forms the pore-region of the channel through which Ca²⁺ ions can flow and is encoded by the CACNA1A gene. Ca_{V} channel β subunits (Ca_{V}β) are intracellular proteins endogenously associated with Ca_{V} channel α1 subunits. There are four subfamilies of Ca_{V}βs (Ca_{V}β1-Ca_{V}β4), each with splice variants encoded by four distinct genes, Cacnb 1-4. The four Ca_{V}β genes have 14 exons except Cacnb3, which has 13, and each Ca_{V}β has 2 or more splice variants. Human Ca_{V}β have five distinct domains, NH2 terminus, the SH3 domain, the HOOK, the GK domain, and the COOH terminus.

Methods and compositions described herein require a Ca_{V} channel β subunit, such as Ca_{V}β1. As used herein, the “Calcium Voltage-Gated Channel Auxiliary Subunit Beta 1” refers to a protein that is a calcium channel beta subunit family member. CACNB1, the gene that encodes this protein, plays an important role in the calcium channel by modulating G protein inhibition, increasing peak calcium current, controlling the alpha-1 subunit membrane targeting and shifting the voltage dependence of activation and inactivation. Alternative splicing occurs at this locus and three transcript variants encoding three distinct isoforms have been identified. Sequences for CACNB1, also known as CACNLB1; CAB1; CCHLB1, are known for a number of species, e.g., human CACNB1 (NCBI Gene ID: 782) polypeptide (e.g., NCBI Ref Seq NP_000714.3) and mRNA (e.g., NCBI Ref Seq NM_000723.5). CACNB1 can refer to human CACNB1, including naturally occurring variants, molecules, and alleles thereof. The human nucleic acid sequence of SEQ ID NO: 12 comprises the nucleic sequence which encodes Ca_{V}β1. The human polypeptide sequence of SEQ ID NO: 8 comprises the polypeptide sequence of Ca_{V}β1.

Methods and compositions described herein require a Ca_{V} channel β subunit, such as Ca_{V}β2. As used herein, the “Calcium Voltage-Gated Channel Auxiliary Subunit Beta 2” refers to a protein that is a voltage-gated calcium channel superfamily member. Ca_{V}β2 was originally identified as an antigen target in Lambert-Eaton myashenic syndrome, an autoimmune disorder. Mutations in this gene are associated with Brugada syndrome. Alternatively splice variants encoding different isoforms have been described. Sequences for CACNB2, also known as CACNLB2; MYSB; CAB2; CAVB2, are known for a number of species, e.g., human CACNB2 (NCBI Gene ID: 783) polypeptide (e.g., NCBI Ref Seq NP 000715.2) and mRNA (e.g., NCBI Ref Seq NM 000724.4). CACNB2 can refer to human CACNB2, including naturally occurring variants, molecules, and alleles thereof. The human nucleic acid sequence of SEQ ID NO: 11 comprises the nucleic sequence which encodes Ca_{V}β2. The human polypeptide sequence of SEQ ID NO: 7 comprises the polypeptide sequence of Ca_{V}β2.

Methods and compositions described herein require a Ca_{V} channel β subunit, such as Ca_{V}β3. As used herein, the “Calcium Voltage-Gated Channel Auxiliary Subunit Beta 3” refers to a protein that is a regulatory beta subunit of the voltage-dependent calcium channel. Beta subunits are composed of five domains, which contribute to the regulation of surface expression and gating of calcium channels and may also play a role in the regulation of transcription factors and calcium transport. Sequences for CACNB3, also known as CACNLB3; CAB3; are known for a number of species, e.g., human CACNB3 (NCBI Gene ID: 784) polypeptide (e.g., NCBI Ref Seq NP 000716.2) and mRNA (e.g., NCBI Ref Seq NM_000725.4). CACNB3 can refer to human CACNB3, including naturally occurring variants, molecules, and alleles thereof. The human nucleic acid sequence of SEQ ID NO: 10 comprises the nucleic sequence which encodes Ca_{V}β3. The human polypeptide sequence of SEQ ID NO: 6 comprises the polypeptide sequence of Ca_{V}β3.

Methods and compositions described herein require a Ca_{V} channel β subunit, such as Ca_{V}β4. As used herein, the “Calcium Voltage-Gated Channel Auxiliary Subunit Beta 4” refers to a protein that is a beta subunit family member of voltage-dependent calcium channel complex proteins. Calcium channels mediate the influx of calcium ions into the cell upon membrane polarization and consist of a complex of alpha-1, alpha-2/delta, beta, and gamma subunits in a 1:1:1:1 ratio. Various version of each of these subunits exits, either expressed from similar genes or the result of alternative splicing. Ca_{V}β4 plays an important role in calcium channel function by modulating G protein inhibition, increasing peak calcium current, controlling the alpha-1 subunit membrane targeting and shifting the voltage dependence of activation and inactivation. Certain mutations in this gene have been associated with idiopathic generalized epilepsy (IGE), juvenile myoclonic epilepsy (JME), and episodic ataxia, type 5. Sequences for CACNB4, also known as CACNLB4; CAB4; EJM4, EIG9, EJM6, EA5, EJM are known for a number of species, e.g., human CACNB4 (NCBI Gene ID: 785) polypeptide (e.g., NCBI Ref Seq NP_001005747.1) and mRNA (e.g., NCBI Ref Seq NM_001005746.4). CACNB4 can refer to human CACNB4, including naturally occurring variants, molecules, and alleles thereof. The human nucleic acid sequence of SEQ ID NO: 9 comprises the nucleic sequence which encodes Ca_{V}β4. The human polypeptide sequence of SEQ ID NO: 5 comprises the polypeptide sequence of Ca_{V}34.

Exemplary sequences for human Ca_{V}β subunits can be found in NCBI with Accession Numbers as listed: Ca_{V}β4 isoform a (NP 001005747.1), Ca_{V}β4 isoform b (NP 000717.2), Ca_{V}β4 isoform c (NP 001005746.1), Ca_{V}β3 isoform 1 (NP 000716.2), Ca_{V}β3 isoform 2 (NP 001193844.1), Ca_{V}β3 isoform 3 (NP 001193845.1), Ca_{V}β2 isoform 1 (NP 000715.2), Ca_{V}β2 isoform 2 (NP 963890.2), Ca_{V}β2 isoform 3 (NP 963887.2), Ca_{V}β1 isoform 1 (NP 000714.3), Ca_{V}β1 isoform 2 (NP 954855.1), and Ca_{V}β1 isoform 3 (NP 954856.1).

In some embodiments, the second domain of the fusion protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a wild-type sequence of a Ca_{V}βsubunit. For example, the second domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a human Ca_{V}β subunit or a homologous or orthologous Ca_{V}β subunit.

In some embodiments, the calcium channel subunit is a Ca_{V}β4 subunit. For example, the second domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to Ca_{V}β4 or a homologous or orthologous Ca_{V}β4. Preferably, the second domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to Ca_{V}β4 (i.e., SEQ ID NO: 5, 45, or 46).

SEQ ID NO: 5 describes the Human amino acid sequence for Ca_{V}β4 (NP_001005747.1).

(SEQ ID NO: 5)
mydnlylhgi edseagsads ytsrpsdsdv sleedreair qereqqaaiq lerakskpva
favktnvsyc galdedvpvp staisfdakd flhikekynn dwwigrlvke gceigfipsp
lrleniriqq eqkrgrfhgg kssgnssssl gemvsgtfra tptstakqkq kvtehippyd
vvpsmrpvvl vgpslkgyev tdmmqkalfd flkhrfdgri sitrvtadis lakrsvlnnp
skraiiersn trsslaevqs eierifelar slqlvvldad tinhpaqlik tslapiivhv
kvsspkvlqr liksrgksqs khlnvqlvaa dklaqcppem fdvildenql edacehlgey
leaywratht tsstpmtpll grnlgstals pyptaisglq sqrmrhsnhs tenspierrs
lmtsdenyhn erarksrnrl ssssqhsrdh yplveedypd syqdtykphr nrgspggysh
dsrhrl

SEQ ID NO: 45 describes the Mouse amino acid sequence for Ca_{V}β4 (NP_001032176.1)

(SEQ ID NO: 45)
MSSSYGKNGAADGPHSPSSQVARGTTTRRSRLKRSDGSTTSTSFILRQG
SADSYTSRPSDSDVSLEEDREAIRQEREQQAAIQLERAKSKPVAFAVKT
NVSYCGALDEDVPVPSTAISFDAKDFLHIKEKYNNDWWIGRLVKEGCEI
GFIPSPLRLENIRIQQEQKRGRFHGGKSSGNSSSSLGEMVSGTFRATPT
TTAKQKQKVTEHIPPYDVVPSMRPVVLVGPSLKGYEVTDMMQKALFDFL
KHRFDGRISITRVTADISLAKRSVLNNPSKRAIIERSNTRSSLAEVQSE
IERIFELARSLQLVVLDADTINHPAQLIKTSLAPIIVHVKVSSPKVLQR
LIKSRGKSQSKHLNVQLVAADKLAQCPPEMFDVILDENQLEDACEHLGE
YLEAYWRATHTSSSTPMTPLLGRNVGSTALSPYPTAISGLQSQRMRHSN
HSTENSPIERRSLMTSDENYHNERARKSRNRLSSSSQHSRDHYPLVEED
YPDSYQDTYKPHRNRGSPGGCSHDSRHRL

SEQ ID NO: 46 describes a modified Mouse amino acid sequence for Ca_{V}β4.

(SEQ ID NO: 46)
MSSSYAKNGAADGPHSPSSQVARGTTTRRSRLKRSDGSTTSTSFILRQG
SADSYTSRPSDSDVSLEEDREAIRQEREQQAAIQLERAKSKPVAFAVKT
NVSYCGALDEDVPVPSTAISFDAKDFLHIKEKYNNDWWIGRLVKEGCEI
GFIPSPLRLENIRIQQEQKRGRFHGGKSSGNSSSSLGEMVSGTFRATPT
TTAKQKQKVTEHIPPYDVVPSMRPVVLVGPSLKGYEVTDMMQKALFDFL
KHRFDGRISITRVTADISLAKRSVLNNPSKRAIIERSNTRSSLAEVQSE
IERIFELARSLQLVVLDADTINHPAQLIKTSLAPIIVHVKVSSPKVLQR
LIKSRGKSQSKHLNVQLVAADKLAQCPPEMFDVILDENQLEDACEHLGE
YLEAYWRATHTSSSTPMTPLLGRNVGSTALSPYPTAISGLQSQRMRHSN
HSTENSPIERRSLMTSDENYHNERARKSRNRLSSSSQHSRDHYPLVEED
YPDSYQDTYKPHRNRGSPGGCSHDSRHRL

SEQ ID NO: 6 describes the Human amino acid sequence for Ca_{V}β3 (NP_000716.2).

(SEQ ID NO: 6)
myddsyvpgf edseagsads ytsrpsldsd vsleedresa rrevesqaqq qlerakhkpv
afavrtnvsy cgvldeecpv qgsgvnfeak dflhikekys ndwwigrlvk eggdiafips
pqrlesirlk qeqkarrsgn psslsdignr rspppslakq kqkqaehvpp ydvvpsmrpv
vlvgpslkgy evtdmmqkal fdflkhrfdg risitrvtad lslakrsvln npgkrtiier
ssarssiaev qseierifel akslqlvvld adtinhpaql aktslapiiv fvkvsspkvl
qrlirsrgks qmkhltvqmm aydklvqcpp esfdvilden qledacehla eylevywrat
hhpapgpgll gppsaipglq nqqllgerge ehsplerdsl mpsdeasess rqawtgssqr
ssrhleedya dayqdlyqph rqhtsglpsa nghdpqdrll aqdsehnhsd rnwqrnrpwp
kdsy

SEQ ID NO: 7 describes the Human amino acid sequence for Ca_{V}β2 (NP_000715.2).

| (SEQ ID NO: 7) | | | | | |
|---|---|---|---|---|---|
| mqccglvhrr | rvrvsygsad | sytsrpsdsd | vsleedreav | rreaerqaqa | qlekaktkpv |
| afavrtnvsy | saaheddvpv | pgmaisfeak | dflhvkekfn | ndwwigrlvk | egceigfips |
| pvklenmrlq | heqrakqgkf | yssksggnss | sslgdivpss | rkstppssai | didatgldae |
| endipanhrs | pkpsansvts | phskekrmpf | fkktehtppy | dvvpsmrpvv | lvgpslkgye |
| vtdmmqkalf | dflkhrfegr | isitrvtadi | slakrsvinn | pskhaiiers | ntrsslaevq |
| seierifela | rtlqlvvlda | dtinhpaqls | ktslapiivy | vkisspkvlq | rliksrgksq |
| akhlnvqmva | adklaqcppe | lfdvildenq | ledacehlad | yleaywkath | ppssslpnpl |
| lsrtlatssl | plsptlasns | qgsqgdqrtd | rsapirsasq | aeeepsvepv | kksqhrssss |
| aphhnhrsgt | srglsrqetf | dsetqesrds | ayvepkedys | hdhvdhyash | rdhnhrdeth |
| gssdhrhres | rhrsrdvdre | qdhnecnkqr | srhkskdryc | ekdgeviskk | rneagewnrd |
| vyirq | | | | | |

SEQ ID NO: 8 describes the Human amino acid sequence for Ca_{V}β1 (NP_000714.3).

| (SEQ ID NO: 8) | | | | | |
|---|---|---|---|---|---|
| mvqktsmsrg | pyppsqeipm | evfdpspqgk | yskrkgrfkr | sdgstssdtt | snsfvrqgsa |
| esytsrpsds | dvsleedrea | lrkeaerqal | aqlekaktkp | vafavrtnvg | ynpspgdevp |
| vqgvaitfep | kdflhikeky | nndwwigrlv | kegcevgfip | spvkldslrl | lqeqklrqnr |
| lgssksgdns | ssslgdvvtg | trrptppasa | kqkqkstehv | ppydvvpsmr | piilvgpslk |
| gyevtdmmqk | alfdflkhrf | dgrisitrvt | adislakrsv | lnnpskhiii | ersntrssla |
| evqseierif | elartlqlva | ldadtinhpa | qlsktslapi | ivyikitspk | vlqrliksrg |
| ksqskhlnvq | iaaseklaqc | ppemfdiild | enqledaceh | laeyleaywk | athppsstpp |
| npllnrtmat | aalaaspapv | snlqgpylas | gdqpleratg | ehasmheypg | elgqppglyp |
| sshppgragt | lralsrqdtf | dadtpgsrns | aytelgdscv | dmetdpsegp | glgdpagggt |
| pparqgswed | eeedyeeelt | dnrnrgrnka | rycaegggpv | lgrnkneleg | wgrgvyir |

SEQ ID NO: 47 describes a modified human amino acid sequence for Ca_{V}β1.

(SEQ ID NO: 47)
MVQKSGMSRGPYPPSQEIPMEVFDPSPQGKYSKRKGRFKRSDGSTSSDTT
SNSFVRQGSAESYTSRPSDSDVSLEEDREALRKEAERQALAQLEKAKTKP
VAFAVRTNVGYNPSPGDEVPVQGVAITFEPKDFLHIKEKYNNDWWIGRLV
KEGCEVGFIPSPVKLDSLRLLQEQTLRQNRLSSSKSGDNSSSSLGDVVTG
TRRPTPPASAKQKQKSTEHVPPYDVVPSMRPIILVGPSLKGYEVTDMMQK
ALFDFLKHRFDGRISITRVTADISLAKRSVLNNPSKHIIIERSNTRSSLA
EVQSEIERIFELARTLQLVALDADTINHPAQLSKTSLAPIIVYIKITSPK
VLQRLIKSRGKSQSKHLNVQIAASEKLAQCPPEMFDIILDENQLEDACEH
LAEYLEAYWKATHPPSSTPPNPLLNRTMATAALAASPAPVSNLQGPYLAS
GDQPLDRATGEHASVHEYPGELGQPPGLYPSNHPPGRAGTLRALSRQDTF
DADTPGSRNSAYTEPGDSCVDMETDPSEGPGPGDPAGGGTPPARQGSWEE
EEDYEEEMTDNRNRGRNKARYCAEGGGPVLGRNKNELEGWGQGVYIR

In some embodiments, the second domain comprises an amino acid sequence having at least 90%, 91%, 92%, 93%, 94%, 95%, 96%, 97%, 98% or 99% identity to an amino acid sequence selected from the group consisting of SEQ ID NO: 5-8 and 45-47. In some embodiments, the second domain comprises an amino acid sequence having at least 95%, 96%, 97%, 98% or 99% identity to an amino acid sequence selected from the group consisting of SEQ ID NO: 5-8 and 45-47. In some embodiments, the second domain comprises an amino acid sequence having at least 97%, 98% or 99% identity to an amino acid sequence selected from the group consisting of SEQ ID NO: 5-8 and 45-47. In some embodiments, the second domain comprises an amino acid sequence having 100% identity to an amino acid sequence selected from the group consisting of SEQ ID NO: 5-8 and 45-47.

In some preferred embodiments, the second domain comprises the amino acid sequence of SEQ ID NO: 5.

In some preferred embodiments, the second domain comprises the amino acid sequence of SEQ ID NO: 46.

In some embodiments, the second domain of the fusion protein is encoded by a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a wild-type sequence of a Ca_{V}βsubunit. For example, the second domain is encoded by a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a human Ca_{V}β subunit or a homologous or orthologous Ca_{V}β subunit.

Exemplary sequences for human Ca_{V}β subunits can be found in NCBI with Accession Numbers as listed: Ca_{V}β4 isoform a (NM 001005747.4), Ca_{V}β4 isoform b (NM 000726.5), Ca_{V}β4 isoform c (NM 001005746.4), Ca_{V}β3 isoform 1 (NM 000725.4), Ca_{V}β3 isoform 2 (NM 001206915.2), Ca_{V}β3 isoform 3 (NM 001206916.2), Ca_{V}β2 isoform 1 (NM 000724.4), Ca_{V}β2 isoform 2 (NM 201596.3), Ca_{V}β2 isoform 3 (NM 201590.3), Ca_{V}β1 isoform 1 (NM 000723.5), Ca_{V}β1 isoform 2 (NM 199247.3), and Ca_{V}β1 isoform 3 (NP 199248.3).

In some embodiments of any one of the aspects, the calcium channel subunit is a Ca_{V}β4 subunit. For example, the second domain is encoded by a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to Ca_{V}β4 or a homologous or orthologous Ca_{V}β4. Preferably, the second domain is encoded by a nucleic acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to Ca_{V}β4 (i.e., SEQ ID NO: 9, 42, or 43).

SEQ ID NO: 9 describes the Human nucleic acid sequence for Ca_{V}β4 (NM_001005747.4).

| (SEQ ID NO: 9) | | | | | |
|---|---|---|---|---|---|
| agtcaccagt | ctgtggttga | ggaagggttt | tgctttttgt | gctctgtatt | gggaaggcag |
| ataaaatgcc | aggtctggac | tgcctctggg | gataacctca | ccctgtggga | tgtgaatgaa |
| tagcatcttg | cctggtaaat | ccacaggaat | tgataaggca | ggcgcagttc | tcccaaacag |
| gccttttctc | tttaagctgt | agctgtggtt | tctgcagcaa | ttttgttttt | gccttgaaag |
| aggtgctctg | gattatcaca | cctccatgta | tgacaatttg | tacctgcatg | gaattgaaga |
| ctcggaggct | ggttcagcgg | attcctacac | aagcaggccg | tctgactccg | atgtctcttt |
| ggaagaggac | cgggaagcaa | ttcgacagga | gagagaacag | caagcagcta | tccagcttga |
| gagagcaaag | tccaaacctg | tagcatttgc | cgtgaagaca | aatgtgagct | actgcggcgc |
| cctggacgag | gatgtgcctg | ttccaagcac | agctatctcc | tttgatgcta | aagactttct |
| acatattaaa | gagaaatata | acaatgattg | gtggatagga | aggctggtga | aagagggctg |
| tgaaattggc | ttcattccaa | gtccactcag | attggagaac | atacggatcc | agcaagaaca |
| aaaaagagga | cgttttcacg | gagggaaatc | aagtggaaat | tcttcttcaa | gtcttggaga |
| aatggtatct | gggacattcc | gagcaactcc | cacatcaaca | gcaaaacaga | agcaaaaagt |
| gacggagcac | attcctcctt | acgatgttgt | gagagcaata | attgaacgtt | tgttagtggg |
| gccgtcactg | aaaggttacg | aggtaacaga | accgtcaatg | cgtccggtgg | ttgatttcct |
| gaagcacagg | tttgatggga | ggatttcaat | catgatgcag | aaagccctct | tttctcttgc |
| taagaggtct | gtcctaaata | atcccagcaa | aacgagagtg | acagctgaca | cgaacacccg |
| gtccagctta | gcggaagtac | aaagtgaaat | tgaaagaatc | tttgagttgg | caagatcttt |
| gcaactggtt | gttcttgatg | cagacaccat | caatcaccca | gcacaactta | taaagacttc |
| cttagcacca | attattgttc | atgtaaaagt | ctcatctcca | aaggttttac | agcggttgat |
| taaatctaga | ggaaagtcac | aaagtaaaca | cttgaatgtt | caactggtgg | cagctgataa |
| acttgcacaa | tgccccccag | aaatgtttga | tgttatattg | gatgaaaatc | agcttgagga |
| tgcatgtgaa | catctagggg | agtacctgga | ggcgtactgg | cgtgccaccc | acacaaccag |
| tagcacaccc | atgaccccgc | tgctgggaag | gaatttgggc | tccacggcac | tctcaccata |
| tcccacagca | atttctgggt | tacagagtca | gcgaatgagg | cacagcaacc | actccacaga |
| gaactctcca | attgaaagac | gaagtctaat | gacctctgat | gaaaattatc | acaatgaaag |
| ggctcggaag | agtaggaacc | gcttgtcttc | cagttctcag | catagccgag | atcattaccc |
| tcttgtggaa | gaagattacc | ctgactcata | ccaggacact | tacaaacccc | ataggaaccg |
| aggatcacct | gggggatata | gccatgactc | ccgacatagg | ctttgagtct | aatgaaacaa |
| aaaatattca | tctgttgaca | atttgccata | gcagtgctag | gataaaccaa | tcatcttaac |
| ttggctaaca | tagcacagta | tttactgtgc | taatgggctg | ctgtcatttt | atgctaagta |
| aggggcaaaa | aaaaaaatta | cattatgccc | ttgagtctag | atggatatta | gatgcccgat |
| catatagata | tttttaagtg | caacatttac | atgataacag | tacattttgt | tttcttcata |
| gatttagaca | catcaatttg | taatttaggg | tacttacaag | gcacatataa | aataatttcc |
| catgctggaa | attccaaatg | accagttcca | gttggaacca | atttataaac | caattcctgt |
| tggaatttgg | gtgaattgac | tagaaagtct | acttgagcat | gttgtaatca | actgaaaaat |
| ctggggggaa | aaaaaaaaaa | ctaataagaa | aatcgaaata | ccaatagaag | ccaaatccag |
| ctttggtatt | tatttgctgg | aagctaaatt | tacacttaaa | gttggagtgt | ataaacattt |
| taatatatgt | taatgttgcc | agaatgactt | ttcagactta | ccaaatgtat | tgatagtgcc |
| aaaaaaaaaa | tctcagaaat | cttaatgcag | aaattatata | gtctcctcag | attttaatat |
| ttttatttct | ttcctggcac | agctgttgta | ttcaaagtgt | tttgcttttt | gtttggtcag |
| tactgttagc | tcatttgaag | ttggcaaaaa | caaatacaaa | caagcaaaaa | acctcaaatt |
| tttttcagca | gtgagagaga | atagtaaaga | gaggcccaag | catagctggt | tatgtgaaat |
| ataaaatggg | gtgttgttct | ccctttaatc | aatctgagca | caactacata | catccaggaa |
| tctgaagtca | gatagtaagg | atgtggtacc | aatcctcccg | agtgcaatgc | taaacacttc |
| caggtgggaa | ttcatctatt | tcacttaaca | caaacgcagg | gtctgtattt | cctgccaaca |
| agattgagta | tctttccatt | gcttctctca | gtctctgtct | ttgaatatct | gaatgttctg |
| gcccttcaac | aaattcttta | aatcatgcta | aggaatttcg | ttttatgtgg | acacactcaa |
| ctttgatatt | caggagatgt | ttttacaaaa | tgactaattg | aaaaaagttg | aaacaaatga |
| ttttaaaagc | ttgcagtaaa | aaataaactc | atagattcag | caaatatttg | ttgactaaaa |
| ttatagcatt | tttaatgcag | aaaattataa | agaatttgga | gattgagctg | ccttatttct |
| aatgaacact | cctcttcatc | ttattaagtc | ttcacagttt | ttccagtggc | tataattatg |
| gaattggtga | cctatgtttt | atattatcaa | ggaagcctta | gattcactct | gatctaagaa |
| attattgaaa | ttgacccttt | ttacacaaat | gtctttaaaa | aaaaaaactt | atttctaaat |
| gtccatagtg | catgcatttt | ctaaaacccc | aataagtcca | gccataaaag | ttcttttctg |
| ctagtacatg | tcatgaagcc | tatttttgaa | aataatgata | attaatttta | tagtatcttc |
| ttttctcccc | aagcctttca | gtattcaggc | actcaaagca | tgcaccagtt | cagtaatctt |
| cttgaccatg | gaaataagtt | ccacattact | gatgagttat | atatatatct | tgaaccccat |
| tacaacctca | ccacatcccc | ctgcatttat | gttcagtgtt | tgtaccccat | tatgaacctg |
| tttagctgat | aatgaggata | cctataattc | tgtaagtcat | ttcaaatatt | tgatacaatt |
| aaaaaatatg | ggcttagtaa | attttaaaag | ttaacatgtg | atactggctt | gtgagattct |
| tctgtcatct | agtgaagctt | ataaaacttt | cagttaagac | attttaaaag | agttatatcc |
| attaggcact | ttaataaaca | tataacctgc | cattactgaa | tactttgtta | gaacacagaa |
| gagcatcttc | cagcaaacaa | aaatatattt | attacatgta | cagcacatac | ttgcatgaga |
| aagaaaacat | tagccaaaac | atttttctat | atgctttact | ggtttcttgt | ttatgcgcat |
| taaagtattt | aattgcaaag | agtgcaccaa | ggtgtacaaa | tttagactag | acagctgttc |
| agctttttct | gtagtagcat | tagcagtttg | gtcatttaac | tactttaaga | tacttacatt |
| ttatggcagg | tagctgtaac | agcatttaaa | tatatttcct | agtcatcaag | attaccatct |
| tctttcaact | ttttaaaagt | cacttgtttt | taaaggtttt | tcaaagcctt | gtttttagtc |
| ttccatgtca | tgccgataaa | gagagtctca | ttcaatatct | gtgcacaaaa | ccaatctatc |
| aattaccatc | agatgtccat | acctgttttg | ctttgcattc | ttattcaaaa | gctgacaatc |
| ttggaaaggg | tataatgtat | attttattga | tggatatatt | taaatgttta | ctttaatttg |
| gaatattctt | tccccttctt | tctgtaataa | gtttatcttc | ctcaaataag | gggatataca |
| tcttattttt | tttaaagact | taagagttta | ttgtgtaaat | gatacttaaa | atctagatat |
| atactaaatg | aaaagtttta | tttttgccaa | gcacaccaag | cacctttgaa | aaatacatac |
| tccatacttt | gtttttaatt | aatttcattt | aaagatagca | aatgattata | agcaagagaa |
| acaagtcttt | tactgttttg | aggctgtttt | aacttacagt | ataagccaca | tactagaatt |
| ctgtaatttc | agtcacagac | atataataat | gtggactcat | gtgacaatgt | ggaaatgtag |
| ttctggaaaa | aaacgaatgc | aatttgtcaa | tcctgatgtt | aattctcaag | ggtataatgt |
| cactctaata | ttataaccaa | caacattatc | ttgtttaata | tttaaagcaa | cacttagaaa |
| tgtttataaa | tgatgattct | ctcttatctt | tatacagttc | tattggagag | aaggttgaaa |
| ttgttcacaa | tttagatgat | ggtctttcct | aataagagtc | ttaattttgt | gactatataa |
| gtcattgcag | ttagtcttta | atgaaatgaa | ttttattaat | gtaaaacaaa | ttctgagaca |
| attatttgtt | tcctttaaat | aatctgctat | ttaattatta | ttgtcgttgt | ttgcaaaatg |
| tatttttgga | atgttaacct | cttttcatac | ctgcatactt | gaacttgtct | tttgtgtggg |
| ggccttaaaa | ttcataatag | gaagtggagg | cagtgaaagg | aagtaagaaa | aggaaattat |
| gctaaaagat | ggtttgcaaa | tttaaattct | ttaaatgaac | atatcacaaa | atgttgttaa |
| gacccaggat | gtccgttaag | aatgtgtctt | ggcaacctta | atttgcatgt | tagtatatat |
| tttcttagtt | acacagtttt | attttggtat | ttgcttaaaa | aatgtaatac | cattgtagaa |
| attaccctat | gtactgatat | atttactacc | tcttctctag | agaagacaat | ctgtctttaa |
| agggaaaaac | actggtcaat | aagtagtgat | caaaattatc | actattctgg | ctaaccttgc |
| tgcaaagcta | aatacataga | ttattaaaat | atgccttccc | tttagatttt | aaactgtttc |
| caaaatgctg | tttggcaccc | tttcatttct | tccacgttag | tgaagttttc | tttgaattgg |
| ttcacagttt | atcatgaggc | aaggttaatt | taacaaataa | gtcatcatag | ggaacaaaac |
| aaactgcaag | ggaattgcct | ttgtgtgcaa | cttgttataa | tatctagttc | taagtctaaa |
| ggagtaaaag | tccaaagact | ttatgtttaa | gagcagctgg | tttgcaaatt | cacattttgt |
| tggtccttga | cttccacaga | gcatgactta | gccatttact | gggtaaggat | atgcataatc |
| ttggactccc | atgtaattgg | aataaacttc | atatcaatgt | ttcaaataaa | tgctatgtac |
| cccttattcc | ttttacattt | ctgcctttgt | cttttatgac | aatgttgcat | gttgtgtttt |
| tttaaattat | ataaaactca | tccttgaatg | actgtatgaa | tgaaaaattt | ataggaaatt |
| atgtaataaa | atctgtttta | ttagtttctc | atacctttaa | atactgtcaa | tactataact |
| gttagtaagt | tacttactca | agctgtgtct | atggggcaga | gaacagtcct | cttctttaaa |
| ataatcatga | tcaaaccagt | caagtcaatc | caattagctg | ttagacagac | aataaggata |
| accttctttt | tgacttaagc | agtcaagtta | gtcaaaatag | aaaaattggc | ttatatgggc |
| acccagaact | acagcattca | aaaattactt | ttaaaagttt | cttcttttat | taaaatggac |
| tttottaaat | aaaatacatc | ctctgatagc | agctatctct | ttttacttgt | aatgattaat |
| tctaaatgct | tcatgtaatt | gagggacatt | atgagaagac | tagaaagtat | ctcatcagat |
| aatcctaagg | gctgtaactt | ctacttgcca | taaacaaagt | ataattaaga | gtgagtagca |
| attggtttgg | ggagtcgtag | ttatatatga | aactttcttt | tgtttctcat | gatgtatgta |
| aaatgtttgc | agaaggatgg | tagaaaatgt | tactaatgaa | tttagagtgg | tttgggagaa |
| tacactgata | cttcctcatt | tgaaaattat | aaggagttac | aaggggaact | aagaaccaga |
| agtggctggc | ctctagcttt | tcaatacctg | ataagtgttc | ttggttatgt | tttgaacact |
| taaatttctc | aacaaatggc | tatgtctata | tttatgtagg | ggccaccctt | gccctacttg |
| catgccactt | tgacttttaa | ctgaaatcag | tgtgaattct | gtatgcataa | gggatgtaat |
| aaagaggcgg | gatcattaac | gctatatcgt | tgcatactat | ttttcatatg | ccttacaata |
| ttttctggga | tgttattaaa | atgaaagcat | tttattgcca | aaataaatct | atgttgttac |
| ttataatctg | cattagcagt | agaaaaataa | tgtagcaaaa | acatattgta | cagtacaaaa |
| ttataaaata | tatattttgc | caataatata | aaaatagatt | gaggagaaag | aagcacattt |
| gttatagcac | aaacttataa | caaaatccac | cgttaaattt | ggcagtcata | ttttttaaaa |
| gtatgtcgat | ttagttctaa | gttttgtcag | atttacttca | tctaccatct | ttttaacttg |
| ctgcttgata | cttttccagc | aatttatttc | atagccttgc | aatctgtgga | tgtaatttct |
| tttgttttat | tcccttccat | aaaatttatt | aaattttatt | ttgtgataac | actcatacca |
| tattttctcc | tcttatttct | tctctttcaa | taacaacagc | agaaagtcac | agcaagtgag |
| ctcatagggc | tcctctgaat | tttggacatg | tgtgagattt | ttgtattttt | cacagcctcg |
| agtcagaatg | atcaataaag | atgtcccaac | cacaaaaaat | tatattagtt | agggattctt |
| taatttgttt | tgtactgaat | actactttga | aacataagtt | aaagccatta | gctgtgtttt |
| tgtagggatg | gtgggaagga | ataaaaaata | aatgtattaa | cataaaacat | actaaaacag |
| aaatatggca | aaatgatgta | aaaagatttt | tcctactatt | ctgagggctg | ttgaaatccc |
| aggctattgc | tatatctcca | ttgacaaagt | gggataaaaa | atgcctttgt | caaataattt |
| tttttctttc | tttaaatttt | agttatttat | tttaataatt | tgtccccttt | tatttttctt |
| atctaggtat | gtgtagcatt | ctacaaggca | tgctgggtaa | aatatactat | gcaaatgtgt |
| acagtgctgc | atgttttgtt | ctccctctgc | tttagtggct | ttaaagaaaa | aaacgtggtg |
| ttatttttgt | gaataaaaac | attaacaaag | aaaatgcaga | gtgcatgcat | agagtatcct |
| catttctatg | gatttgtttt | atggaattta | aatgtgtaca | aaacaactgc | attacttttt |
| ttcttttata | taataaatga | aaaaaacact | gaaa | | |

SEQ ID NO: 42 describes the Mouse nucleic acid sequence for Ca_{V}β4 (NM 001037099.3).

(SEQ ID NO: 42)
atgtcgtcctcgtacggcaagaacggggcggcggacgggccgcactcccccagctcacaggtggcccgag
gcaccacgacccggaggagcaggttgaaaagatcggatggcagcaccacttctaccagcttcatcctcag
acaggggtcagcagattcctatacaagcaggccatcggactccgatgtctccttggaagaggaccgggaa
gcgatccggcaagagcgagaacagcaagcagctatccagcttgagagagcgaagtccaaacctgtagctt
ttgcggtgaaaacgaacgtgagctactgtggtgccctggacgaagatgtgcccgttcccagcacagccat
ctcctttgacgccaaggactttcttcacattaaagagaaatataacaatgattggtggataggaagactg
gtaaaagagggctgtgagattggcttcatcccaagtccactgcgcttggagaatattcggattcaacagg
aacagaaaagaggccgttttcatggcgggaaatcgagtggaaactcttcctccagtctgggagaaatggt
atcaggaacattccgagcaactcccacaacaacagcaaaacagaagcagaaagtgacggagcacattcct
ccgtatgacgtcgtgccgtcaatgcgtcctgtggtgttagtggggccatcactgaaaggttatgaggtaa
cagacatgatgcagaaagccctctttgatttcctgaagcacaggtttgatgggaggatatcaataacaag
agtgacagctgacatttctcttgctaagagatctgtcctcaacaatcctagcaagagagcaataattgaa
cgttccaacaccagatccagcttagcggaagtacaaagtgaaattgaaagaatttttgagttggcaagat
ctttgcaattggttgttcttgatgcagacaccatcaaccacccagcacagctgataaagacatccttagc
acccatcatcgtccacgtgaaggtctcgtccccaaaggttttacagcggctgattaagtccagaggaaag
tcccaaagcaaacacttgaatgttcaactggtggcggccgataaactggcccagtgcccgcctgaaatgt
ttgatgttatattagatgagaatcaacttgaggatgcctgtgaacatctgggagagtacctggaggcata
ctggcgtgccacccacacgagcagtagcacccctatgaccccattactggggcggaacgtgggctccaca
gccctctcaccatatcccacagcaatctctggattacagagtcagcgaatgagacacagcaaccattcta
cagagaattctccaattgaaagacgaagcctaatgacctcggatgaaaattaccacaatgagagggcccg
caagagtaggaaccgcttgtcttccagctcccagcacagccgagaccactaccctctggtggaagaagat
tacccggactcgtaccaggacacttataagccccataggaaccgaggatcgcccggggggtgcagccatg
actcccgacataggctttga

SEQ ID NO: 43 describes a modified Mouse nucleic acid sequence for Ca_{V}β4.

(SEQ ID NO: 43)
atgtcgtcctcctacgccaagaacggggcggcggacgggccgcactcccccagctcgcaggtggcccgag
gcaccacgacccggaggagcaggttgaaaagatccgatggcagcaccacttccaccagcttcatcctcag
acaggggtcagcagattcctatacaagcaggccatcggactccgatgtctctttggaagaggaccgggaa
gcgattcggcaagagagagaacagcaagcagctatccagcttgagagagcgaagtccaaacctgtagctt
ttgcggtgaaaacgaatgtgagctactgtggtgccctggatgaggatgtgcccgttcccagcacagccat
ctcctttgacgccaaggactttcttcacattaaagagaaatataacaatgattggtggataggaaggctg
gtgaaggagggctgtgagatcggcttcatcccaagtccactgcgtttggagaacatacggattcaacagg
aacagaaaagaggccgttttcatggcgggaaatcgagtggaaactcttcctccagtctgggagaaatggt
gtcaggaacatttcgagcaactcccacaacaacagcaaaacagaagcagaaagtgacggagcacattcct
ccgtatgatgttgtgccatcaatgcgtcctgtggtgttagtagggccatcactgaaaggttatgaggtaa
cagacatgatgcagaaagccctctttgatttcctgaagcacaggtttgatgggaggatatcaataaccag
agtgacagctgacatttctcttgctaagaggtctgtcctcaacaatcctagcaagagagcaataattgaa
cgttcaaacaccagatccagcctagcggaagtacagagtgaaattgaaagaattttcgagttggcaagat
ctttgcaattggttgttctcgatgcagacaccatcaatcacccagcacagctgataaagacatcgttagc
gcccatcatcgtccacgtgaaggtctcgtccccaaaggttttacagcggctgattaagtccagaggaaag
tcccagagtaaacacttgaatgttcaactggtggcagctgataaactggcccagtgtccccctgaaatgt
ttgatgttatattagatgagaatcaacttgaggatgcctgtgaacatctgggagagtacctggaggcata
ttggcgtgccacccacacaagcagtagcacccctatgaccccattgctggggaggaacgtgggctccaca
gctctctcaccgtatcccacagcaatctctggactacagagtcagcggatgaggcatagcaaccactcta
cagagaattctccaattgaaagacgaagcctaatgacctctgatgaaaattaccacaatgagagggcccg
caagagtaggaaccgcttgtcctccagctcccagcacagccgagatcactaccctctggtggaagaagat
tatcctgactcataccaggacacttacaaaccccataggaaccgaggctcacctgggggatgcagccatg
actcccgacataggctttga

SEQ ID NO: 10 describes the Human nucleic acid sequence for Ca_{V}β3 (NM 000725.4).

| (SEQ ID NO: 10) | | | | | |
|---|---|---|---|---|---|
| ccccttcctc | cccgccgcca | cggccccgta | ggtgctcggg | gacccacctt | ccacctagca |
| cgggttcgtt | cccctctccc | ggcctggccc | gggctccccg | gtggccgccg | ccccctcgcc |
| gcccccgcct | tctcccgggg | agggggtcag | gtggggggc | actattgttg | taggagccgg |
| cgccagattc | ctcagccgcg | ctcggggtgg | gaccggctgg | gtttgggggg | gtggggtggg |
| gggagcggtg | atctgagctc | cgagcagctg | gtcttcgcgg | ctcgctccct | ccttcgcgct |
| ctctcgctcc | ctgccgccgc | ccgcagggct | gcggggctcg | gtggcatctc | ccgggcgcgg |
| cccgcagtcc | ttgcccctgc | ctccgggccg | ctcccgcccc | cggcgccgct | cgctcccccg |
| acccggactc | ccccatgtat | gacgactect | acgtgcccgg | gtttgaggac | tcggaggcgg |
| gttcagccga | ctcctacacc | agccgcccat | ctctggactc | agacgtctcc | ctggaggagg |
| accgggagag | tgcccggcgt | gaagtagaga | gccaggctca | gcagcagctc | gaaagggcca |
| agcacaaacc | tgtggcattt | gcggtgagga | ccaatgtcag | ctactgtggc | gtactggatg |
| aggagtgccc | agtccagggc | tctggagtca | actttgaggc | caaagatttt | ctgcacatta |
| aagagaagta | cagcaatgac | tggtggatcg | ggcggctagt | gaaagaggg | ggggacatcg |
| ccttcatccc | cagcccccag | cgcctggaga | gcatccggct | caaacaggag | cagaaggcca |
| ggagatctgg | gaacccttcc | agcctgagtg | acattggcaa | ccgacgctcc | cctccgccat |
| ctctagccaa | gcagaagcaa | aagcaggcgg | aacatgttcc | cccatatgac | gtggtgccct |
| ccatgcggcc | tgtggtgctg | gtgggaccct | ctctgaaagg | ttatgaggtc | acagacatga |
| tgcagaaggc | tctcttcgac | ttcctcaaac | acagatttga | tggcaggatc | tccatcaccc |
| gagtcacagc | cgacctctcc | ctggcaaagc | gatctgtgct | caacaatccg | ggcaagagga |
| ccatcattga | gcgctcctct | gcccgctcca | gcattgcgga | agtgcagagt | gagatcgagc |
| gcatatttga | gctggccaaa | tccctgcage | tagtagtgtt | ggacgctgac | accatcaacc |
| acccagcaca | gctggccaag | acctcgctgg | cccccatcat | cgtctttgtc | aaagtgtcct |
| caccaaaggt | actccagcgt | ctcattcgct | cccgggggaa | gtcacagatg | aagcacctga |
| ccgtacagat | gatggcatat | gataagctgg | ttcagtgccc | accggagtca | tttgatgtga |
| ttctggatga | gaaccagctg | gaggatgcct | gtgagcacct | ggctgagtac | ctggaggttt |
| actgggggc | cacgcaccac | ccagcccctg | gccccggact | tctgggtcct | cccagtgcca |
| tccccggact | tcagaaccag | cagctgctgg | gggagcgtgg | cgaggagcac | tccccccttg |
| agcgggacag | cttgatgccc | tctgatgagg | ccagcgagag | ctcccgccaa | gcctggacag |
| gatcttcaca | gcgtagctcc | cccacctgg | aggaggacta | tgcagatgcc | taccaggacc |
| tgtaccagcc | tcaccgccaa | cacacctcgg | ggctgcctag | tgctaacggg | catgaccccc |
| aagaccggct | tctagcccag | gactcagagc | acaaccacag | tgaccggaac | tggcagcgca |
| accggccttg | gcccaaggat | agctactgac | agcctcctgc | tgccctaccc | tggcaggcac |
| aggcgcagct | ggctgggggg | cccactccag | gcagggtggc | gttagactgg | catcaggctg |
| gcactaggct | cagcccccaa | aaccccctgc | ccagccccag | cttcagggct | gcctgtggtc |
| ccaaggttct | gggagaaaca | ggggaccccc | tcacctcctg | ggcagtgacc | cctactaggc |
| tcccattcca | ggtactagct | gtgtgttctg | cacccctggc | accttcctct | cctcccacac |
| aggaagctgc | cccactgggc | agtgccctca | ggccaggatc | cccttagcag | ggtccttccc |
| accagactca | gggaagggat | gccccattaa | agtgacaaaa | gggtggggtg | tgggcaccat |
| ggcatgagga | agaaacaagg | tccctgagca | ggcacaagtc | ctgacagtca | agggactgct |
| ttggcatcca | gggcctccag | tcacctcact | gccatacatt | agaaatgaga | caatcaaagc |
| ccccccaggg | tggcacaccc | atctgtttgc | tggggtgtgg | cagccacatc | caagactgga |
| gcagcaggct | ggccacgctc | gggccagaga | gagctcacag | ctgaagctct | tggagggaag |
| ggctctcctc | accctgccag | gaagcttctt | aacatgtgac | aggaccaggg | accaggagca |
| tggtgaagcc | aagtggcaga | tgggagccaa | cctggatggg | ggtttgggga | aggagggcat |
| gtgtagcaga | gaacttaggg | gggcctcctt | gcctttctca | ttcttttgcc | ctgcatcctg |
| tcatttctgt | tcttgtccct | catacatctt | tggagaaccg | ggctccagac | tttgttccct |
| gactcatagc | tgccgcttgt | taggttaggg | ttagatgggg | agagacaggg | cacagaggac |
| ctgtctcccc | ggctactctt | gccttatggc | tctagtgtgt | gacctacaga | gcatgctcca |
| caagcccctg | cctcacctca | ctgtcatcac | taataaacat | catgcacagt | c |

SEQ ID NO: 11 describes the Human nucleic acid sequence for Ca_{V}β2 (NM 000724.4).

| (SEQ ID NO: 11) | | | | | |
|---|---|---|---|---|---|
| gcattgtgaa | gaaggcaatc | ataggcgagc | agccgcggga | gcaggaacag | cagcgtgcta |
| agaagcagtc | acataaacag | cagcaggagt | aggcctcctg | cttttcaaaa | gcagagtact |
| gcagggtcgc | gaaatgcaag | acactcagat | gtttgaaaat | ctcccgagtt | gagaatggct |
| actgtaaaag | cgtcaccaag | aaactctgac | gatctggaca | gtcctaactc | tgtgttagca |
| atacttactt | ccggaaaatt | aatgctactt | cttgtagatt | tttgcaaata | ggaaaccccc |
| ttgaagaaga | tctcaaatta | cgccccccac | ccccaaaaaa | agacaaacag | gggagaacaa |
| agttttggca | tgcctgcagg | aacggtggct | tttttagaaa | ctacctagga | ggcagaagct |
| aagtgatttg | ctcatgcctc | ttacctggga | gtagaaggtg | ggaagaaatg | gaccgaggct |
| gtgacgagaa | gacaaggcac | agtgcagctt | ggtgaagcca | cacgctgact | gcgttctgcc |
| ccctcttcat | gcagtgctgc | gggctggtgc | atcgccggcg | agtacgggtg | tcctatggtt |
| cggcagactc | ctacactagc | cgtccatccg | attccgatgt | atctctggag | gaggaccggg |
| aggcagtgcg | cagagaagcg | gagcggcagg | cccaggcaca | gttggaaaaa | gcaaagacaa |
| agcccgttgc | atttgcggtt | cggacaaatg | tcagctacag | tgcggcccat | gaagatgatg |
| ttccagtgcc | tggcatggcc | atctcattcg | aagcaaaaga | ttttctgcat | gttaaggaaa |
| aatttaacaa | tgactggtgg | atagggcgat | tggtaaaaga | aggctgtgaa | atcggattca |
| ttccaagccc | agtcaaacta | gaaaacatga | ggctgcagca | tgaacagaga | gccaagcaag |
| ggaaattcta | ctccagtaaa | tcaggaggaa | attcatcatc | cagtttgggt | gacatagtac |
| ctagttccag | aaaatcaaca | cctccatcat | ctgctataga | catagatgct | actggcttag |
| atgcagaaga | aaatgatatt | ccagcaaacc | accgctcccc | taaacccagt | gcaaacagtg |
| taacgtcacc | ccactccaaa | gagaaaagaa | tgcccttctt | taagaagaca | gagcacactc |
| ctccgtatga | tgtggtacct | tccatgcgac | cagtggtcct | agtgggccct | tctctgaagg |
| gctacgaggt | cacagatatg | atgcaaaaag | cgctgtttga | ttttttaaaa | cacagatttg |
| aagggcggat | atccatcaca | agggtcaccg | ctgacatctc | gcttgccaaa | cgctcggtat |
| taaacaatcc | cagtaagcac | gcaataatag | aaagatccaa | cacaaggtca | agcttagcgg |
| aagttcagag | tgaaatcgaa | aggatttttg | aacttgcaag | aacattgcag | ttggtggtcc |
| ttgacgcgga | tacaattaat | catccagctc | aactcagtaa | aacctccttg | gcccctatta |
| tagtatatgt | aaagatttct | tctcctaagg | ttttacaaag | gttaataaaa | tctcgaggga |
| aatctcaagc | taaacacctc | aacgtccaga | tggtagcagc | tgataaactg | gctcagtgtc |
| ctccagagct | gttcgatgtg | atcttggatg | agaaccagct | tgaggatgcc | tgtgagcacc |
| ttgccgacta | tctggaggcc | tactggaagg | ccacccatcc | tcccagcagt | agcctcccca |
| accctctcct | tagccgtaca | ttagccactt | caagtctgcc | tcttagcccc | accctagcct |
| ctaattcaca | gggttctcaa | ggtgatcaga | ggactgatcg | ctccgctcct | atccgttctg |
| cttcccaagc | tgaagaagaa | cctagtgtgg | aaccagtcaa | gaaatcccag | caccgctctt |
| cctcctcagc | cccacaccac | aaccatcgca | gtgggacaag | tcgcggcctc | tccaggcaag |
| agacatttga | ctcggaaacc | caggagagtc | gagactctgc | ctacgtagag | ccaaaggaag |
| attattccca | tgaccacgtg | gaccactatg | cctcacaccg | tgaccacaac | cacagagacg |
| agacccacgg | gagcagtgac | cacagacaca | gggagtcccg | gcaccgttcc | cgggacgtgg |
| atcgagagca | ggaccacaac | gagtgcaaca | agcagcgcag | ccgtcataaa | tccaaggatc |
| gctactgtga | aaaggatgga | gaagtgatat | caaaaaaacg | gaatgaggct | ggggagtgga |
| acagggatgt | ttacatccgc | caatgagttt | tgcccgtttg | tgtttttttt | tttttttttt |
| tgaagtcttg | tataactaac | agcatcccca | aaacaaagtc | tttggggtct | acactgcaat |
| catatgtgat | ctgtcttgta | atattttgta | ttattgctgt | tgcttgaata | gcaatagcat |
| ggatagagta | ttgagatact | ttttcttttg | taagtgctac | ataaattggc | ctggtatggc |
| tgcagtcctc | cggttgcata | ctggactctt | caaaaactgt | tttgggtagc | tgccacttga |
| acaaaatctg | ttgccaccca | ggtgatgtta | gtgttttaag | aaatgtagtt | gatgtatcca |
| acaagccaga | atcagcacag | ataaaaagtg | gaatttcttg | tttctccaga | tttttaatac |
| gttaatacgc | aggcatctga | tttgcatatt | cattcatgga | ccactgtttc | ttgcttgtac |
| ctctggctga | ctaaatttgg | ggacagattc | agtcttgcct | tacacaaagg | ggatcataaa |
| gttagaatct | attttctatg | tactagtact | gtgtactgta | tagacagttt | gtaaatgtta |
| tttctgcaaa | caaacacctt | cttattatat | atataatata | tatatatatc | agtttgatca |
| cactatttta | gagtcttaat | gccaagtcag | cagatttgct | ttatgaatta | cagggactag |
| aaatgcccac | attcaggaaa | tttgtaataa | cattgtctag | acacctatcc | tcattctagt |
| agaaagtgtg | tacatactgt | aaatatgtgt | gattgcttga | cttgaaaagg | tttgaattct |
| gaatgttata | ccatccttgt | aagtaagttt | gtaatttcca | ccataaatta | tggtaaatat |
| aaaactccag | aggttgttct | actccataca | gttcacactg | attgtgacac | attcttagta |
| gctagtgtct | gttctagtca | ctgcactgga | gtctacgagc | cggaactcgc | tatatgcacg |
| tgtgtgtgtc | cgtatgtaag | aaagtgtgca | ccgagtgact | gaatggttga | gatgaattgg |
| aatgctgaag | actaacgaag | aaactagaga | ctgatatcga | gcattctgcc | cacctcgctc |
| tgtatttaat | taattgtgct | atatgttgct | ttaacaaccc | attgagcagt | cagggaatgt |
| gagtaagctt | gctgccaaag | gtaactagga | aagcattcat | ctgctgcctc | cttgtttttg |
| ctcctagaga | gtgaaaatac | aggcaatttt | actgtgagtg | tttcactgga | aatgtacaat |
| ctttgtgtgt | tagagtattt | gttttagtaa | gaaatgttta | cacagcttgt | ggaattattt |
| cgtgggaaaa | taaattttta | taacttctcc | cacttcaatt | tctaaccttg | cctattgttc |
| ctgttgtttg | tttacctccc | agttacctac | cattcctccc | caccaccgac | tccagcaggt |
| tcactgtctg | tcagaaccca | gaagtgcttc | ttataaccaa | agtttctgtt | cttcagaaga |
| aatcagggca | aaatggggtg | acttgaagtg | aataaaatgt | taagaatatt | atcctacata |
| agacatgtac | acagaagggg | aaccttgaag | acattatctc | catgcctcaa | ttacactgct |
| gtaagaagct | tacaatgtca | tcatgtttaa | ggctaagacc | ttttccgtgt | ctcaagtgta |
| tattttgtcc | agttataact | ggatggtaag | acagtattaa | gagtgcaagt | acctggcact |
| tgaagtttgt | cccaggaaaa | tgcctgtgta | taattaccta | acttcagatc | tgcacattaa |
| cttatttaac | aaaataaatc | agccgggcgc | agtggctcat | gcctgtaatc | ccagcaattt |
| gggaggctga | ggagggtgga | tcacctgagg | tcaggagttc | aagaccagcc | tggccaacgt |
| ggcgaaaccc | cgtctctact | aaaaagataa | aaaattagcc | aggtgtggtg | gtgcacacct |
| gtaatcccag | ctacttggga | ggctgaggca | ggagaattgc | ttgaacccag | gaggtggagc |
| ctgcagtaag | ccaagatcac | accactgtac | tccagcctgg | gtgacagagt | gagactccat |
| ctccaaaaaa | gaaaaaaaaa | caaaaaaaa | aaaaaactat | ccagcaaaat | tataaaatct |
| actttgtttt | gcttccttga | ttattccaag | ttcttaccaa | atattcttag | ccttttataa |
| gtaaaactgt | tttacattta | actccttatt | tcccttaccc | ccaagaaaac | gcaatattaa |
| aaatgattaa | gctggggtgg | tagagtatac | ctgtagaccc | agctacttgg | gaggctgaga |
| taggaggatc | ccttgagccc | aggaggtgga | ggctgcagtg | aaccgtgact | gtaccaatgc |
| actccagcct | ggtgacaacc | tggtgtttaa | aaaaagaaaa | taaaaattag | ttacatatat |
| aatgttttat | cagcttatat | atgaaaaaat | ttccctcagt | tcgttaatta | gccctacact |
| gtttacataa | atttatttac | tgagtataaa | atcagtagtc | ataataaact | tgacaattct |
| tacattggta | ggaaaccata | ttctaattaa | aattgaattt | ataaaatatt | tctgataaaa |
| cttttgtcat | ggtaagagat | tcacaattat | tagttcaatc | ctttattatt | tcatcccaat |
| taatgatttt | agaatattta | aattcttgaa | acatagctag | tatttatctt | acttgctgct |
| atcattaatc | cctttcaaaa | agtagtggtg | agagtttcca | tcttttttat | tttgtatcta |
| gattcttagc | ttgcacttta | ctgtagaaca | tattagtgca | aatcagaata | ttcctcaaag |
| aactagcttg | aaggatttga | cataagagcc | gctatatgtg | aaaaactgta | tagtggacat |
| atgtgtaaac | tgtgtagtaa | atctgtaaat | gaggaaataa | tctcaagggc | aatgggatat |
| ttactgacct | gcggaatgta | aagttacagt | cttttcacac | aacaaagget | taaccttaac |
| ctactcagtt | gtagagccat | ttttgtagtc | aacctagaaa | atgctggaaa | tgtatttaat |
| agtttttttt | tttttttttt | ttggtcatat | ccatttcagt | ctttcctatg | ctctttctct |
| actgctcatt | taagttactg | ttacaaaaag | gtgctgctaa | atgtaggatg | tcttagtcat |
| gttgtacttt | gggacaatgc | cacattttta | acatggtctg | ctatgcattc | ctgttaaaca |
| ttcactgtca | gctacactga | actgtctaac | aaacctggtt | taaagtaatt | catataaaac |
| aaataaagag | ctggcttctg | cactgtttat | tagtagtagt | attaattgcc | atgttagaaa |
| tgattcatgt | agttgagact | atattaaata | ttcaacactt | cccagctgca | gggcatcttt |
| cttggggcaa | tgctaaatac | tttatgttcc | tttagtaatc | ttcctattat | gtctgattaa |
| aaaaaaagat | acctccacgt | agataaaata | ttttattcaa | cgtgagctct | agcagaataa |
| tctgggtcag | ttatgagttc | tggatcaaga | gtttaatttc | tgctgcgctg | tacttttaac |
| tatttgtact | ttgcttcatt | atttaaaaca | tgacacaggg | ttttaaagta | aatgtactca |
| ctgttgaact | taagtacttc | ttctgtttca | tagaaatgtt | aatattaaat | gttaaaatat |
| atattaattt | tccctcaca | | | | |

SEQ ID NO: 12 describes the Human nucleic acid sequence for Ca_{V}β1 (NM_000723.5).

| (SEQ ID NO: 12) | | | | | |
|---|---|---|---|---|---|
| agtggagaga | gcgggaggag | cgagggaagg | caggaaggag | gcagccgaag | gccgagctgg |
| gtggctggac | cgggtgctgg | ctgcgccgcg | ctgctttegg | ctcccacggc | ctctcccatg |
| cgctgaggga | gcccggctgg | gccgggccgg | cggcgggagg | ggaggctcct | ctccatggtc |
| cagaagacca | gcatgtcccg | gggcccttac | ccaccctccc | aggagatccc | catggaggtc |
| ttcgacccca | gcccgcaggg | caaatacagc | aagaggaaag | ggcgattcaa | acggtcagat |
| gggagcacgt | cctcggatac | cacatccaac | agctttgtcc | gccagggctc | agcggagtcc |
| tacaccagcc | gtccatcaga | ctctgatgta | tctctggagg | aggaccggga | agccttaagg |
| aaggaagcag | agcgccaggc | attagcgcag | ctcgagaagg | ccaagaccaa | gccagtggca |
| tttgctgtgc | ggacaaatgt | tggctacaat | ccgtctccag | gggatgaggt | gcctgtgcag |
| ggagtggcca | tcaccttcga | gcccaaagac | ttcctgcaca | tcaaggagaa | atacaataat |
| gactggtgga | tcgggcggct | ggtgaaggag | ggctgtgagg | ttggcttcat | tcccagcccc |
| gtcaaactgg | acagccttcg | cctgctgcag | gaacagaagc | tgcgccagaa | ccgcctcggc |
| tccagcaaat | caggcgataa | ctccagttcc | agtctgggag | atgtggtgac | tggcacccgc |
| cgccccacac | cccctgccag | tgccaaacag | aagcagaagt | cgacagagca | tgtgcccccc |
| tatgacgtgg | tgccttccat | gaggcccatc | atcctggtgg | gaccgtcgct | caagggctac |
| gaggttacag | acatgatgca | gaaagcttta | tttgacttct | tgaagcatcg | gtttgatggc |
| aggatctcca | tcactcgtgt | gacggcagat | atttccctgg | ctaagcgctc | agttctcaac |
| aaccccagca | aacacatcat | cattgagcgc | tccaacacac | gctccagcct | ggctgaggtg |
| cagagtgaaa | tcgagcgaat | cttcgagctg | gcccggaccc | ttcagttggt | cgctctggat |
| gctgacacca | tcaatcaccc | agcccagctg | tccaagacct | cgctggcccc | catcattgtt |
| tacatcaaga | tcacctctcc | caaggtactt | caaaggctca | tcaagtcccg | aggaaagtct |
| cagtccaaac | acctcaatgt | ccaaatagcg | gcctcggaaa | agctggcaca | gtgcccccct |
| gaaatgtttg | acatcatcct | ggatgagaac | caattggagg | atgcctgcga | gcatctggcg |
| gagtacttgg | aagcctattg | gaaggccaca | cacccgccca | gcagcacgcc | acccaatccg |
| ctgctgaacc | gcaccatggc | taccgcagcc | ctggctgcca | gccctgcccc | tgtctccaac |
| ctccagggac | cctaccttgc | ttccggggac | cagccactgg | aacgggccac | cggggagcac |
| gccagcatgc | acgagtaccc | aggggagctg | ggccagcccc | caggccttta | ccccagcagc |
| cacccaccag | gccgggcagg | cacgctacgg | gcactgtccc | gccaagacac | ttttgatgcc |
| gacacccccg | gcagccgaaa | ctctgcctac | acggagctgg | gagactcatg | tgtggacatg |
| gagactgacc | cctcagaggg | gccagggctt | ggagaccctg | cagggggcgg | cacgccccca |
| gcccgacagg | gatcctggga | ggacgaggaa | gaagactatg | aggaagagct | gaccgacaac |
| cggaaccggg | gccggaataa | ggcccgctac | tgcgctgagg | gtgggggtcc | agttttgggg |
| cgcaacaaga | atgagctgga | gggctgggga | cgaggcgtct | acattcgctg | agaggcaggg |
| gccacacggc | gggaggaagg | gctctgagcc | caggggaggg | gagggagcga | ggggctcaca |
| cctgacatgt | attcgcctcc | agggggcgct | gtctccctcc | tttcagatgc | ctttgctcaa |
| agcttggggt | ttctttggtg | ttaccatccc | agctcccggg | aggcccttaa | gccccagctg |
| tcggttttta | cctgcctgtt | gtggatggat | gggggatacc | cacctttctg | aagtgtcccc |
| tttctcccat | cttaaggggc | tctcctccct | caccctccta | gagaaaaggt | gcacttcctt |
| aactctttct | actcggggcc | ctaagtgacg | gtcctaagtg | ggatggctct | ccttctccca |
| agctgcagta | ctggggaagg | gctgggcgct | tttcctggaa | agggaggcca | cagattcttt |
| cccatggggg | ctctcttccc | cagaccccag | atccaaggtc | cctcaccctg | cctgcccctt |
| cctcccagct | tcctggcagc | atcgtctggt | cggtgaaagc | catagcatgg | acaccccatg |
| gggagcttgt | cttggggagg | gttctgggtg | gaagctggca | ggcatacagc | accctctacc |
| ctccgtggcc | atggcaacgt | ccagggccca | gaaccctgag | gagtgagcgg | ccgagacgct |
| gctccccacc | ccccacctcc | atgcctcagc | ctttgcctac | cccaggaatg | agcttggcct |
| ccaacatccc | ttgcctgctg | ccattagtag | agggggcccc | tctgcatctg | agccccccat |
| ccctgtgcca | cctgggtgtg | gagcccatgg | aacactctgg | tccgcctcat | tttaaaccaa |
| aaaactgctc | cttcaccctc | accctgaggc | cccaggggag | aggacccgtg | ggatggtgcc |
| caggggttgc | cttgggacct | cggatctcct | ctggggggct | tggctgctgc | tgttgctgct |
| ctgtatttgc | ctctcgtgat | tctgtttgtt | acccatgttc | acttccccca | ggagaggcct |
| tggtaccccc | tctcccctgg | ggcatccctt | tgccttggca | tecctgtage | ccagcaaccc |
| tgcccctccc | cagcatccca | gctgggccag | agagagccga | gtgtgccaac | aaggactggg |
| gcctgcccgg | ctgcccgcct | cagggatggg | cacctcatgc | ctgtctcgcc | acctcctgtg |
| ccaatgtccc | accctccacc | tgggggggg | gtgcagcttc | cacttactga | ttagaagaca |
| ccactgccct | cccttccccc | ctccctgtct | ggtgtcctgt | gcccccatct | gtctgtctat |
| atttgtctgt | actcccctag | gagaagtatt | ttgccatata | taaaaccact | gtcctgtcct |
| ttgtggctgc | ctcccaagcc | tgcttctttg | tcctcgccac | atagtcgtca | gcgtaggcac |
| ctgggagctg | ctgatatgca | cggggagttg | aaagggtggg | tgcctgaaga | tgttgtgccc |
| tgagtcattg | actcaaaaga | aaagatgatc | ctttgatttt | ggccctctga | tgtattgtgc |
| ccaagccagg | agctgcttgg | gcagtcccag | ctccacactg | gccctgagcc | ccttcactta |
| cctgtctctc | cacaagtaga | gccaaaggca | atgggaagct | caatgttgct | cagtgggtga |
| gatccagacc | cactggtgca | atgtcttaaa | tacacatgac | tgtttttctg | c |

SEQ ID NO: 44 describes a modified human nucleic acid sequence for Ca_{V}β1.

(SEQ ID NO: 44)
atggtccagaagagcggcatgtcccggggcccttacccaccttcccaagagatccctatggaggtcttcg
accccagcccacagggcaaatacagcaagaggaaagggcggttcaaaaggtcagatgggagcacctcctc
agataccacatccaacagctttgtccgtcagggctcagcagagtcctacacgagccggccgtcagactcc
gacgtgtccctggaggaggaccgggaagccttaaggaaggaggcagagcgccaggccttagcccagctcg
agaaagccaagaccaaaccagtggcctttgctgttcggacaaatgttggctacaatccgtctccaggaga
tgaggtgccagtgcagggagtggccatcacctttgagcccaaggacttcctgcacatcaaggagaaatac
aataatgactggtggatcgggaggctggtgaaggaaggctgtgaggttggtttcatccccagccctgtca
aactggacagccttcgcctgctgcaggaacagacgctgcgccaaaaccgcctcagctccagcaagtcagg
tgacaactccagttccagtctgggagatgtggtgactggcacccgccgccccacaccccccgccagtgcc
aaacagaagcagaagtcgacagagcacgtgcccccctatgacgtggtgccttccatgaggcccatcatcc
tggtgggaccatcgctcaagggctatgaggtaactgacatgatgcagaaggcgttgtttgacttcttgaa
gcatcggtttgatggcaggatttccatcacccgggtaacggctgacatttccctggccaagcgctcggtc
ctcaacaaccccagcaaacacattatcatcgagcgctccaacactcgctccagcctggctgaggtacaga
gtgaaatcgagcggatcttcgagctggcccggaccttgcagctggttgccctggacgccgacaccatcaa
ccacccagcccagctctctaaaacctcgctggcccccatcattgtttacatcaagatcacatcccccaag
gtactgcagaggctcatcaaatcccgagggaagtctcaatccaaacacctcaatgtccaaatagcagcct
cggagaagctggcacagtgtccccccgaaatgtttgacataatcctggacgagaaccaattggaagatgc
ctgcgagcacctggcggagtacttggaagcctactggaaggccacacacccgcctagcagcacgccaccc
aatccgctgctgaaccgcaccatggctaccgccgctctggctgccagccctgcccccgtctccaacctcc
agggaccctaccttgcttccggggaccagccgctggaccgggccactggggagcatgccagtgtgcacga
gtaccccggggagctgggccagcccccaggcctttaccccagcaaccacccacctggccgggcaggcacc
ctgcgggcgctatcccgccaagacacctttgatgctgacacccccggcagccgaaactctgcctacacgg
agccaggagactcgtgtgtggacatggagacagacccctcagagggcccagggcctggagaccctgcagg
gggaggtacaccaccagctcggcagggctcctgggaagaggaggaagattatgaggaggagatgaccgac
aacaggaaccggggccggaataaggcccgctactgtgcggagggtggtgggccggttctggggcgcaata
agaatgagctggagggctggggacaaggcgtctacatccgctga

In some embodiments, the second domain is encoded by a nucleic acid sequence having at least 90%, 91%, 92%, 93%, 94%, 95%, 96%, 97%, 98% or 99% identity to a nucleic acid sequence selected from the group consisting of SEQ ID NO: 9-12 and 42-44. In some embodiments, the second domain is encoded by a nucleic acid sequence having at least 95%, 96%, 97%, 98% or 99% identity to a nucleic acid sequence selected from the group consisting of SEQ ID NO: 9-12 and 42-44. In some embodiments, the second domain is encoded by a nucleic acid sequence having at least 97%, 98% or 99% identity to a nucleic acid sequence selected from the group consisting of SEQ ID NO: 9-12 and 42-44. In some embodiments, the second domain is encoded by a nucleic acid sequence having 100% identity to a nucleic acid sequence selected from the group consisting of SEQ ID NO: 9-12 and 42-44.

In some preferred embodiments, the second domain is encoded by a nucleic acid sequence of SEQ ID NO: 9.

In some preferred embodiments, the second domain is encoded by a nucleic acid sequence of SEQ ID NO: 42.

In some embodiments, a polypeptide, e.g., the fusion protein, as described herein can comprise at least one peptide bond replacement. For example, the fusion protein as described herein can comprise at least one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide, e.g., the fusion protein, as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). In some embodiments, the fusion protein as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a polypeptide, e.g. the fusion protein, can be modified, e.g. by the addition of a moiety to one or more of the amino acids that together comprise the peptide. In some embodiments, a polypeptide as described herein can comprise one or more moiety molecules, e.g., 1 or more moiety molecules per polypeptide, 2 or more moiety molecules per polypeptide, 5 or more moiety molecules per polypeptide, 10 or more moiety molecules per polypeptide or more moiety molecules per polypeptide. In some embodiments, a polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a polypeptide can be increased by the addition of moieties, e.g. PEG, albumin, or other fusion partners (e.g. Fc fragment of an immunoglobulin).

In some embodiments, the fusion protein described herein can be optimized for delivery to any cell.

In some embodiments, the fusion protein described herein can be optimized for delivery to neuronal or non-neuronal cells. In some embodiments, the neuronal or non-neuronal cells are part of the central nervous system. In some embodiments, the neuronal or non-neuronal cells are part of the peripheral nervous system. Non-neuronal cells include but are not limited to glial cells, e.g., astrocytes and oligodendrocytes, insulin producing cells, e.g., islet or beta cells.

In some embodiments, the fusion protein described herein can be optimized for delivery to endocrine glands. Endocrine glands include but are not limited to pituitary gland, pineal gland, thymus gland, thyroid gland, adrenal gland, and the pancreas.

### Modification for Crossing BBB

For optimal delivery to neuronal or non-neuronal cells in the brain, the fusion protein described herein can be optimized to cross the blood brain barrier (BBB). In some embodiments, the fusion protein is linked to an agent that is endogenously transported across the BBB, e.g., insulin, transferrin, insulin like growth factor (IGF), leptin, low density lipoprotein (LDL) and fragments or peptidomimetics or derivatives thereof, which can undergo receptor-mediated transport (RMT) across the BBB in vivo.

In some embodiments, the fusion protein is linked to a peptidomimetic monoclonal antibody (MAb) of an agent that is endogenously transported across the BBB, e.g., mAbs for the insulin receptor, the transferrin receptor, the IGF receptor, the leptin receptor, or the LDL receptor. In some embodiments of any of the aspects, the fusion protein is linked to a cationic substance that can cross the BBB by adsorption-mediated transcytosis or endocytosis.

In some embodiments, the fusion protein is linked to a cell penetrating peptide (CPP). CPPs are short peptides that facilitate cellular intake and uptake of molecules through endocytosis. CPPs typically have an amino acid composition that either contains a high relative abundance of positively charged amino acids such as lysine or arginine or has sequences that contain an alternating pattern of polar, charged amino acids and non-polar, hydrophobic amino acids. These two types of structures are referred to as polycationic or amphipathic, respectively. A third class of CPPs are the hydrophobic peptides, containing only apolar residues with low net charge or hydrophobic amino acid groups that are crucial for cellular uptake. In some embodiments, the CPP is selected from pVEC, SynB3, Tat 47-57, transportan 10.

In some embodiments, the CPP is Rabies Virus Glycoprotein, which is a 29-amino-acid cell penetrating peptide derived from a rabies virus glycoprotein that can cross the blood-brain barrier (BBB) and enter brain cells. RVG peptide is successfully used to carry a variety of cargos into brain cells such as plasmids, siRNAs, proteins, and nanoparticles; see e.g., US Patent Publication 2018-0028677A1, the content of which is incorporated herein by reference in its entirety.

In some embodiments, the fusion protein is linked to a BBB-shuttle. BBB-shuttles are peptides designed to target BBB receptors in order to gain access to the brain by transcytosis. In some embodiments, the BBB-shuttle is selected from one of SEQ ID NOs: 13-37 or an amino acid sequence that is at least 80%, at least 85%, at least 90%, at least 91%, at least 92%, at least 93%, at least 94%, least 95%, at least 96%, at least 97%, at least 98%, at least 99%, at least 99.5% or more identical to one of SEQ ID NOs: 13-37 that maintains the same function (e.g., BBB transcytosis) (see e.g., Table 1). In some embodiments of any of the aspects, nanoparticles, comprising the fusion protein, and the nanoparticles are linked to at least one BBB-shuttle. See e.g., McCully et al., Curr Pharm Des. 2018 April, 24(13): 1366-1376, the content of which is incorporated herein by reference in its entirety.

TABLE 3
Exemplary BBB-shuttles

| BBB- | | | SEQ ID |
|---|---|---|---|
| Shuttle | Target | Sequence | NO |
| Ang-2 | LDR 1 | TFFYGGSRGKRNNFKTEEY | 13 |
| | | Yeetkfnnrkgrsggyfft | 14 |
| ApoE | LDLR | LRKLRKRLLR | 15 |
| (141-150) | | | |
| B6 | hTrR | CGHKAKGPRK | 16 |
| Cyclic- | Integrin | RGDfK (cyclized peptide) | 17 |
| RGD | R | | |
| CDX | nAchR | FKESWREARGTRIERG | 18 |
| ^{D}CDX | nAchR | GreirtGraerwsekf | 19 |
| Enk | Opioid | YGGFLGGYTGFLS-O-beta-glucoside | 20 |
| Gly-copep | receptor | | |
| g7 | | GFtGFLS-(monosaccharide) | 21 |
| gH625 | | HGLASTLTRWAHYNALIRAFGGG | 22 |
| Gluthatione | Mrp/ | GSH | 23 |
| | Abcc | | |
| LPFFD | RAGE | LPFFD | 24 |
| MiniAp-4 | | Dap-KAPETALD (cyclized peptide); | 25 |
| | | Dap stands for diaminopropionic acid | |
| Penetratin | CPP | RQIKIWFQNRRMKWKK | 26 |
| RDP | nAchR | KSVRTWNEIIPSKGCLRVGGRCHPHVNGGGRRRRRRRRR | 27 |
| peptide | | | |
| RVG29 | nAchR | YTWMPENPRPGTPCDIFTNSRGKRASNGGGGGGC | 28 |
| CTX | MMP-2, | MCMPCFTTDHQMARKCDDCCGGKGRGKCYGPQCLCR | 29 |
| | Annexin | | |
| | A, Cl- | | |
| | channels | | |
| T7-HAI | TfR | HAIYPRH | 30 |
| TAT | AME | GRKKRRQRRRPPQGWC | 31 |
| | | YGRKKRRQRRR | 32 |
| TATre | AME | rrrqrrkkrGy | 33 |
| TGN | | TGNYKALHPHNG | 34 |
| THR | TfR | THRPPMWSPVWP | 35 |
| THRre | TfR | pwvpswmpprht | 36 |
| Peptide-22 | LDLR | C-MPRLRGC (cyclized peptide) | 37 |

The disclosure also provides a synthetic nucleic acid encoding a fusion protein described herein. The skilled person will understand that, due to the degeneracy of the genetic code, a given polypeptide can be encoded by different polynucleotides. These “variants” are encompassed herein. In some embodiments, the polynucleotide is mRNA.

Also provided herein is an expression cassette comprising the synthetic nucleic acid encoding a fusion protein described herein.

In some embodiments, a nucleic acid (e.g., mRNA encoding the fusion protein) is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of nucleic acid compounds useful in the embodiments described herein include, but are not limited to nucleic acids containing modified backbones or no natural internucleoside linkages. Nucleic acids having modified backbones include, among others, those that do not have a phosphorus atom in the backbone. For the purposes of this specification, and as sometimes referenced in the art, modified nucleic acids that do not have a phosphorus atom in their internucleoside backbone can also be considered to be oligonucleosides. In some embodiments of any of the aspects, the modified nucleic acid will have a phosphorus atom in its internucleoside backbone.

Modified nucleic acid backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified nucleic acid backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2-O—N(CH3)-CH2-, —CH2-N(CH3)-N(CH3)-CH2- and —N(CH3)-CH2-[wherein the native phosphodiester backbone is represented as —O—P—O—CH2-].

In other nucleic acid mimetics, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA).

In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The nucleic acid can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol. Canc. Ther. 6(3):833-843; Grunweller, A. et al., (2003) Nucleic Acids Research 31(12):3185-3193).

Modified nucleic acids can also contain one or more substituted sugar moieties. The nucleic acids described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO] mCH3, O(CH2)nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, nucleic acids include one of the following at the 2′ position: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleic acid, or a group for improving the pharmacodynamic properties of a nucleic acid, and other substituents having similar properties. In some embodiments of any of the aspects, the modification includes a 2′ methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O-CH2-O-CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the nucleic acid, particularly the 3′ position of the sugar on the 3′ terminal nucleotide or in 2′-5′ linked dsRNAs and the 5′ position of 5′ terminal nucleotide. Nucleic acids may also have sugar mimetics such as cyclobutyl moieties in place of the pentofuranosyl sugar.

A nucleic acid can also include nucleobase (often referred to in the art simply as “base”) modifications or substitutions. As used herein, “unmodified” or “natural” or “canonical” nucleobases include the purine bases adenine (A) and guanine (G), and the pyrimidine bases thymine (T), cytosine (C) and uracil (U). Modified or “non-canonical” nucleobases can include other synthetic and natural nucleobases including but not limited to as inosine, isocytosine, isoguanine, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. In some embodiments of any of the aspects, modified nucleobases can include d5SICS and dNAM, which are a non-limiting example of unnatural nucleobases that can be used separately or together as base pairs (see e.g., Leconte et. al. J. Am. Chem. Soc. 2008, 130, 7, 2336-2343; Malyshev et. al. PNAS. 2012. 109 (30) 12005-12010). In some embodiments of any of the aspects, the nucleic acid comprises any modified nucleobases known in the art, i.e., any nucleobase that is modified from an unmodified and/or natural nucleobase.

The preparation of the modified nucleic acids, backbones, and nucleobases described above are well known in the art.

Another modification of a nucleic acid featured in the invention involves chemically linking to the nucleic acid to one or more ligands, moieties or conjugates that enhance the activity, cellular distribution, pharmacokinetic properties, or cellular uptake of the nucleic acid. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

### Vectors, Viral Vectors, and Cells

Various aspects described herein are directed to vectors or viral vectors comprising a nucleic acid sequence encoding the fusion protein described herein, that are encoding the fusion protein described herein, or comprising the expression cassette described herein.

In some embodiments, the nucleic acid or expression cassette described herein is expressed in a recombinant expression vector or plasmid. As used herein, the term “vector” refers to a polynucleotide sequence suitable for transferring transgenes into a host cell. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc. See, for example, U.S. Pat. Nos. 4,980,285; 5,631,150; 5,707,828; 5,759,828; 5,888,783 and, 5,919,670, and, Sambrook et al, Molecular Cloning: A Laboratory Manual, 2nd Ed., Cold Spring Harbor Press (1989).

A “plasmid,” refers to a circular double stranded DNA loop into which additional DNA segments are ligated. Another type of vector is a viral vector; wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., pi-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

In some embodiments, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments, the vector or polynucleotide described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell. In some embodiments, the vector and/or nucleic acid sequence described herein is codon-optimized for expression in a bacterial cell. In some embodiments, the vector and/or nucleic acid sequence described herein is codon-optimized for expression in an E. coli cell.

In some embodiments of any of the aspects, the vector further comprises a promoter that is operatively linked to the nucleic acid sequence encoding the fusion protein. As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the fusion protein described herein is expressed in a cell, a variety of transcription control sequences (e.g., promoter/enhancer sequences) can be used to direct its expression. The promoter can be a native promoter.

In some embodiments the promoter can be constitutive, i.e., the promoter is unregulated allowing for continual transcription of its associated gene. Non-limiting examples of constitutive promoters include: cytomegalovirus (CMV) promoter, the strong synthetic CAG promoter, human elongation factor-1 alpha (EF-1alpha), silencing-prone spleen focus forming virus (SFFV), beta actin/ACTB promoter and the like.

In some embodiments, the nucleic acid encoding the fusion protein is operatively linked to an inducible promoter, which is active in the presence of the promoter activator or the absence of the promoter repressor, and inactive in the absence of the promoter inducer or the presence of the promoter repressor. Non-limiting examples of inducible promoters include: a doxycycline-inducible promoter, the lac promoter, the lacUV5 promoter, the tac promoter, the trc promoter, the T5 promoter, the T7 promoter, the T7-lac promoter, the araBAD promoter, the rha promoter, the tet promoter, an isopropyl β-D-1-thiogalactopyranoside (IPTG)-dependent promoter, an AlcA promoter, a LexA promoter, a temperature inducible promoter (e.g., Hsp70 or Hsp90-derived promoters), or a light inducible promoter (e.g., pDawn/YFI/FixK2 promoter/CI/pR promoter system).

In some embodiments, the promoter comprises a tissue-specific promoter, e.g., specific to the brain, the central nervous system, the peripheral nervous system, the pancreas, the pituitary, the pineal, the thymus, the thyroid, the adrenal glands, neurons, glia, islet cells, and the like. In some embodiments, the promoter comprises a nervous tissue-specific promoter. In some embodiments of any of the aspects, the nervous tissue-specific promoter is a neuron-specific promoter. In some embodiments, the nervous tissue-specific promoter is a non-neuronal promoter. In some embodiments, the promoter is a glia cell promoter. In some embodiments of any of the aspects, the neuron-specific promoter is the synapsin promoter (e.g., Human synapsin 1 promoter) or the caMK2a promoter (e.g., human Calcium/Calmodulin Dependent Protein Kinase II Alpha promoter). The synapsin I promoter has been used to achieve highly neuron-specific long-term transgene expression in vivo. The CaMK2a promoter is a neuron-specific promoter with expression restricted to excitatory neurons in the neocortex and hippocampus, including pyramidal neurons. In some embodiments, the promoter comprises an endocrine system-specific promoter. In some embodiments, the endocrine system-specific promoter is specific for pancreatic cells. In some embodiments, the endocrine system-specific promoter is insulin-promoter-factor 1. In some embodiments, the endocrine system-specific promoter is specific for the thyroid. In some embodiments, the endocrine system-specific promoter is specific for the thymus. In some embodiments, the endocrine system-specific promoter is specific for the pituitary gland. In some embodiments, the endocrine system-specific promoter is specific for the pineal gland. In some embodiments, the endocrine system-specific promoter is specific for the adrenal gland. Organ and cell specific promoters are known to those skilled in the art.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

In some embodiments, the vector is a viral vector. Accordingly, in one aspect, described herein is a viral vector comprising a vector or nucleic acid as described herein, e.g., encoding at least the fusion protein. In some embodiments, the viral vector is an adenovirus-associated virus (AAV). AAV vectors are non-enveloped 25 nm particles with a foreign DNA packaging capacity of 4.6 kb. They have been clinically demonstrated to be safe in the CNS, and certain serotypes display strong neural tropism. In some embodiments, the AAV is serotype AAV2/1, which is a hybrid of serotypes 2 and 1 and exhibits neuronal tropism and expression. In some embodiments, the AAV is AAV1, which is an efficient viral vector in various brain regions and leads to extensive anterograde and retrograde expression. In some embodiments, the AAV is AAV2, which in the brain, is strongly neuron-specific and can also transduce the thyroid. In some embodiments, the AAV is serotype AAV2/1, AAVDJ8, AAV9, AAV8, AAVDJ9, or AAV1, which have tropism for primary murine astrocyte and neuronal cell cultures, as well as pancreatic cells. In some embodiments, the AAV is serotype AAV2/1, AAVDJ8, or AAV9, which have tropism for the olfactory bulb, striatum, cortex, hippocampus, substantia nigra (SN) and cerebellum, as well as pancreatic cells. In some embodiments, the AAV is AAV serotype 6 (AAV6), which is retrogradely transported from terminals to neuronal cell bodies. In some embodiments, the AAV is AAV7. In some embodiments, the AAV is AAV8. In some embodiments, the AAV is AAV9. Infusion (e.g., through the cisterna magna) (CM) of either AAV7 or AAV9 is associated with a high level of cell transduction distributed throughout brain cortex and along the spinal cord, including dorsal root ganglia, corticospinal tracts, astrocytes, and neurons. In some embodiments, the AAV is a rhesus monkey AAV, designated as “AAVrh,” which exhibits CNS-tropism. In some embodiments, the AAV is AAVrh.10, AAVrh.39, rAAVrh.43, which are capable of crossing the blood-brain barrier (BBB). In some embodiments, the AAV has tropism for the brain and/or neurons, thus allowing delivery of the nucleic acid across the BBB and into the brain, e.g., where the fusion protein can be expressed under the control of the operatively linked promoter. See e.g., Hammond et al., PLoS One. 2017; 12(12): e0188830, the content of which is incorporated herein by reference in its entirety. In some embodiments, the AAV is used to transduce peripheral nervous system cells. In some embodiments, the AAV is used to transduce the endocrine system. In some embodiments, the AAV is used to transduce cells in the pituitary, pineal, thymus, thyroid, adrenal glands, or the pancreas. In some embodiments, the AAV is used to transduce insulin-producing cells.

In some embodiments, the viral vector is a herpes simplex virus (e.g., HSV-1). Herpes simplex virus type 1 (HSV-1) vectors are enveloped 100 nm particles with a foreign DNA packaging capacity of more than 100 kb. The greatest advantages are the high packaging capacity and natural neurotropism via retrograde axonal transport. In some embodiments of any of the aspects, the viral vector is a lentivirus (e.g., human immunodeficiency virus (HIV) or a self-inactivating (SIN) lentiviral vector). Lentiviral vectors are enveloped 100 nm particles with a foreign DNA packaging capacity of 9 kb. In some embodiments of any of the aspects, the lentivirus is pseudotyped with a glycoprotein that targets neurons or glial cells. Non-limiting examples of such glycoproteins include the glycoproteins from neurotropic virus such as vesicular stomatitis virus G (VSV-G), lymphocytic choriomeningitis virus (LCMV), rabies, or Mokola lyssavirus. See e.g., Gray et al., Ther Deliv. 2010, 1(4): 517-534, the content of which is incorporated herein by reference in its entirety. In some embodiments of any of the aspects, the lentivirus targets the peripheral nervous system cells. In some embodiments of any of the aspects, the lentivirus targets the endocrine system. In some embodiments of any of the aspects, the lentivirus targets cells in the pituitary, pineal, thymus, thyroid, adrenal glands, or the pancreas. In some embodiments, the lentivirus targets insulin producing cells.

In some embodiments, one or more of the recombinantly expressed nucleic acids encoding the fusion protein can be integrated into the genome of the cell. A nucleic acid molecule that encodes the fusion protein as described herein can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the fusion protein can may be accomplished by integrating the nucleic acid molecule into the genome or through stable episomes. For example, AAV is a virus that can be maintained in an extrachromosomal form (i.e., episome) in the nucleic of transduced cells. Vector integration of AAV has also been observed in various experimental settings, either at non-homologous sites where DNA damage may have occurred or by homologous recombination

Accordingly, in one aspect described herein is a cell, any nucleic acid as described herein, any expression cassettes described herein, any vector as described herein, or any viral vector as described herein, any of which comprise, encode, or express the fusion protein as described herein.

In one embodiment, the nucleic acid or expression cassette is transiently expressed in the cell. In one embodiment, the nucleic acid or expression cassette is constitutively expressed in the cell.

In some embodiments, the cell is a neuronal cell. In some embodiments, the cell is a neuronal cell (e.g., SH-SY5Y neuroblastoma cells; NT2 cells, such as NTERA-2 CL.D1 (NT2/D1) ATCC® CRL-1973™; PC-12 cells (e.g., ATCC® CRL-1721™)). In some embodiments, the cell is a primary neuronal culture cell (e.g., murine, rat, non-human primate, or human primary neuronal cultures). In some embodiments, the cell is a hippocampal cell. In some embodiments, the cell is a pyramidal cell. In some embodiments, the cell is a CA1 pyramidal cell. In some embodiments, the cell is an interneuron (e.g., a PV-IN or CCK-IN). In some embodiments, the cell is a CNS neuron. In some embodiments, the cell is a peripheral nervous system (PNS) neuron. In some embodiments, the cell is a motor neuron. In some embodiments of, the cell is a sensory neuron. In some embodiments, the cell is a dorsal root ganglion neuron. In some embodiments, the cell is a neuron from the enteric nervous system. In some embodiments, the cell is a neuroendocrine cell. In some embodiments, the cell is a non-neuronal cell. In some embodiments, the cell is a pancreatic islet cell (e.g., that is polarizable). In some embodiments, the cell is a pituitary cell. In some embodiments, the cell is a pineal cell. In some embodiments, the cell is a thymus cell. In some embodiments, the cell is a pituitary adrenal. In some embodiments, the cell is a thyroid cell. In some embodiments, the cell is a pancreatic cell. In some embodiments, the cell is a central nervous system (CNS) glial cell selected from microglia, astrocytes, oligodendrocytes, radial glial cells, and ependymal cells. In some embodiments, the cell is a PNS glial cell selected from Schwann cells, enteric glial cells, and satellite glial cells.

### Composition and Formulation

In multiple aspects, described herein are pharmaceutical compositions comprising any of the fusion proteins, or any of the nucleic acid, expression cassettes, vector, or viral vector that encodes for the fusion protein described herein, and a pharmaceutically acceptable carrier. Also described herein is a pharmaceutical composition comprising a cell that expresses any of the fusion proteins, or any of the nucleic acid, expression cassettes, vector, or viral vector that encodes for the fusion protein described herein, and a pharmaceutically acceptable carrier.

In some embodiments, the active ingredients of the pharmaceutical composition comprise, consist of, or consist essentially of the fusion protein as described herein or a nucleic acid, expression cassette, vector, or viral vector encoding the fusion protein as described herein.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. the fusion protein as described herein or a nucleic acid, vector, or viral vector encoding the fusion protein as described herein.

In some embodiments, the pharmaceutical composition is formulated for delivery to the central nervous system (CNS). In some embodiments, the pharmaceutical composition is formulated for delivery to the peripheral nervous system (PNS). In some embodiments, the pharmaceutical composition is formulated for delivery to the endocrine system. In some embodiments, the pharmaceutical composition is formulated for delivery across the blood-brain barrier. In some embodiments, the pharmaceutical composition is formulated for delivery to the brain. In some embodiments, the pharmaceutical composition is formulated for delivery to neuronal cells. In some embodiments, the pharmaceutical composition is formulated for delivery to non-neuronal cells. In some embodiments, the pharmaceutical composition is formulated for delivery to insulin-producing cells. As used herein, the term “formulated for” refers to formulations that permit delivery of the pharmaceutical compositions described herein to the specific locations, organs, tissues, or cells indicated, e.g., across the tightly controlled barrier of the blood-brain barrier and into the CNS. The central nervous system (CNS) functions in a tightly controlled and stable environment. This is maintained by highly specialized blood vessels that physically seal the CNS and control substance influx/efflux, known as the “blood brain barrier” (BBB). Specialized tight junctions between endothelial cells comprising a single layer that lines the CNS capillaries are the physical seal between blood and brain. BBB selectivity is facilitated by an array of endothelial transporters responsible for the supply of nutrients and for the clearance of waste or toxins. In concert with pericytes and astrocytes, the BBB protects the brain from various toxins and pathogens and provides the proper chemical composition for synaptic transmissions. Accordingly, provided herein are exemplary formulations for delivery across the blood-brain barrier and/or delivery to the brain. Non-limiting examples of formulations which permit delivery of pharmaceutical compositions across the BBB and into the brain include: direct injection or infusion into the CNS; formulation as a solution, e.g., comprising a carrier protein; formulation as a nanoparticle; formulation as a liposome; formulation as a nucleic acid; formulation as a CNS-tropic viral vector; formulation with or linkage to an agent that is endogenously transported across the BBB; formulation with or linkage to a cell penetrating peptide (CPP); formulation with or linkage to a BBB-shuttle; formulation with or linkage to an agent that increases permeability of the BBB. In embodiments wherein the fusion protein is linked to another agent (e.g., cationic substrate; an agent that is endogenously transported across the BBB; a cell penetrating peptide (CPP); a BBB-shuttle; or an agent that increases permeability of the BBB), the N-terminus and/or the C-terminus of the fusion protein can be linked to the other agent; as non-limiting examples, such a linkage can be a flexible amino acid linker (e.g., a Gly-Ser motif), or a cleavage linker as known in the art.

In some embodiments of any of the aspects, the therapeutic described herein (i.e., any of the fusion proteins, pharmaceutical compositions, nucleic acids, expression cassettes, cells, vectors, or viral vectors) is administered e.g., to the CNS, PNS, or endocrine system. In some embodiments, the therapeutic described herein is administered intracranially, epidurally, intrathecally, intraparenchymally, intraventricularly, or subarachnoidly. In some embodiments, the therapeutic described herein is administered intranasally. In some embodiments, the therapeutic described herein is administered in a formulation that crosses the blood-brain barrier, as described further herein. In some embodiments, the therapeutic described herein is administered via direct injection into the CNS or brain. In some embodiments, the therapeutic described herein is administered via infusion into the CNS or brain, e.g., via a shunt. In some embodiments, the therapeutic described herein is administered into the brain using an invasive method, such as the use of polymers or microchip systems, stereotactically guided drug insertion through a catheter, or transient disruption of the BBB. In some embodiments, the therapeutic described herein is administered to the PNS via direct injection. In some embodiments, the therapeutic described herein is administered via infusion into the PNS. In some embodiments, the therapeutic described herein is administered to the endocrine system via direct injection. In some embodiments, the therapeutic described herein is administered via infusion into the endocrine system.

In some embodiments, the therapeutic described herein is formulated as a solution comprising the fusion protein, wherein the solution is a liquid pharmaceutically acceptable carrier, as described herein or known in the art. In some embodiments, the solution is saline (e.g., PBS). In some embodiments, the solution further comprises a carrier protein, such as BSA. In some embodiments, the solution further comprises a carrier protein that increases delivery across the BBB, such as the carrier protein CRM197, which is the non-toxic mutant of diphtheria toxin that uses the membrane-bound precursor of heparin-binding epidermal growth factor (HBEGF) as its transport receptor, which is constitutively expressed on the blood-brain barrier. In some embodiments, the fusion protein is at a concentration of at least 0.1 nM/mL, at least 1 nM/mL, at least 10 nM/mL, at least 100 nM/mL, at least 1 μM/mL, at least 10 μM/mL, at least 100 μM/mL, at least 1 mM/mL, at least 10 nM/mL, at least 100 mM/mL or more.

In some embodiments, the therapeutic described herein is formulated as a nanoparticle, e.g., that can cross the BBB, or is directed to the PNS or endocrine system. Non-limiting examples of such nanoparticle formulations include liposomes, polymeric nanoparticles, carbon nanotubes, nanofibers, dendrimers, micelles, inorganic nanoparticles made of iron oxide, or gold nanoparticles. In some embodiments of, the therapeutic described herein is formulated as a liposome, polyarginine, protamine, or cyclodextrin-based nanoparticle. In some embodiments, the therapeutic described herein is formulated as liposomes. Liposomes are roughly nano- or microsize vesicles consisting of one or more lipid bilayers surrounding an aqueous compartment. In some embodiments, the liposomes comprise DMPC, dimyristoylphosphatidylcholine; DMPG, dimyristoylphosphatidylglycerol; DOPC, dioleoylphosphatidylcholine; DPPG, dipalmitoylphosphatidylglycerol; DSPC, distearoylphosphatidylcholine; DSPE, distearoylphosphatidylethanolamine; DSPG, distearoylphosphatidylglycerol; EPC, egg phosphatidylcholine; HSPC, hydrogenated soy phosphatidylcholine; PEG, polyethylene glycol; DSPE-PEG2,000; cholesterol; and/or triolein. In some embodiments of any of the aspects, the liposome is cationized. In some embodiments, the fusion protein is linked to a poly-cationic polymer such as poly-ethyleneimine, or otherwise incorporated into a liposomal delivery system. In some embodiments, the liposome comprising a targeting ligand (e.g., CNS, PNS, or endocrine system-targeted aptamers or antibodies, such as the cell-penetrating peptides or BBB-shuttles, as described further herein, or known in the art). In some embodiments, the liposome can be triggered to release the fusion protein, e.g., using external stimuli, such as variations in magnetic field, temperature, ultrasound intensity, light or electric pulses, and others. See e.g., Vieira and Gamarra, Int J Nanomedicine. 2016; 11: 5381-5414, the content of which is incorporated herein by reference in its entirety.

Another aspect herein provides a method comprising administering to a cell an effective amount of any therapeutic described herein (e.g., any of the fusion proteins described herein, any of the synthetic nucleic acids described herein, any of the expression cassettes described herein, any of the vectors described herein, any of the cells described herein, or any of the pharmaceutical compositions described herein). Examples of cells targeted for administration include but are not limited to neurons, glia, astrocytes, oligodendrocytes, ependymal cells, microglia, schwann cells, satellite cells, pancreatic islet cells, beta cells, thyroid follicular cells, parathyroid epithelial cells, cells of the hypothalamus, cells of the pituitary gland, cells of the pineal gland, and cells of the adrenal gland. Exemplary cells include, but are not limited to a liver cell, a cardiac cell, a kidney cell, a spleen cell, a lung cell, a vascular cell, a stomach cell, a bladder cell, muscle cell, a skeletal muscle cell, an epithelial cell, a blood cell, a stem cell, a neutrophil, an immune cell (e.g., a T cell or a B cell), a bone cell, a skin cells, a monocyte, a lymphocyte, adipose cell, a platelet, a endothelium cell,

In some embodiments, the therapeutic described herein can be in a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of the fusion protein as described herein are well known to those skilled in the art. Examples include, without limitation: sterile water; water for injection USP; saline solution; glucose solution; aqueous vehicles such as but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Pharmaceutical compositions comprising the fusion protein as described herein or a nucleic acid, vector, or viral vector encoding the fusion protein as described herein can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, a pharmaceutical composition comprising the fusion protein described herein or a nucleic acid, vector, or viral vector encoding the fusion protein as described herein can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropyl methylcellulose, other polymer matrices, gels, permeable membranes, osmotic systems (such as OROS® (Alza Corporation, Mountain View, Calif. USA)), or a combination thereof to provide the desired release profile in varying proportions.

In some embodiments, the therapeutic described herein further comprises at least one agent that increases the permeability of the blood-brain barrier, e.g., so as to allow the fusion protein described herein to cross the BBB and enter the CNS. In some embodiments, the pharmaceutical composition is co-administered with at least one agent that increases the permeability of the blood-brain barrier. Non-limiting examples of agents that increase the permeability of the blood-brain barrier include: claudin-5 and/or occludin inhibitors; peptides derived from zonula occludens toxin; synthetic peptides targeting the extracellular loops of tight junctions; adenosine 2A receptors (A2AR) agonists; an inhibitor of a gene or gene expression product selected from the group consisting of: Mfsd2A; Slco1C1; Slc38A5; LRP8; Slc3A2; Slc7A5; Slc7A1; Slc6A6; IGFBP7; Glut1; Slc40A1; and Slc30A1; See e.g., US20160120893A1, the content of which is incorporated herein by reference in its entirety.

The term “enhancing or repairing” as used herein refers to an improvement of neural activity and/or synaptic transmission. It is well within the ability of one skilled in the art to determine enhanced synaptic function, e.g., by measuring neurotransmission and neurotransmitter release, by measuring synaptic activity, or firing rates through brain scans or through electrophysiological measurements standard in the art. Enhance or repair would refer to if one or more of the signals or outputs or synaptic function as described herein are altered in a beneficial manner, or even ameliorated, or a desired response is induced e.g., by at least 10% following treatment according to the methods described herein.

### Treatment of Neurological and Endocrine Disorder

Described herein are methods that utilize administration of the any of the therapeutics described herein. One aspect provides a method of repairing or enhancing synaptic function in a subject, the method comprising administering to a subject in need thereof an effective amount of any therapeutic described herein (e.g., any of the fusion proteins described herein, any of the synthetic nucleic acids described herein, any of the expression cassettes described herein, any of the vectors described herein, any of the cells described herein, or any of the pharmaceutical compositions described herein).

Another aspect herein provides a method of treating a neurological disorder in a subject, the method comprising administering to a subject in need thereof an effective amount of any therapeutic described herein (e.g., any of the fusion proteins described herein, any of the synthetic nucleic acids described herein, any of the expression cassettes described herein, any of the vectors described herein, any of the cells described herein, or any of the pharmaceutical compositions described herein).

In some embodiments, the administration enhances neurotransmitter secretion in the subject. As used herein, “neurotransmitter secretion” refers to a synaptic process by which vesicles containing neurotransmitters fuse to the presynaptic membrane and release their contents thereby transmitting a synaptic signal through the nervous system. In some embodiments, synaptic function and neurotransmitter secretion is increased by at least 10%, at least 20%, at least 30%, at least 40%, at least 50%, at least 60%, at least 70%, at least 80%, at least 90%, at least 95%, at least 100% or more compared to a subject that is not administered a pharmaceutical composition, nucleic acid, vector, or viral vector as described herein.

In some embodiments, a subject in need of enhancing or repairing synaptic function is any subject that has the desire or need to enhance or repair synaptic function. In some embodiments, a subject in need of increasing or repairing synaptic function is a subject with a neurological disorder, e.g., learning disability, a neurodegenerative disease or disorder, or another memory-associated disorder (e.g., amnesia, dementia, Alzheimer's disease, mild cognitive impairment, vascular cognitive impairment, or hydrocephalus).

Subjects having neurological disorders can be identified by a physician using current methods of diagnosing neurological disorders know in the art. Symptoms and/or complications of neurological disorders which characterize these conditions and aid in diagnosis are well known in the art and include but are not limited to, memory loss, confusion, restlessness, personality and behavior changes, problems with judgment, problems communicating with others, inability to follow directions, or lack of emotion. Tests that may aid in a diagnosis of a neurological disorders include, but are not limited to, the Mini-Mental State Exam (MMSE) and the Mini-Cog test. A family history of a neurological disorders, or exposure to risk factors for a neurological disorders (e.g., nutritional deficiency, lower education level, older age, history of head trauma, illness, medications (including alcohol or illicit drugs), vision or hearing impairment, uncontrolled chronic medical conditions, stroke, or psychological factors such as depression and stress) can also aid in determining if a subject is likely to have a neurological disorders or in making a diagnosis of a neurological disorders.

In some embodiments, administering an effective amount of any therapeutic described herein alleviates at least one symptom of a neurological disorder. As used herein, “alleviating a symptom of a neurological disorders” is ameliorating any condition or symptom associated with the neurological disorders. As compared with an equivalent untreated control, such reduction is by at least 5%, 10%, 20%, 40%, 50%, 60%, 80%, 90%, 95%, 99% or more as measured by any standard technique.

In some embodiments, the neurological disorder is a learning disability. Non-limiting examples of learning disabilities include dyscalculia, dysgraphia, dyslexia, a non-verbal leaning disability, an oral and/or written language disorder and specific reading comprehension deficit, attention deficit hyperactivity disorder (ADHD), attention deficit disorder (ADD), dyspraxia, an executive mal-functioning, an auditory processing disorder, a language processing disorder, a visual perceptual/visual motor deficit, and the like.

Subjects having a learning disability can be identified by a physician using current methods of diagnosing learning disabilities. Symptoms and/or complications of learning disabilities which characterize these conditions and aid in diagnosis are well known in the art and include but are not limited to, problems reading and/or writing, problems with math, poor memory, problems paying attention, trouble following directions, clumsiness, trouble telling time, problems staying organized, and hyperactivity. Tests that may aid in a diagnosis of learning disabilities include, but are not limited to, Woodcock-Johnson Tests of Achievement (WJ), the Wechsler Individual Achievement Test (WIAT), the Wide Range Achievement Test (WRAT), and the Kaufman Test of Educational Achievement (KTEA). A family history of learning disabilities, or exposure to risk factors for learning disabilities (e.g. poor fetal growth in the uterus (e.g., severe intrauterine growth restriction), exposure to alcohol or drugs before being born, premature birth, very low birthweight, psychological trauma, physical trauma (e.g., head injuries or nervous system infections), environmental exposure to high levels of toxins, such as lead) can also aid in determining if a subject is likely to have a learning disability or in making a diagnosis of a learning disability.

In some embodiments, the neurological disorder is a neurodegenerative disease. Non-limiting examples of neurodegenerative diseases or disorders include Alzheimer's disease, Parkinson's disease, Huntington's disease, amyotrophic lateral sclerosis (ALS), frontotemporal dementia, chronic traumatic encephalopathy (CTE), multiple sclerosis, and neuroinflammation, among others.

Subjects having a neurodegenerative disease or disorder can be identified by a physician using current methods of diagnosing neurodegenerative diseases or disorders. Symptoms and/or complications of neurodegenerative diseases or disorders, which characterize these conditions and aid in diagnosis are well known in the art and include but are not limited to memory loss, forgetfulness, apathy, anxiety, agitation, a loss of inhibition, or mood changes. Tests that may aid in a diagnosis of a neurodegenerative disease or disorder include, but are not limited to, imaging (e.g., of the brain by a CT scan, PET scan, MRI, or the like), genetic testing for associated disease markers, cognitive testing (e.g., the clock-drawing test for neurodegenerative diseases or disorders), behavioral testing, physical stamina testing, etc. A family history of a neurodegenerative disease or disorder, or exposure to risk factors for a neurodegenerative disease or disorder can also aid in determining if a subject is likely to have a neurodegenerative disease or disorder or in making a diagnosis of a neurodegenerative disease or disorder.

In some embodiments, the neurological disorder is epilepsy or seizures. Non-limiting examples of epilepsy include: focal seizures without loss of consciousness (simple partial seizures); focal seizures with impaired awareness (complex partial seizures); absence seizures (petit mal seizures); tonic seizures; atonic seizures; clonic seizures; myoclonic seizures; or tonic-clonic seizures, among others.

Subjects having epilepsy can be identified by a physician using current methods of diagnosing epilepsy. Symptoms and/or complications of epilepsy, which characterize these conditions and aid in diagnosis are well known in the art and include but are not limited to temporary confusion, staring spells, stiff muscles, uncontrollable jerking movements of the arms and legs, loss of consciousness or awareness, and/or psychological symptoms such as fear, anxiety, or deja vu. Tests that may aid in a diagnosis of epilepsy include, but are not limited to, neurological exams, blood tests, electroencephalogram (EEG), high-density EEG, brain imaging (e.g., computerized tomography (CT) scan, magnetic resonance imaging (MRI), functional MRI (fMRI), positron emission tomography (PET), single-photon emission computerized tomography (SPECT)), neuropsychological tests (e.g., testing thinking, memory, and/or speech skills), statistical parametric mapping (SPM), electrical source imaging (ESI), or magnetoencephalography (MEG), etc. A family history of epilepsy, or exposure to risk factors for epilepsy (e.g., head injury; brain abnormalities (e.g., brain tumors or vascular malformations such as arteriovenous malformations (AVMs) and cavernous malformations; stroke) infections (e.g., Meningitis, HIV, viral encephalitis and some parasitic infections (e.g., Taenia solium, the pork tapeworm)); pre-natal brain injury (e.g., caused by infection in the mother, poor nutrition, or oxygen deficiencies); developmental disorders such as autism; high fevers in children; etc.) can also aid in determining if a subject is likely to have epilepsy or in making a diagnosis of epilepsy.

Yet another aspect herein provides a method of treating a secretory disease or endocrine disease in a subject, the method comprising administering to a subject in need thereof an effective amount of any therapeutic described herein (e.g., any of the fusion proteins described herein, any of the synthetic nucleic acids described herein, any of the expression cassettes described herein, any of the vectors described herein, any of the cells described herein, or any of the pharmaceutical compositions described herein). For example, secretory disorders are defined by increased or decreased secretion, i.e. insulin secretion or neurotransmitter secretion. Endocrine disorders are defined as disorders of the endocrine system. Examples of endocrine disorders include but are not limited to glucose homeostasis disorders, thyroid disorders, calcium homeostasis disorders, pituitary gland disorders, and sex hormone disorders. Glucose homeostasis disorders include but are not limited to diabetes, hypoglycemia, and glucagonoma. Thyroid disorders include but are not limited to goitre, hyperthyroidism, hypothyroidism, thyroiditis, thyroid cancer, and thyroid hormone resistance. Calcium homeostasis disorders include but are not limited to parathyroid gland disorders, osteoporosis, osteitis deformans, rickets, and osteomalacia. Pituitary gland disorders include but are not limited to diabetes insipidus, syndrome of inappropriate antidiuretic hormone, hypopituitarism, and pituitary tumors. Sex hormone disorders include but are not limited to hermaphroditism, gonadal dysgenesis, androgen insensitivity syndromes, hypogonadism, Kallmann syndrome, Klinefelter syndrome, Turner syndrome, ovarian failure, testicular failure, delayed or precocious puberty, amenorrhea, and polycystic ovary syndrome. Subjects having a condition, e.g., diabetes can be identified by a physician using current methods of diagnosing diabetes. Symptoms and/or complication of diabetes which characterize these conditions and aid in diagnosis are well known in the art and include but are not limited to, weight loss, slow healing, plyuria, polydipsia, polyphagiam headaches, itchy skin, and fatigue. Tests that may aid in a diagnosis of, e.g. diabetes include, but are not limited to, blood tests (e.g., for fasting glucose levels). A family history of endocrine diseases, or exposure to risk factors for endocrine diseases (e.g. overweight) can also aid in determining if a subject is likely to have an endocrine disease or in making a diagnosis of an endocrine disease.

The term “effective amount” as used herein refers to the amount of any therapeutic described herein (e.g., any of the fusion proteins described herein, any of the synthetic nucleic acids described herein, any of the expression cassettes described herein, any of the vectors described herein, any of the cells described herein, or any of the pharmaceutical compositions described herein) needed to, e.g., enhance synaptic function, or alleviate at least one or more symptom of the disease or disorder, and relates to a sufficient amount of composition to provide the desired effect. The term “therapeutically effective amount” therefore refers to an amount of the therapeutic described herein that is sufficient to provide, e.g., a particular enhanced synaptic function or anti-neurological disorder effect when administered to a typical subject. An effective amount as used herein, in various contexts, would also include, e.g., an amount sufficient to enhance synaptic activity, delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the fusion protein, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assays or tests for synaptic activity. The dosage can be determined by a physician and adjusted, as necessary, to suit observed effects of the treatment.

In embodiments wherein the administration is in the form of a nucleic acid, vector, or viral vector encoding at least one fusion protein, effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for synaptic activity or neurotransmitter release. The dosage can be determined by a physician and adjusted, as necessary, to suit observed effects of the treatment.

In certain embodiments, an effective dose of a composition comprising at least the fusion protein as described herein can be administered to a patient once. In certain embodiments, an effective dose of a composition comprising at least the fusion protein can be administered to a patient repeatedly. For systemic administration, subjects can be administered a therapeutic amount of a composition comprising at least the fusion protein, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a learning disability or a neurodegenerative disease or disorder, e.g. by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the fusion protein. The desired dose or amount can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising fusion protein or nucleic acids, vectors, or viral vectors encodings at least one fusion protein can be administered over a period of time, such as over a 5 minute, 10 minute, 15 minute, 20 minute, or 25 minute period.

The dosage ranges for the administration of the fusion protein pharmaceutical compositions, or nucleic acids, vectors, or viral vectors encoding at least the fusion protein, according to the methods described herein depend upon, for example, the form of the fusion protein (e.g., polypeptide or nucleic acid; specific pharmaceutically acceptable carrier) its potency, and the extent to which symptoms, markers, or indicators of a condition described herein are desired to be reduced, for example the percentage reduction desired for symptoms of a neurological disorder, or the extent to which, for example, synaptic function are desired to be induced. The dosage should not be so large as to cause adverse side effects, such as overstimulation of the brain. Generally, the dosage will vary with the age, condition, and sex of the patient and can be determined by one of skill in the art. The dosage can also be adjusted by the individual physician in the event of any complication.

The efficacy of the fusion protein pharmaceutical compositions or nucleic acids, vectors, or viral vectors encodings at least the fusion protein in, e.g. the treatment of a condition described herein, or to induce a response as described herein (e.g. enhanced synaptic function) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. learning or memory acuity. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms as described herein; or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. synaptic activity). It is well within the ability of one skilled in the art to monitor efficacy of administration and/or treatment by measuring any one of such parameters, or any combination of parameters. Efficacy can be assessed in animal models of a condition described herein, for example treatment of a neurological disorder. When using an experimental animal model, efficacy of treatment is evidenced when a statistically significant change in a marker is observed, e.g. RIMS levels in the CNS or cerebrospinal fluid (CSF) and or synaptic activity, among others.

In vitro and animal model assays are provided herein which allow the assessment of a given dose of the fusion protein pharmaceutical composition or a nucleic acid, vector, or viral vector encodings at least the fusion protein. A non-limiting example of an in vitro assay that can be performed to test efficacy or dosage includes: (1) exposure of a neuronal cell line (e.g., SH-SY5Y neuroblastoma cells; NT2 cells, such as NTERA-2 CL.D1 (NT2/D1) ATCC® CRL-1973™; PC-12 cells (e.g., ATCC® CRL-1721™)) or a primary neuronal culture (e.g., murine, rat, non-human primate, or human primary neuronal cultures) to the fusion protein pharmaceutical composition or a nucleic acid, vector, or viral vector encoding at least the fusion protein; and (2) assaying for neuronal activity using electrophysiology techniques such as patch-clamping. In some embodiments, the primary neuronal cultures can be isolated from the animal models described herein, e.g., for Alzheimer's disease, ADHD, Parkinson's disease, Huntington's disease, amyotrophic lateral sclerosis, multiple sclerosis, or epilepsy. In some embodiments, the neuronal cultures can be derived from human stem cells (e.g., induced pluripotent stem cells (iPSCs), e.g., from a human patient with a condition described herein). The neuronal cells can be monitored for signs that indicate efficacy of the treatment, including enhanced synaptic activity. The cells can also be monitored for viability, e.g., using live-dead staining; an optimal dose would exhibit a minimal or no decrease in viability, coupled with signs of efficacy in the neurons.

The efficacy of a given dosage combination can also be assessed in an animal model, e.g. for a neurological disorder. A non-limiting example of an in vivo assay that can be performed to test efficacy or dosage includes: (1) administration of the fusion protein pharmaceutical composition or a nucleic acid, vector, or viral vector encoding at least the fusion protein to the animal; and (2) assaying for enhanced synaptic function. In some embodiments, the animal can be a mouse, rat, or non-human primate. In some embodiments, the animal can be a human in a clinical trial, e.g., using dosages determined in non-human animal trials. The animals can also be monitored for morbidity and mortality; an optimal dose would exhibit no mortality and minimal morbidity, coupled with signs of efficacy in the animals. See e.g., Drummond ad Wisniewski, Acta Neuropathol. 2017, 133(2): 155-175; Löscher, Neurochem Res. 2017, 42(7):1873-1888; Russell et al., Behav Brain Funct. 2005; 1: 9; Ramaswamy, ILAR J. 2007, 48(4):356-73; Morris et al., Neural Regen Res. 2018, 13(12): 2050-2054; Procaccini et al., Eur J Pharmacol. 2015, 759:182-91; Konnova and Swanberg. Chapter 5, Animal Models of Parkinson's Disease, Parkinson's Disease: Pathogenesis and Clinical Aspects, Codon Publications, 2018; the contents of each of which are incorporated herein by reference in their entireties. Administration and Treatment Met

A variety of kits and components can be prepared for use in the methods described herein, depending upon the intended use of the kit. Accordingly, in another aspect, provided herein is a kit comprising a fusion protein described herein or a nucleic acid encoding a fusion protein described herein. A kit is any manufacture (e.g., a package or container) comprising a fusion protein or a polynucleotide encoding a fusion protein described herein. The manufacture can be promoted, distributed, or sold as a unit for performing the methods described herein.

The kits described herein can optionally comprise additional components and reagents. As will be appreciated by one of skill in the art, components of the kit can be provided in any desired form, e.g., in a lyophilized form, a liquid form, a solid form, or a concentrated. In some embodiments of the various aspects described herein, the kit can comprise ampoules, syringes, or the like.

In some embodiments, the kit can comprise informational material. The informational material can be descriptive, instructional, marketing or other material that relates to the methods described herein. The informational material of the kits is not limited in its form. In some embodiments, the informational material can include information about production of the reagents, concentration, date of expiration, batch or production site information, and so forth. In one embodiment, the informational material relates to methods for using or administering the components of the kit.

It is notes that the components of a kit can provided singularly or in any combination as a kit. Such a kit includes the components described herein and packaging materials thereof.

In some embodiments, the compositions in a kit can be provided in a watertight or gas tight container which in some embodiments is substantially free of other components of the kit. For example, the reagents described herein can be supplied in more than one container, e.g., it can be supplied in a container having sufficient reagent for a predetermined number of applications, e.g., 1, 2, 3 or greater. One or more components as described herein can be provided in any form, e.g., liquid, dried or lyophilized form. Liquids or components for suspension or solution of the reagents can be provided in sterile form and should not contain microorganisms or other contaminants. When the components described herein are provided in a liquid solution, the liquid solution preferably is an aqueous solution.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of viral infection. A subject can be male or female.

A subject can be one who has been previously diagnosed with or identified as suffering from or having a condition in need of treatment for repairing or enhancing synaptic function or a neurological disorder, one or more complications related to the same, and optionally, have already undergone treatment for the same Alternatively, a subject can also be one who has not been previously diagnosed as having a need for repairing or enhancing synaptic function or a neurological disorder or one or more complications related the same. For example, a subject can be one who exhibits one or more risk factors for a neurological disorder or one or more complications related to a neurological disorder or a subject who does not exhibit risk factors. A “subject in need” of testing for a particular condition can be a subject having that condition, diagnosed as having that condition, or at risk of developing that condition.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

As used herein, the terms “protein” and “polypeptide” are used interchangeably to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

The terms “wild-type” or “wt” or “native” as used herein is meant an amino acid sequence or a nucleotide sequence that is found in nature, including allelic variations. A wild-type protein, polypeptide, antibody, immunoglobulin, IgG, polynucleotide, DNA, RNA, and the like has an amino acid sequence or a nucleotide sequence that has not been intentionally modified.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

The term “amino acid substitution” refers to the replacement of at least one existing amino acid residue in a predetermined or native amino acid sequence with a different “replacement” amino acid. A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

The term “amino acid insertion” refers to the insertion of one or more additional amino acids into a predetermined or native amino acid sequence. The insertion can be one, two, three, four, five, or up to twenty amino acid residues.

The term “amino acid deletion” refers to removal of at least one amino acid from a predetermined or native amino acid sequence. The deletion can be one, two, three, four, five, or up to twenty amino acid residues.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity according to the assays described herein (i.e., enhancing synaptic activity). A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan to generate and test artificial variants.

The term “nucleic acid” refers to a deoxyribonucleotide or ribonucleotide and polymers thereof in either single strand or double strand form. The term “nucleic acid” is used interchangeably with gene, nucleotide, polynucleotide, cDNA, DNA, and mRNA. The polynucleotides can be in the form of RNA or DNA. Polynucleotides in the form of DNA, cDNA, genomic DNA, nucleic acid analogs, and synthetic DNA are within the scope of the present invention. Unless specifically limited the term encompasses nucleic acids containing known analogues of natural nucleotides that have similar binding propertied as the natural nucleic acid. Unless specifically limited, a particular nucleotide sequence also encompasses conservatively modified variants thereof (for example, those containing degenerate codon substitutions) and complementary sequences as well as the as well as the sequences specifically described.

The polynucleotides can be composed of any polyribonucleotide or polydeoxyribonucleotide, which can be unmodified RNA or DNA or modified RNA or DNA. For example, polynucleotides can be composed of single or double stranded regions, mixed single or double stranded regions. In addition, the polynucleotides can be triple stranded regions containing RNA or DNA or both RNA and DNA. Modified polynucleotides include modified bases, such as tritylated bases or unusual bases such as inosine. A variety of modification can be made to RNA and DNA, thus polynucleotide includes chemically, enzymatically, or metabolically modified forms.

The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence, which sequence, as a result of the redundancy or degeneracy of the genetic code, encodes the same polypeptides as the DNA provided herein.

A variant DNA or amino acid sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments of the various aspects described herein, a polypeptide, nucleic acid, or cell as described herein can be engineered. As used herein, “engineered” refers to the aspect of having been manipulated by the hand of man. For example, a polynucleotide is considered to be “engineered” when at least one aspect of the polynucleotide, e.g., its sequence, has been manipulated by the hand of man to differ from the aspect as it exists in nature.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The term “statistically significant” or “significantly” refers to statistical significance and generally means a two standard deviations (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean 1%. In some embodiments of the various aspects described herein, the term “about” when used in connection with percentages can mean±5%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

### Example 1: Reconstructing Essential Active Zone Functions within a Synapse

### Abstract

Active zones are molecular machines that control neurotransmitter release through synaptic vesicle docking and priming, and through coupling of these vesicles to Ca²⁺ entry. The complexity of active zone machinery has made it challenging to determine which mechanisms drive these roles in release. Here, we induce RIM+ELKS knockout to eliminate active zone scaffolding networks, and then reconstruct each active zone function. Re-expression of RIM1-Zn fingers positioned Munc13 on undocked vesicles and rendered them release-competent. Reconstitution of release-triggering required docking of these vesicles to Ca²⁺ channels. Fusing RIM1-Zn to Ca_{V}β4-subunits sufficed to restore docking, priming and release-triggering without reinstating active zone scaffolds. Hence, exocytotic activities of the 80 kDa Ca_{V}β4-Zn fusion protein bypassed the need for megadalton-sized secretory machines. These data define key mechanisms of active zone function, establish that fusion competence and docking are mechanistically separable, and reveal that active zone scaffolding networks are not required for release.

### Introduction

Essential insight into the functioning of synaptic exocytotic machinery has come from rebuilding the fusion process in vitro. Reconstitution assays have revealed that the minimal machinery for Ca²⁺-triggered exocytosis consists of SNARE proteins, Munc18, Munc13 and synaptotagmin (Hu et al., 2003; Ma et al., 2013; Tucker et al., 2004). However, fusion speed in these assays is orders of magnitude slower than at synapses, and spatial precision of exocytosis with its exact targeting towards receptors on target cells cannot be studied in these in vitro systems. These functions are carried out by the active zone, a molecular machine that is attached to the presynaptic plasma membrane and is composed of many megadalton-sized protein assemblies (Emperador-Melero and Kaeser, 2020; Südhof, 2012).

Central functions of the active zone are the generation of releasable vesicles and the positioning of these vesicles close the Ca²⁺ channels for rapid fusion-triggering (Augustin et al., 1999; Biederer et al., 2017; Deng et al., 2011; Imig et al., 2014; Kaeser et al., 2011; Liu et al., 2011). Active zones are composed of families of scaffolding proteins including RIM, ELKS, Munc13, RIM-BP, Bassoon/Piccolo and Liprin-α (Südhof, 2012). Each of these proteins is encoded by multiple genes and the individual proteins are large, ranging from 125 to 420 kDa, forming complex protein networks. Mechanisms for their assembly are not well understood, but recent studies suggest the involvement of liquid-liquid phase separation for active zone formation and plasma membrane attachment (Emperador-Melero et al., 2021; McDonald et al., 2020; Wu et al., 2019, 2021).

Decades of gene knockout and related studies have uncovered loss-of-function phenotypes for individual active zone proteins. In essence, these studies established that each protein, in one way or another, participates in the control of each key exocytotic parameter. For example, roles in vesicle docking and priming have been described for RIM (Calakos et al., 2004; Deng et al., 2011; Han et al., 2011; Kaeser et al., 2011; Koushika et al., 2001), Munc13 (Aravamudan et al., 1999; Augustin et al., 1999; Deng et al., 2011; Imig et al., 2014; Richmond et al., 1999), Liprin-α (Emperador-Melero et al., 2021; Spangler et al., 2013; Wong et al., 2018), ELKS (Dong et al., 2018; Held et al., 2016; Kawabe et al., 2017; Matkovic et al., 2013), Piccolo/Bassoon (Parthier et al., 2018), and RIM-BP (Brockmann et al., 2019). Similarly, the control of Ca²⁺ secretion-coupling is mediated by the same proteins, with established roles for RIM, RIM-BP, Bassoon, and ELKS (Acuna et al., 2015; Davydova et al., 2014; Dong et al., 2018; Grauel et al., 2016; Han et al., 2011; Kaeser et al., 2011; Kittel et al., 2006; Liu et al., 2014, 2011). A true mechanistic understanding of the active zone, however, has been difficult to achieve. This is in part because reconstitution assays, powerful for untangling mechanisms of the fusion reaction itself (Hu et al., 2003; Ma et al., 2013; Tucker et al., 2004), are not possible for the active zone due to its molecular complexity. Furthermore, the redundancy of the scaffolding and release mechanisms has made it challenging to distinguish effects on active zone assembly and function, and the steps of build-up of release machinery for exocytosis of a vesicle have remained uncertain. Ultimately, it has not been possible to define which of the many candidate mechanisms at the active zone suffice to drive fast, action potential-triggered release.

Simultaneous conditional knockout of RIM and ELKS leads to disassembly of the active zone with loss of RIM, ELKS, RIM-BP, Piccolo, Bassoon and Munc13, a near-complete absence of vesicle docking, and a strong reduction in action-potential evoked exocytosis (Wang et al., 2016). Remarkably, general features of synaptic structure including the formation of boutons, accumulation of vesicles, and generation of synaptic contacts remain intact. This has established that RIM and ELKS form a scaffolding complex that holds the active zone together.

Here, we use this active zone disruption through RIM+ELKS knockout to develop an approach for reconstructing hallmark functions of these secretory machines within synapses. Our overall goal was to develop a deep mechanistic understanding of active zone function and to define which of the many mechanisms are sufficient to drive release. We experimentally identify the positioning of activated Munc13 to vesicles as a mechanism that induces fusion-competence. Notably, vesicles can be rendered release-competent by Munc13 without tethering them to the target membrane. Docking of these vesicles next to Ca²⁺ channels was required, however, to restore action potential-triggering of release. We achieved this using a single artificial fusion-protein consisting of the RIM zinc finger domain and CaVβ4-subunits, which leads to recovery of fusion strength, speed and spatial precision after active zone disruption. These findings establish the minimal requirements for active zone function, define key protein domains sufficient to mediate these requirements, and identify mechanisms that drive assembly of this minimal release machinery. Data described herein provides proof-of-concept for how an 80 kDa protein can be used to bypass the need for a megadalton-sized protein machine.

### Results

RIM Restores Synaptic Structure and Function after Active Zone Disruption

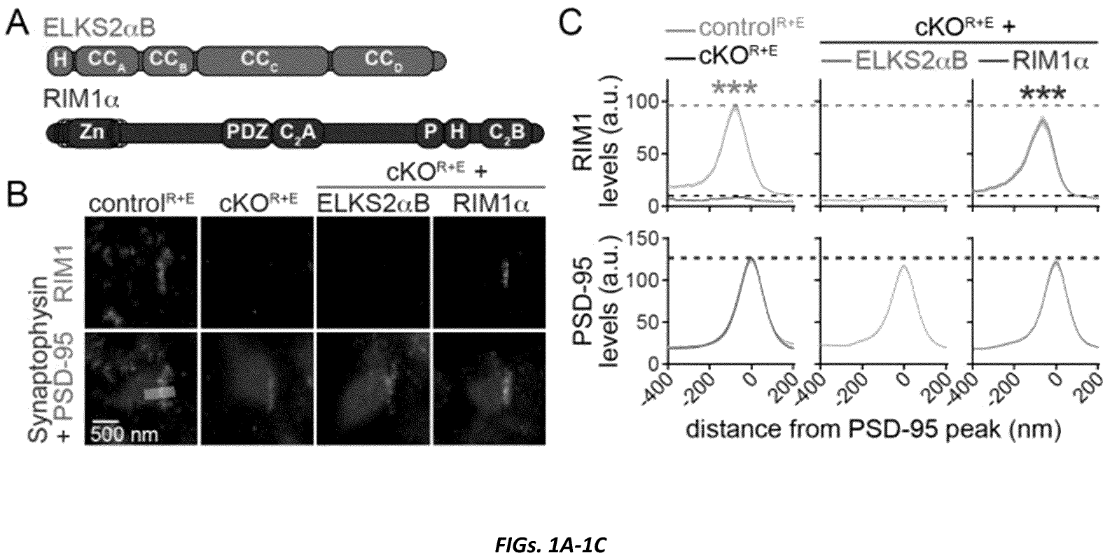

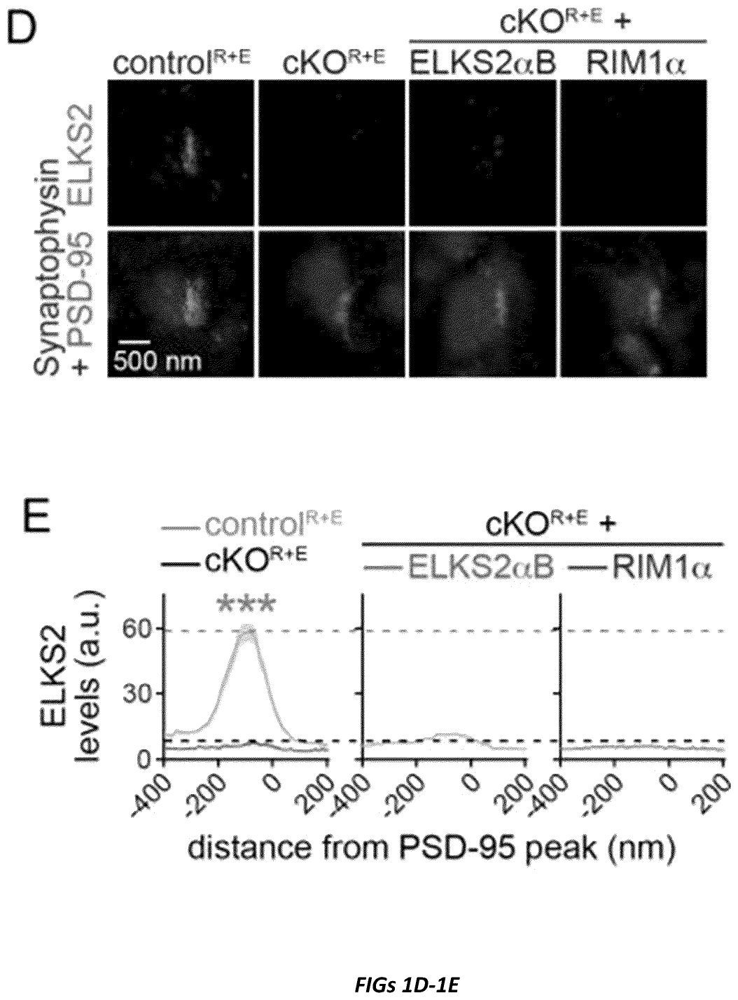

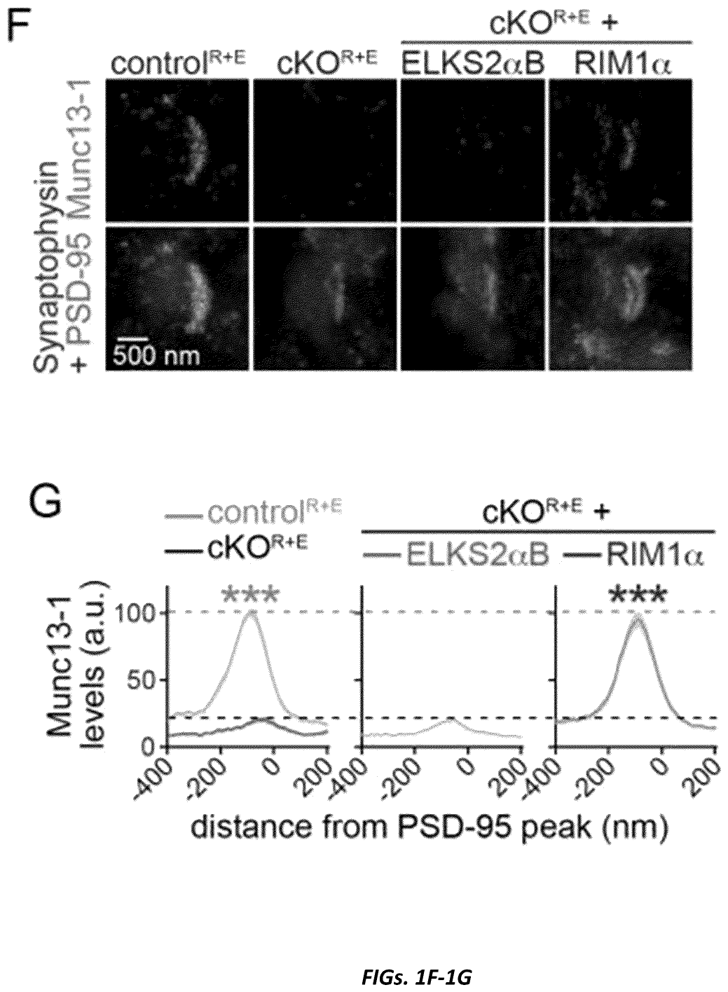

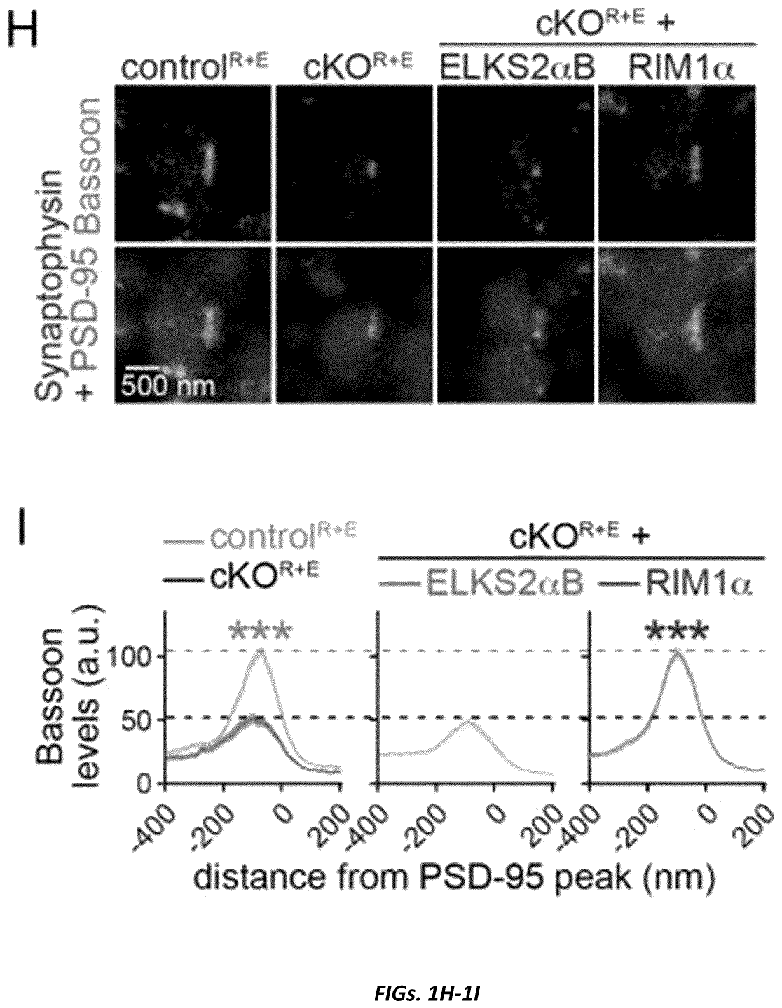

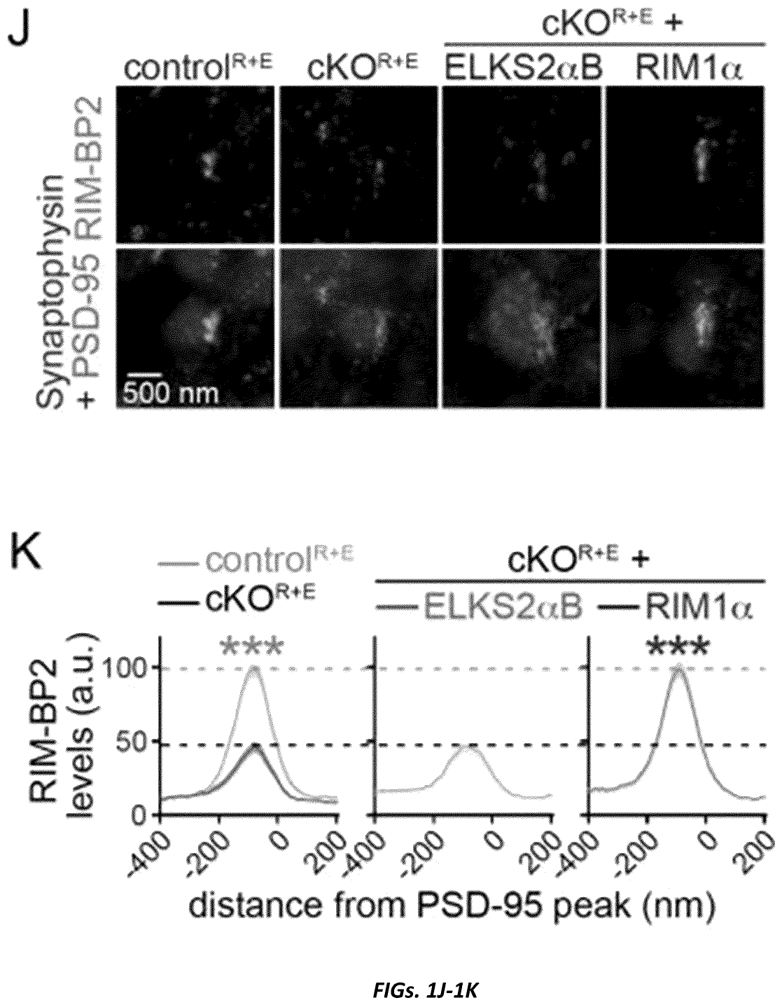

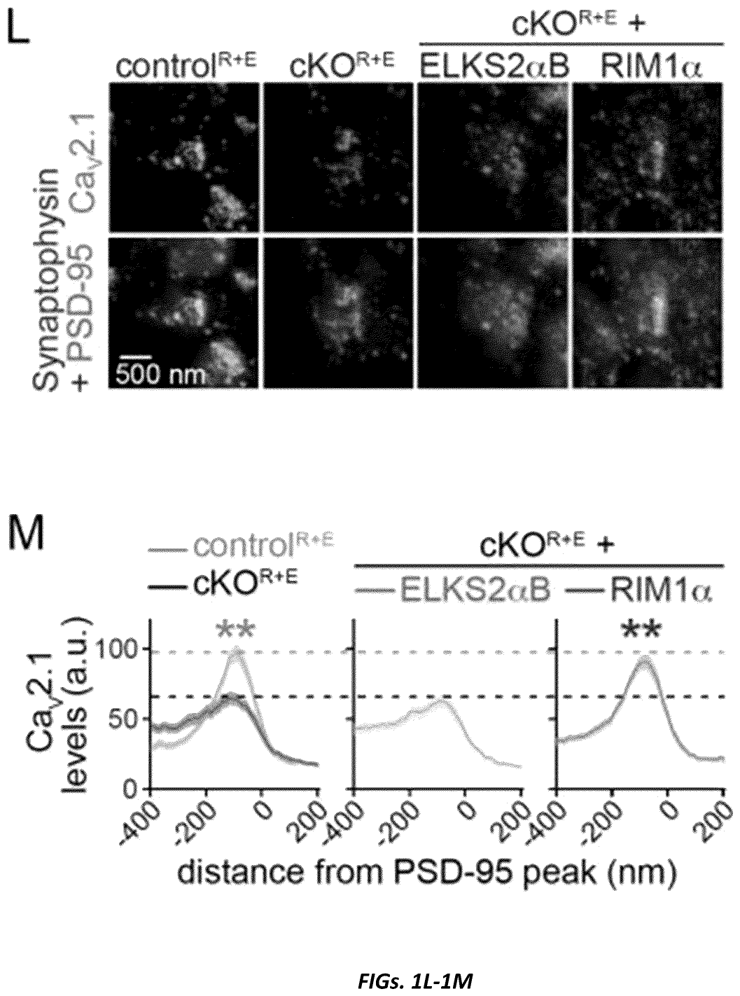

We first used stimulated emission depletion (STED) superresolution microscopy to evaluate active zone disruption induced by conditional knockout of RIM and ELKS, which strongly impaired extent, speed and precision of neurotransmitter release (Wang et al., 2016). Cultured hippocampal neurons with floxed alleles for RIM1, RIM2, ELKS1 and ELKS2 were infected with cre-expressing lentiviruses to generate knockout (cKO^{R+E}) neurons or with control viruses (to generate control^{R+E} neurons). We then used a previously established workflow (Held et al., 2020; de Jong et al., 2018; Nyitrai et al., 2020; Wong et al., 2018) to assess active zone structure at 15-19 days in vitro (DIV). We identified side-view synapses in immunostainings by a bar-like postsynaptic density (PSD, marked by PSD-95, STED) that was aligned at one edge of a synaptic vesicle cloud (Synaptophysin, confocal), and assessed localization of target proteins (STED) relative to these markers in line profiles (FIGS. S1A-S1D). The cKO^{R+E} synapses had disrupted active zones with near-complete loss of ELKS2, RIM1 and Munc13-1, and strong reductions in Bassoon and RIM-BP2 (FIGS. 1A-1K). In addition, we observed a partial loss of CaV2.1 (FIGS. 1L, 1M), the α1-subunit of the voltage-gated Ca²⁺ channels that trigger release at these synapses (Held et al., 2020). Removal of these active zone complexes, however, did not affect the PSDs (marked by PSD-95, FIG. 1C), and led to increased levels of Liprin-α3 (FIGS. S1E, S1F), a protein that connects active zone assembly pathways to structural plasticity (Emperador-Melero et al., 2021; Wong et al., 2018).

With the overall goal to rebuild active zone function using the minimally required protein domains and interactions, we first tested whether either RIM or ELKS mediate recovery of active zone structure and function on their own. We re-expressed RIM1α or ELKS2αB using

lentiviruses (FIGS. 1A, SIG), and found that RIM1α was targeted correctly (FIGS. 1B, IC) and was able to reestablish normal levels and active zone positioning of Munc13-1, Bassoon, RIM-BP2, CaV2.1 and Liprin-α3 (FIGS. 1F-1M, S1E, S1F). In contrast, rescue ELKS2αB did not localize to the target membrane area (FIGS. 1D, 1E) and did not restore other proteins (FIGS. 1F-1M, S1E, S1F), even though it was expressed efficiently (FIG. S1G). To assess protein levels upon rescue with an independent approach and to determine whether RIM-mediated active zone protein recruitment depends on the levels of RIM expression, we performed additional confocal microscopy experiments. Notably, re-expression of low or high RIM1α levels mediated recovery of the other proteins dose-dependently. Higher levels of RIM1α at synapses were driving the recruitment of higher levels of Munc13-1, CaV2.1, and RIM-BP2 (FIG. S2), indicating that RIM is able to titrate the presynaptic levels of interacting active zone proteins.

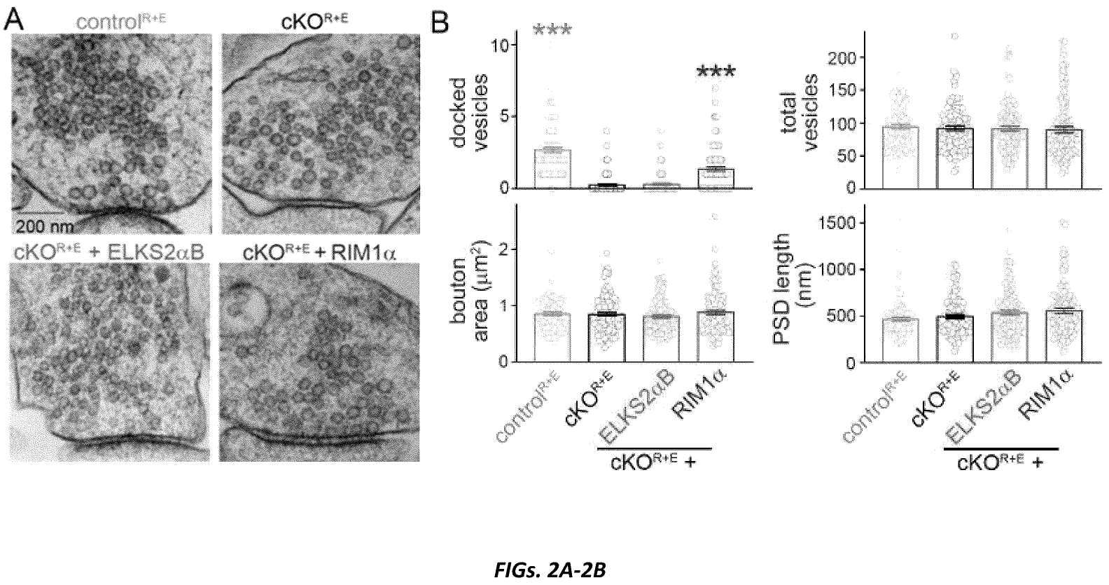

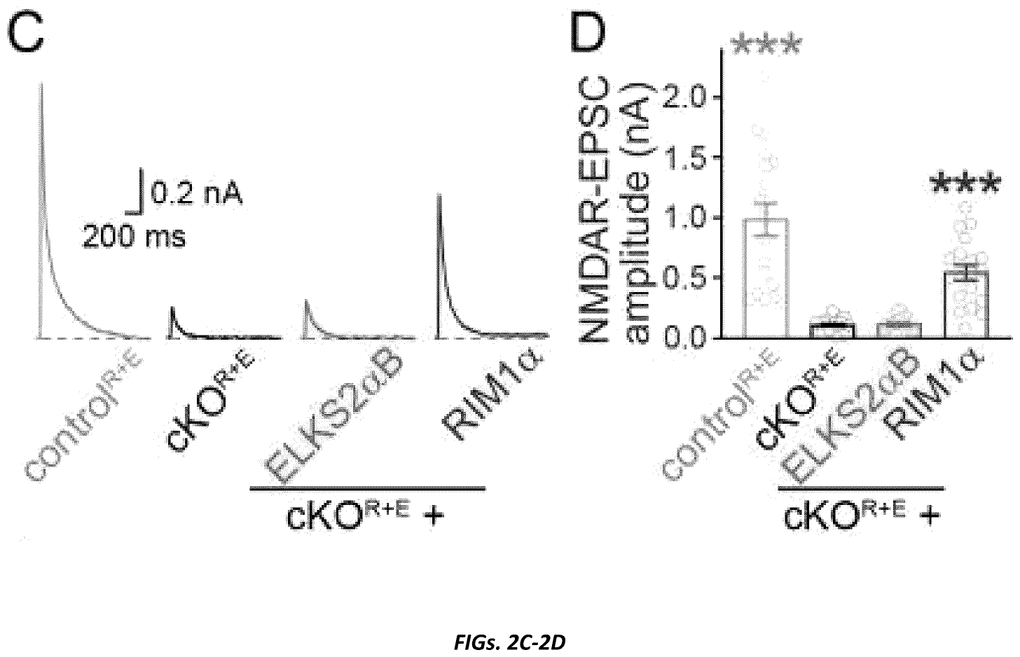

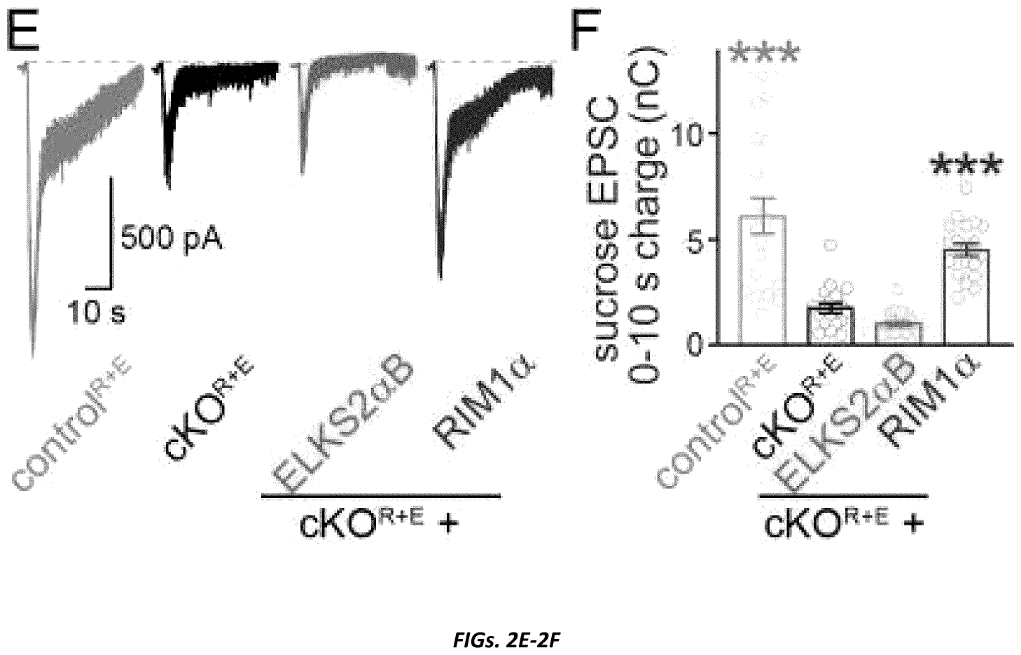

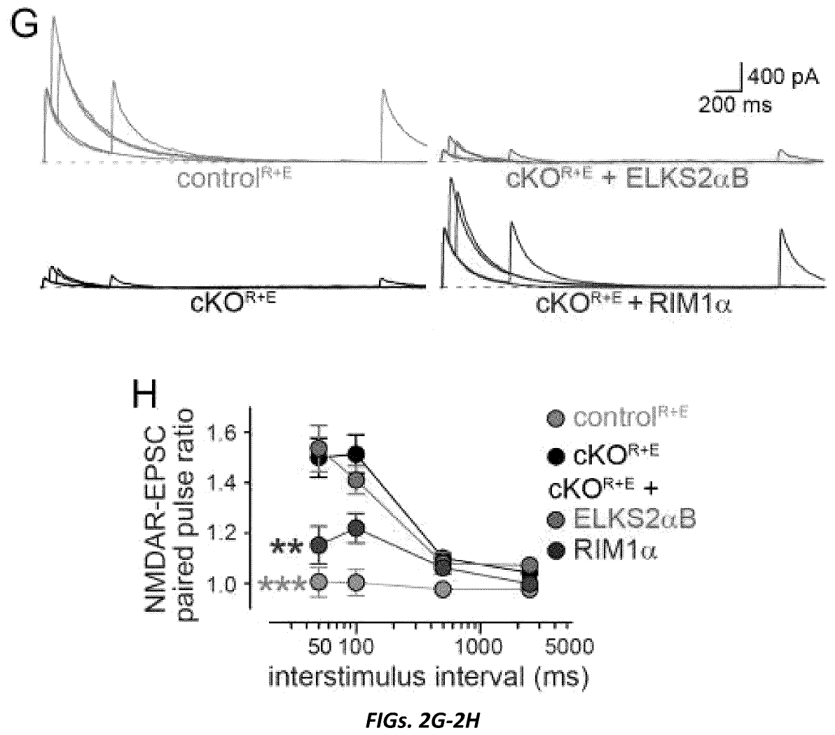

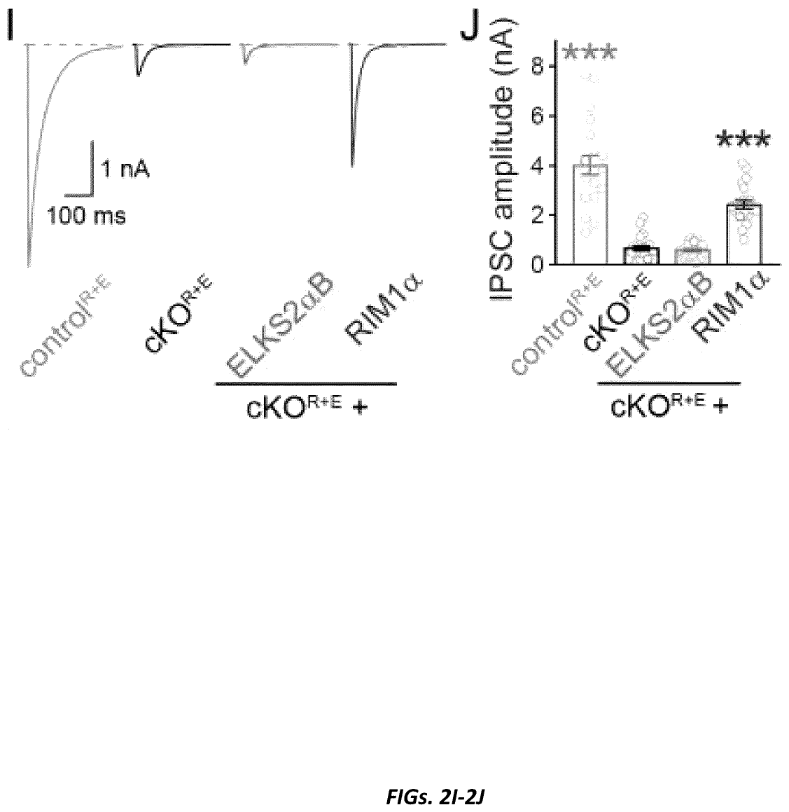

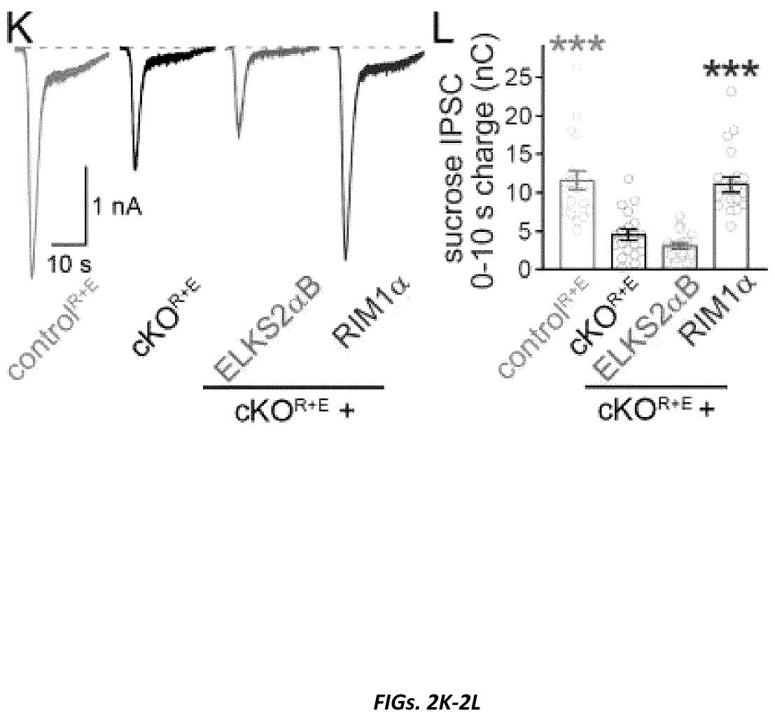

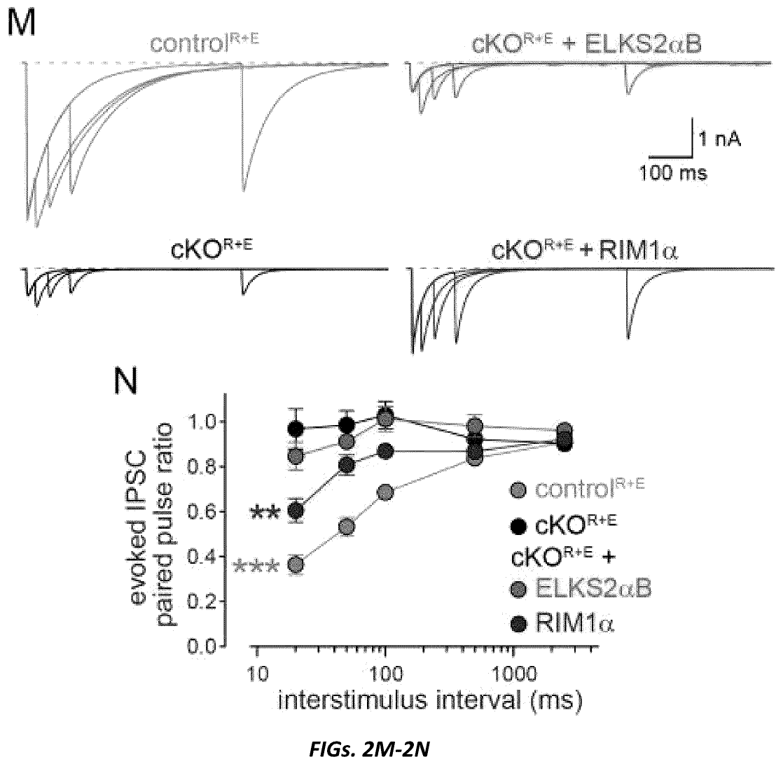

We next tested whether RIM1α re-expression restored key active zone functions, synaptic vesicle docking and release (FIG. 2). To assess vesicle docking and synaptic ultrastructure, we fixed neurons with high pressure-freezing and analyzed electron microscopic images of synapses. Most cKO^{R+E} synapses lacked docked vesicles entirely (assessed as vesicles for which the electron density of the vesicle membrane merges with that of the active zone target membrane), but other parameters including PSD length, bouton size and total vesicle numbers were unaffected. RIM1α restored vesicle docking to 51% of its initial levels, while ELKS2αB expression did not improve docking (FIGS. 2A, 2B).

The active zone controls synaptic strength by generating a readily releasable pool (RRP) of vesicles and by setting the release probability p of each RRP vesicle. We measured synaptic strength and estimated these constituents, p and RRP, at both excitatory and inhibitory synapses using electrophysiology (FIGS. 2C-2N). p is inversely proportional to the ratio of release in response to paired pulses at short interstimulus intervals (Zucker and Regehr, 2002), and application of hypertonic sucrose was used to estimate the RRP (Kaeser and Regehr, 2017; Rosenmund and Stevens, 1996). RIM1α mostly restored excitatory (FIGS. 2C-2H) and inhibitory (FIGS. 2I-2N) synaptic strength, and both RRP and p were recovered to a large extent at both synapse types. In contrast, ELKS2αB had no rescue activity on its own, consistent with the STED and electron microscopy data. Excitatory evoked transmission was monitored via NMDA receptors (NMDARs) to avoid confounding effects of network activity triggered by AMPA receptor activation. Decreasing initial p by lowering extracellular Ca²⁺ or the use of low affinity NMDAR antagonists confirmed that paired pulse ratios provide an accurate estimate of changes in p as a consequence of genetic manipulations under our conditions (FIG. S3). Together, these data establish that RIM is an important presynaptic organizer for the control of active zone protein levels, positioning and function.

### RIM Zinc Fingers Localize to the Synaptic Vesicle Cloud

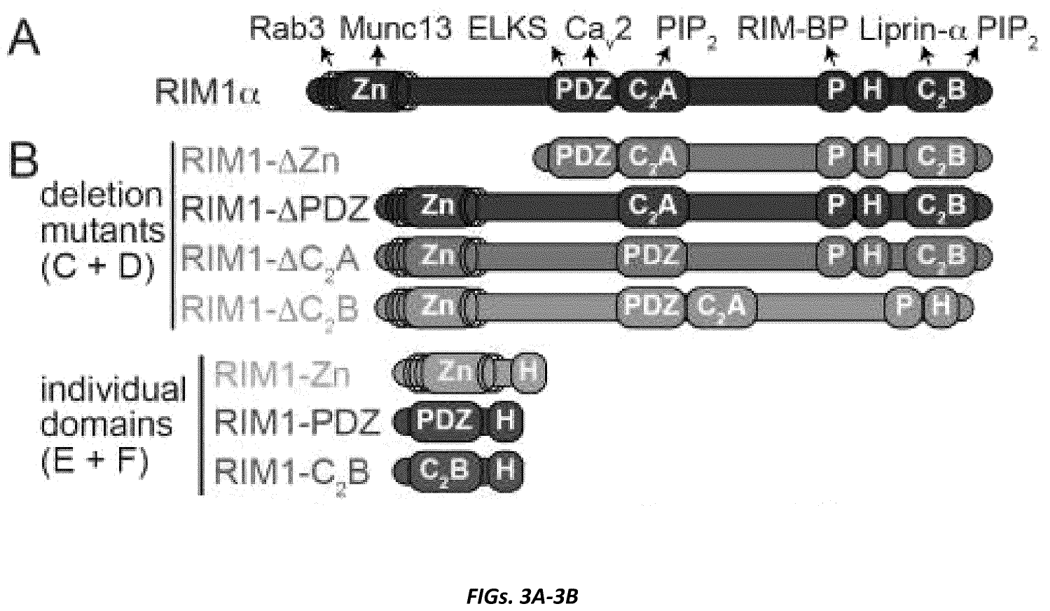

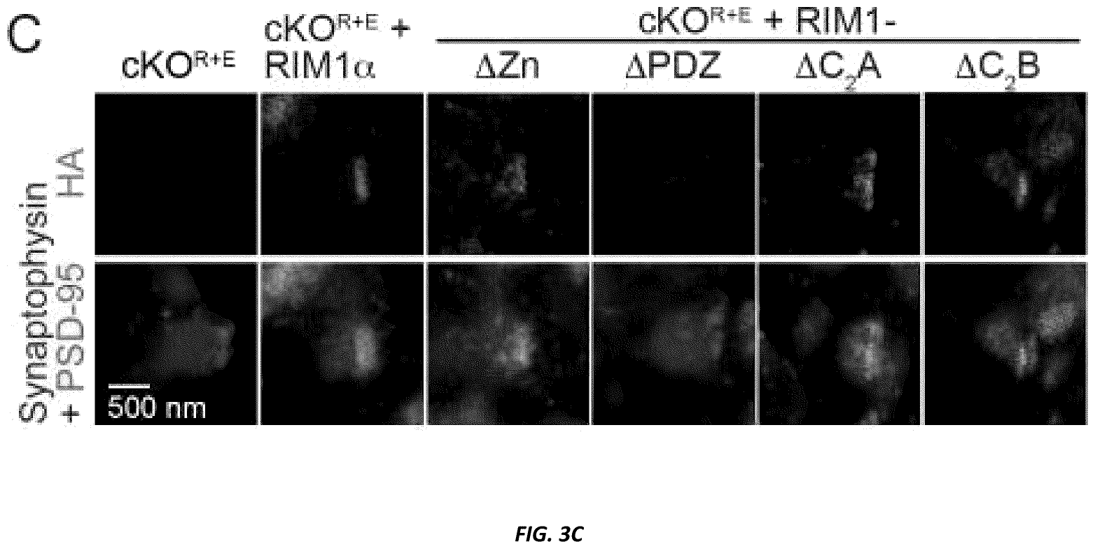

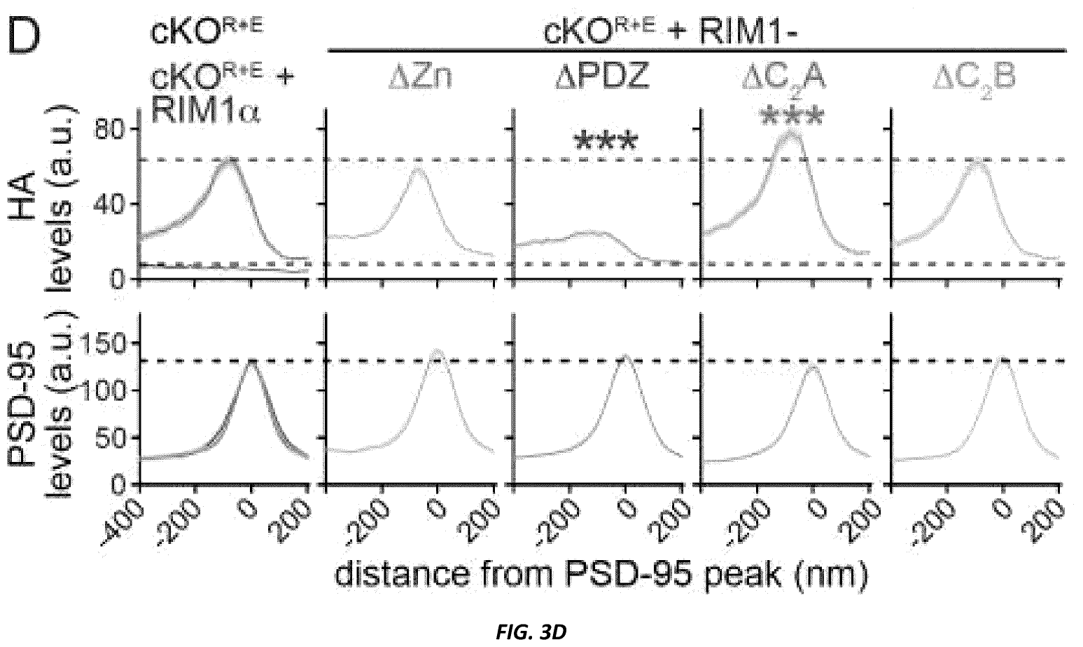

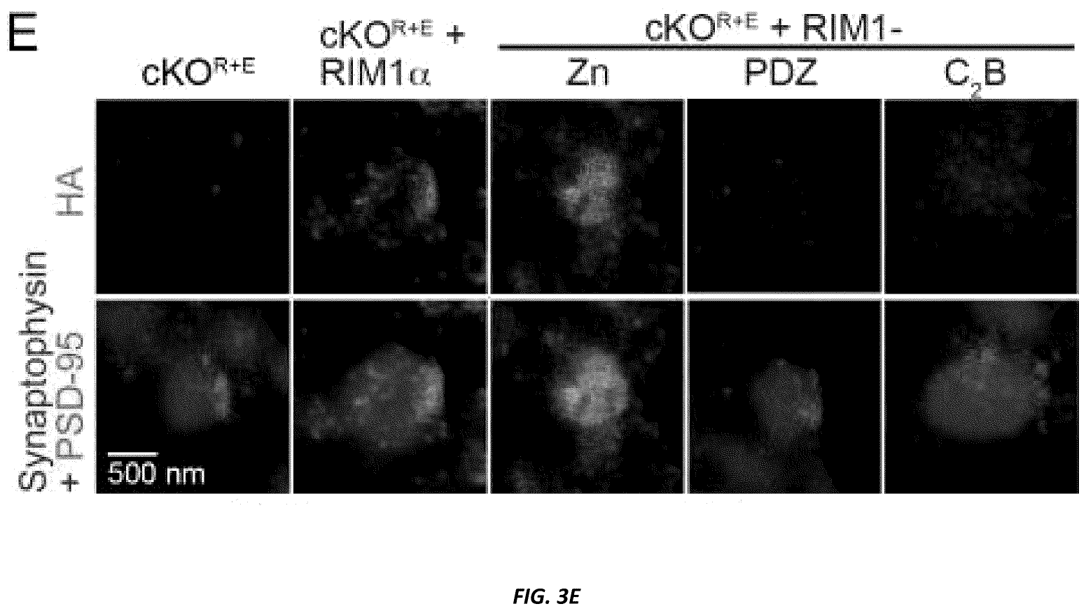

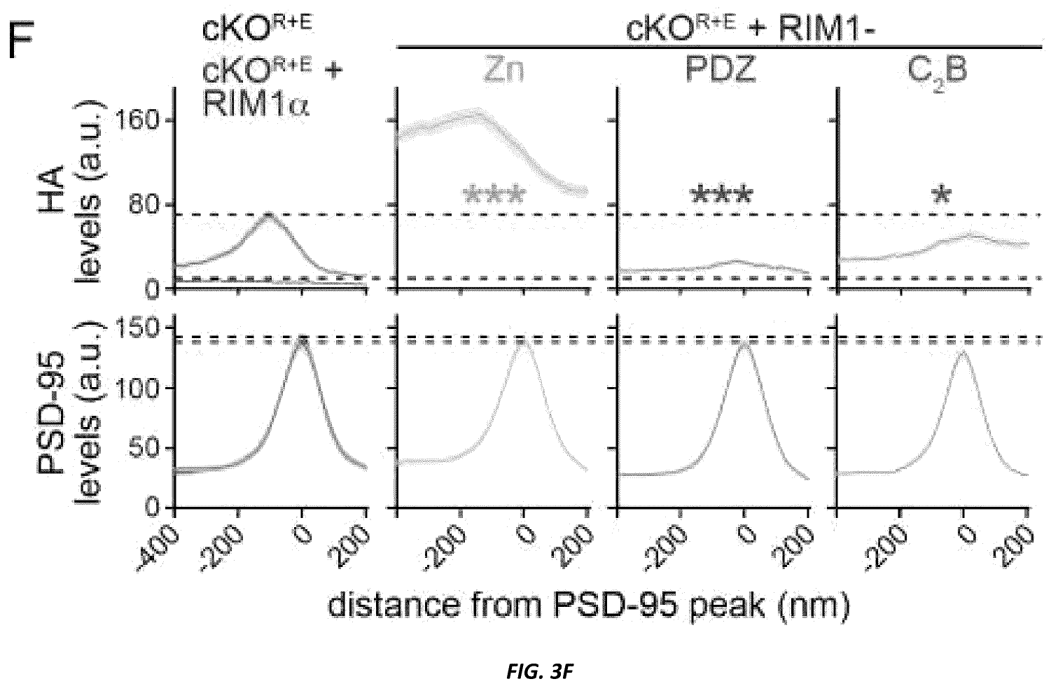

For building a minimal recovery system, we next needed to distinguish between RIM domains that mediate active zone targeting of RIM from those that are important for its functions in scaffolding other proteins and in mediating vesicle docking and release. We generated lentiviral constructs (FIGS. 3A, 3B) in which we either deleted individual RIM domains (RIM1-ΔZn, -ΔPDZ, -ΔC₂A and -ΔC₂B) or that contained only one domain at a time (RIM1-Zn, -PDZ and —C₂B; the tested C₂A domain constructs were not efficiently expressed and hence C₂A domains could not be assessed in these experiments). cKO^{R+E} neurons were transduced with each individual virus for rescue, and each protein was efficiently expressed (FIGS. S4A, S4B).

Assessment of RIM active zone targeting using STED line profile analyses revealed that the PDZ domain was necessary for RIM target membrane localization after active zone disruption, as removing the PDZ domain abolished RIM active zone targeting (FIGS. 3C, 3D). Other domain deletions did not impair RIM localization. Notably, no single domain of RIM was targeted to the plasma membrane opposed to the PSD when expressed alone (FIGS. 3E, 3F). Hence, while multiple RIM domains cooperated for RIM active zone targeting, the RIM PDZ domain is essential for such targeting. It is noteworthy that in neurons lacking only RIM rather than RIM and ELKS, most active zone proteins remain localized to synapses, and the PDZ domain is not essential for RIM localization to nerve terminals (de Jong et al., 2018; Kaeser et al., 2011). In active zone disrupted cKO^{R+E} synapses, this redundancy is lost and the PDZ domain is essential (FIGS. 3C, 3D). Hence, the highly interconnected active zone protein networks rely on redundant scaffolding mechanisms that include ELKS (Held and Kaeser, 2018).

Notably, the RIM1 zinc finger alone, while not localized to the active zone, was strongly enriched within nerve terminals (FIGS. 3E, 3F). RIM1-Zn localization highly overlapped with the synaptic vesicle protein Synaptophysin, suggesting that this domain may be associated with vesicles when expressed on its own. Since the RIM zinc finger interacts with the vesicular GTPases Rab3 and Rab27 (Dulubova et al., 2005; Fukuda, 2003; Wang et al., 1997), it is likely that RIM1-Zn associates through these interactions with synaptic vesicles. The complementary protein fragment, a version of RIM that lacks the zinc finger domain termed RIM1-ΔZn, localized to the active zone area apposed to the PSDs (FIGS. 3C, 3D), establishing that the zinc finger domain is not required for the synaptic delivery of RIM.

### RIM1 Zinc Fingers Recruit Munc13 for Establishing Release-Competence of Non-Docked Vesicles

The differential localization of RIM1-Zn (to vesicles) and RIM1-ΔZn (to the target membrane) may be related to their roles in release. Previous studies in RIM knockout synapses have suggested that RIM zinc finger domains prime synaptic vesicles while the C-terminal domains within RIM1-ΔZn tether CaV2 channels and interact with the target membrane for fast release triggering (Deng et al., 2011; de Jong et al., 2018; Kaeser et al., 2011). We tested these models by assessing the molecular roles (recruitment of Munc13 and CaV2s) and functional roles (priming, docking and releasing of vesicles) of RIM1-Zn and RIM1-ΔZn after active zone disruption.

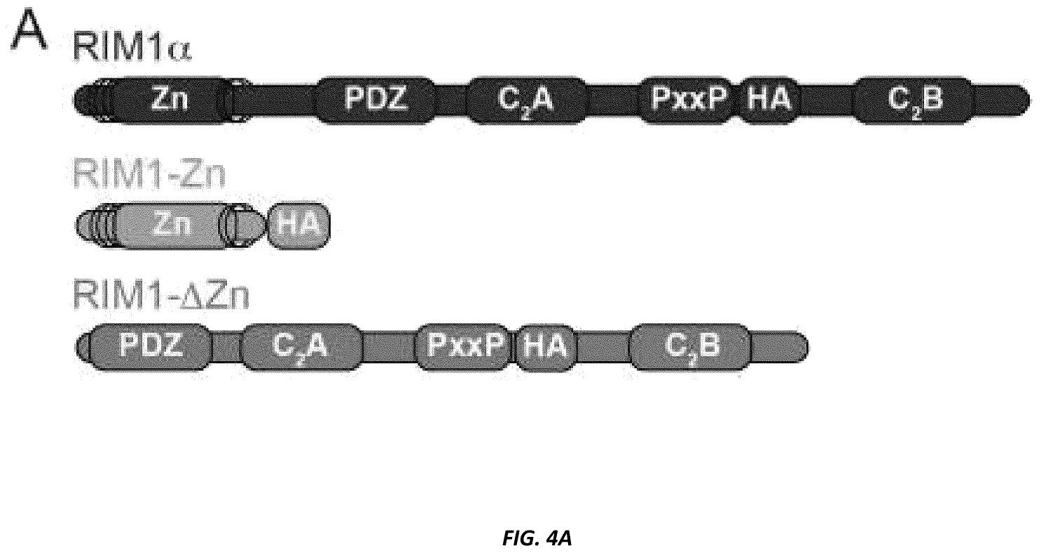

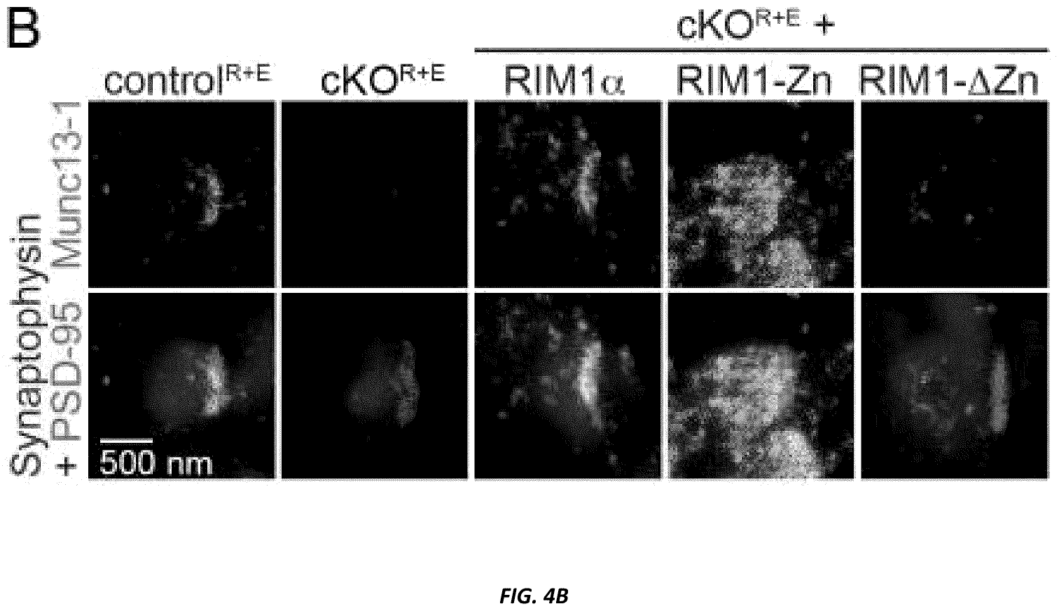

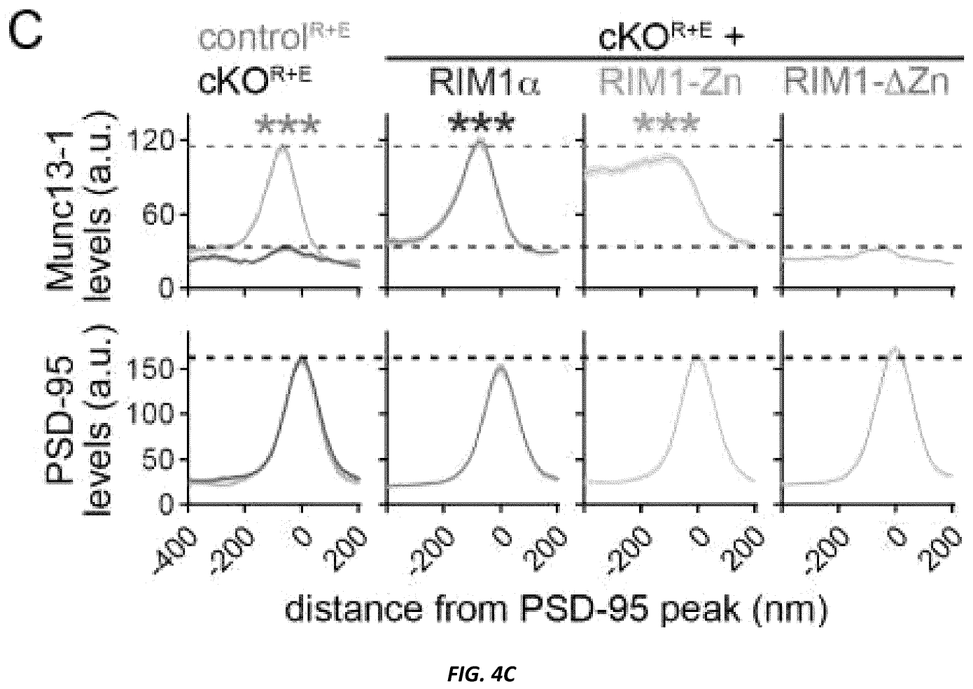

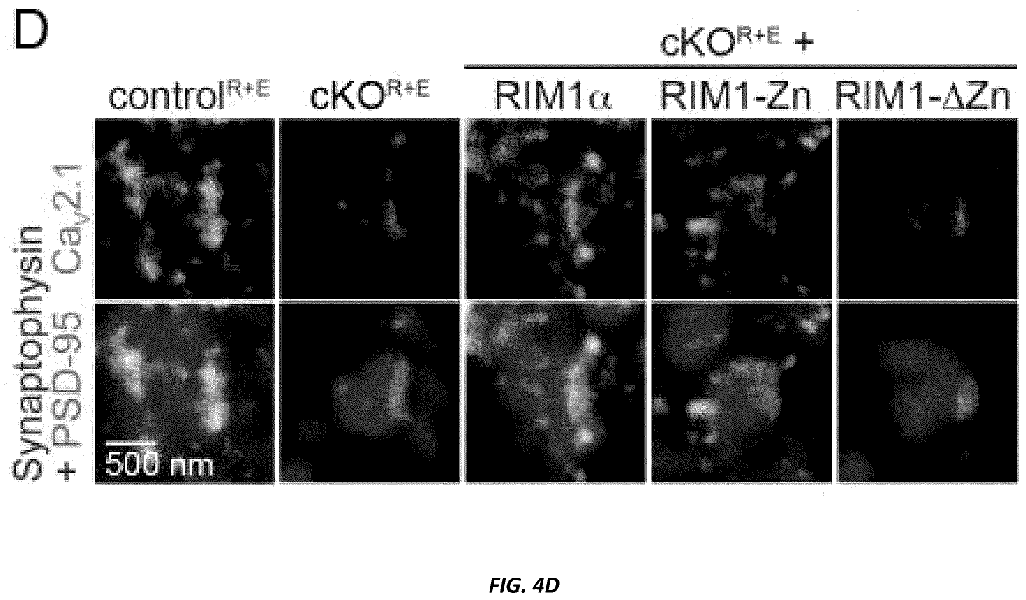

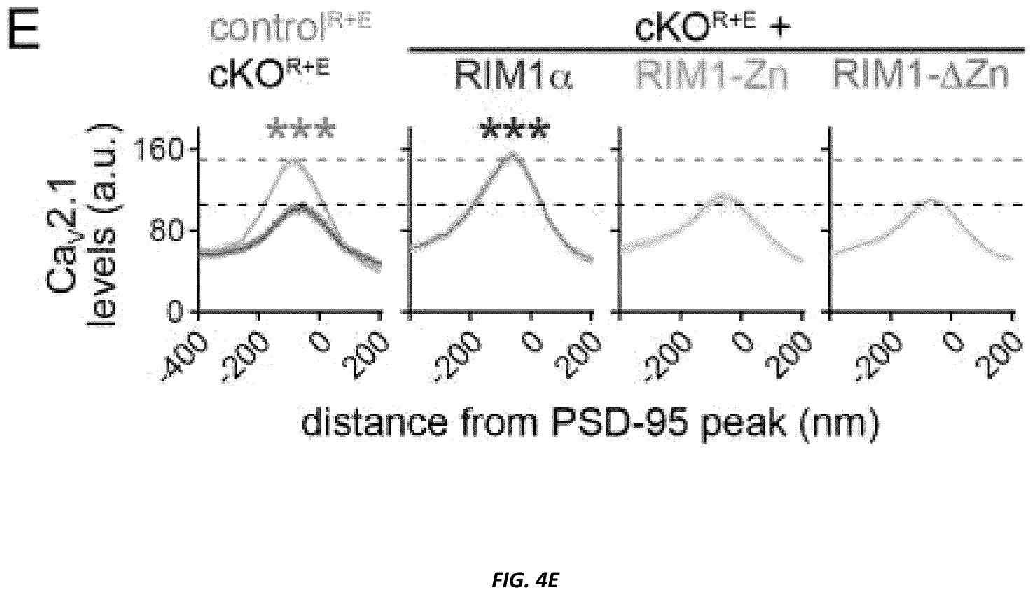

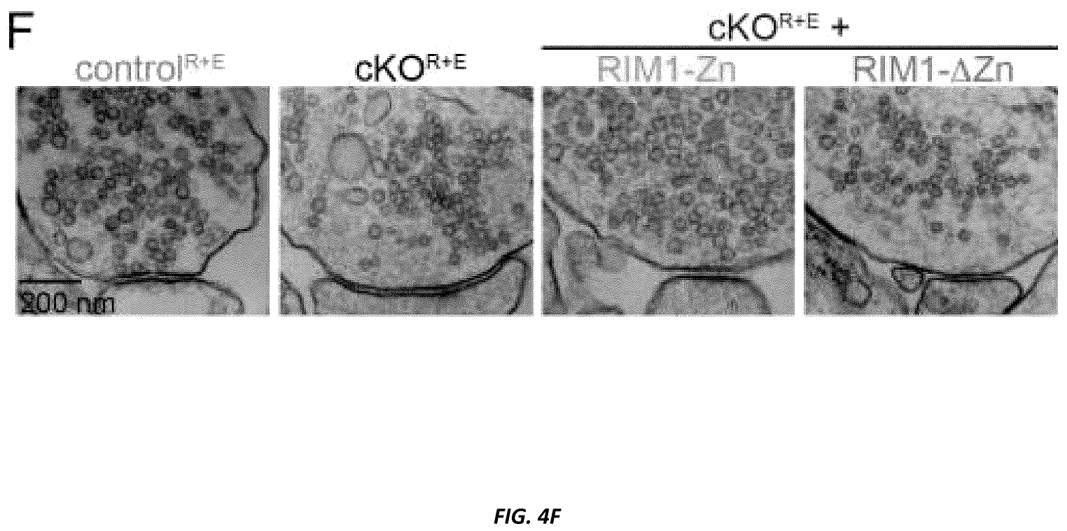

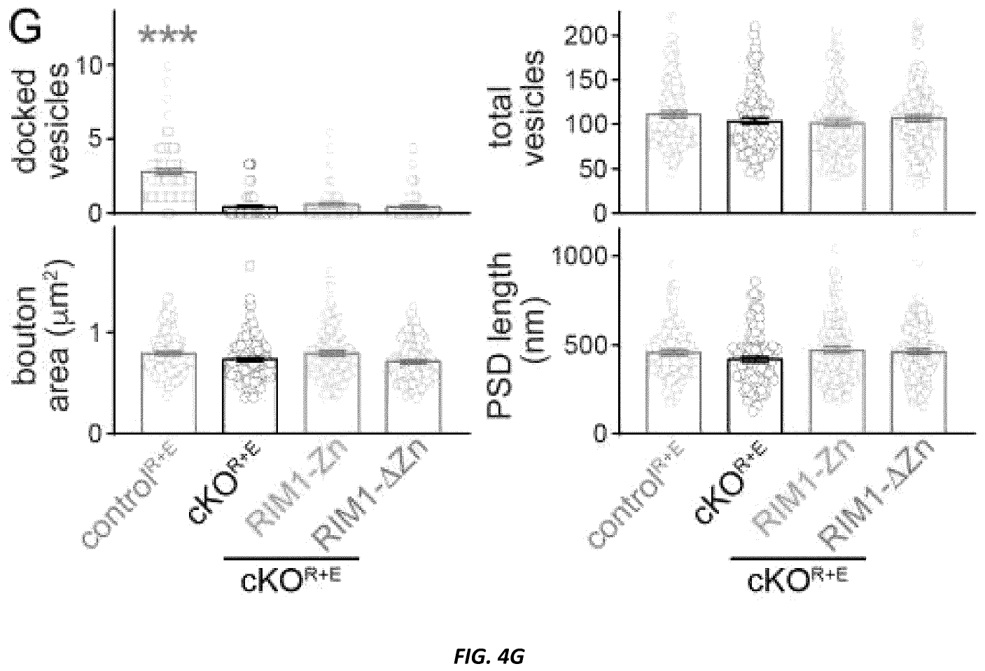

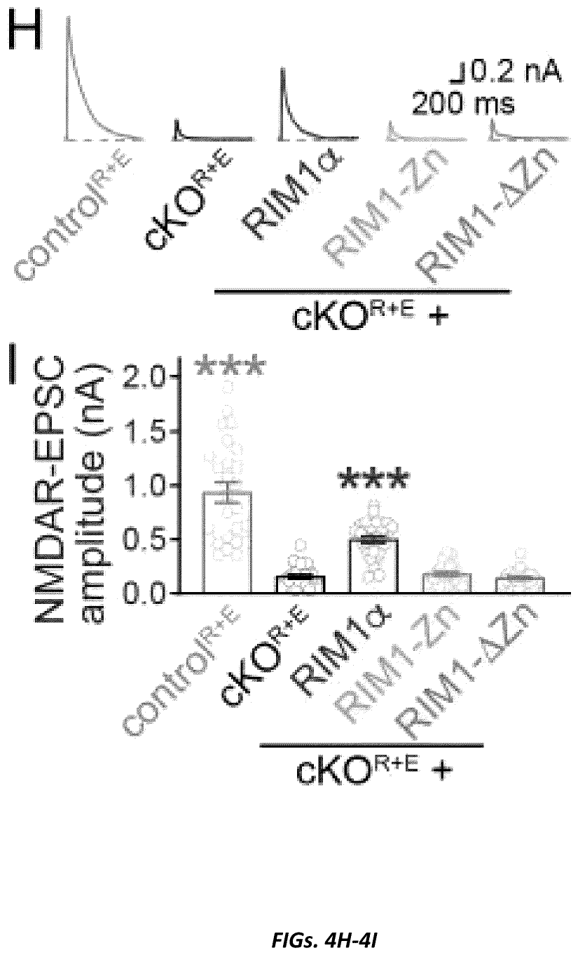

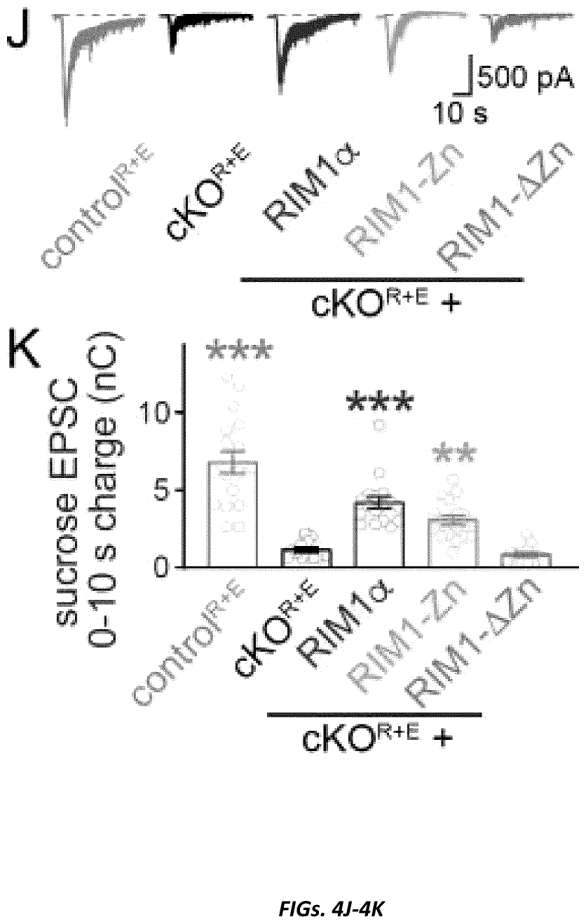

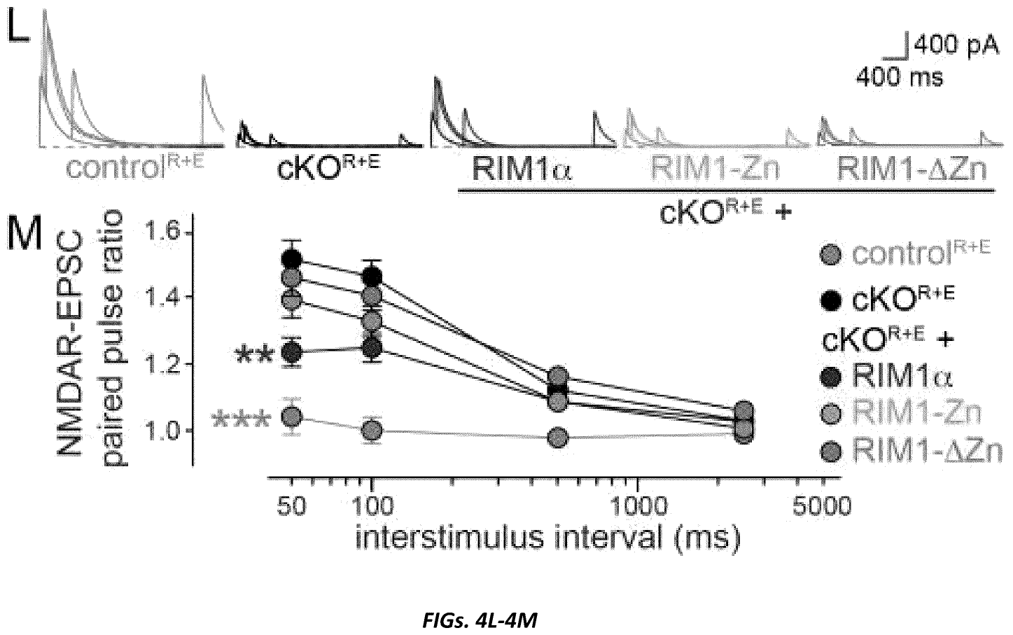

Strikingly, RIM1-Zn co-recruited Munc13 in a pattern mimicking the wide-spread localization of RIM1-Zn (FIGS. 4B, 4C). In contrast, RIM1-ΔZn, which contains the RIM scaffolding domains (PDZ, C₂A, PxxP, C₂B) and localizes to the active zone area (FIGS. 3A-3D), was unable to enhance CaV2s or Munc13 (FIGS. 4B-4E). This suggests that RIM1-Zn, which binds to Munc13 and Rab3 (Betz et al., 2001; Dulubova et al., 2005), recruits Munc13 to synapses, stabilizes it, and turns is into a protein associated with synaptic vesicles rather than the target membrane. We next analyzed high pressure-frozen neurons using electron microscopy from the same rescue conditions. Both RIM1-Zn and RIM1-ΔZn lacked rescue activity, and synaptic vesicles remained undocked in both conditions (FIGS. 4F, 4G). In conclusion, Munc13, a protein implicated in synaptic vesicle docking (Imig et al., 2014; Siksou et al., 2009), was not targeted to the presynaptic plasma membrane upon RIM1-Zn re-expression but was instead associated with the vesicle cloud. Recovering its presence in the nerve terminal was not sufficient to mediate docking.

Electrophysiological recordings of excitatory (FIGS. 4H-4M) and inhibitory (FIGS. S4C-S4H) transmission revealed that RIM1-Zn and RIM1-ΔZn failed to restore action potential-triggered synaptic transmission and release probability of excitatory synapses (FIGS. 4H-4M), and only mild rescue of these parameters was observed at inhibitory synapses (FIGS. S4C-S4H). This is different from rescue experiments after RIM knockout only (instead of cKO^{R+E}), where RIM1-ΔZn is sufficient to restore Ca²⁺ entry and mediates an increase in p (Deng et al., 2011; Kaeser et al., 2011). Hence, RIM C-terminal scaffolding domains need ELKS or N-terminal RIM sequences (FIGS. 1 and 2) to execute their roles in release, but are sufficient to mediate target membrane localization of RIM (FIGS. 3C, 3D).

Remarkably, however, RIM1-Zn strongly enhanced vesicle fusogenicity measured via application of hypertonic sucrose, nearly as efficiently as full-length RIM1α (FIGS. 4J, 4K, S4E, S4F). These data support the model that RIM zinc fingers activate Munc13 for vesicle priming by recruiting and monomerizing it via binding to Munc13 C₂A domains (Camacho et al., 2017; Deng et al., 2011) and—strikingly—this function can be executed on non-docked vesicles distant from the target membrane. These vesicles, however, are inaccessible to action potential-triggering. We conclude that undocked vesicles can become release-competent by positioning activated Munc13 on them. Hence, Munc13 enhances fusogenicity even if it is not localized to release sites at the target membrane, and these “molecularly” primed vesicles do not need to be docked. This may explain why some priming remains when the active zone is disrupted and docking is abolished (Wang et al., 2016), as some Munc13 may be recruited to vesicles via direct interactions (Quade et al., 2019).

### Docking of Release-Competent Vesicles to Ca²⁺ Channels Restores Fast Release in the Absence of Active Zone Scaffolds

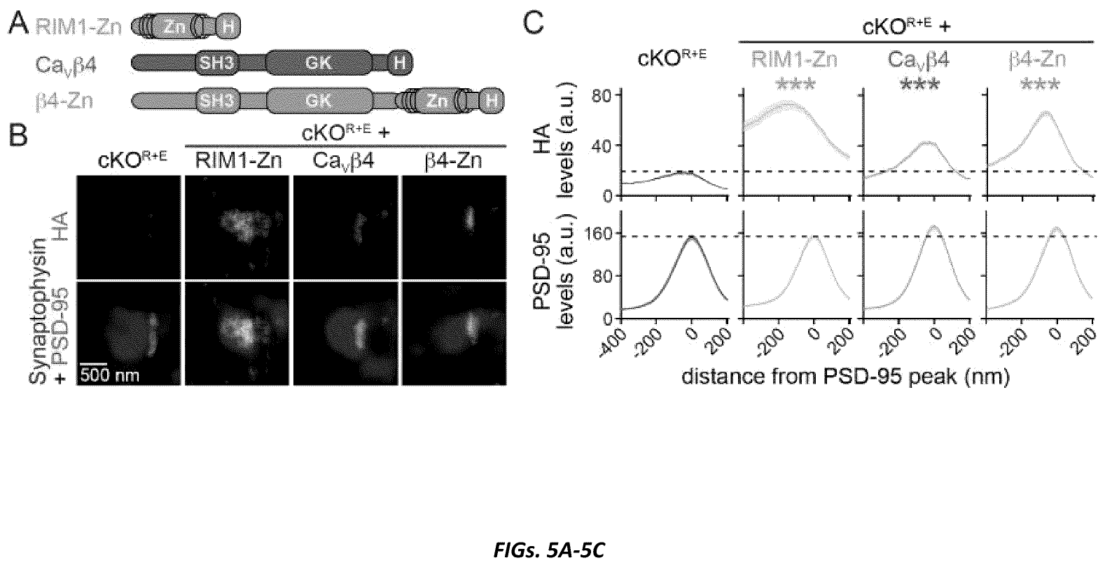

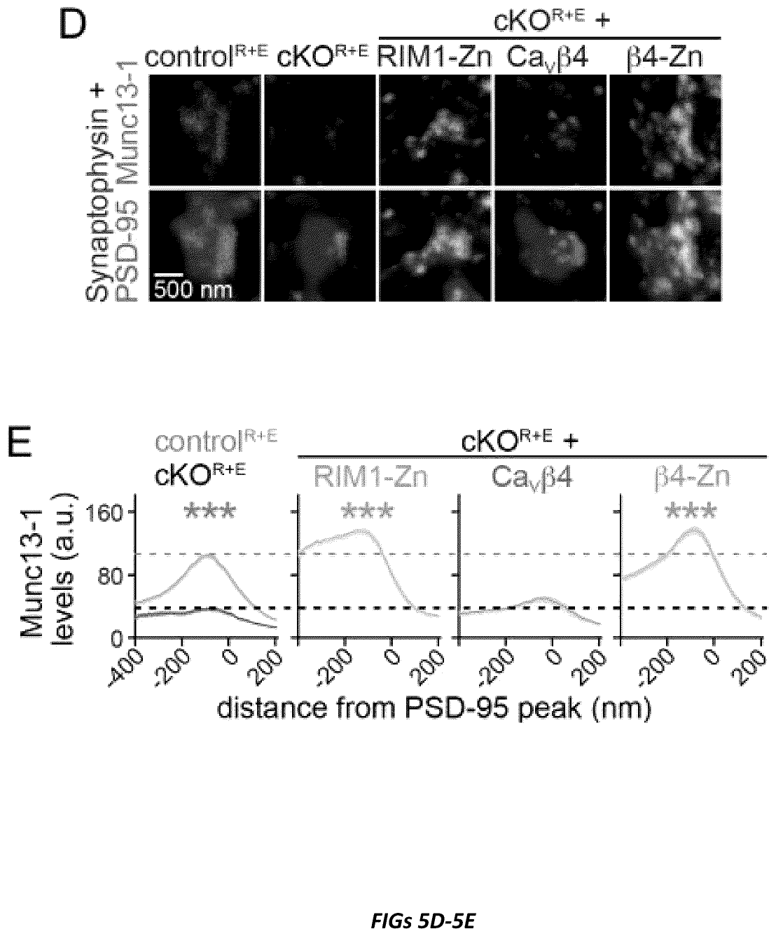

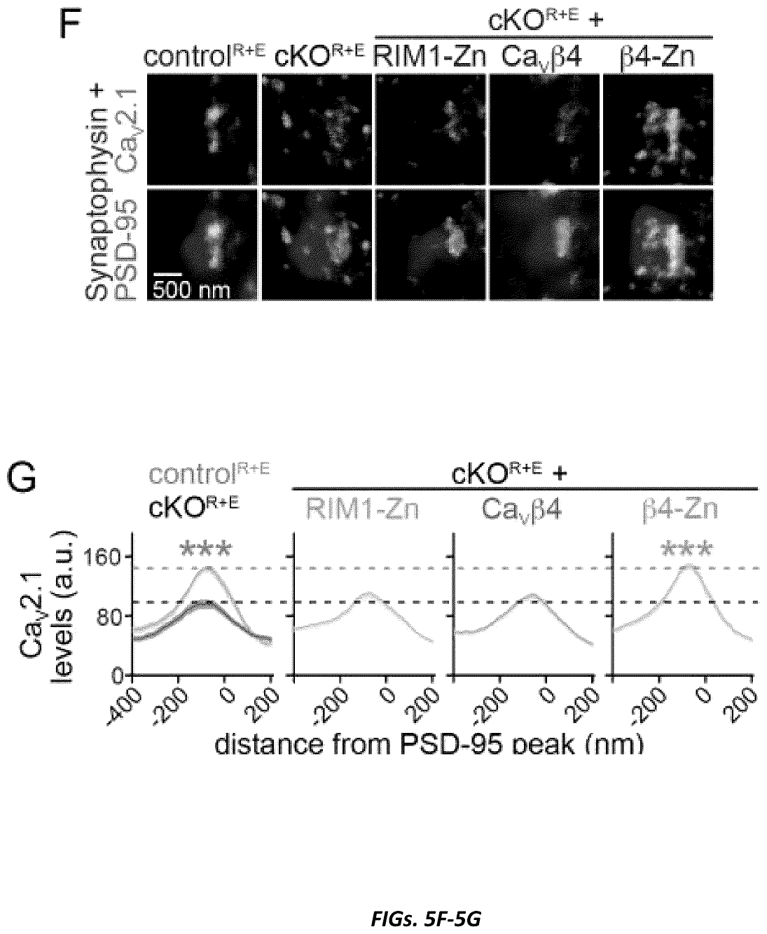

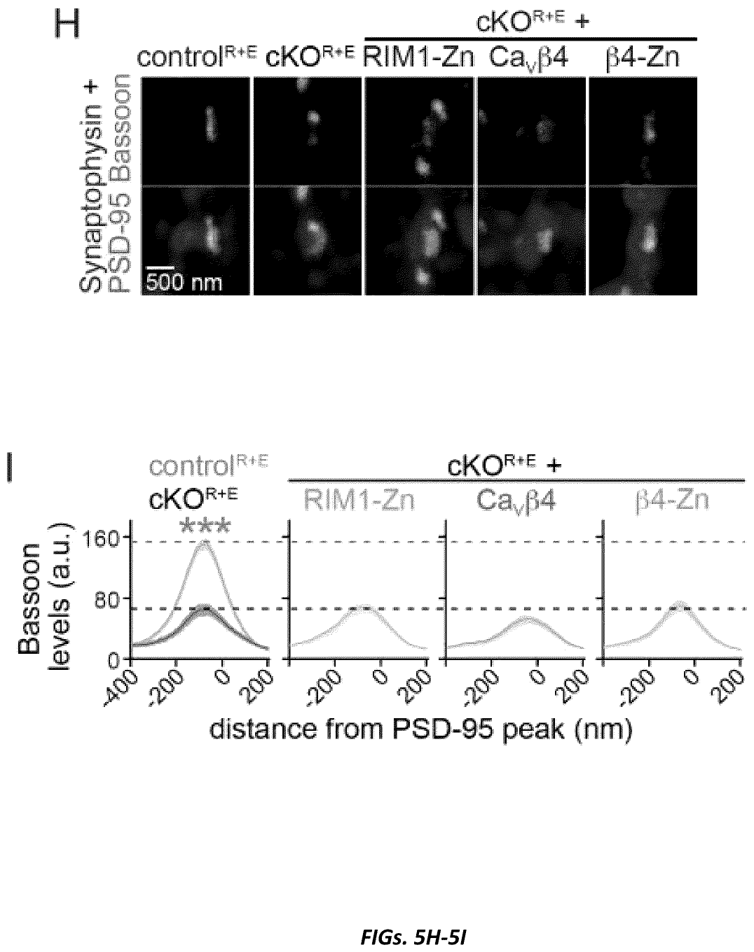

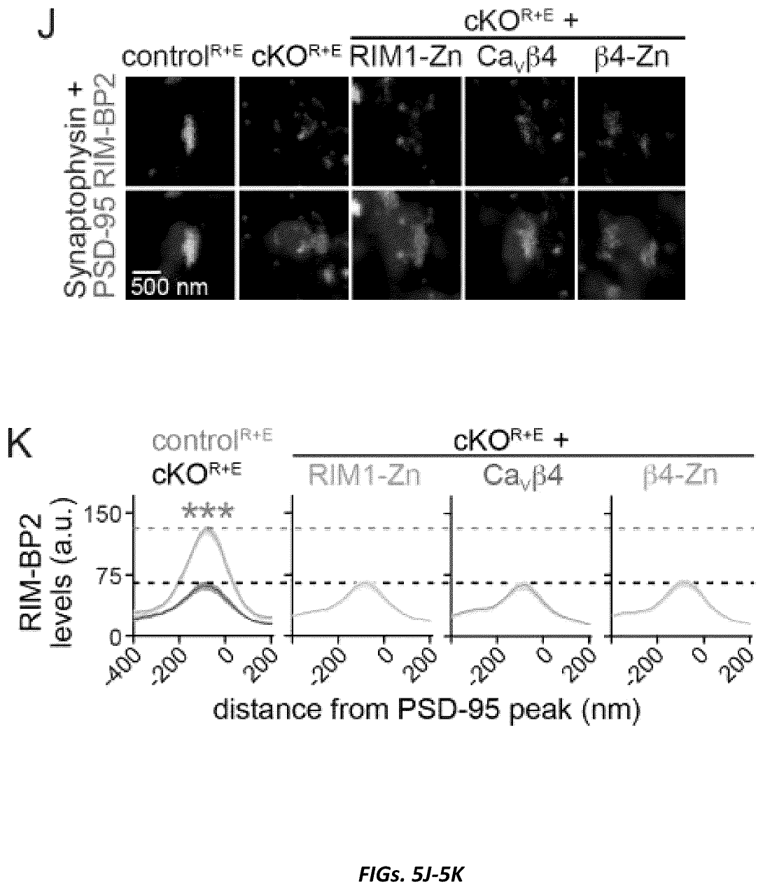

With the goal to selectively rebuild active zone mechanisms without restoring the vast scaffolding structure, we aimed at positioning the release-competent vesicles close to Ca²⁺ entry. We screened eight fusion-proteins of the RIM zinc finger domain to other proteins or protein fragments associated with the target membrane (FIG. S5A). Fusions with Ca_{V}β1 Ca²⁺ channel subunits appeared to efficiently restore evoked transmission and release probability (FIGS. S5B-S5E), suggesting that they may do so by co-localizing vesicle priming and Ca²⁺-entry. We selected the fusion of the RIM1 zinc finger to Ca_{V}β4 (FIG. 5A, β4-Zn) for a full characterization because of its strong tendency to rescue and because endogenous Ca_{V}β4 is localized to active zones (FIGS. S5F, S5G). β4-Zn was efficiently expressed (FIG. S5H) and concentrated in a bar-shaped structure at the target membrane (FIGS. 5A-5C). The β4-Zn protein efficiently recruited Munc13-1 to the target membrane (FIGS. 5D, 5E), and also enhanced Ca_{V}2.1 active zone levels back to control levels (FIGS. 5F, 5G). The effects on Ca_{V}2.1 were notably absent when Ca_{V}β4 or RIM1-Zn were expressed on their own. Importantly, β4-Zn did not enhance or restore the levels of the other active zone scaffolds as assessed by staining and quantification of Bassoon and RIM-BP2 (FIGS. 5H-5K). Hence, β4-Zn targets the priming-complex of the RIM zinc finger and Munc13 to Ca²⁺ channels in the absence of the megadalton-sized scaffolding network that consists of full-length RIM, ELKS, RIM-BP and Bassoon.

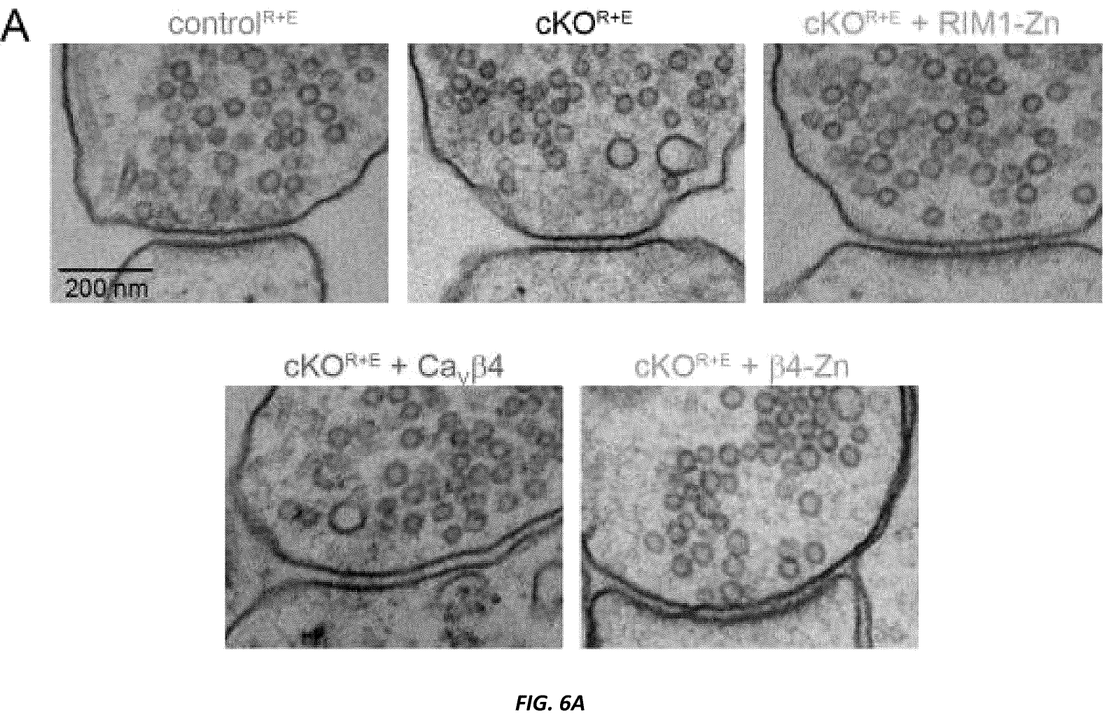

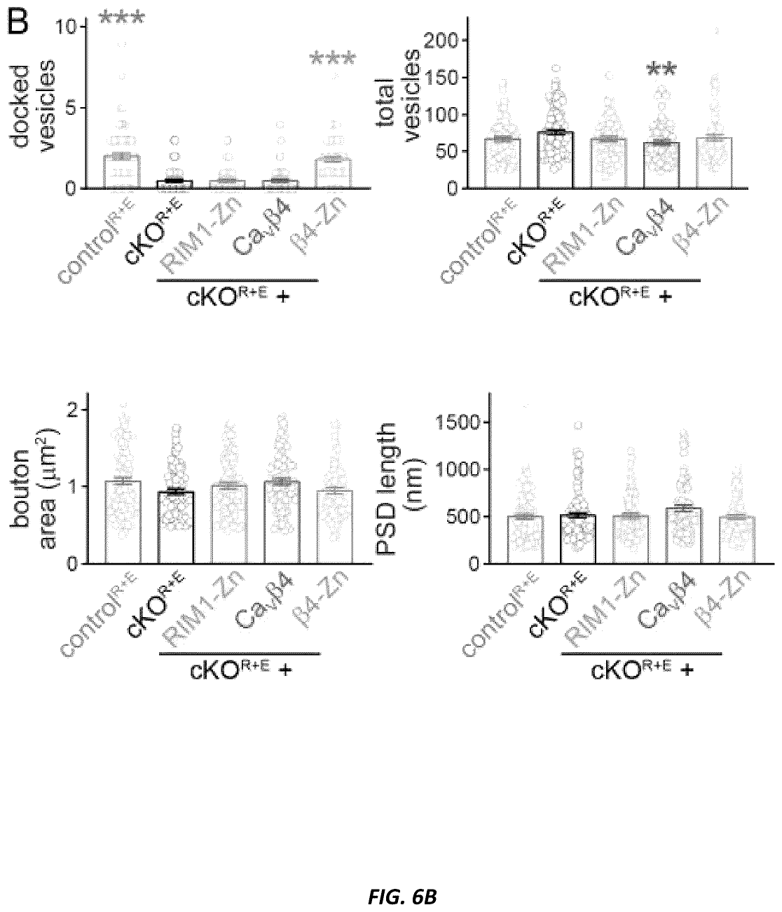

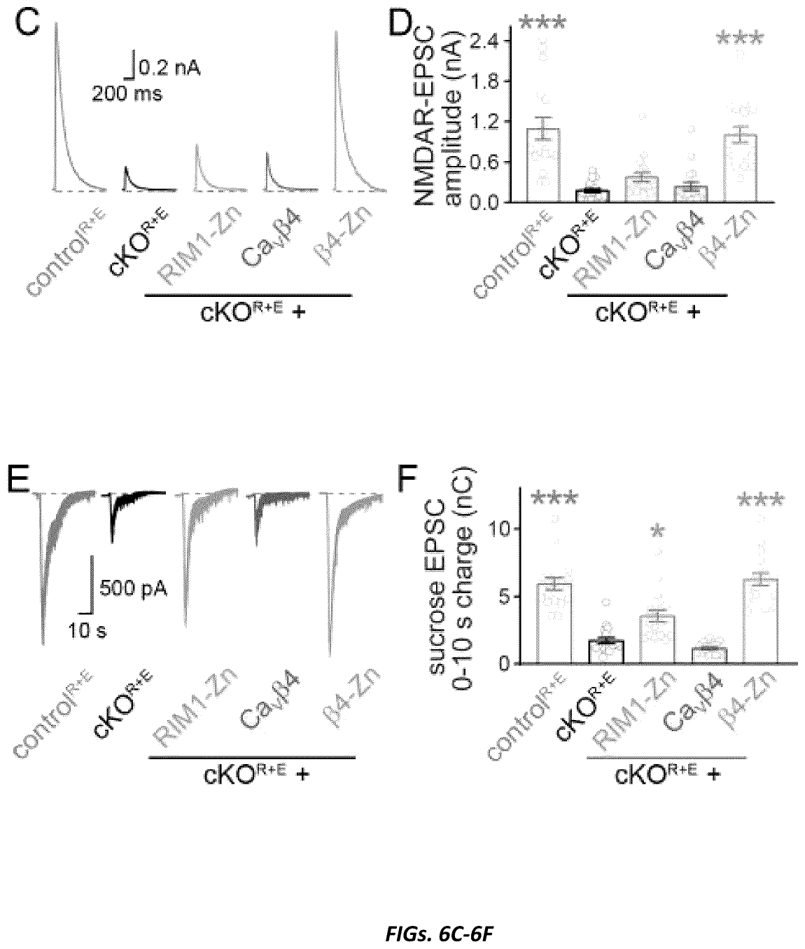

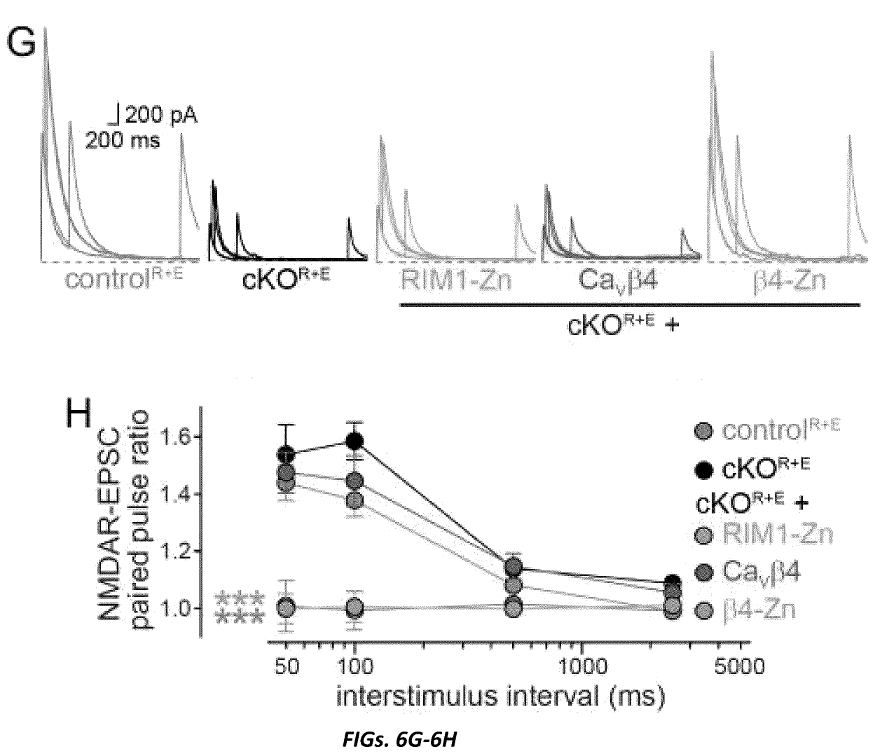

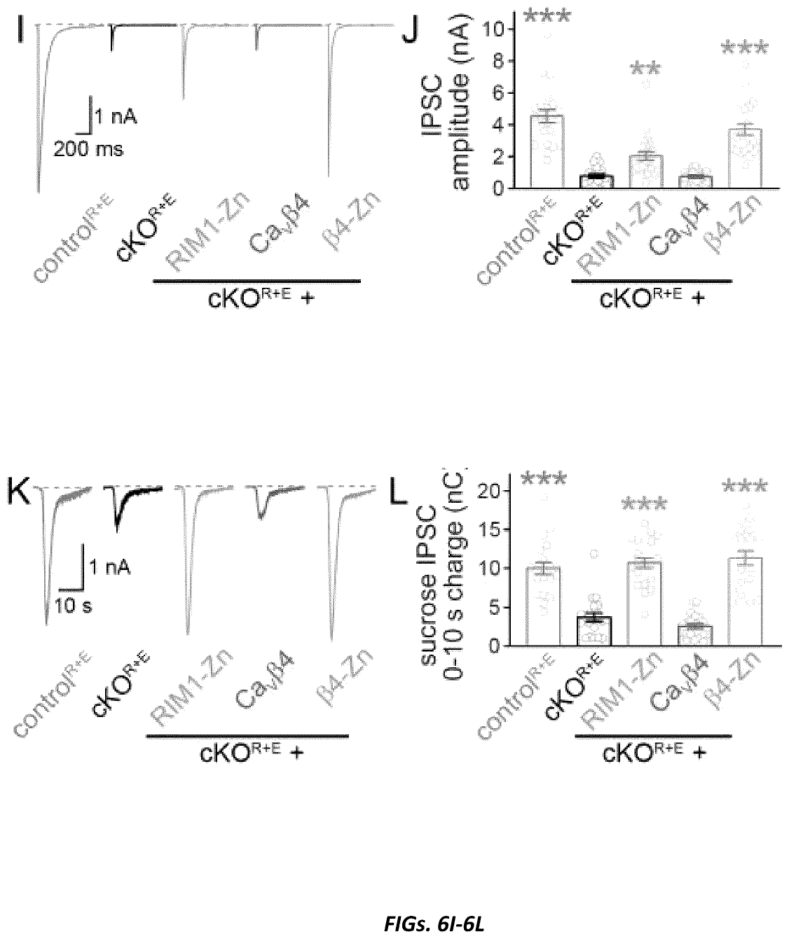

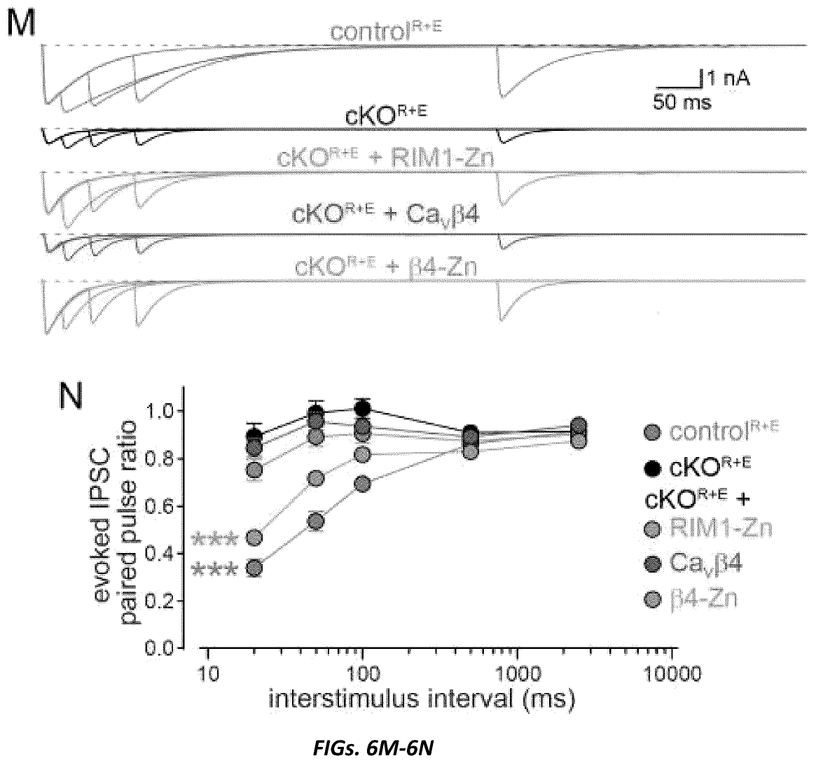

When we assessed these synapses using electron microscopy, we found that β4-Zn fully restored synaptic vesicle docking (FIGS. 6A, 6B). This is different from expression of CaVβ4 or RIM1-Zn alone, which did not enhance docking (FIGS. 6A, 6B), and much more robust than rescue efficacy of full-length RIM1α (FIGS. 2A, 2B). In electrophysiological recordings, we detected a full recovery of excitatory (FIGS. 6C, 6D) and inhibitory (FIGS. 6I, 6J) synaptic transmission. This included restoration of RRP (FIGS. 6E, 6F, 6K, 6L) and p (FIGS. 6G, 6H, 6M, 6N) back to levels indistinguishable from control^{R+E} neurons at both types of synapses. Hence, β4-Zn expression results in Munc13 recruitment and vesicle docking to the active zone close to Ca²⁺ channels such that extent and spatiotemporal precision of release are fully restored. Importantly, CaVβ4 or RIM1-Zn alone did not mediate these functions except for partial RRP recovery by RIM1-Zn (FIG. 4, S4, 6), and β4-Zn recovered docking and release without reinstating broader active zone scaffolds (FIG. 5). These data establish that vesicle docking close to Ca²⁺ channels enhances p of vesicles that are primed by RIM1-Zn and Munc13. They further suggest that vesicle docking close to CaV2s might not only enhance p, but also stabilize Ca²⁺ channels. Alternatively, interactions of CaVβ4 with vesicles and Munc13 may help CaV2 delivery to synapses.

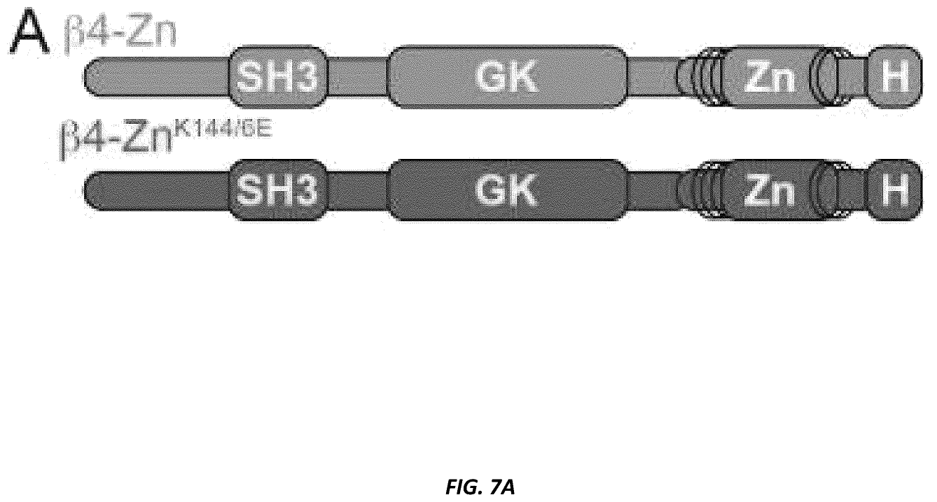

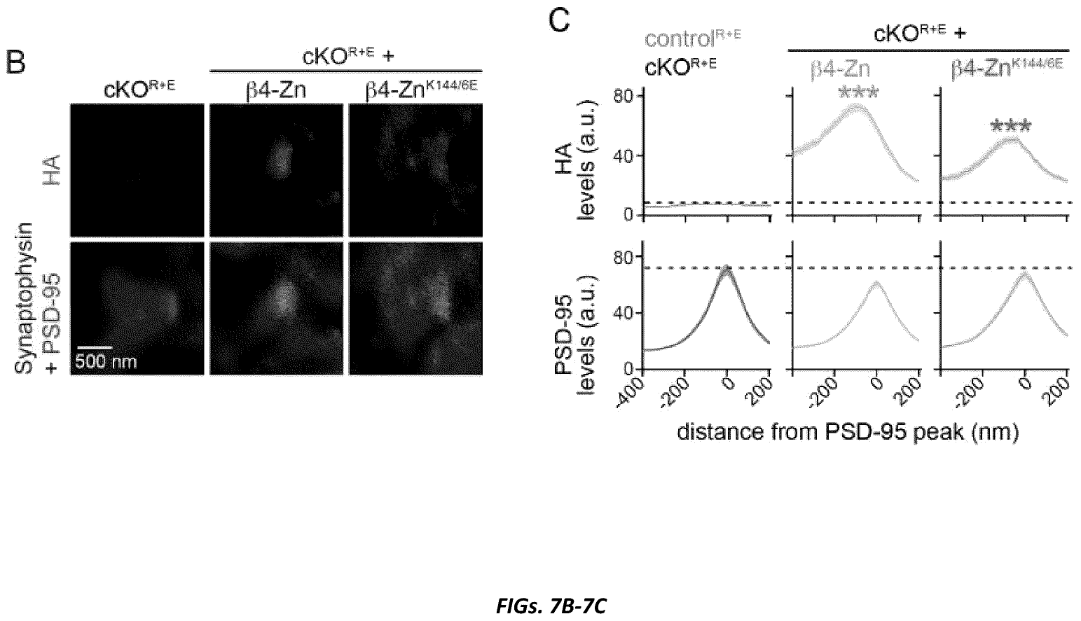

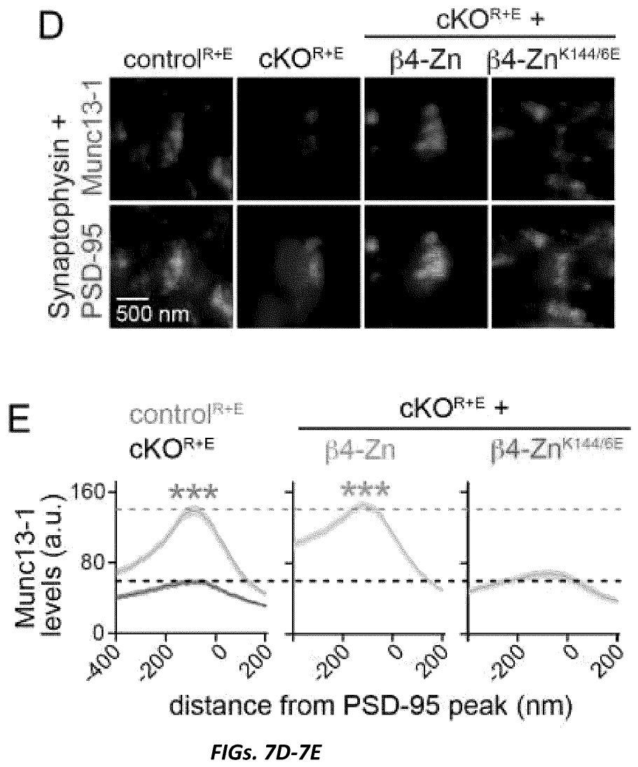

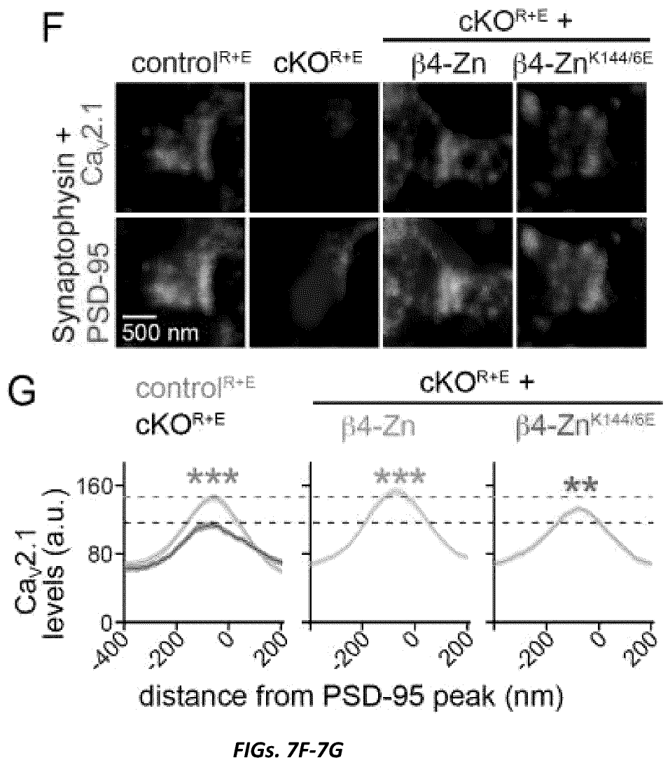

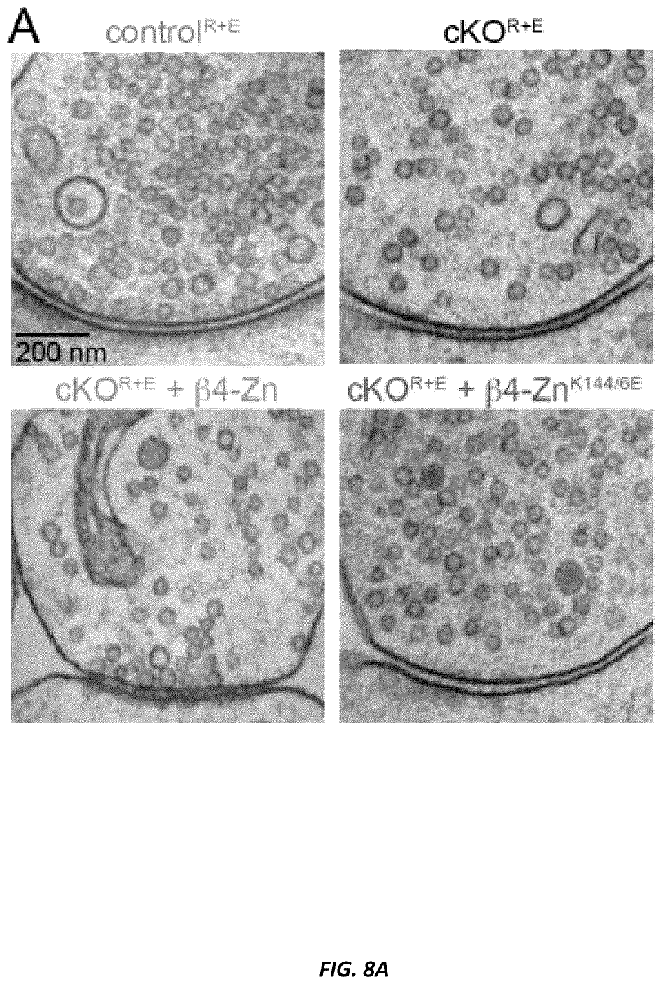

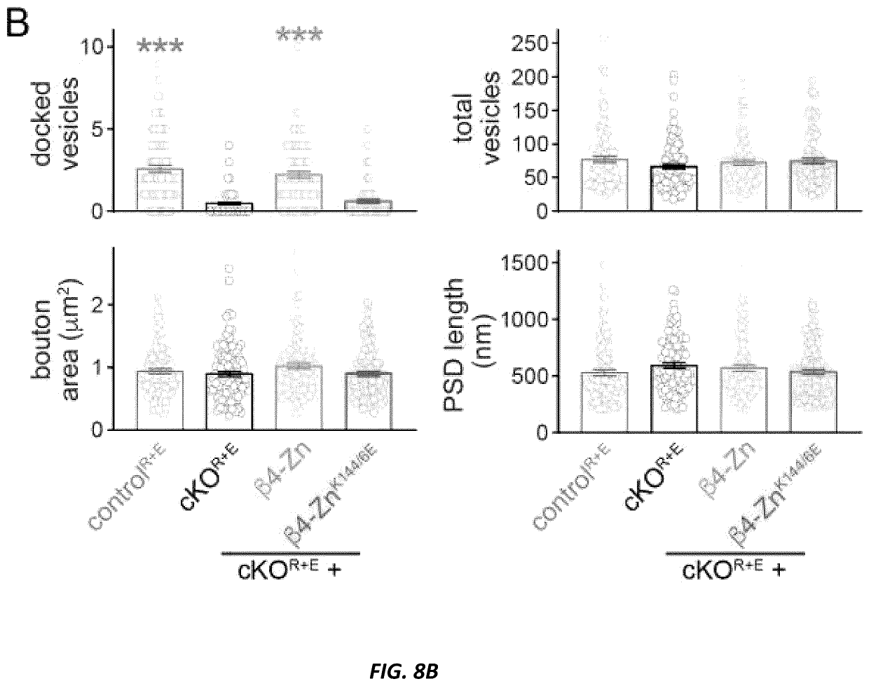

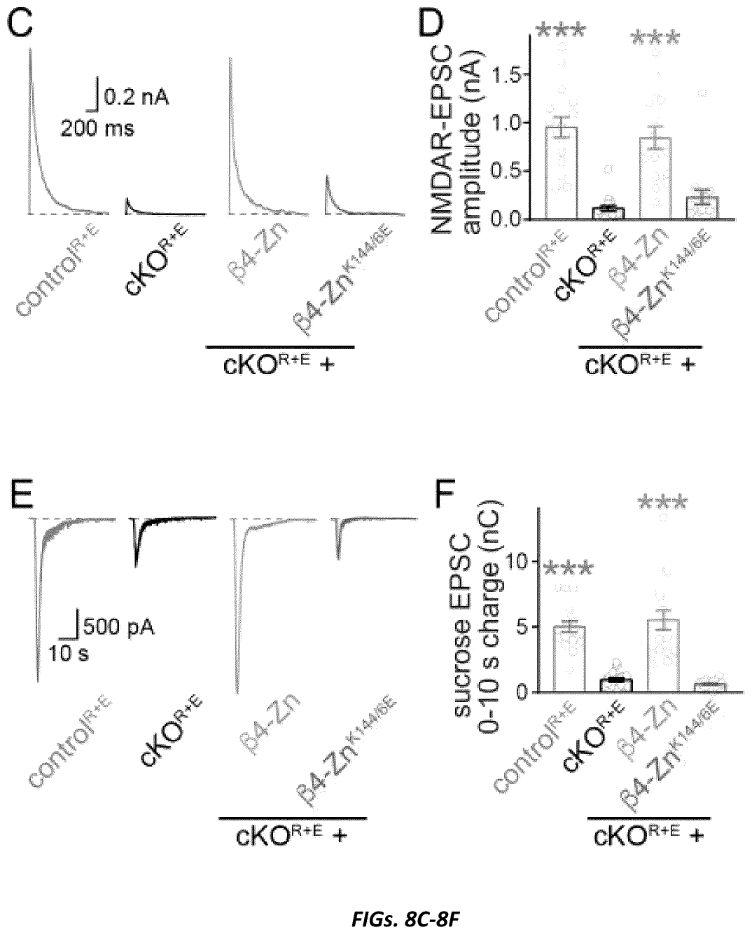

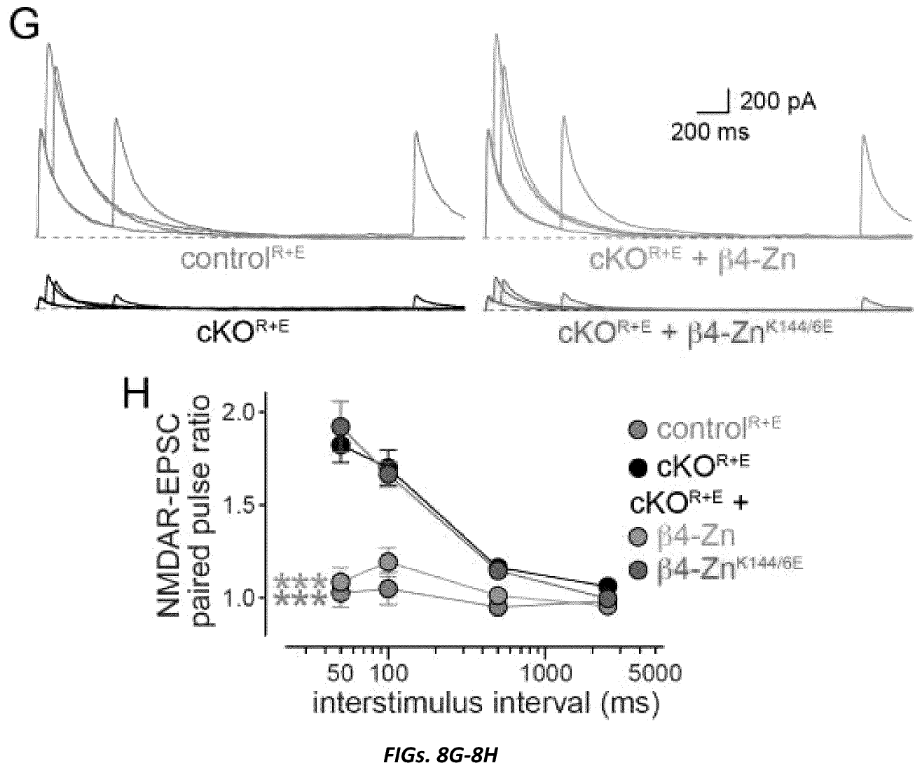

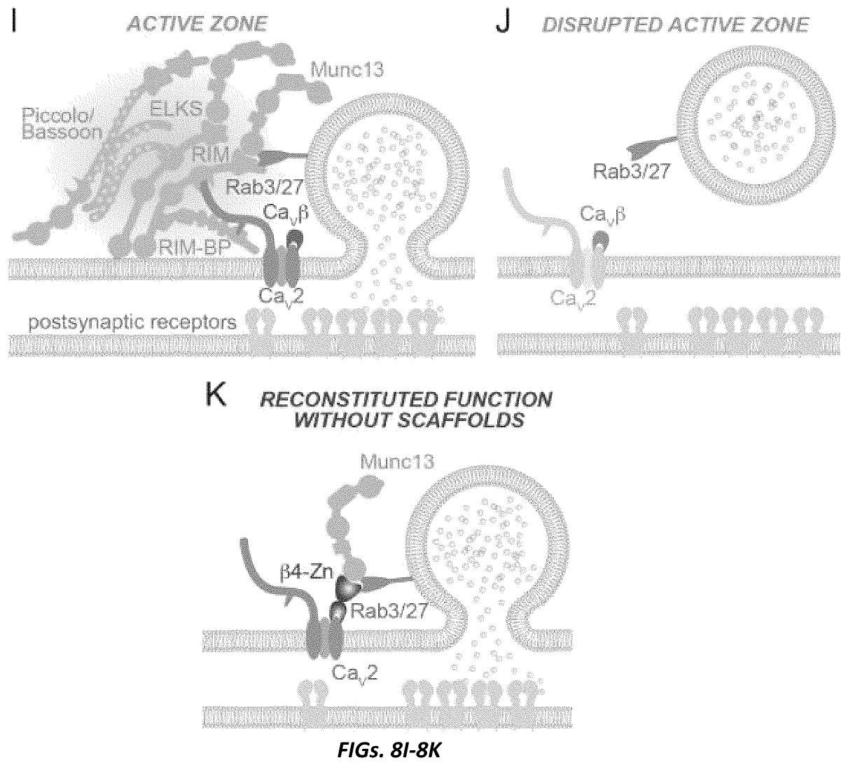

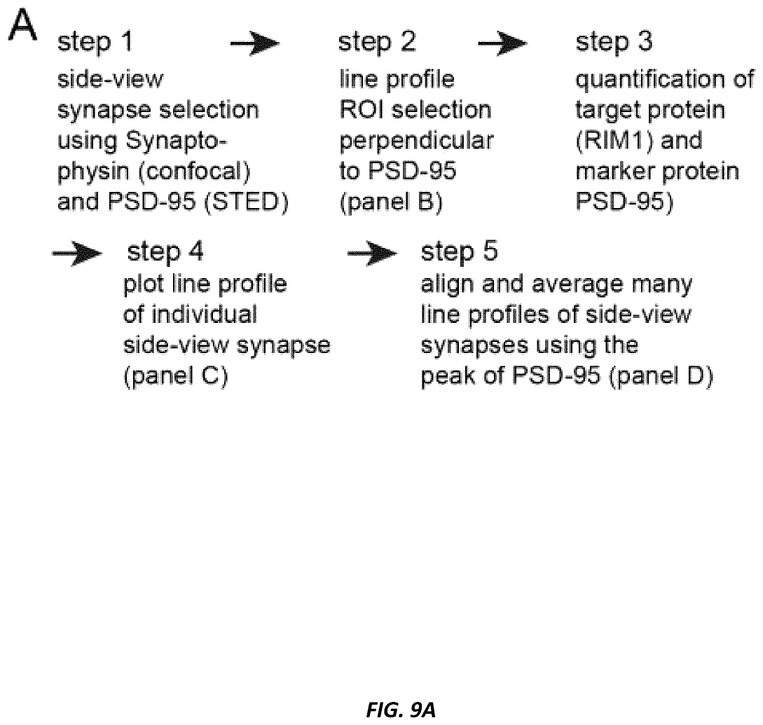

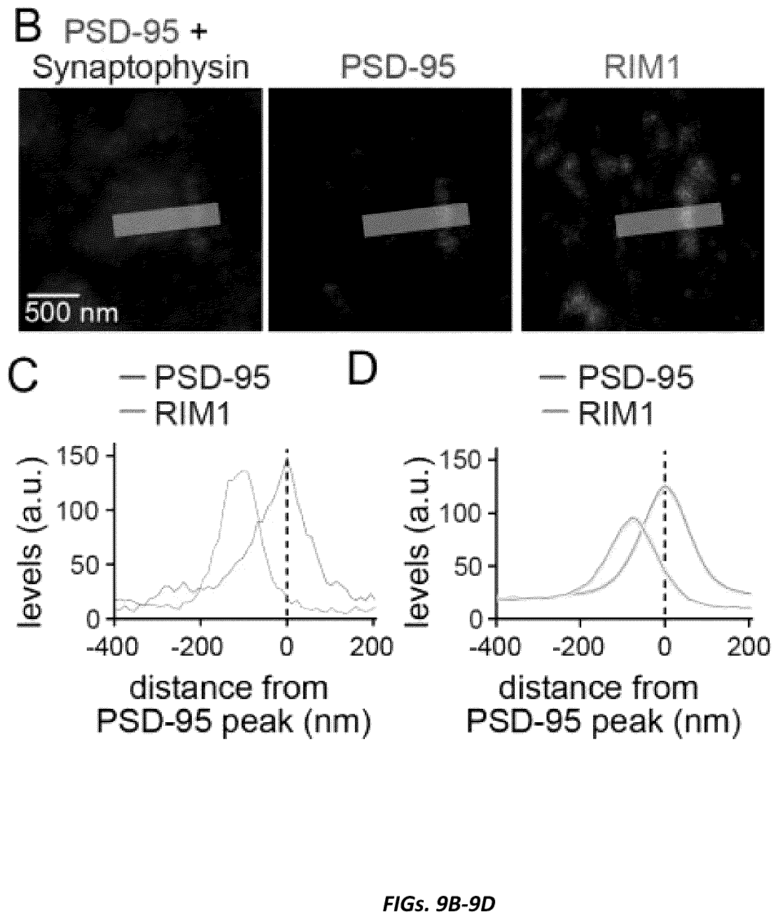

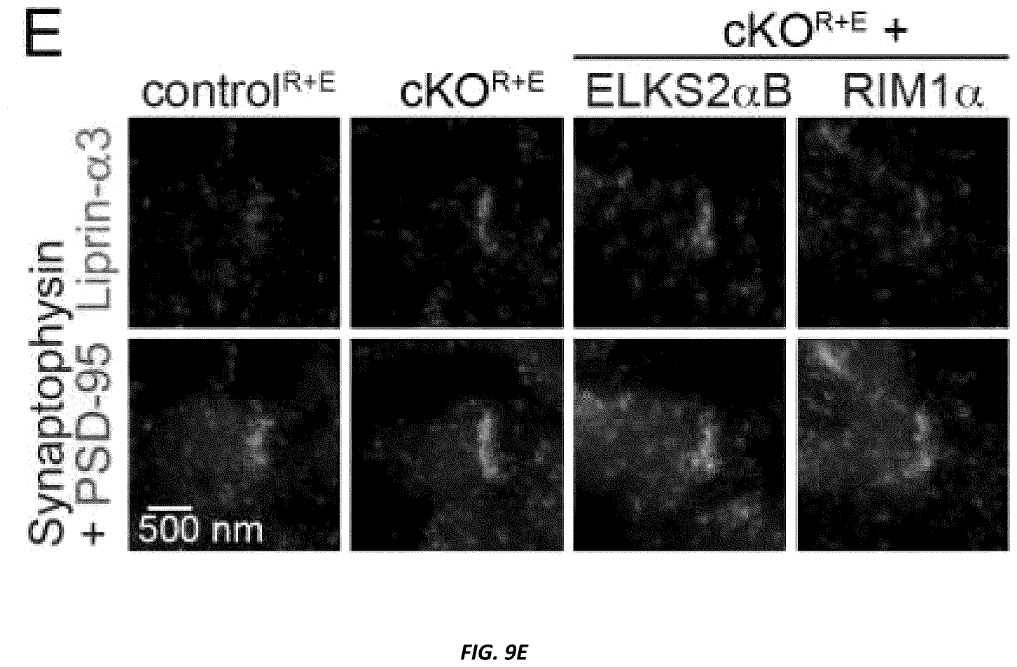

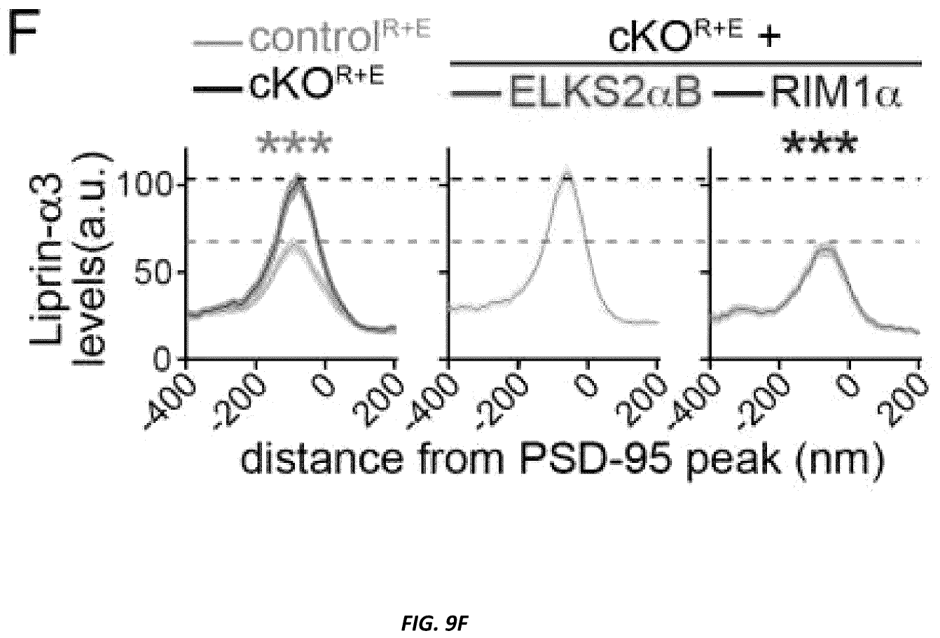

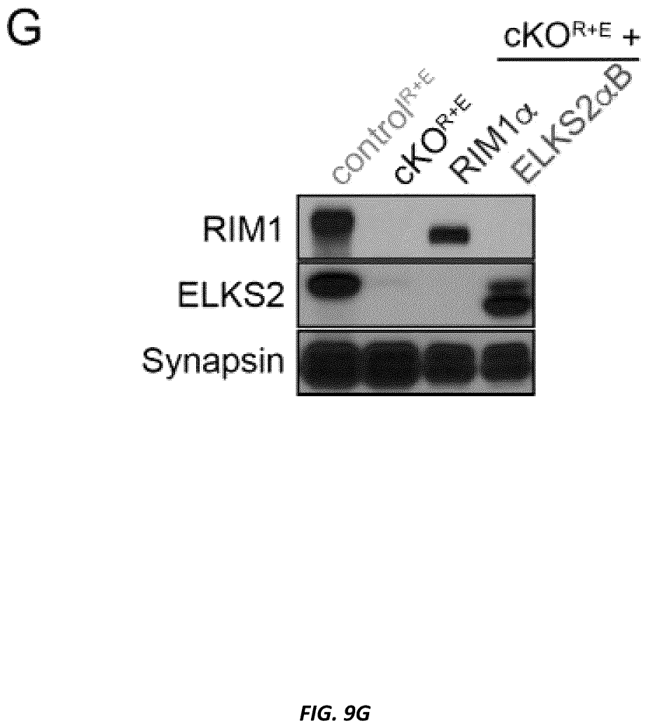

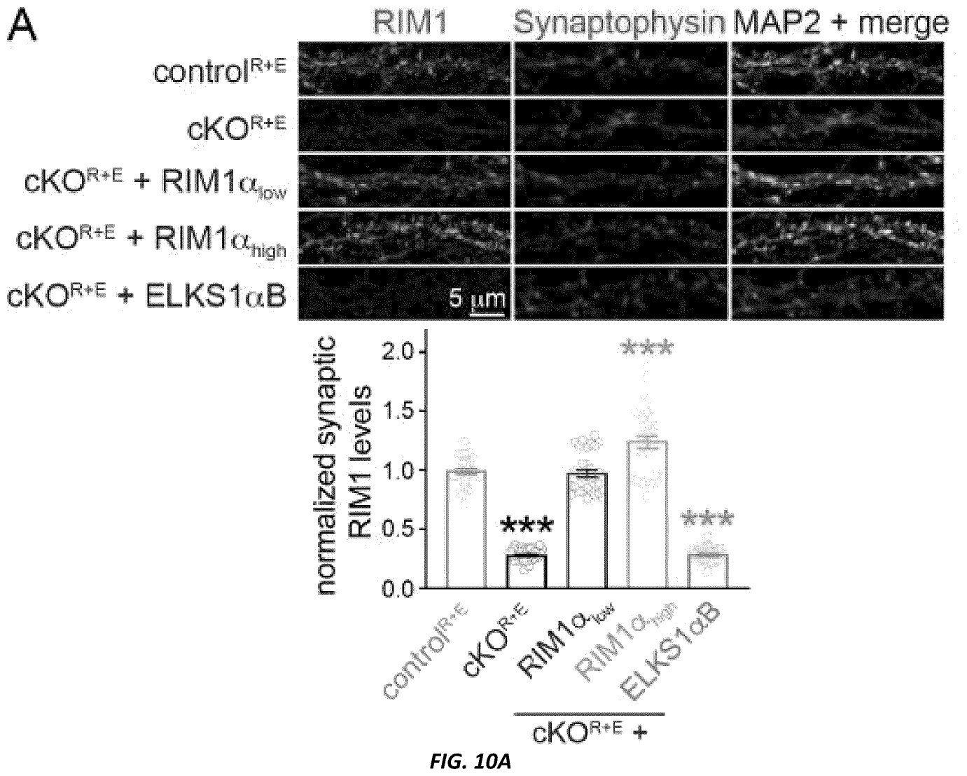

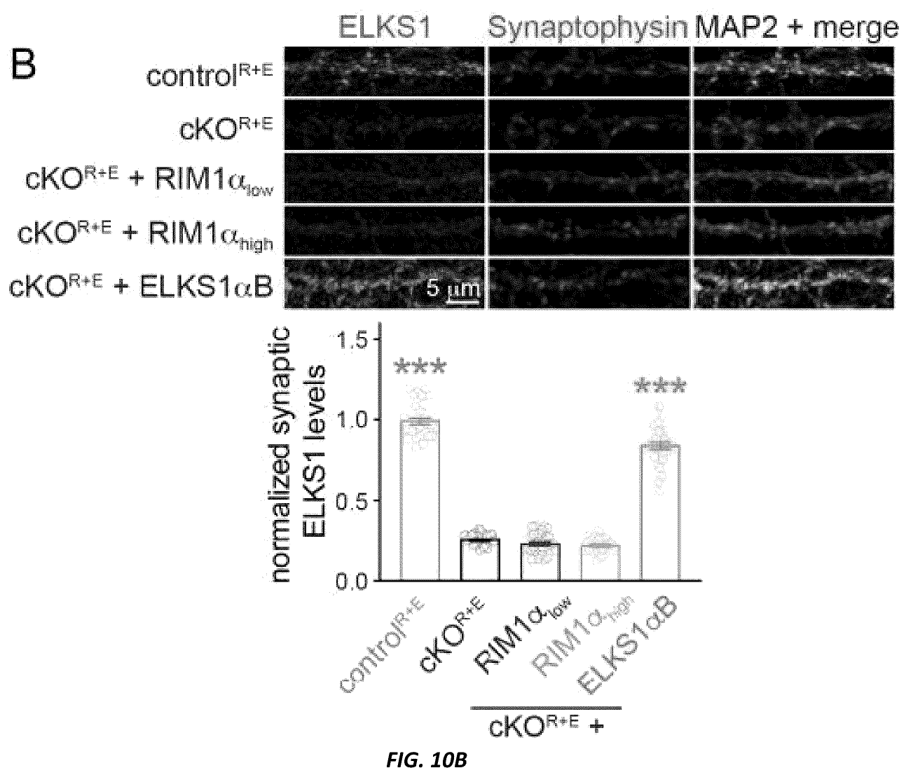

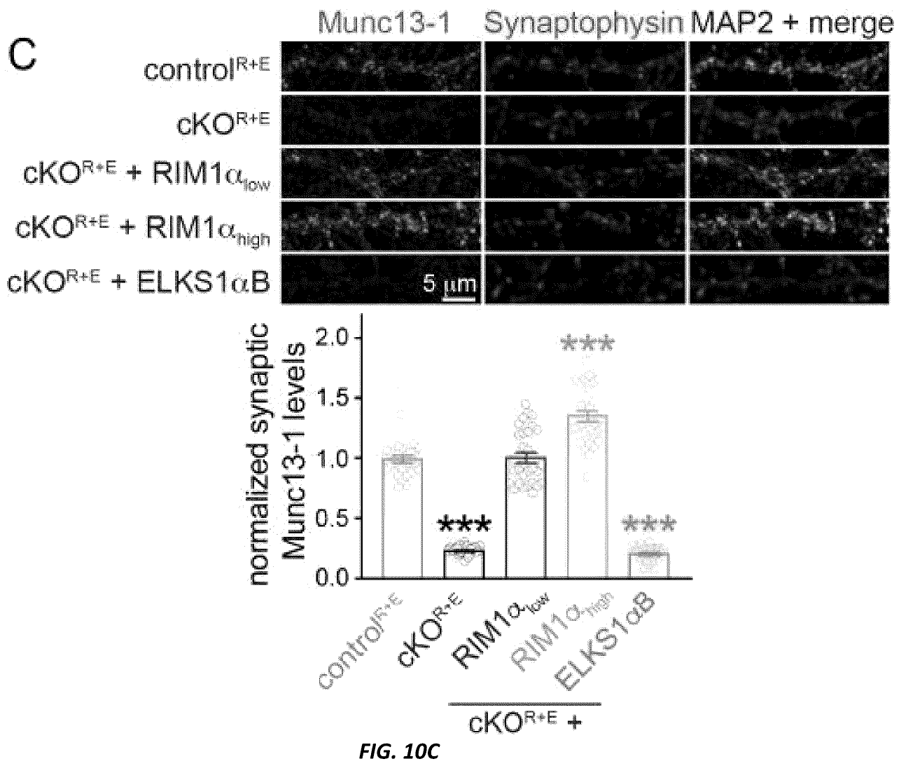

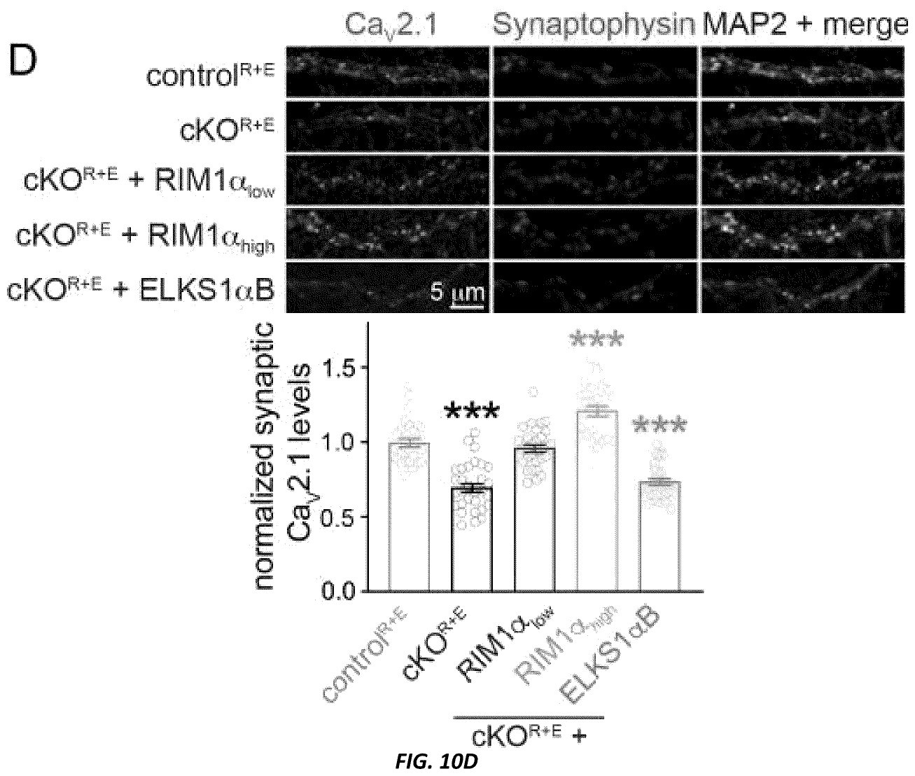

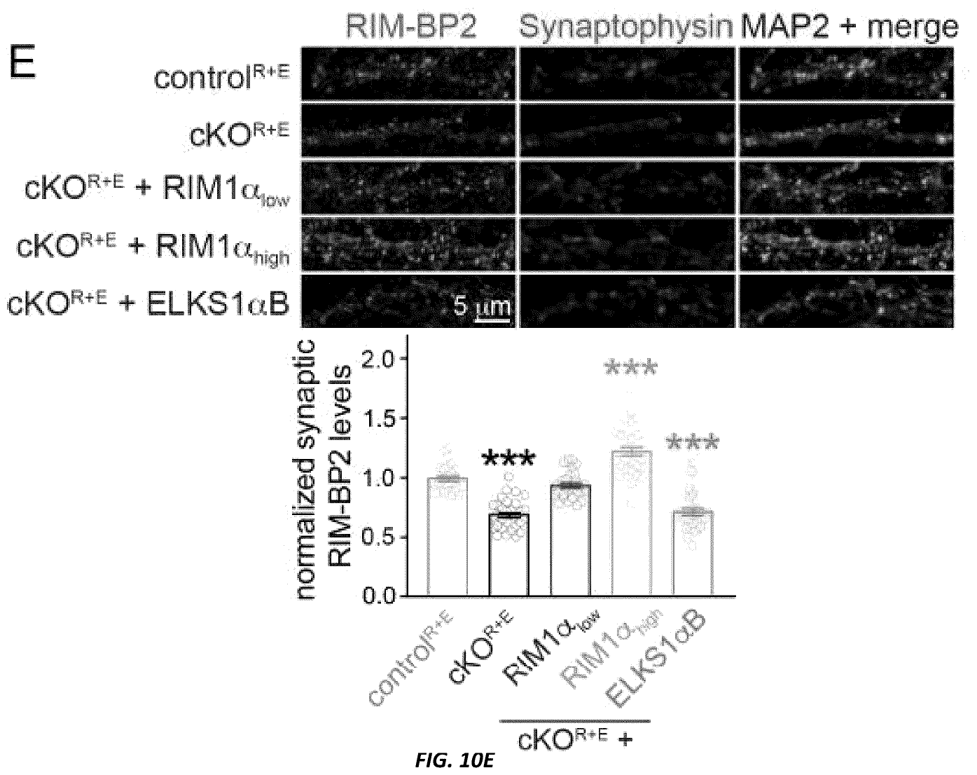

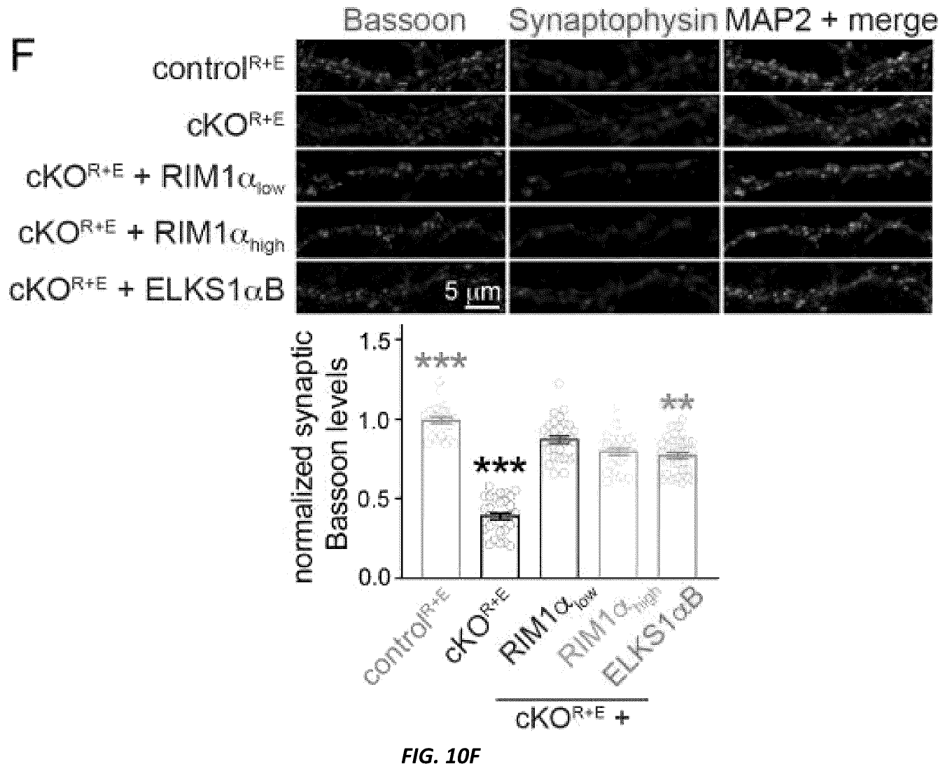

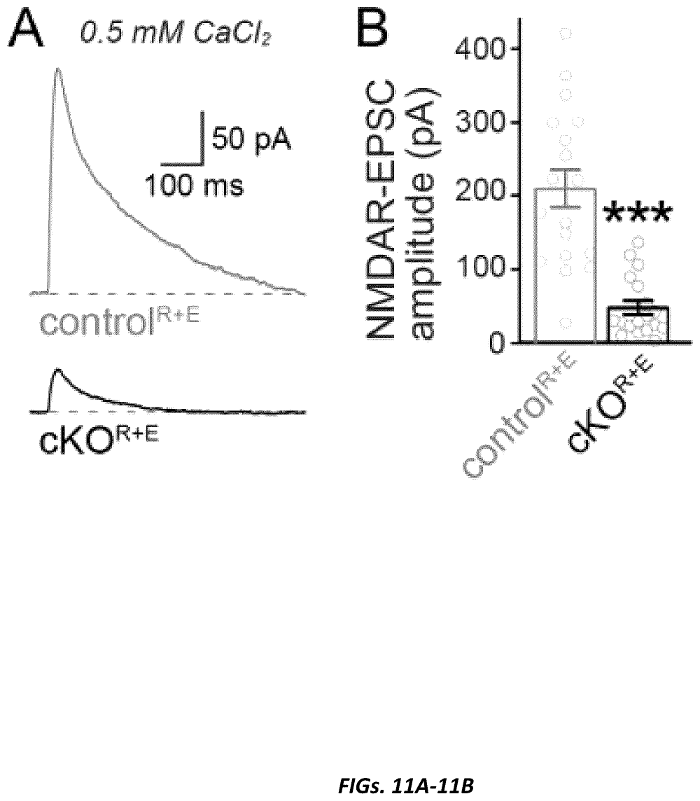

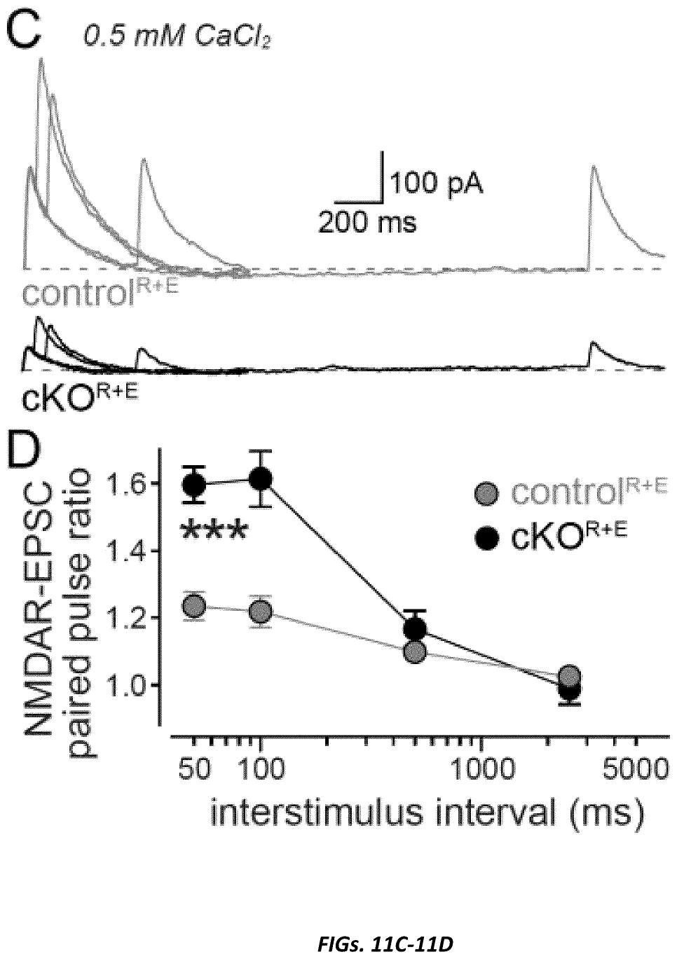

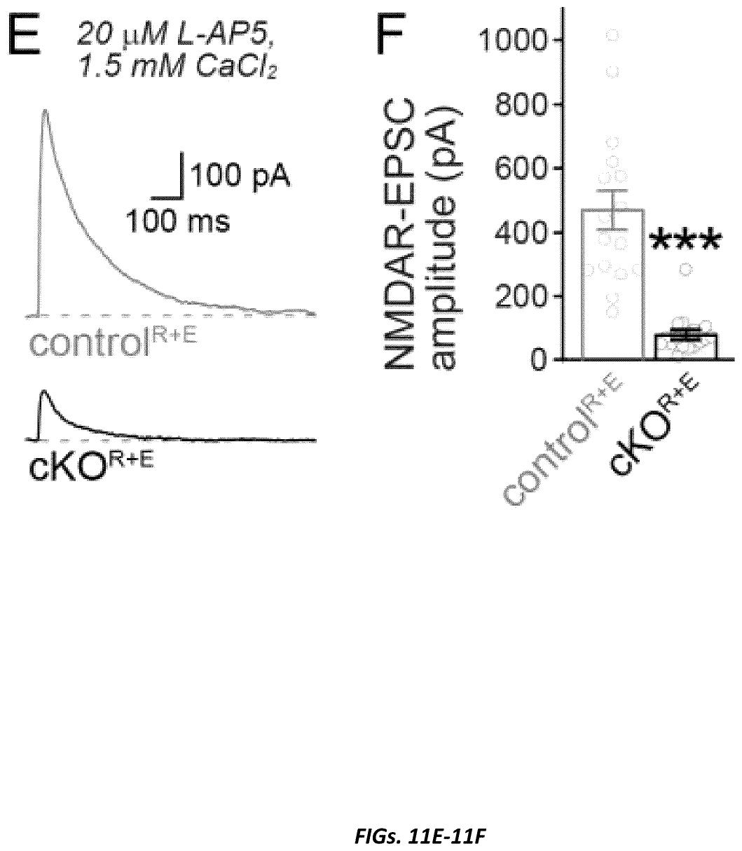

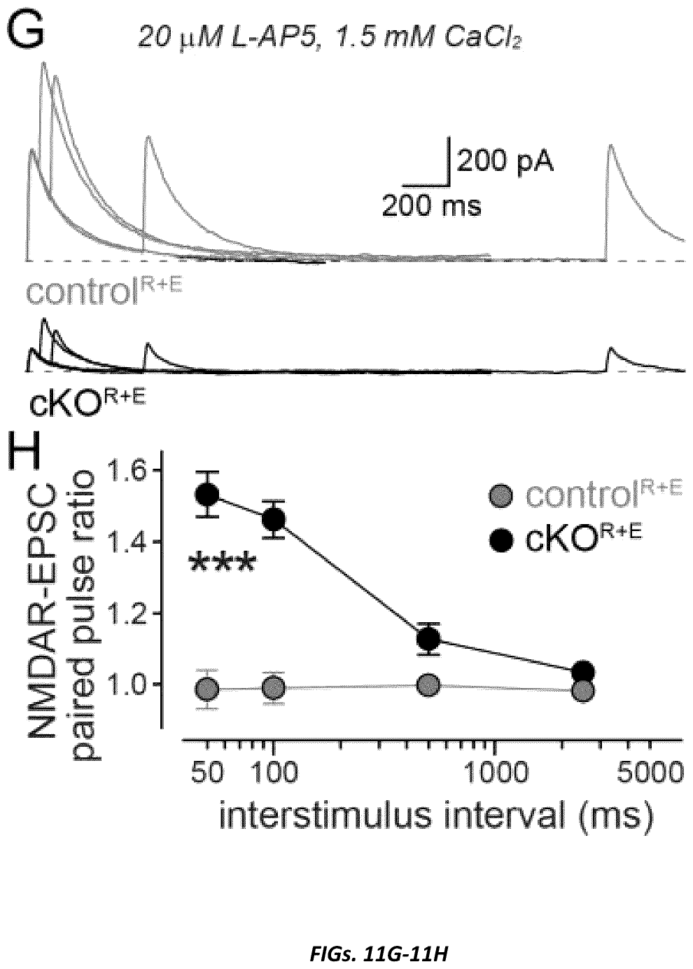

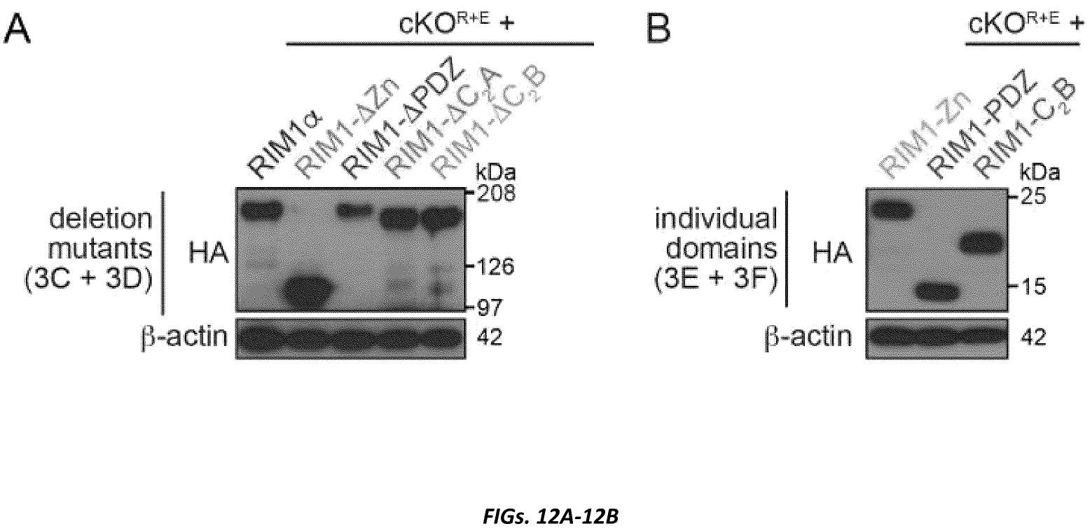

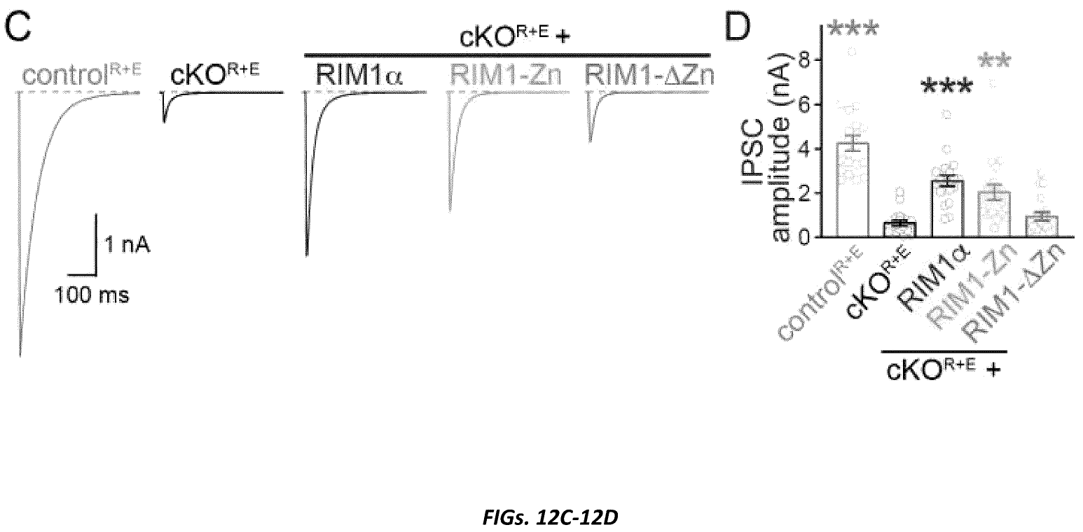

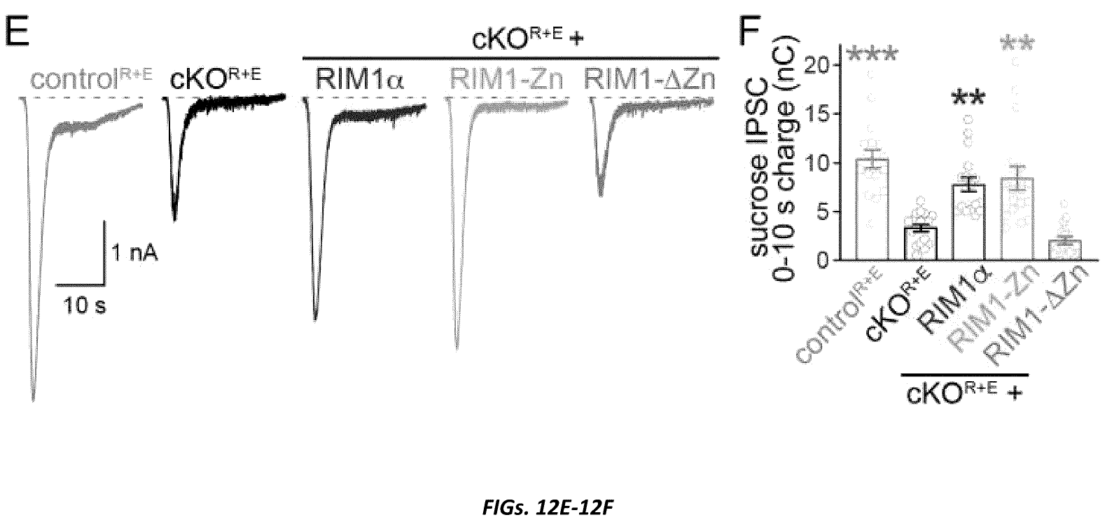

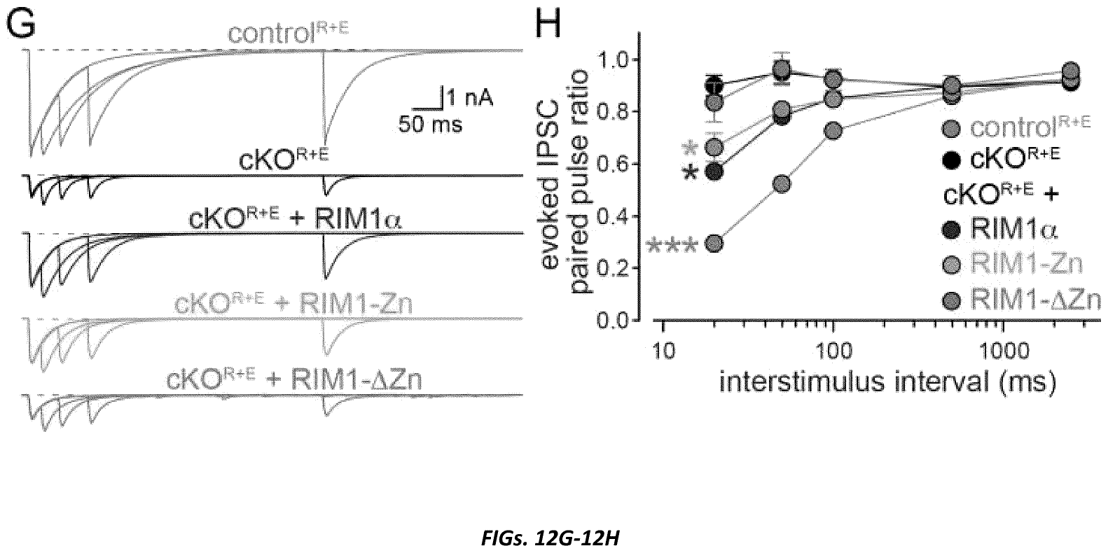

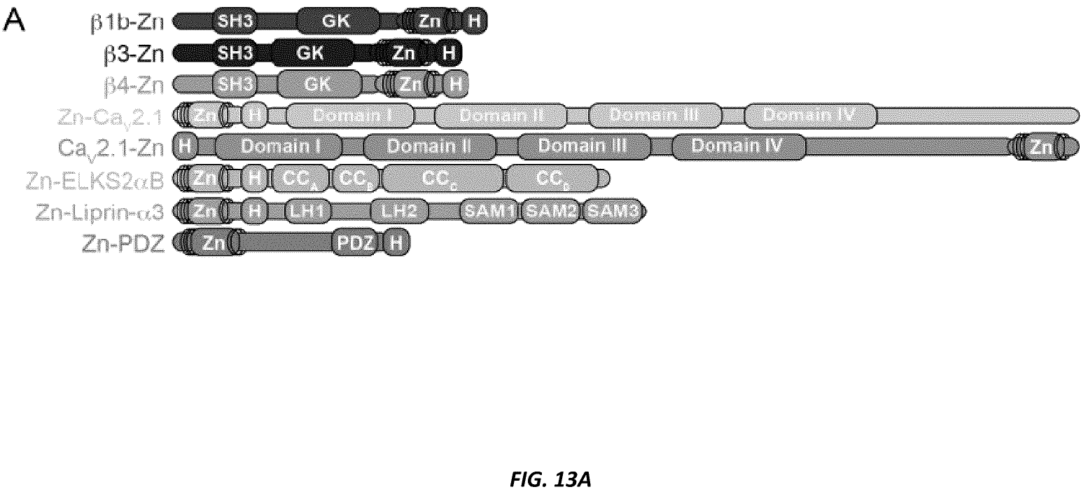

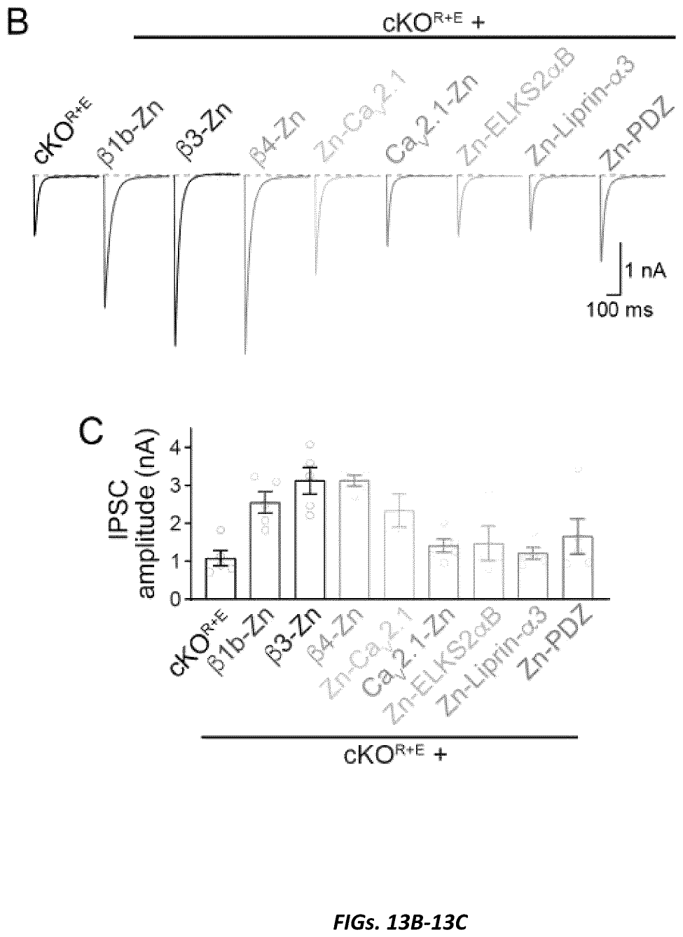

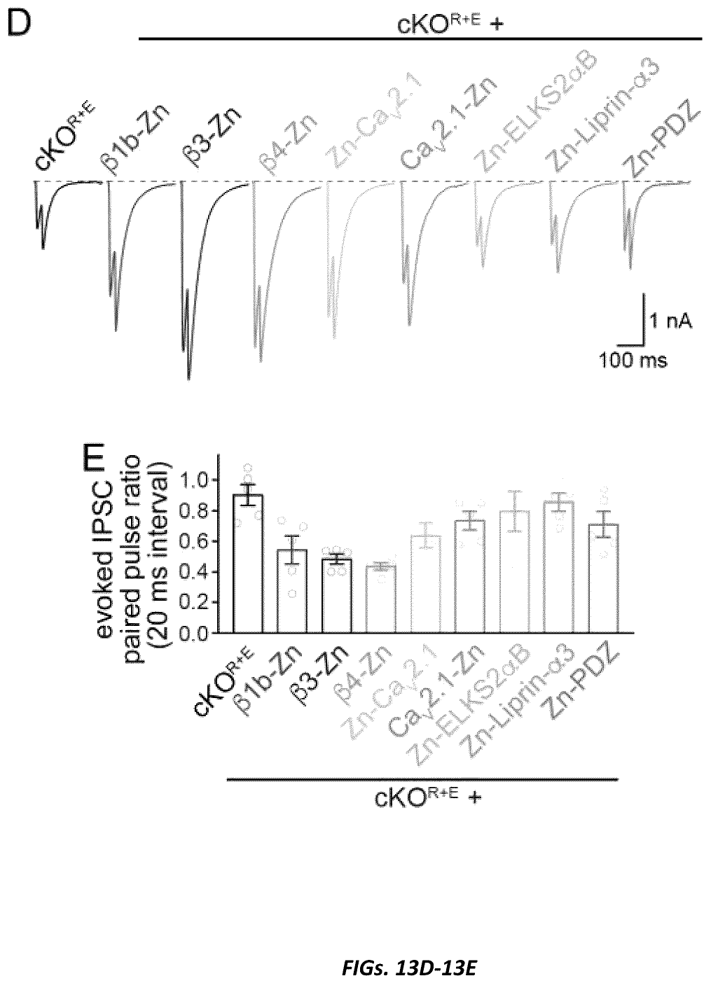

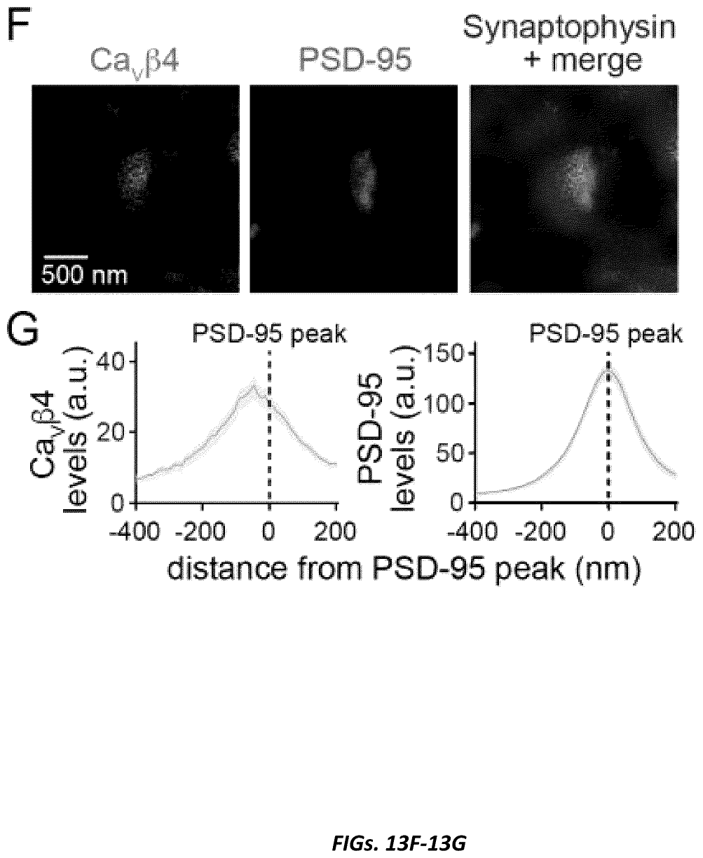

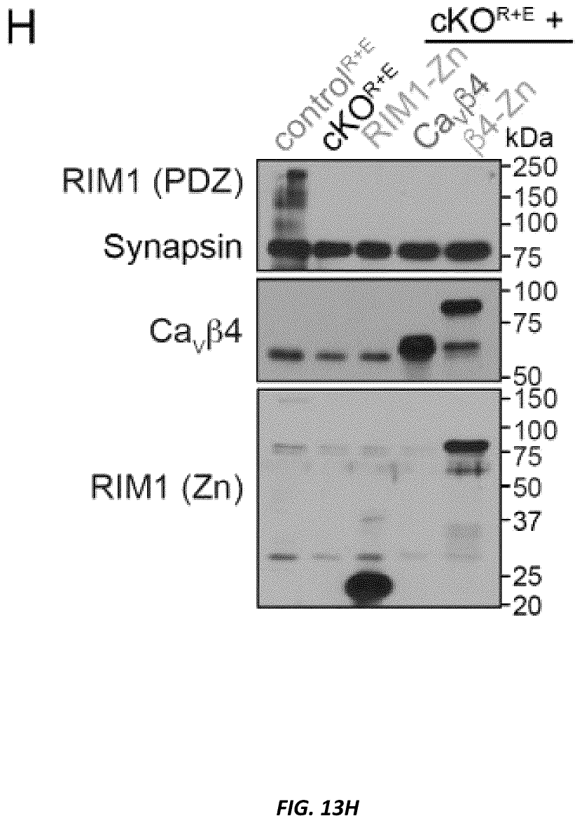

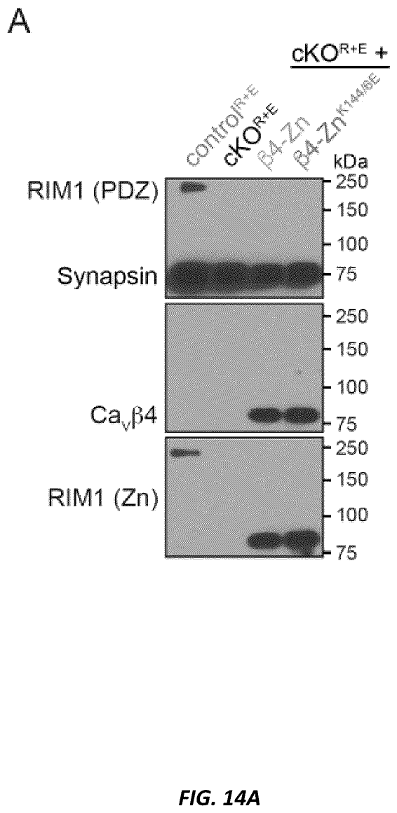

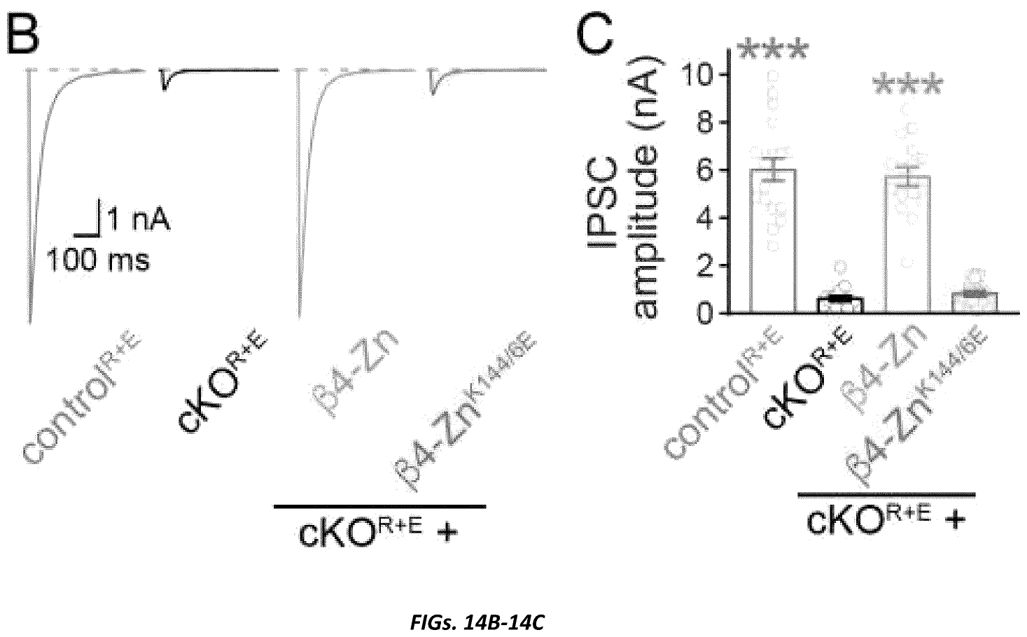

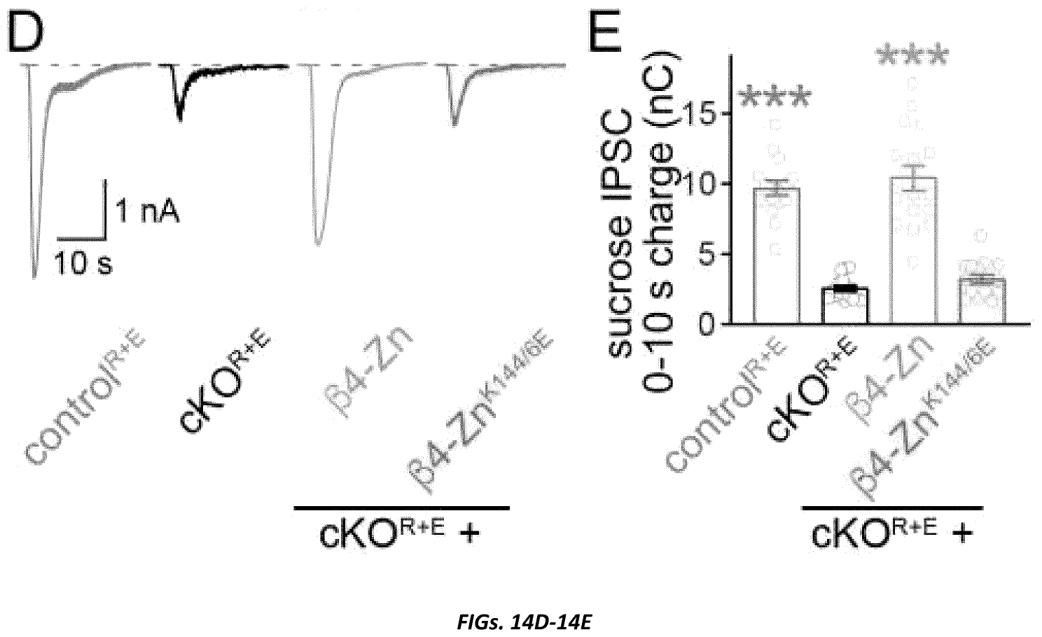

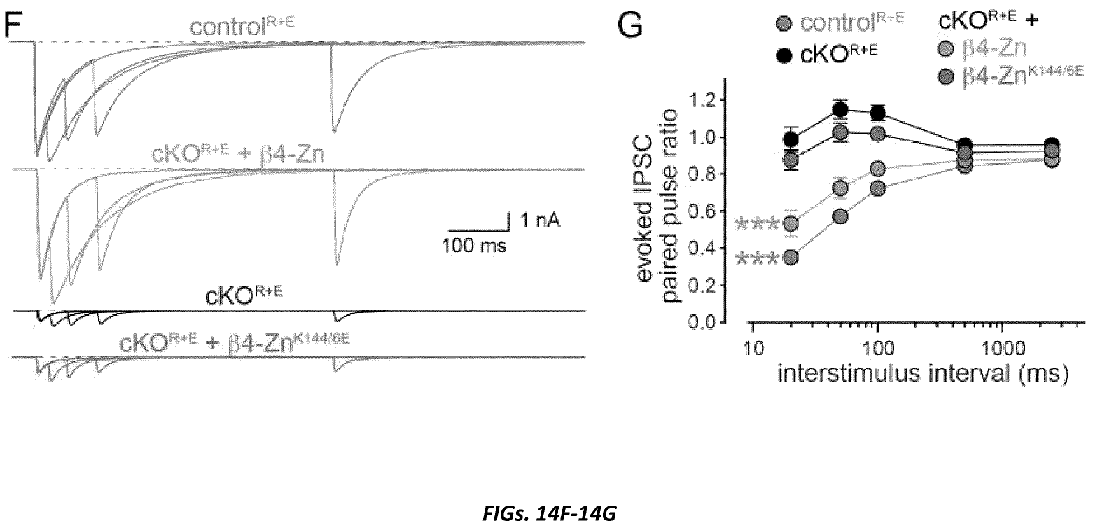

To test the overall model that β4-Zn restores synaptic strength through docking of release-competent vesicles close to Ca²⁺ channels, we introduced K144E+K146E point mutations into the RIM1 zinc finger domain of β4-Zn (generating β4-Zn^{F(144/6E)}. It was previously established that this mutation selectively abolishes binding of the RIM zinc finger to Munc13 (Deng et al., 2011; Dulubova et al., 2005). β4-Zn^{F(14/6E} was efficiently expressed and localized to the active zone area of the plasma membrane (FIGS. 7A-7C, S6A). Abolishing Munc13 binding in β4-ZnF(144/6E resulted in a loss of Munc13-1 recruitment to the target membrane (FIGS. 7D, 7E). While β4-Zn^{F(144/6E} was still sufficient to mediate some enhancement of CaV2.1 levels, it appeared less efficient than in β4-Zn (FIGS. 7F, 7G), quantitatively matching with the somewhat lower β4-Zn^{F(144/6E} active zone levels (FIGS. 7B, 7C). Notably, disrupting binding of β4-Zn to Munc13 completely abolished rescue of vesicle docking (FIGS. 8A, 8B) and of synaptic strength, RRP and p at both excitatory (FIGS. 8C-8H) and inhibitory (FIGS. S6B-S6G) synapses. These data strongly support the model that β4-Zn restores exocytosis via recruitment of Munc13 and vesicle docking. Altogether, our results establish that the active zone protein network can be bypassed with an 80 kDa protein that docks fusion-competent vesicles close to Ca²⁺ channels, and the vast active zone scaffolding mechanisms are not necessary for fusion itself.

### Discussion

The active zone is a molecular machine that is important for synaptic signaling (Emperador-Melero and Kaeser, 2020; Südhof, 2012), and many brain disorders are associated with mutations in active zone proteins or defective active zone function (Benarroch, 2013; Bucan et al., 2009; Johnson et al., 2003; Krumm et al., 2015; O'Roak et al., 2012; Thevenon et al., 2013; Verhage and Sorensen, 2020). However, understanding its mechanisms and restoring its functions has remained challenging because of its molecular complexity. Mouse knockout experiments have revealed that each active zone protein contributes to each active zone function, and it has remained uncertain which proteins and which specific mechanisms drive its roles in release. Here, we develop a reconstitution approach within a synapse after we remove the active zone protein machinery. Our work establishes that synaptic vesicle release can be restored by positioning the RIM zinc finger, a single, small protein domain that recruits the priming protein Munc13, to the Ca²⁺ channels that mediate release triggering. Remarkably, doing so bypasses the need for the complex active zone scaffolding network. Our work further reveals that if the RIM zinc finger is localized to vesicles, Munc13 is recruited and can render these vesicles fusion-competent in the absence of docking. Ultimately, these findings present a straightforward way to restore efficacy and spatiotemporal precision of neurotransmitter release at central synapses with a single, small protein.

Our results mechanistically defined the two fundamental presynaptic processes: vesicle fusogenicity can be generated by activated Munc13 independent of its active zone positioning, and Ca²⁺-secretion coupling is mediated by docking of Munc13-associated synaptic vesicles next to Ca²⁺ channels. Past studies have discovered that these processes rely on many proteins, and each active zone protein has contributed to each active zone function (Acuna et al., 2015; Aravamudan et al., 1999; Augustin et al., 1999; Brockmann et al., 2019; Davydova et al., 2014; Deng et al., 2011; Dong et al., 2018; Emperador-Melero et al., 2021; Grauel et al., 2016; Held et al., 2016; Imig et al., 2014; Kaeser et al., 2011; Kawabe et al., 2017; Kittel et al., 2006; Koushika et al., 2001; Lipstein et al., 2013; Liu et al., 2014, 2011; Matkovic et al., 2013; Richmond et al., 1999; Schoch et al., 2002; Varoqueaux et al., 2002; Wong et al., 2018; Zhen and Jin, 1999). These findings reflect that the active zone is a complex protein network with built-in redundancy, and knockout studies may lead to alterations of the entire network and not necessarily reveal highly specific mechanisms of isolated proteins. Hence, it has been difficult to define which proteins and mechanisms drive vesicle priming, docking and release. Our approach establishes that these functions can be executed in the absence of most of these proteins. Vesicle priming can be almost entirely mediated by the RIM1 zinc finger domain, which is sufficient to recruit, stabilize and activate Munc13. When this mechanism is positioned close to Ca²⁺ channels, release is restored.

Models of neurotransmitter release propose that vesicle docking either precedes vesicle priming or occurs simultaneously with it (Hammarlund et al., 2007; Imig et al., 2014; Rosenmund and Stevens, 1996; Schikorski and Stevens, 2001; Sudhof, 2004), and that Munc13 mediates both roles through the control of SNARE complex assembly (Basu et al., 2005; Imig et al., 2014; Ma et al., 2013; Siksou et al., 2009; Südhof, 2012). In view of this literature, it is surprising that vesicle fusogenicity can be generated in the absence of docking (FIG. 4), dissociating the linear docking-priming model that relies on SNARE-complex assembly. Our data instead reveal that the generation of fusion-competent vesicles and vesicle docking are molecularly separable processes, and that vesicles away from the active zone can be activated for fusion. We propose that the rate limiting step is not SNARE-complex assembly, as this necessitates the coincidence of docking and priming, but instead the availability and activation of Munc13. This is supported by the observation that fusion-competent vesicles can be generated by positioning Munc13 on undocked vesicles (FIGS. 3 and 4), by the notion that some fusion-competent vesicles remain when Munc13 is displaced from the active zone (Wang et al., 2016), and by the finding that knockout of RIM, which is upstream of Munc13's role in vesicle priming, leads to strong impairments in RRP (Calakos et al., 2004; Deng et al., 2011; Han et al., 2011). Hence, Munc13 mediates both synaptic vesicle docking and priming (Augustin et al., 1999; Imig et al., 2014; Varoqueaux et al., 2002), but generating fusion competence and vesicle docking are molecularly separable. We propose that, while many RRP vesicles are docked (Borges-Merjane et al., 2020; Imig et al., 2014; Schikorski and Stevens, 2001), the presence and activation of Munc13 embody the bottleneck for vesicle priming. Munc13-mediated SNARE complex-assembly may not be rate-limiting for vesicle release and can occur before or during fusion.

Mechanisms and hierarchy of active zone protein recruitment have remained difficult to establish. Our work reveals that the RIM PDZ domain is important for recruitment of RIM to the active zone, as removing it prevents RIM active zone targeting. Furthermore, our data indicate that RIM drives recruitment of presynaptic protein machinery. RIM re-expression restores levels of all other active zone proteins and of Ca²⁺ channels, and, remarkably, does so in a dose-dependent manner such that more RIM drives the presence of more Munc13, Ca²⁺ channels and other active zone proteins. Recent work proposed that liquid-liquid phase separation of RIM, RIM-BP and CaV2s mediates active zone assembly (Wu et al., 2019, 2021). While our work does not directly test this model, it is consistent with it, but indicates that phase

condensation of RIM and RIM-BP into liquid droplets may not be necessary for neurotransmitter release, because simply fusing the RIM zinc finger domain to CaVβ4 restores release in the absence of most RIM and RIM-BP sequences necessary for phase condensation. In principle it is possible that other liquid phases, which may or may not incorporate CaV2s, could be at play. In this context, it is interesting that Liprin-α3 levels at the active zone increase upon active zone disruption. Liprin-α proteins undergo phase condensation, and participate in the regulation of active zone structure (Emperador-Melero et al., 2021; McDonald et al., 2020). It is possible, and perhaps likely, that the two phases compete or are in equilibrium with one another at a synapse, and that removing one enhances the other. This is supported by the enhanced presence of Liprin-α3 upon disruption of the active zone protein complex between RIM, ELKS, RIM-BP, CaV2s, Munc13 and Bassoon (FIG. 1). Conversely, at Liprin-α2/3 knockout synapses, enhanced levels of CaV2 proteins are present (Emperador-Melero et al., 2021). RIM may link the two phases together or participate in both, as its active zone recruitment is decreased upon Liprin-α ablation.

An interesting observation is that the artificial β4-Zn fusion protein enhances CaV2 active zone levels together with restoring vesicle docking and release. One possibility is that vesicle docking stabilizes the CaV2 protein complex at active zones. This model is supported by the observation that abolishing the docking function of the β4-Zn fusion protein by preventing its binding to Munc13 appears to revert this function at least partially. Another possibility is that the β4-Zn fusion protein enhances the delivery of CaV2s to the active zone, and that Munc13 binding is required for this function. Ultimately, our data may suggest that a stable release site contains a docked vesicle, that release sites that do not contain docked vesicles are subject to dynamic rearrangements, and that CaV2s of unoccupied release sites may be more mobile (Schneider et al., 2015). An alternative model is that CaV2s and exocytotic protein machinery such as Munc13 are in different proteins complexes (Rebola et al., 2019). In this model RIM proteins would participate in distinct assemblies: one may define secretory sites and contain at least RIM and Munc13 (Deng et al., 2011; Emperador-Melero et al., 2021; Reddy-Alla et al., 2017; Sakamoto et al., 2018; Tang et al., 2016), and another controls Ca²⁺ channel clustering and contains RIM, RIM-BP and CaV2s (Acuna et al., 2016; Held et al., 2020; Hibino et al., 2002; Kaeser et al., 2011; Kushibiki et al., 2019; Liu et al., 2011; Oh et al., 2021). In this model, proteins like RIM or ELKS could bridge the complexes, and our reconstitution would account for both functions (FIG. 8K). Future studies should address these models.

In aggregate, our data suggest that the release machinery assembly requirements are remarkably simple: RIM zinc fingers recruit Munc13 to prime vesicles, and if positioned next to CaV2 channels, these vesicles can be rapidly and precisely released (FIGS. 8I-8K). We propose that synaptic strength can be controlled through reconstituting these key mechanisms, and that the other protein domains and liquid phases mediate regulatory functions (Emperador-Melero and Kaeser, 2020; Emperador-Melero et al., 2021). Some secretory systems, for example those for striatal dopamine release, may make use of these relatively simple, streamlined mechanisms (Banerjee et al., 2021; Liu et al., 2018).

### A Small Protein for Rebuilding the Function of a Complex Machine

Neurotransmitter secretion is often impaired in brain disease, ranging from highly specific associations of gene mutations in active zone proteins to more generalized breakdown of secretion and transmitter signaling (Benarroch, 2013; Bucan et al., 2009; Johnson et al., 2003; Krumm et al., 2015; O'Roak et al., 2012; Thevenon et al., 2013; Verhage and Sorensen, 2020). Advances in AAV-based gene therapy strategies have spurred new hope for developing treatments for brain disorders (Hudry and Vandenberghe, 2019; Sun and Roy, 2021). However, a key limitation is that synaptic and secretory genes often exceed the packaging size of AAVs (Wu et al., 2010). A recent way to work past this limitation is the use of dual or triple AAVs for expression of fragments that are then spliced to generate whole proteins, for example for restoration of hearing (Akil et al., 2019; Al-Moyed et al., 2019). Another possibility is to find smaller proteins to restore function. Our reconstitution approach identifies a single 80 kDa-protein, the CaVβ4-RIM zinc finger fusion, that is well within packaging limits of gene therapy viruses (Hudry and Vandenberghe, 2019; Wu et al., 2010). It is remarkable that this relatively small protein can strongly enhance synaptic efficacy and is sufficient to mediate spatiotemporal precision of release. Our approach may serve as proof-of-concept for reconstructing functions of a complex molecular machine with relatively simple “pieces”. Ultimately, this finding may be leveraged to develop new approaches for enhancing transmitter secretion in neurological and endocrine diseases.

### REFERENCES

Acuna, C., Liu, X., Gonzalez, A., and Südhof, T. C. (2015). RIM-BPs Mediate Tight Coupling of Action Potentials to Ca(2+)-Triggered Neurotransmitter Release. Neuron 87, 1234-1247.
Acuna, C., Liu, X., and Südhof, T. C. (2016). How to Make an Active Zone: Unexpected Universal Functional Redundancy between RIMs and RIM-BPs. Neuron 91, 792-807.
Akil, O., Dyka, F., Calvet, C., Emptoz, A., Lahlou, G., Nouaille, S., Boutet de Monvel, J., Hardelin, J.-P., Hauswirth, W. W., Avan, P., et al. (2019). Dual AAV-mediated gene therapy restores hearing in a DFNB9 mouse model. Proc. Natl. Acad. Sci. U.S.A 116, 4496-4501.
Al-Moyed, H., Cepeda, A. P., Jung, S., Moser, T., Kiigler, S., and Reisinger, E. (2019). A dual-AAV approach restores fast exocytosis and partially rescues auditory function in deaf otoferlin knock-out mice. EMBO Mol. Med. 11, 4496-4501.
Aravamudan, B., Fergestad, T., Davis, W. S., Rodesch, C. K., and Broadie, K. (1999). Drosophila UNC-13 is essential for synaptic transmission. Nat. Neurosci. 2, 965-971.
Augustin, I., Rosenmund, C., Südhof, T. C., and Brose, N. (1999). Munc13-1 is essential for fusion competence of glutamatergic synaptic vesicles. Nature 400, 457-461.
Banerjee, A., Imig, C., Balakrishnan, K., Kershberg, L., Lipstein, N., Uronen, R.-L., Wang, J., Cai, X., Benseler, F., Rhee, J. S., et al. (2021). Molecular and functional architecture of striatal dopamine release sites. Neuron.
Basu, J., Shen, N., Dulubova, I., Lu, J., Guan, R., Guryev, O., Grishin, N. V, Rosenmund, C., and Rizo, J. (2005). A minimal domain responsible for Munc13 activity. Nat Struct Mol Biol 12, 1017-1018.
Benarroch, E. E. (2013). Synaptic vesicle exocytosis: Molecular mechanisms and clinical implications. Neurology 80, 1981-1988.
Betz, A., Thakur, P., Junge, H. J., Ashery, U., Rhee, J. S., Scheuss, V., Rosenmund, C., Rettig, J., and Brose, N. (2001). Functional interaction of the active zone proteins Munc13-1 and RIM1 in synaptic vesicle priming. Neuron 30, 183-196.
Biederer, T., Kaeser, P. S., and Blanpied, T. A. (2017). Transcellular Nanoalignment of Synaptic Function. Neuron 96, 680-696.
Borges-Merjane, C., Kim, O., and Jonas, P. (2020). Functional Electron Microscopy, “Flash and Freeze,” of Identified Cortical Synapses in Acute Brain Slices. Neuron 105, 992-1006.e6.
Brodbeck, J., Davies, A., Courtney, J.-M., Meir, A., Balaguero, N., Canti, C., Moss, F. J., Page, K. M., Pratt, W. S., Hunt, S. P., et al. (2002). The ducky mutation in Cacna2d2 results in altered Purkinje cell morphology and is associated with the expression of a truncated alpha 2 delta-2 protein with abnormal function. J. Biol. Chem. 277, 7684-7693.
Brockmann, M. M., Maglione, M., Willmes, C. G., Stumpf, A., Bouazza, B. A., Velasquez, L. M., Grauel, M. K., Beed, P., Lehmann, M., Gimber, N., et al. (2019). RIM-BP2 primes synaptic vesicles via recruitment of Munc13-1 at hippocampal mossy fiber synapses. Elife 8.
Bucan, M., Abrahams, B. S., Wang, K., Glessner, J. T., Herman, E. I., Sonnenblick, L. I., Alvarez Retuerto, A. I., Imielinski, M., Hadley, D., Bradfield, J. P., et al. (2009). Genome-wide analyses of exonic copy number variants in a family-based study point to novel autism susceptibility genes. PLoS Genet. 5, e1000536.
Calakos, N., Schoch, S., Sudhof, T. C., Malenka, R. C., Südhof, T. C., Malenka, R. C., Sudhof, T. C., and Malenka, R. C. (2004). Multiple roles for the active zone protein RIM1alpha in late stages of neurotransmitter release. Neuron 42, 889-896.
Camacho, M., Basu, J., Trimbuch, T., Chang, S., Pulido-Lozano, C., Chang, S.-S., Duluvova, I., Abo-Rady, M., Rizo, J., and Rosenmund, C. (2017). Heterodimerization of Munc13 C₂A domain with RIM regulates synaptic vesicle docking and priming. Nat. Commun. 8, 15293.
Davydova, D., Marini, C., King, C., Klueva, J., Bischof, F., Romorini, S., Montenegro-Venegas, C., Heine, M., Schneider, R., Schröder, M. S., et al. (2014). Bassoon specifically controls presynaptic P/Q-type Ca(2+) channels via RIM-binding protein. Neuron 82, 181-194.
Deng, L., Kaeser, P. S., Xu, W., and Südhof, T. C. (2011). RIM proteins activate vesicle priming by reversing autoinhibitory homodimerization of Munc13. Neuron 69, 317-331.
Dong, W., Radulovic, T., Goral, R. O., Thomas, C., Suarez Montesinos, M., Guerrero-Given, D., Hagiwara, A., Putzke, T., Hida, Y., Abe, M., et al. (2018). CAST/ELKS Proteins Control Voltage-Gated Ca2+ Channel Density and Synaptic Release Probability at a Mammalian Central Synapse. Cell Rep. 24, 284-293.e6.
Dulubova, I., Lou, X., Lu, J., Huryeva, I., Alam, A., Schneggenburger, R., Sudhof, T. C., and Rizo, J. (2005). A Munc13/RIM/Rab3 tripartite complex: from priming to plasticity? EMBO J. 24, 2839-2850.
Emperador-Melero, J., and Kaeser, P. S. (2020). Assembly of the presynaptic active zone. Curr. Opin. Neurobiol. 63, 95-103.
Emperador-Melero, J., Wong, M. Y., Wang, S. S. H., de Nola, G., Nyitrai, H., Kirchhausen, T., and Kaeser, P. S. (2021). PKC-phosphorylation of Liprin-α3 triggers phase separation and controls presynaptic active zone structure. Nat. Commun. 12, 3057.
Fukuda, M. (2003). Distinct Rab binding specificity of Rim1, Rim2, rabphilin, and Noc2. Identification of a critical determinant of Rab3A/Rab27A recognition by Rim2. J. Biol. Chem. 278, 15373-80.
Grauel, M. K., Maglione, M., Reddy-Alla, S., Willmes, C. G., Brockmann, M. M., Trimbuch, T., Rosenmund, T., Pangalos, M., Vardar, G., Stumpf, A., et al. (2016). RIM-binding protein 2 regulates release probability by fine-tuning calcium channel localization at murine hippocampal synapses. Proc. Natl. Acad. Sci. 113, 11615-11620.
Hammarlund, M., Palfreyman, M. T., Watanabe, S., Olsen, S., and Jorgensen, E. M. (2007). Open syntaxin docks synaptic vesicles. PLoS Biol. 5, e198.
Han, Y., Kaeser, P. S., Südhof, T. C., and Schneggenburger, R. (2011). RIM determines Ca²⁺ channel density and vesicle docking at the presynaptic active zone. Neuron 69, 304-316.
Held, R. G., and Kaeser, P. S. (2018). ELKS active zone proteins as multitasking scaffolds for secretion. Open Biol. 8, 170258.
Held, R. G., Liu, C., and Kaeser, P. S. (2016). ELKS controls the pool of readily releasable vesicles at excitatory synapses through its N-terminal coiled-coil domains. Elife 5.
Held, R. G., Liu, C., Ma, K., Ramsey, A. M., Tarr, T. B., De Nola, G., Wang, S. S. H., Wang, J., van den Maagdenberg, A. M. J. M., Schneider, T., et al. (2020). Synapse and Active Zone Assembly in the Absence of Presynaptic Ca2+ Channels and Ca2+ Entry. Neuron 107, 667683.e9.
Hibino, H., Pironkova, R., Onwumere, O., Vologodskaia, M., Hudspeth, A. J., and Lesage, F. (2002). RIM binding proteins (RBPs) couple Rab3-interacting molecules (RIMs) to voltage-gated Ca(2+) channels. Neuron 34, 411-423.
Hu, C., Ahmed, M., Melia, T. J., Söllner, T. H., Mayer, T., Rothman, J. E., Sollner, T. H., Mayer, T., and Rothman, J. E. (2003). Fusion of cells by flipped SNAREs. Science 300, 1745-1749. Hudry, E., and Vandenberghe, L. H. (2019). Therapeutic AAV Gene Transfer to the Nervous System: A Clinical Reality. Neuron 101, 839-862.
Imig, C., Min, S. W., Krinner, S., Arancillo, M., Rosenmund, C., Südhof, T. C., Rhee, J. S., Brose, N., and Cooper, B. H. (2014). The Morphological and Molecular Nature of Synaptic Vesicle Priming at Presynaptic Active Zones. Neuron 84, 416-431.
Johnson, S., Halford, S., Morris, A. G., Patel, R. J., Wilkie, S. E., Hardcastle, A. J., Moore, A. T., Zhang, K., and Hunt, D. M. (2003). Genomic organisation and alternative splicing of human RIM1, a gene implicated in autosomal dominant cone-rod dystrophy (CORD7). Genomics 81, 304-314.
de Jong, A. P. H., Roggero, C. M., Ho, M.-R., Wong, M. Y., Brautigam, C. A., Rizo, J., and Kaeser, P. S. (2018). RIM C₂B Domains Target Presynaptic Active Zone Functions to PIP2-Containing Membranes. Neuron 98, 335-349.e7.
Kaeser, P. S., and Regehr, W. G. (2017). The readily releasable pool of synaptic vesicles. Curr. Opin. Neurobiol. 43, 63-70.
Kaeser, P. S., Kwon, H. B., Chiu, C. Q., Deng, L., Castillo, P. E., and Sudhof, T. C. (2008). RIM1alpha and RIM1beta are synthesized from distinct promoters of the RIM1 gene to mediate differential but overlapping synaptic functions. J. Neurosci. 28, 13435-13447.
Kaeser, P. S., Deng, L., Chavez, A. E., Liu, X., Castillo, P. E., and Südhof, T. C. (2009). ELKS2alpha/CAST deletion selectively increases neurotransmitter release at inhibitory synapses. Neuron 64, 227-239.
Kaeser, P. S., Deng, L., Wang, Y., Dulubova, I., Liu, X., Rizo, J., and Südhof, T. C. (2011). RIM proteins tether Ca2+ channels to presynaptic active zones via a direct PDZ-domain interaction. Cell 144, 282-295.
Kawabe, H., Mitkovski, M., Kaeser, P. S., Hirrlinger, J., Opazo, F., Nestvogel, D., Kalla, S., Fejtova, A., Verrier, S. E. S. E., Bungers, S. R. S. R., et al. (2017). ELKS1 localizes the synaptic vesicle priming protein bMunc13-2 to a specific subset of active zones. J. Cell Biol. 216, 1143-1161.
Kittel, R. J., Wichmann, C., Rasse, T. M., Fouquet, W., Schmidt, M., Schmid, A., Wagh, D. A., Pawlu, C., Kellner, R. R., Willig, K. I., et al. (2006). Bruchpilot promotes active zone assembly, Ca2+ channel clustering, and vesicle release. Science 312, 1051-1054.
Koushika, S. P., Richmond, J. E., Hadwiger, G., Weimer, R. M., Jorgensen, E. M., and Nonet, M. L. (2001). A post-docking role for active zone protein Rim. Nat. Neurosci. 4, 997-1005.
Krumm, N., Turner, T. N., Baker, C., Vives, L., Mohajeri, K., Witherspoon, K., Raja, A., Coe, B. P., Stessman, H. A., He, Z.-X., et al. (2015). Excess of rare, inherited truncating mutations in autism. Nat. Genet. 47, 582-588.
Kushibiki, Y., Suzuki, T., Jin, Y., and Taru, H. (2019). RIMB-1/RIM-Binding Protein and UNC-10/RIM Redundantly Regulate Presynaptic Localization of the Voltage-Gated Calcium Channel in Caenorhabditis elegans. J. Neurosci. 39, 8617-8631.
Lipstein, N., Sakaba, T., Cooper, B. H., Lin, K.-H., Strenzke, N., Ashery, U., Rhee, J.-S., Taschenberger, H., Neher, E., and Brose, N. (2013). Dynamic control of synaptic vesicle replenishment and short-term plasticity by Ca(2+)-calmodulin-Munc13-1 signaling. Neuron 79, 82-96.
Liu, C., Bickford, L. S., Held, R. G., Nyitrai, H., Südhof, T. C., and Kaeser, P. S. (2014). The active zone protein family ELKS supports Ca2+ influx at nerve terminals of inhibitory hippocampal neurons. J. Neurosci. 34, 12289-12303.
Liu, C., Kershberg, L., Wang, J., Schneeberger, S., and Kaeser, P. S. (2018). Dopamine Secretion Is Mediated by Sparse Active Zone-like Release Sites. Cell 172, 706-718.e15.
Liu, K. S. Y., Siebert, M., Mertel, S., Knoche, E., Wegener, S., Wichmann, C., Matkovic, T., Muhammad, K., Depner, H., Mettke, C., et al. (2011). RIM-binding protein, a central part of the active zone, is essential for neurotransmitter release. Science 334, 1565-1569.
Ma, C., Su, L., Seven, A. B., Xu, Y., and Rizo, J. (2013). Reconstitution of the Vital Functions of Munc18 and Munc13 in Neurotransmitter Release. Science 339, 421-425.
Matkovic, T., Siebert, M., Knoche, E., Depner, H., Mertel, S., Owald, D., Schmidt, M., Thomas, U., Sickmann, A., Kamin, D., et al. (2013). The Bruchpilot cytomatrix determines the size of the readily releasable pool of synaptic vesicles. J. Cell Biol. 202, 667-683.
McDonald, N. A., Fetter, R. D., and Shen, K. (2020). Assembly of synaptic active zones requires phase separation of scaffold molecules. Nature 588, 454-458.
Nyitrai, H., Wang, S. S. H., and Kaeser, P. S. (2020). ELKS1 Captures Rab6-Marked Vesicular Cargo in Presynaptic Nerve Terminals. Cell Rep. 31, 107712.
O'Roak, B. J., Vives, L., Girirajan, S., Karakoc, E., Krumm, N., Coe, B. P., Levy, R., Ko, A., Lee, C., Smith, J. D., et al. (2012). Sporadic autism exomes reveal a highly interconnected protein network of de novo mutations. Nature 485, 246-250.
Oh, K. H., Krout, M. D., Richmond, J. E., and Kim, H. (2021). UNC-2 Ca_{V}2 Channel Localization at Presynaptic Active Zones Depends on UNC-10/RIM and SYD-2/Liprin-α in Caenorhabditis elegans. J. Neurosci. 41, 4782-4794.
Page, K. M., Rothwell, S. W., and Dolphin, A. C. (2016). The Ca_{V}β Subunit Protects the I-II Loop of the Voltage-gated Calcium Channel Ca_{V}2.2 from Proteasomal Degradation but Not Oligoubiquitination. J. Biol. Chem. 291, 20402-20416.
Parthier, D., Kuner, T., and Körber, C. (2018). The presynaptic scaffolding protein Piccolo organizes the readily releasable pool at the calyx of Held. J. Physiol. 596, 1485-1499.
Quade, B., Camacho, M., Zhao, X., Orlando, M., Trimbuch, T., Xu, J., Li, W., Nicastro, D., Rosenmund, C., and Rizo, J. (2019). Membrane bridging by Munc13-1 is crucial for neurotransmitter release. Elife 8.
Rebola, N., Reva, M., Kirizs, T., Szoboszlay, M., Lörincz, A., Moneron, G., Nusser, Z., and DiGregorio, D. A. (2019). Distinct Nanoscale Calcium Channel and Synaptic Vesicle Topographies Contribute to the Diversity of Synaptic Function. Neuron 104, 693-710.e9.
Reddy-Alla, S., Bohme, M. A., Reynolds, E., Beis, C., Grasskamp, A. T., Mampell, M. M., Maglione, M., Jusyte, M., Rey, U., Babikir, H., et al. (2017). Stable Positioning of Unc13 Restricts Synaptic Vesicle Fusion to Defined Release Sites to Promote Synchronous Neurotransmission. Neuron 95, 1350-1364.e12.
Richmond, J. E., Davis, W. S., and Jorgensen, E. M. (1999). UNC-13 is required for synaptic vesicle fusion in C. elegans. Nat. Neurosci. 2, 959-964.
Rosenmund, C., and Stevens, C. F. (1996). Definition of the readily releasable pool of vesicles at hippocampal synapses. Neuron 16, 1197-1207.
Sakamoto, H., Ariyoshi, T., Kimpara, N., Sugao, K., Taiko, I., Takikawa, K., Asanuma, D., Namiki, S., and Hirose, K. (2018). Synaptic weight set by Munc13-1 supramolecular assemblies. Nat. Neurosci. 21,41-49.
Schikorski, T., and Stevens, C. F. (2001). Morphological correlates of functionally defined synaptic vesicle populations. Nat. Neurosci. 4, 391-395.
Schneider, R., Hosy, E., Kohl, J., Klueva, J., Choquet, D., Thomas, U., Voigt, A., and Heine, M. (2015). Mobility of Calcium Channels in the Presynaptic Membrane. Neuron 86, 672-679.
Schoch, S., Castillo, P. E., Jo, T., Mukherjee, K., Geppert, M., Wang, Y., Schmitz, F., Malenka, R. C., and Südhof, T. C. (2002). RIM1alpha forms a protein scaffold for regulating neurotransmitter release at the active zone. Nature 415, 321-326.
Siksou, L., Varoqueaux, F., Pascual, O., Triller, A., Brose, N., and Marty, S. (2009). A common molecular basis for membrane docking and functional priming of synaptic vesicles. Eur. J. Neurosci. 30, 49-56.
Spangler, S. A., Schmitz, S. K., Kevenaar, J. T., de Graaff, E., de Wit, H., Demmers, J., Toonen, R. F., and Hoogenraad, C. C. (2013). Liprin-alpha2 promotes the presynaptic recruitment and turnover of RIM1/CASK to facilitate synaptic transmission. J. Cell Biol. 201, 915-928.
Sternberg, S. R. (1983). Biomedical Image Processing. Computer (Long. Beach. Calif). 16, 22-34.
Sudhof, T. C. (2004). The synaptic vesicle cycle. Annu. Rev. Neurosci. 27, 509-547.
Südhof, T. C. (2012). The Presynaptic Active Zone. Neuron 75, 11-25.
Sun, J., and Roy, S. (2021). Gene-based therapies for neurodegenerative diseases. Nat. Neurosci.
Tang, A.-H., Chen, H., Li, T. P., Metzbower, S. R., MacGillavry, H. D., and Blanpied, T. A. (2016). A trans-synaptic nanocolumn aligns neurotransmitter release to receptors. Nature 536, 210-214.
Thevenon, J., Callier, P., Andrieux, J., Delobel, B., David, A., Sukno, S., Minot, D., Mosca Anne, L., Marle, N., Sanlaville, D., et al. (2013). 12p13.33 microdeletion including ELKS/ERC1, a new locus associated with childhood apraxia of speech. Eur. J. Hum. Genet. 21, 82-88.
Tucker, W. C., Weber, T., and Chapman, E. R. (2004). Reconstitution of Ca2+-regulated membrane fusion by synaptotagmin and SNAREs. Science 304, 435-438.
Varoqueaux, F., Sigler, A., Rhee, J. S., Brose, N., Enk, C., Reim, K., and Rosenmund, C. (2002). Total arrest of spontaneous and evoked synaptic transmission but normal synaptogenesis in the absence of Munc13-mediated vesicle priming. Proc. Natl. Acad. Sci. U.S.A 99, 9037-9042.
Verhage, M., and Sorensen, J. B. (2020). SNAREopathies: Diversity in Mechanisms and Symptoms. Neuron.
Wang, S. S. H., Held, R. G., Wong, M. Y., Liu, C., Karakhanyan, A., and Kaeser, P. S. (2016). Fusion Competent Synaptic Vesicles Persist upon Active Zone Disruption and Loss of Vesicle Docking. Neuron 91, 777-791.
Wang, Y., Okamoto, M., Schmitz, F., Hofmann, K., and Sudhof, T. C. (1997). Rim is a putative Rab3 effector in regulating synaptic-vesicle fusion. Nature 388, 593-598.
Wong, M. Y., Liu, C., Wang, S. S. H., Roquas, A. C. F., Fowler, S. C., and Kaeser, P. S. (2018). Liprin-α3 controls vesicle docking and exocytosis at the active zone of hippocampal synapses. Proc. Natl. Acad. Sci. U.S.A 115, 2234-2239.
Wu, X., Cai, Q., Shen, Z., Chen, X., Zeng, M., Du, S., and Zhang, M. (2019). RIM and RIM-BP Form Presynaptic Active-Zone-like Condensates via Phase Separation. Mol. Cell 73, 971984.e5.
Wu, X., Ganzella, M., Zhou, J., Zhu, S., Jahn, R., and Zhang, M. (2021). Vesicle Tethering on the Surface of Phase-Separated Active Zone Condensates. Mol. Cell 81, 13-24.e7.
Wu, Z., Yang, H., and Colosi, P. (2010). Effect of genome size on AAV vector packaging. Mol. Ther. 18, 80-86.
Zhen, M., and Jin, Y. (1999). The liprin protein SYD-2 regulates the differentiation of presynaptic termini in C. elegans. Nature 401, 371-375.
Zucker, R. S., and Regehr, W. G. (2002). Short-term synaptic plasticity. Annu. Rev. Physiol. 64, 355-405.

### Materials and Methods

Mice. The quadruple homozygote floxed mice for RIM1αβ (Kaeser et al., 2008) (RRID: IMSR_JAX:015832), RIM2αβγ (Kaeser et al., 2011) (RRID: IMSR_JAX:015833), ELKS1α (Liu et al., 2014) (RRID: IMSR_JAX:015830) and ELKS2a (Kaeser et al., 2009) (RRID: IMSR_JAX:015831) were previously described (Wang et al., 2016). All animal experiments were performed according to institutional guidelines at Harvard University.

Cell culture and lentiviral infection. Primary mouse hippocampal cultures were generated from newborn conditional quadruple floxed pups as described before (Held et al., 2020; Wang et al., 2016). Mice were anesthetized on ice slurry within 24 h after birth and the hippocampus was dissected out. Cells were dissociated and plated onto glass coverslips in tissue culture medium composed of Minimum Essential Medium (MEM) with 0.5% glucose, 0.02% NaHCO₃, 0.1 mg/mL transferrin, 10% Fetal Select bovine serum (Atlas Biologicals FS-0500-AD), 2 mM L-glutamine, and 25 μg/mL insulin. Cultures were maintained in a 37° C.-tissue culture incubator, and after ˜24 h the plating medium was exchanged with growth medium composed of MEM with 0.5% glucose, 0.02% NaHCO₃, 0.1 mg/mL transferrin, 5% Fetal Select bovine serum (Atlas Biologicals FS-0500-AD), 2% B-27 supplement (Thermo Fisher 17504044), and 0.5 mM L-glutamine. At DIV3 or DIV4, depending on growth, 50% or 75% of the medium were exchanged with growth medium supplemented with 4 μM Cytosine β-D-arabinofuranoside (AraC) to inhibit glial cell growth. Lentiviruses expressing EGFP-tagged cre recombinase (to generate cKO^{R+E} neurons, made using pFSW EGFP cre) or a truncated, enzymatically inactive EGFP-tagged cre protein (to generate control^{R+E} neurons, made using pFSW EGFP Acre) were produced in HEK293T cells by Ca²⁺-phosphate transfection. Expression in lentiviral constructs was driven by the human Synapsin promoter to restrict expression to neurons (Liu et al., 2014; Wang et al., 2016) except for the RIM1αhigh condition (which was done using FUGW-RIM1α with a ubiquitin promoter). For cre-expressing and control virus, neurons were infected with HEK cell supernatant at DIV5 as described (Liu et al., 2014; Wang et al., 2016). For rescue with the various protein variants (ELKS1αB, ELKS2αB, RIM1α, RIM1 mutants, CaVβ4, β4-Zn and other RIM1-Zn fusion constructs), neurons were infected with rescue virus at DIV3 (a virus made using pFSW without a cDNA inserted in the multiple cloning site was used in the control conditions instead of a rescue virus) and with cre or Acre virus at DIV5. Analyses were performed at DIV15-19.

Rescue constructs. For full-length RIM1α (all residue numbering is provided according to Uniprot ID Q9JIR4), the open reading frame (ORF) was subcloned into lentiviral backbones and expression was driven by either a synapsin promoter (pFSW RIM1α-HA, p592) for lower expression or a ubiquitin promoter (pFUGW RIM1α-HA, p591, described in (de Jong et al., 2018)) for higher expression.

The synapsin promoter was used in all other rescue constructs. For all experiments, RIM zinc finger refers to residues M1-D213, RIM PDZ to H597-R705, RIM C₂A to Q754-Q882, and RIM C₂B to G1447-S1615. All RIM1 individual domains (pFSW RIM1-Zn-HA, p654; pFSW RIM1-PDZ-HA, p648; pFSW RIM1-C₂B-HA, p647) and domain deletion mutants (pFSW RIM1-ΔZn-HA, p640; pFSW RIM1-ΔPDZ-HA, p639; pFSW RIM1-ΔC₂A-HA, p637; pFSW RIM1-ΔC₂B-HA, p638) span or lack these residues, except for the pFSW RIM1-ΔZn-HA, which spans H597-S1615. In RIM1α and in domain deletion mutants, an HA-tag was inserted between residues E1379-S1380. In RIM1 individual domains, an HA-tag was inserted at the C-terminus. The splice variant of full-length RIM1α was lacking alternatively spliced exons (N83-W105, H1084-R1169, A1207-T1378) identical to previous experiments (Deng et al., 2011; de Jong et al., 2018; Kaeser et al., 2011; Tang et al., 2016). For pFSW HA-ELKS1αB (p311) and pFSW HA-ELKS2αB (p314), an HA-tag was inserted at the N-terminus (Held et al., 2016; Nyitrai et al., 2020). The plasmids for expression of zinc finger fusion-proteins were newly generated based on the following cDNAs: pMT2 CaVβ1b GFP (gift from Annette Dolphin obtained through Addgene, plasmid #89893; (which can be found on the world wide web at http://addgene.org/89893); RRID:Addgene_89893 (Page et al., 2016)), Ca_{V}β3 (gift from Diane Lipscombe; (which can be found on the world wide web at http://addgene.org/26574 RRID:Addgene_26574) and pMT2 CaVβ4 (gift from Annette Dolphin obtained through Addgene, plasmid #107426; (which can be found on the world wide web at http://addgene.org/107426); RRID:Addgene_107426 (Brodbeck et al., 2002)). The cDNAs of Liprin-α3 (Wong et al., 2018), Ca_{V}2.1 (Held et al., 2020) and RIM1-Zn^{K144/6E} (Deng et al., 2011) were described before. For pFSW β4-Zn-HA (p661), an HA-tag followed by RIM1-Zn was inserted at the C-terminus of CaVβ4, with the stop codon in CaVβ4 and start codon in RIM1-Zn deleted. For all other RIM1-Zn fusion-proteins, similar strategies were used as shown in FIG. S5A.

STED imaging. Neurons cultured on 0.17 mm thick 12 mm diameter (#1.5) coverslips were washed two times with warm PBS, and then fixed in 4% PFA for 10 min unless noted otherwise. For Ca_{V}2.1 staining, cultures were fixed in 2% PFA+4% sucrose (in PBS) for 10 min. After fixation, coverslips were rinsed twice in PBS+50 mM glycine, then permeabilized in PBS+0.1% Triton X-100+3% BSA (TBP) for 1 hour. Primary antibodies were diluted in TBP and stained for 24-48 h at 4° C. The following primary antibodies were used: guinea pig anti-Synaptophysin (1:500, RRID: AB_1210382, A106), mouse anti-PSD-95 (1:200, RRID: AB_10698024, A149), rabbit anti-RIM1 (1:500, RRID: AB_887774, A58), rabbit anti-ELKS2a (serum E3-1029, 1:100, custom made, A136, (Held et al., 2016)), rabbit anti-Munc13-1 (1:500, RRID: AB_887733, A72), rabbit anti-Ca_{V}2.1 (1:200, RRID: AB_2619841, A46), rabbit anti-RIM-BP2 (1:500, RRID: AB_2619739, A126), rabbit anti-Liprin-α3 (serum 4396, 1:2000, gift from Dr. T. Südhof, A35), rabbit anti-Synaptophysin (1:500, RRID: AB_887905, A64), guinea pig anti-Bassoon^{C} (C-terminal, 1:500, RRID: AB_2290619, A67) and mouse anti-HA (1:500, RRID: AB_2565006, A12). After primary antibody staining, coverslips were rinsed twice and washed 34 times for 5 minutes in TBP. Alexa Fluor 488 (anti-guinea pig, RRID: AB_2534117, S3; anti-rabbit, RRID: AB_2576217, S5; anti-mouse IgG1, RRID: AB_2535764, S7), 555 (anti-mouse IgG2a, RRID: AB_1500824, S20), and 633 (anti-rabbit, RRID: AB_2535731, S33; anti-guinea pig, RRID: AB_2535757, S34) conjugated antibodies were used as secondary antibodies at 1:200 (Alexa Fluor 488 and 555) or 1:500 (Alexa Fluor 633) dilution in TBP, incubated for 24 h at 4° C. followed by rinsing two times and washing 3-4 times 5 minutes in TBP. Stained coverslips were post-fixed for 10 minutes with 4% PFA in PBS (for CaV2.1 staining, 4% PFA+4% sucrose in PBS was used for post-fixation), rinsed two times in PBS+50 mM glycine and once in deionized water, and air-dried and mounted on glass slides. STED images were acquired with a Leica SP8 Confocal/STED 3× microscope with an oil immersion 100×1.44 numerical aperture objective and gated detectors as described in (Wong et al., 2018). 46.51×46.51 μm² areas were taken as regions of interest (ROIs) and were scanned at a pixel density of 4096×4096 (11.358 nm/pixel). Alexa Fluor 633, Alexa Fluor 555, and Alexa Fluor 488 were excited with 633 nm, 555 nm and 488 nm using a white light laser at 2-5% of 1.5 mW laser power. The Alexa Fluor 633 channel was acquired first in confocal mode using 2× frame averaging. Subsequently, Alexa Fluor 555 and Alexa Fluor 488 channels were acquired in STED mode, depleted with 660 nm (50% of max power, 30% axial depletion) and 592 nm (80% of max power, 30% axial depletion) depletion lasers, respectively. Line accumulation (2-10×) and frame averaging (2×) were applied during STED scanning. Identical settings were applied to all samples within an experiment. Synapses within STED images were selected in side-view, defined as synapses that contained a synaptic vesicle cluster labeled with Synaptophysin and associated with an elongated PSD-95 bar along the edge of the vesicle cluster as described (Held et al., 2020; de Jong et al., 2018; Wong et al., 2018). For intensity profile analyses, side-view synapses were selected using only the PSD-95 signal and the vesicle signal for all experiments. An ROI was manually drawn around the PSD-95 signal and fit with an ellipse to determine the center position and orientation. An ˜1200 nm long, 200 nm wide rectangle was then selected perpendicular and across the center of the elongated PSD-95 structure. Intensity profiles were obtained for all three channels within this ROI. To align individual profiles, the PSD-95 signal only was smoothened using a moving average of 5 pixels, and the smoothened signal was used to define the peak position of PSD-95. All three channels (vesicle marker, test protein, and smoothened PSD-95) were then aligned to the PSD-95 peak position and averaged across images. All analyses were performed on raw images without background subtraction, and all adjustments and were done identically for all experimental conditions. Representative images were brightness and contrast adjusted to facilitate inspection, and these adjustments were made identically for images within an experiment. The experimenter was blind to the condition/genotype for image acquisition and analyses.

Confocal imaging of cultured neurons. Neurons cultured on glass coverslips were washed with warm PBS and fixed in PFA for 20 min. Neurons were the permeabilized in TBP for 1 h, and then incubated in primary antibodies at 4° C. overnight. The following primary antibodies were used: rabbit anti-RIM1 (1:1000, RRID: AB_887774, A58), rabbit anti-ELKSα (1:500, RRID: AB_869944, A55), rabbit anti-Munc13-1 (1:500, RRID: AB_887733, A72), rabbit anti-Ca_{V}2.1 (1:1000, RRID: AB_2619841, A46), rabbit anti-RIM-BP2 (1:500, RRID: AB_2619739, A126), mouse anti-Bassoon (1:500, RRID: AB_11181058, A85), mouse anti-MAP2 (1:500, RRID: AB_477193, A108), rabbit anti-MAP2 (1:1000, RRID: AB 2138183, A139), guinea pig anti-Synaptophysin (1:500, RRID: AB_1210382, A106). After staining with primary antibodies, coverslips were rinsed twice and washed 3-4 times for 5 min in TBP. Alexa Fluor 488 (for detection of the protein of interest, anti-rabbit, RRID: AB_2576217, S5; anti-mouse IgG1, RRID: AB_2535764, S7), 546 (for detection of MAP2, anti-mouse IgG, RRID: AB_2535765, S12; anti-rabbit, RRID: AB_2534093, S16), and 633 (for detection of Synaptophysin, anti-guinea pig, RRID: AB_2535757, S34) conjugated secondary antibodies were used at 1:500 dilution in TBP. Secondary antibody staining was done for 2 h at room temperature followed by rinsing two times and washing 3-4 times 5 min in TBP. Coverslips were rinsed once with deionized water and mounted on glass slides. Images were taken on an Olympus FV1200 confocal microscope using identical settings per condition in a given experiment with a 60× oil-immersion objective and single confocal sections were analyzed in ImageJ. For quantitative analyses of synaptic protein levels, the Synaptophysin signal was used to define synaptic puncta as ROIs, and the signal intensity of the protein of interest was quantified within these ROIs. For each image, the “rolling ball” ImageJ plugin was set to a diameter of 1.4 μm for local background subtraction (Sternberg, 1983). Representative images were brightness and contrast adjusted to facilitate inspection, and adjustments were made identically across conditions. The experimenter was blind to the condition/genotype for image acquisition and analyses.

Electrophysiology. Electrophysiological recordings in cultured hippocampal neurons were performed as described (Held et al., 2020; Liu et al., 2014; Wang et al., 2016) at DIV15-19. The extracellular solution contained (in mM): 140 NaCl, 5 KCl, 2 MgCl2, 1.5 CaCl₂), 10 glucose, 10 HEPES-NaOH (pH 7.4, ˜300 mOsm), for FIGS. S3A-S3D, the CaCl₂) concentration was 0.5 mM instead of 1.5 mM, all recordings were performed at room temperature (20-24° C.). To assess action potential-triggered excitatory transmission, NMDAR-mediated excitatory postsynaptic currents (EPSCs) were measured to avoid network activity induced by AMPA receptor activation. For NMDAR-EPSCs, picrotoxin (PTX, 50 μM) and 6-Cyano-7-nitroquinoxaline-2,3-dione (CNQX, 20 μM) were present in the extracellular solution, for FIGS. S3E-S3H, 20 μM L-AP5 was added to the extracellular solution. Inhibitory postsynaptic currents (IPSCs) were recorded in the presence of D-amino-5-phosphonopentanoic acid (D-APV, 50 μM) and CNQX (20 μM) in the extracellular solution. Action potentials were elicited with a bipolar focal stimulation electrode fabricated from nichrome wire. Paired pulse ratios were calculated as the amplitude of the second PSC divided by the amplitude of the first PSC. The baseline value for the second PSC was taken immediately after the second stimulus artifact. For sucrose-induced EPSC recordings, TTX (1 μM), PTX (50 μM), and D-APV (50 μM) were added to the

extracellular solution, and for sucrose-induced IPSC recordings, TTX (1 μM), CNQX (20 μM), and D-APV (50 μM) were added. The RRP was estimated by application of 500 mM sucrose in extracellular solution applied via a microinjector syringe pump for 10 s at a rate of 10 pl/min through a tip with an inner diameter of 250 μm. Glass pipettes were pulled at 2-5 M92 and filled with intracellular solutions containing (in mM) for EPSC recordings: 120 Cs-methanesulfonate, 2 MgCl2, 10 EGTA, 4 Na2-ATP, 1 Na-GTP, 4 QX314-Cl, 10 HEPES-CsOH (pH 7.4, −300 mOsm) and for IPSC recordings: 40 CsCl, 90 K-gluconate, 1.8 NaCl, 1.7 MgCl2, 3.5 KCl, 0.05 EGTA, 2 Mg-ATP, 0.4 Na2-GTP, 10 phosphocreatine, 4 QX314-Cl, 10 HEPES-CsOH (pH 7.2, −300 mOsm). Cells were held at +40 mV for NMDAR-EPSC recordings and at −70 mV for sucrose EPSC, eIPSC and sucrose IPSC recordings. Access resistance was monitored during recordings and compensated to 3-5 M92, and cells were discarded if the uncompensated access exceeded 15 M92. Data were acquired at 5 kHz and lowpass filtered at 2 kHz with an Axon 700B Multiclamp amplifier and digitized with a Digidata 1440A digitizer. All data acquisition and analysis was done using pClamp10. For electrophysiological experiments, the experimenter was blind to the genotype throughout data acquisition and analysis.

High-pressure freezing and electron microscopy. Neurons cultured on 6 mm matrigel-coated sapphire coverslips were frozen using a Leica EM ICE high-pressure freezer in extracellular solution containing (in mM): 140 NaCl, 5 KCl, 2 MgCl2, 2 CaCl₂), 10 HEPES-NaOH (pH 7.4), 10 Glucose (−310 mOsm) with CNQX (20 μM), D-AP5 (50 μM) and PTX (50 μM) added to block synaptic transmission. After freezing, samples were first freeze-substituted (AFS2, Leica) in 1% glutaraldehyde, 1% osmium tetroxide, 1% water in anhydrous acetone as follows: −90° C. for 5 h, 5° C. per h to −20° C., −20° C. for 12 h, and 10° C. per hour to 20° C. Following freeze substitution, samples were Epon infiltrated, and baked for 48 h at 60° C. followed by 80° C. overnight before sectioning at 50 nm. For ultrathin sectioning, the sapphire coverslip was removed from the resin block by plunging the sample first in liquid nitrogen and followed by warm water several times until the sapphire was completely detached. The resin block containing the neurons was then divided into four pieces, and one piece was mounted for sectioning.

Ultrathin sectioning was performed on a Leica EM UC7 ultramicrotome, and the 50 nm sections were collected on a nickel slot grid (2×1 mm) with a carbon coated formvar support film. The samples were counterstained by incubating the grids with 2% lead acetate solution for 10 seconds, followed by rinsing with distilled water. Images were taken with a transmission electron microscope (JEOL 1200 EX at 80 kV accelerating voltage) and processed with ImageJ. The total number of vesicles, the number of docked vesicles, the length of the PSD, and the area of the presynaptic bouton were analyzed in each section using a custom-written Matlab code. Bouton size was calculated from the measured perimeter of each synapse. Docked vesicles were defined as vesicles touching the presynaptic plasma membrane opposed to the PSD, and only vesicles for which the electron densities of the vesicular membrane and the presynaptic plasma membrane merged such that they were not separated by less electron dense space were considered docked. Due to the laborious nature of these experiments, it was not possible to include a RIM1α full-length rescue condition in each experiment. Instead, we always included control^{R+E} and cKO^{R+E} neurons as essential controls for comparison. Experiments and analyses were performed by an experimenter blind to the genotype.

Western blotting. For assessment of rescue protein expression in cultured neurons, Western blotting was used to detect target proteins in cell lysates from select coverslips of every culture that was used for electrophysiology or electron microscopy. At DIV15-19, cultured neurons were harvested in 20 μl 1×SDS buffer per coverslip and run on standard SDS-Page gels followed by transfer on nitrocellulose membranes. Membranes were blocked in filtered 10% nonfat milk/5% goat serum for 1 h at room temperature and incubated with primary antibodies in 5% nonfat milk/2.5% goat serum overnight at 4° C., and HRP-conjugated secondary antibodies (1:10,000, anti-mouse, RRID: AB_2334540; anti-rabbit, RRID: AB_2334589) were used. Anti-Synapsin or -β-actin antibodies were used as a loading controls. The following primary antibodies were used: rabbit anti-RIM1 (1:1000, RRID: AB_887774, A58), rabbit anti-ELKS2αB (1:500, RRID: AB_731499, A143), rabbit anti-Munc13-1 (1:1000, RRID: AB_887733, A72), rabbit anti-RIM1-Zn (1:500, gift from Dr 1. Südhof, A148), mouse anti-HA (1:1000, RRID: AB_2565006, A12), mouse anti-Synapsin (1:4000, RRID: AB_2617071, A57), mouse anti-β-actin (1:2000, RRID: AB_476692, A127), mouse anti-CaVβ4 (1:50, RRID: AB_10671176, A123). For illustration in figures, images were adjusted for brightness and contrast to facilitate visual inspection.

Statistics. Data are displayed as mean±SEM, statistics were performed in GraphPad Prism 9, and significance is presented as *P<0.05, **P<0.01, and ***P<0.001. Parametric tests were used for normally distributed data (assessed by Shapiro-Wilk tests) or when sample size was n≥30. One-way ANOVA followed by Dunnett's multiple comparisons post-hoc tests were used for datasets with equal variance. When variances were unequal, Brown-Forsythe ANOVA followed by Games-Howell's multiple comparisons post hoc tests (for n≥50) or Dunnett's T3 multiple comparisons post hoc tests (for n<50) were used. For non-normally distributed data, nonparametric tests were used (Mann-Whitney tests or Kruskal-Wallis analysis of variance followed by Dunn's multiple comparisons post-hoc tests). For paired pulse ratios, two-way ANOVA with Dunnett's tests was used. For STED side-view analyses, two-way ANOVA with Dunnett's tests was used on a 200 nm-window centered around the active zone peak. For each dataset, the specific tests used are stated in the figure legends.

What is claimed is:

1. A fusion protein comprising:
a) a zinc-finger domain (ZNF) of Regulating Synaptic Membrane Exocytosis Protein (RIMS); and
b) a Ca_{V}β Ca²⁺ channel subunit.

2. The fusion protein of claim 1, wherein the RIMS is Regulating Synaptic Membrane Exocytosis Protein 1 (RIMS1) or Regulating Synaptic Membrane Exocytosis Protein 2 (RIMS2).

3. The fusion protein of claim 1, wherein the Ca_{V}β Ca²⁺ channel subunit is Ca_{V}β1, Ca_{V}β2, Ca_{V}β3, or Ca_{V}β4.

4. The fusion protein of claim 1, wherein the first domain, the ZNF, comprises a sequence selected from SEQ ID NO: 1-4 and 38-41.

5. The fusion protein of claim 1, wherein the second domain, the Ca_{V}β Ca²⁺ channel subunit, comprises a sequence selected from SEQ ID NO: 5-8 and 42-47.

6. A synthetic nucleic acid encoding the fusion protein of claim 1.

7. A vector encoding the fusion protein of claim 1.

8. The vector of claim 7, wherein the vector is a DNA or RNA nucleic acid vector.

9. The vector of claim 7, wherein the vector further comprises a promoter that is operatively linked to the nucleic acid.

10. The vector of claim 9, wherein the promoter is a constitutive promoter and/or a nervous tissue-specific promoter.

11. The vector of claim 7, wherein the vector is a viral vector.

12. The vector of claim 11, wherein the viral vector is selected from of the group consisting of: an adeno associated virus (AAV), adenovirus, lentivirus vector, and a herpes simplex virus (HSV).

13. A cell expressing the fusion protein of claim 1.

14. The cell of claim 13, wherein the cell is a neuronal cell.

15. A pharmaceutical composition comprising the fusion protein of claim 1.

16. The pharmaceutical composition of claim 15, wherein the formulation of the pharmaceutical composition is selected from the group consisting of: direct injection or infusion into the central nervous system (CNS); formulation as a solution comprising a carrier protein; formulation as a nanoparticle; formulation as a liposome; formulation as a nucleic acid; formulation as a CNS-tropic viral vector; formulation with or linkage to an agent that is endogenously transported across the BBB; formulation with or linkage to a cell penetrating peptide (CPP); formulation with or linkage to a BBB-shuttle; and formulation with or linkage to an agent that increases permeability of the BBB.

17. The pharmaceutical composition of claim 15, wherein the pharmaceutical composition is formulated for delivery across the blood-brain barrier (BBB) and/or delivery to the brain.

18. A method of repairing or enhancing synaptic function in a subject, the method comprising administering to a subject in need thereof an effective amount of the fusion protein of claim 1.

19. A method of treating a neurological or secretory disorder in a subject, the method comprising administering to a subject in need thereof an effective amount of the fusion protein of claim 1.

20. The method of 19, wherein administrations is performed intracranially, epidurally, intrathecally, intraparenchymally, intraventricularly, or subarachnoidly.


SEQUENCE LISTING (47 sequences)

SEQ ID NO: 1 - protein, 1692 aa, Homo sapiens
MSSAVGPRGPRPPTVPPPMQELPDLSHLTEEERNIIMAVMDRQKEEEEKEEAMLKCVVRDMAKPAACKTPRNAENQPHQPSPRLHQQFESYKEQVRKIGEEARRYQGEHKDDAPTCGICHKTKFADGCGHLCSYCRTKFCARCGGRVSLRSNNEDKVVMWVCNLCRKQQEILTKSGAWFFGSGPQQTSQDGTLSDTATGAGSEVPREKKARLQERSRSQTPLSTAAASSQDAAPPSAPPDRSKGAEPSQQALGPEQKQASSRSRSEPPRERKKTPGLSEQNGKGALKSERKRVPKTSAQPVEGAVEERERKERRESRRLEKGRSQDYPDTPEKRDEGKAADEEKQRKEEDYQTRYRSDPNLARYPVKPPPEEQQMRMHARVSRARHERRHSDVALPRTEAGAALPEGKAGKRAPAAARASPPDSPRAYSAERTAETRAPGAKQLTNHSPPAPRHGPVPAEAPELKAQEPLRKQSRLDPSSAVLMRKAKREKVETMLRNDSLSSDQSESVRPSPPKPHRSKRGGKKRQMSVSSSEEEGVSTPEYTSCEDVELESESVSEKGDLDYYWLDPATWHSRETSPISSHPVTWQPSKEGDRLIGRVILNKRTTMPKDSGALLGLKVVGGKMTDLGRLGAFITKVKKGSLADVVGHLRAGDEVLEWNGKPLPGATNEEVYNIILESKSEPQVEIIVSRPIGDIPRIPESSHPPLESSSSSFESQKMERPSISVISPTSPGALKDAPQVLPGQLSVKLWYDKVGHQLIVNVLQATDLPARVDGRPRNPYVKMYFLPDRSDKSKRRTKTVKKILEPKWNQTFVYSHVHRRDFRERMLEITVWDQPRVQEEESEFLGEILIELETALLDDEPHWYKLQTHDESSLPLPQPSPFMPRRHIHGESSSKKLQRSQRISDSDISDYEVDDGIGVVPPVGYRSSARESKSTTLTVPEQQRTTHHRSRSVSPHRGNDQGKPRSRLPNVPLQRSLDEIHPTRRSRSPTRHHDASRSPVDHRTRDVDSQYLSEQDSELLMLPRAKRGRSAECLHTTRHLVRHYKTLPPKMPLLQSSSHWNIYSSILPAHTKTKSVTRQDISLHHECFNSTVLRFTDEILVSELQPFLDRARSASTNCLRPDTSLHSPERERGRWSPSLDRRRPPSPRIQIQHASPENDRHSRKSERSSIQKQTRKGTASDAERVLPTCLSRRGHAAPRATDQPVIRGKHPARSRSSEHSSIRTLCSMHHLVPGGSAPPSPLLTRMHRQRSPTQSPPADTSFSSRRGRQLPQVPVRSGSIEQASLVVEERTRQMKMKVHRFKQTTGSGSSQELDREQYSKYNIHKDQYRSCDNVSAKSSDSDVSDVSAISRTSSASRLSSTSFMSEQSERPRGRISSFTPKMQGRRMGTSGRSIMKSTSVSGEMYTLEHNDGSQSDTAVGTVGAGGKKRRSSLSAKVVAIVSRRSRSTSQLSQTESGHKKLKSTIQRSTETGMAAEMRKMVRQPSRESTDGSINSYSSEGNLIFPGVRLGADSQFSDFLDGLGPAQLVGRQTLATPAMGDIQIGMEDKKGQLEVEVIRARSLTQKPGSKSTPAPYVKVYLLENGACIAKKKTRIARKTLDPLYQQSLVFDESPQGKVLQVIVWGDYGRMDHKCFMGVAQILLEELDLSSMVIGWYKLFPPSSLVDPTLTPLTRRASQSSLESSTGPPCIRS

SEQ ID NO: 2 - protein, 1349 aa, Homo sapiens
MSAPVGPRGRLAPIPAASQPPLQPEMPDLSHLTEEERKIILAVMDRQKKEEEKEQSVLKKLHQQFEMYKEQVKKMGEESQQQQEQKGDAPTCGICHKTKFADGCGHNCSYCQTKFCARCGGRVSLRSNKVMWVCNLCRKQQEILTKSGAWFYNSGSNTPQQPDQKVLRGLRNEEAPQEKKPKLHEQTQFQGPSGDLSVPAVEKSRSHGLTRQHSIKNGSGVKHHIASDIASDRKRSPSVSRDQNRRYDQREEREEYSQYATSDTAMPRSPSDYADRRSQHEPQFYEDSDHLSYRDSNRRSHRHSKEYIVDDEDVESRDEYERQRREEEYQSRYRSDPNLARYPVKPQPYEEQMRIHAEVSRARHERRHSDVSLANADLEDSRISMLRMDRPSRQRSISERRAAMENQRSYSMERTREAQGPSSYAQRTTNHSPPTPRRSPLPIDRPDLRRTDSLRKQHHLDPSSAVRKTKREKMETMLRNDSLSSDQSESVRPPPPKPHKSKKGGKMRQISLSSSEEELASTPEYTSCDDVEIESESVSEKGDMDYNWLDHTSWHSSEASPMSLHPVTWQPSKDGDRLIGRILLNKRLKDGSVPRDSGAMLGLKVVGGKMTESGRLCAFITKVKKGSLADTVGHLRPGDEVLEWNGRLLQGATFEEVYNIILESKPEPQVELVVSRPIGDIPRIPDSTHAQLESSSSSFESQKMDRPSISVTSPMSPGMLRDVPQFLSGQLSSQSLSRRTTPFVPRVQIKLWFDKVGHQLIVTILGAKDLPSREDGRPRNPYVKIYFLPDRSDKNKRRTKTVKKTLEPKWNQTFIYSPVHRREFRERMLEITLWDQARVREEESEFLGEILIELETALLDDEPHWYKLQTHDVSSLPLPHPSPYMPRRQLHGESPTRRLQRSKRISDSEVSDYDCDDGIGVVSDYRHDGRDLQSSTLSVPEQVMSSNHCSPSGSPHRVDVIGRTRSWSPSVPPPQSRNVEQGLRGTRTMTGHYNTISRMDRHRVMDDHYSPDRDRDCEAADRQPYHRSRSTEQRPLLERTTTRSRSTERPDTNLMRSMPSLMTGRSAPPSPALSRSHPRTGSVQTSPSSTPVAGRRGRQLPQLPPKGTLDRKAGGKKLRSTVQRSTETGLAVEMRNWMTRQASRESTDGSMNSYSSEGNLIFPGVRLASDSQFSDFLDGLGPAQLVGRQTLATPAMGDIQVGMMDKKGQLEVEIIRARGLVVKPGSKTLPAPYVKVYLLDNGVCIAKKKTKVARKTLEPLYQQLLSFEESPQGKVLQIIVWGDYGRMDHKSFMGVAQILLDELELSNMVIGWFKLFPPSSLVDPTLAPLTRRASQSSLESSTGPSYSRS

SEQ ID NO: 3 - DNA, 7984 bp, Homo sapiens
  source 1..7984: mol_type = unassigned DNA
aaattggaggcagcagagcctgggagcagcctccacggcggcagcggccgccccagtcccagccccagccccagccccagccccgcccccgccccgcgccccgccggagacgcccggatcggcggagcctggcgcgagccctcgccccctcgcctctcccgccgcgcctccgcctgcccgcccccgccggccgaggctgggctgcgggaggcggccgggcggccccgagcttcgctagggcgaccaaaacaaaggcagcatccggggctgggtggatgcaaacaaccatgaaagactgggttctcgctctccccggctctgctgctgctgctgctgccgccgccgccgctgctcctcctcctgccgccgccgctagggctccgctgtgagggggaagcaggggcgcagctgctgggcgtgcatccgaaaggtgagagccagagagcgagcagagggggcgggcaggccacgaaaatgtcctcggccgtggggccccgcggtcctcgcccacccacggtgcctccccccatgcaagagctgcccgacctgagccacctgaccgaagaggagaggaacattatcatggcagtgatggaccggcagaaggaagaggaggaaaaagaagaagccatgctcaagtgtgttgtcagggacatggcgaagcctgctgcctgcaaaacaccaagaaatgctgaaaaccagccccaccaaccttcaccgagattgcatcaacagtttgaaagctataaggaacaagtgagaaaaataggggaagaagcgcggcgttaccagggcgagcacaaagacgatgctccgacttgtggaatctgtcataaaacaaagtttgctgatgggtgcggtcatctctgctcctattgtcgcactaagttctgtgcgcgctgcggaggccgcgtgtctctacggtcaaacaacgaggacaaagtggttatgtgggtatgcaatttatgtcgaaagcaacaagaaatcttaaccaaatctggggcatggttctttggaagtggccctcagcagacaagtcaggatggaaccctgagtgatacagctacaggtgctggctctgaggtaccaagagaaaagaaagcacgactccaagagcgatcgcggtctcagacacccctaagcacagcagctgcctcctcccaggatgctgctcctcccagcgcaccaccagacaggagcaaaggggctgagccctcgcagcaagccttggggcctgaacagaagcaggcttcatccaggtctagaagtgaacctcctagagagagaaagaagaccccagggctttccgagcagaatggcaaaggagccctgaagagcgagcggaaacgcgtgccaaagacctcagcgcagcccgtggagggggccgtcgaagaacgggagcgcaaagaaaggcgggaaagccgaaggcttgagaaagggcgatcacaggattacccagacacgccggaaaaacgggatgagggcaaagcggcggatgaggaaaagcaaagaaaagaggaggattatcagaccaggtaccgcagcgacccgaacctagctcggtacccggtgaaaccgccgcctgaggagcagcagatgcgcatgcacgcccgggtgtcccgcgccaggcacgagcggcgccacagcgacgtggcgctcccgcgcaccgaggcgggcgcggcgctgccggagggcaaggccggcaaacgcgcgccggcggcagccagggcctcgccgccggactcgccgcgggcttactcggctgagagaactgcggagaccagggcgccgggcgccaagcagctaacgaaccacagcccgccggcgcccagacatgggccggttcccgcagaagccccggagctcaaagcccaggagcccctcaggaagcagagccgcctggaccccagctcggcggtcctcatgcggaaggccaagcgcgagaaggtggagaccatgctgcggaacgactctttgagctcagaccagtccgagtcggtgcggccgtccccgcccaagccgcaccggtccaagagaggcggcaagaagcggcagatgtcggtgagcagctctgaggaggagggcgtgtcgacgcccgagtacaccagctgcgaggacgtggagctggagagcgagagcgtcagcgagaaaggtgatttggattattactggttggatcctgccacgtggcacagccgggagacatcacctattagttcgcatcctgtaacgtggcaaccatctaaagagggggaccgattaattggacgtgttattcttaacaagagaacaaccatgcccaaagactcaggtgcattgctgggtctgaaagttgttggaggaaaaatgactgacttaggacgacttggtgctttcatcaccaaagtaaagaagggtagcctagcagatgtagttggacacctaagagcaggggatgaagttctagaatggaatggtaaacccctgccgggagctacaaatgaagaagtttacaacattattttagaatcaaaatcagaacctcaagttgaaattattgtttcaaggcctattggtgacattccccggattcctgagagctcccaccctccactggagtccagttcaagttcctttgaatctcagaagatggaaaggccttccatttctgttatttctccaacaagtcctggagctctaaaagatgccccacaagtcttaccagggcaactttctgtgaagttgtggtatgataaagtgggacaccagctgattgtaaatgttctgcaagcaacagatctacctgctagagtagatggacgtcctcgaaatccctatgtaaaaatgtattttcttccagatagaagtgataaaagtaaaaggaggaccaaaacagtaaagaaaatactagaaccaaaatggaatcaaacttttgtctattcacatgtacatcgtagagattttagagaacgaatgttagaaataactgtgtgggaccaaccaagagtgcaagaagaagaaagtgaatttcttggagagatcctcatagaattggagacagcgcttttagatgatgaaccgcattggtataaacttcagacacatgatgagtcttcactacctctgcctcagccatcacctttcatgccaaggcgacatattcatggagaaagctctagcaaaaagctacaaagatctcagcgaatcagtgatagtgacatctcagattatgaggttgatgatggtattggcgtagttcctccagtaggctataggtctagtgctagagaaagtaaatctacaacattaactgtgccagaacagcaaagaacaactcatcaccgctcacgttcagtatctcctcatcgcggcaatgatcagggaaagccgcgttcacgtttaccaaatgtgccattacagaggagtttagatgaaattcatccaacaagaaggtcacgttctccaaccagacaccatgatgcctcccgaagtccagttgatcatagaaccagagatgtggatagtcagtatttatcagaacaagacagtgagcttcttatgctgcccagagcaaaacgaggacgaagtgcagaatgcctacatactaccagacatcttgttaggcactataaaacattacctcccaagatgcctttattacagagcagttctcactggaatatttacagctcaattctgcctgcacatactaagaccaaatcagtgactagacaggacatttcccttcatcatgaatgctttaactcaacagtattgagatttactgatgaaatactggttagtgaactgcagccctttcttgacagggctaggagtgctagtaccaactgcttgagaccagatactagtttgcattcaccagaacgagaaaggggtagatggtccccctccctagataggagacgacctcctagtcccaggattcaaatccagcatgcgtctccggagaatgacaggcactccagaaagtctgaaagatctagcatccaaaaacagactaggaaaggcactgcctctgatgcagaaagggttctcccaacatgtctttctagaaggggacacgcagccccaagagcaactgatcagccagtcattaggggaaaacatcctgctcgctcaaggtcgagtgagcactctagtatcagaacactgtgttctatgcaccaccttgtccctggagggtcggcgccaccttctccgcttctgacaagaatgcaccgacagagaagtccaacacaatctcctccagcagacacatcgttcagcagtcgcaggggaagacagctcccacaagtgccagtgagaagcggcagtatagaacaagcaagcttagtagtggaggagcgaacaagacagatgaaaatgaaagtgcatcgatttaagcagacaacagggtctggttctagtcaagaacttgatcgcgagcaatattccaagtataacatacataaagatcagtacagaagctgtgataacgtctctgccaaatcatcagatagtgatgtcagtgatgtttccgccatttcccgaaccagcagtgcctcacgcctcagcagcacaagctttatgtcagagcaatctgagcgccccaggggtagaatcagttcatttacccccaaaatgcaaggcagacggatggggacttcaggaagatccatcatgaagagcaccagtgtcagtggagagatgtacacactggagcataatgacggcagccagtcagacacagctgtgggtacagttggagcaggtggaaagaaacggagatccagccttagtgccaaagtggttgccatagtgtctcgaaggagtagaagcacatcccagcttagtcaaacagagtcgggccacaaaaagttaaaaagtaccatccagagaagcacagaaacaggcatggcagctgaaatgagaaagatggtaaggcagccgagccgagagtctactgatggcagcatcaacagttacagctctgagggcaatttaatatttcctggagtgcgactgggagctgacagtcaattcagtgattttcttgatggattgggaccagcccagcttgttggccgccaaacccttgccacccctgcaatgggtgatatacaaataggaatggaggacaaaaagggccaattagaagtggaagtcattagagcacgaagcctcacacaaaagcctggttccaaatctacacctgctccatatgtcaaagtatatcttttggaaaatggggcctgtatagccaagaagaagacaagaattgcacgaaaaacccttgatcctttgtatcagcagtctctggtttttgatgaaagtccacagggtaaagttcttcaggtgattgtctggggagactatggcagaatggaccacaaatgctttatgggtgtggctcagatcttgttggaagaactcgacctgtccagcatggtgatcggatggtacaaattgttcccaccgtcctcactggtggatcccacactcactcccctcacccggcgggcttcccagtcatctctggaaagttcaactgggcctccctgtattcgatcatagtgaactcataccagagtcattccaataaaactctacttttcaggataataatctgaaccagatatttcatgatcgaaagcattgttggagacagacaatcaacttgtgttttgcctgtagtagtttttcaataatatgtcccaattgttatttaaaacatggcttcatatgacagaacaaggcaatctatcaaatttacaggaagaatcaacatgctggtgagagtcactgatgcttctaacaaatagaaaaagaggaaactttaaatccacgcatacacgtacacacacacatgcacacacacacacaccaaattgaacaaactggaaactctcactctgtgaaaagttgtattctacacatttctgcacagaccacaagcagtgttatttccctgatgtttgagttgttttgttcttttgtgtgttttgtttatttgtgtgttgtttgttggttttcgtttcttgagtttttgttttgttttcaccacatctgttatttccactagtttttttttggctgtgtcatgacaggcctcatgatgctaacagagattcttcccttcttttttccaaaagcaccaaaaaaaagggctgaagtagtctctgtagcatctccccaaagtgttaaacagctacataaggtgaagccaaggaagggtttcactagctttcattttataattatttaaagtcttatgtgtggatacccaagagacaaaaagttatcttccaaaataaataatcacagaagtacttttttaatgaagagcctatctgtttgcattctagaaaattaatttatggtttccattgttttgcatggaagacttttaaagaagtcaatctgcaactaatgctggggactaaaactaactgcataattgtactttgaaaacactccttgtaatgcctttttcttcctgatattaaaaaaatgctcatgtgtataaaccatactttgacttcccaaaaatggtgactaggaatggatttgagcctaagacactttgagctatgcccaggcttctcagccatacctcagtgtagacacatccatctgccctggaaagagcatggtatgagctgttcaatattccactggaaattccagttgaaatattctgcactaaactattgtttttattgaattttagtttacatttctttgagattgatgcaagcacaaagcttgattttttaatgcaaagtatcttactttttggggggaaaataaacaaaatgaactttcaatttgcattttcatttagtttctcttggccttgaatttcccttgtgacattctgtacatgtgctacaaactgcagtctctgcaggtgcattgatatgttctccctccttttatgagtcactccacttttgtgattacacaaatagagcagcatgacttggaactgttcagagctgcatgcacattttgtaaaaaaaaaaaattttaaaaagaaaagaaacaaaaggttatttaataatgtatgttagttccacataggccagcttgtatgttgcatgtacttgtacagtttgcttgttaattactataccgaaataaccaagaaatctaagccatccacttttgttatagacattttgcaggttttagccagactcagtctctgggtctgtggtgaaatgtctatagatggactttattttttaccctccggaaaacgtgatgtagtacggaccaaattccttctaataaatattttggtgtagattcctactgtggggctgtaacacatgtagacactgtgtacacactaggttttaattcatagacatactgcctgcaccaagtgaattatttattattatttacacatgccagaattatctgcccatcattccacagggcacagattgaccactgctcaccatgctgagtaggaggaactgaagcattggtgcacttctactagattccgttctgtcttagtggccaacaatcaactgattataattgggtagtatctttctattttgaccacttgtcttaacactcccctgtgcttttaaatgaaattccaattcatgtatgtaaacatgtttaataaaatttacttttcatgtcagtcagtagccatatgtgttctatgtcttgccagatttccattgtgggttctatggtgcaaaatgggagccaggccatgggaaggggaggctggggtttgttgtctgctgcgggcatttgacttgacttgtcactgcactatattatgtcttaatcgatggcccaaagggtactttgcatcacatacaagttttcacttcgtcagcctgtctaagtgagcacatgactatacttctgaaaagtggcattattaaaaataataaatctcagtaagaatcaaatggaaggttgttttctgctgctgtttgaatgtgctaataaaagagacaactctattgtc

SEQ ID NO: 4 - DNA, 8601 bp, Homo sapiens
  source 1..8601: mol_type = unassigned DNA
agttcccctttcccttgaaccgctcacttcacagcccttcgcccccgggaagaagaaacatttcccgaagcgcactcctcagccctccttccccacgcgctcgccctcccctccccctgcttttcttgggggaggggggctgtcgccttggattgaaggccattgatttgtatgtatttgtcccagcgctggaggctgccccagccgccgcgccggtgccgccgctgccagtggagttgcctccccgcttccctagggtggttcggctccaccaaacatgtcggctcctgtcgggccccggggccgcctggctcccatcccggcggcctctcagccgcctctgcagcccgagatgcctgacctcagccacctcacggaggaggagaggaaaatcatcctggccgtcatggataggcagaagaaagaagaggagaaggagcagtccgtgctcaaaaaactgcatcagcagtttgaaatgtataaagagcaggtaaagaagatgggagaagaatcacagcaacagcaagaacagaagggtgatgcgccaacctgtggtatctgccacaaaacaaagtttgctgatggatgtggccataactgttcatattgccaaacaaagttctgtgctcgttgtggaggtcgagtgtcattacgctcaaacaaggttatgtgggtatgtaatttgtgccgaaaacaacaagaaatcctcactaaatcaggagcatggttttataatagtggatctaatacaccacagcaacctgatcaaaaggttcttcgagggctaagaaatgaggaggcacctcaggagaagaaaccaaaactacatgagcagacccagttccaaggaccctcaggtgacttatctgtacctgcagtggagaaaagtcgatctcatgggctcacaagacagcattctattaaaaatgggtcaggcgtgaagcatcacattgccagtgacatagcttcagacaggaaaagaagcccatctgtgtccagagatcagaatagaagatacgaccaaagggaagaaagagaggaatattcacagtatgctacttcggataccgcaatgcctagatctccatcagattatgctgataggcgatctcaacatgaacctcagttttatgaagactctgatcatttaagttatagggactccaacaggagaagtcataggcattccaaagaatatattgtagatgatgaggatgtggaaagcagagatgaatacgaaaggcaaaggagagaggaagagtaccagtcacgctaccgaagtgatccgaatttggcccgttatccagtaaagccacaaccctatgaagaacaaatgcggatccatgctgaagtgtcccgagcacggcatgagagaaggcatagtgatgtttctttggcaaatgctgatctggaagattccaggatttctatgctaaggatggatcgaccatcaaggcaaagatctatatcagaacgtagagctgccatggaaaatcagcgatcttattcaatggaaagaactcgagaggctcagggaccaagttcttatgcacaaaggaccacaaaccatagtcctcctacccccaggaggagtccactacccatagatagaccagacttgaggcgtactgactcactacggaaacagcaccacttagatcctagctctgctgtaagaaaaacaaaacgggaaaaaatggaaacaatgttaaggaatgattctctcagttcagaccagtcagagtcagtgagacctccaccaccaaagcctcataaatcaaagaaaggcggtaaaatgcgccagatttcgttgagcagttcagaggaggaattggcttccacgcctgaatatacaagttgtgatgatgttgagattgaaagtgagagtgtaagtgaaaaaggagacatggattacaactggttggatcatacgtcttggcatagcagtgaggcatccccaatgtctttgcaccctgtaacctggcaaccatctaaagatggagatcgtttaattggtcgcattttattaaataagcgtctaaaagatggaagtgtacctcgagattcaggagcaatgcttggcttgaaggttgtaggaggaaagatgactgaatcaggtcggctttgtgcatttattactaaagtaaaaaaaggaagtttagctgatactgtaggacatcttagaccaggtgatgaagtattagaatggaatggaagactactgcaaggagccacatttgaggaagtgtacaacatcattctagaatccaaacctgaaccacaagtagaacttgtagtttcaaggcctattggagatataccgcgaatacctgatagcacacatgcacaactggagtccagttctagctcctttgaatctcaaaaaatggatcgtccttctatttctgttacctctcccatgagtcctggaatgttgagggatgtcccacagttcttatcaggacaactttcaagccaaagccttagtagaagaacaacgccttttgttcctagggttcagataaaactatggtttgacaaggttggtcaccaattaatagttacaattttgggagcaaaagatctcccttccagggaagatgggaggccaaggaatccttatgttaaaatttactttcttccagacagaagtgataaaaacaagagaagaactaaaacagtaaagaaaacattggaacccaaatggaaccaaacattcatttattctccagtccaccgaagagaatttcgggaacgaatgctagagattaccctttgggatcaagctcgtgttcgagaggaagaaagtgaattcttaggcgagattttaattgaattagaaacagcattattagatgatgagccacattggtacaaacttcagacgcatgatgtctcttcattgccacttccccacccttctccatatatgccacgaagacagctccatggagagagcccaacacggaggttgcaaaggtcaaagagaataagtgatagtgaagtctctgactatgactgtgatgatggaattggtgtagtatcagattatcgacatgatggtcgagatcttcaaagctcaacattatcagtgccagaacaagtaatgtcatcaaaccactgttcaccatcagggtctcctcatcgagtagatgttataggaaggactagatcatggtcacccagtgtccctcctccacaaagtcggaatgtggaacaggggcttcgagggacccgcactatgaccggacattataatacaattagccgaatggacagacatcgtgtcatggatgaccattattctccagatagagacagggattgtgaagcagcagatagacagccatatcacagatccagatcaacagaacaacggcctctccttgagcggaccaccacccgctccagatccactgaacgtcctgatacaaacctcatgaggtcgatgccttcattaatgactggaagatctgcccctccttcacctgccttatcgaggtctcatcctcgtactgggtctgtccagacaagcccatcaagtactccagtcgcaggacgaaggggccgacagcttccacagcttccaccaaagggaacgttggatagaaaagcaggaggtaaaaaactaaggagcactgtccaaagaagtacagaaacaggcctggccgtggaaatgaggaactggatgactcgacaggcaagccgagagtctacagatggtagcatgaacagctacagctcagaaggaaatctgattttccctggtgttcgcttggcctctgatagccagttcagtgatttcctggatggccttggccctgctcagctagtgggacgccagactctggcaacacctgcaatgggtgacattcaggtaggaatgatggacaaaaagggacagctggaggtagaaatcatccgggcccgtggccttgttgtaaaaccaggttccaagacactgccagcaccgtatgtaaaagtgtatctattagataacggagtctgcatagccaaaaagaaaacaaaagtggcaagaaaaacgctggaacccctttaccagcagctattatctttcgaagagagtccacaaggaaaagttttacagatcatcgtctggggagattatggccgcatggatcacaaatcttttatgggagtggcccagatacttttagatgaactagagctatccaatatggtgatcggatggttcaaacttttcccaccttcctccctagtagatccaaccttggcccctctgacaagaagagcttcccaatcatctctggaaagttcaactggaccttcttactctcgttcatagcagctgtaaaaaaattgttgtcacagcaaccagcgttacaaaaaaaaaaaaaaaaatcacaggttgcaaaccctggtaacactgcatgcttaatgttgtgtcttctgagcctgtttctagggatacaaagcaatcctgtgttctcagaggaagttgcacacattgtgccctaaagaaggccctcaggtgaaagagcagagctgtgaagaactatcagatttggaattcaatgacactcgagttctggtccaatctgaagccatggattaatctcaaagaatcagtcagtttcatgcaacagaagcccttttcaatggcacctttatatttttatcattcctttttcttcatttatctaaccccaaagccctgatatgccacagaaatggagctatacagccatgaagcggtgttacaggtgaggagtgtaatcctaggaagcatcaggtgaaaagcaggagaccaaagaagtggtcaggaacaatcatcagccctcctctgggcgggaatcagagcagtcagtccagcaggaagagtggcagactttgtagctccatgggcacgtcaattactaatgctaagatgtgttggactctgaaaaacaaaattctgtggctacactgtactgaatgaaattaaagaaactttttttgcatggacacagattagctgaatacttaaattattttcttggggctgcaacttgcaaaaaaaaaaaaaagaataaaaatcagccattttcaacaatttatattatttttaaaaataaatttcactagtgcatggttttaaaaaggagagagaatgcaacagggtgatacaaagatacaccatgtttattctttaatcatagtctgtgttttggcagacattacaaatggaaatactttctagaagatacttaaaattctctttatgtgacaaataagtataatatattcaatttatttccatgttaaatatacaaatcttatgaagttcaatatgtgcaaatttttcacatctttctccttctctcactttacctcttctccctcttttaaacttttctttctccctgccagagtgaaccttatactaaaaaattacaagttttgatctgatcctctctcataccccatgtttgattcagagctgtagatgcctctgaatttgcgaatttctcaagggaaaattaactttaagagctttctttatttcaagcatgttgaaaaggattttgcaacatgacttgggagtacattaaagtaagtcagcatgtatttgacgaagaagatatttgaacttttgcagtttattgtacagtgcatggtaattttttcacctttaaaattcagtttacaggaaaattctaaaatcatgttgccattgtgatgtccaataaatttgtttttagcaccagcattattcatacaggggttaaagtattatttgtagaaggtcttaggttttgtttgttttttaatcatttaaagcaatttctttagccagtttccatttactatgtgaatagaagcactgctaaaaattgggaaccctgaaacacagggctgtttattaattcatttttctgtagtaaaattcaatttttcacaaattatatttctaaagaaatatagtaaacataaatttgcaacaattttaaagctccagtttttaggtgactcaaagaaagtcattatgcctattaatagttatttgatgccatcaccaaaagtctatgtgaaaatctcctaaagtcaaaacccctgcctttggttttacagacggttattaccattgggtggagctgcaaggtcaaatttctcctaagttcccctatttagaggaaaagtcactggttattgtaataaaccacccatggttctttatgtacattttgataacacattattatagcttgattttaattttttgcattaatttttgaaatccacatacatctcatttgtttaaattaaggccatgcacaaatattttttttagttcagtgctgaccattaaaaactatcatgcttgatacggtgcaaaagttaaaatgagtatcactaaaaatgccttctttttatgtggtgcaatatgaaatacaccaagactgtgtcttgacattctgatggacccaggtaaagttgttaaaagaacgaataaaactttattaaaataatttagacacctgtgtaccagcaacaattgatttaatagacctatagtgtctatactatcccttagaataaaggtttatgattttcctgatactaagatgcagtcacataatcttttgtgcatattcctatacaaattatttctaattttaataagaaggacgtgactacggaatatttgtacatacttgtcattatgcagtatttatttaaaagttggtgtttttttttaattttcacatctgcacctcgacttgtggtttagtcatgtaactagcactatgccagtgaccgttgttgccctgtacatagtatgtttgaaaagtaaagggaattccagttgggaaaaaagggcagattagtcctgtaatgaacaccaactaatgtaaatcaaattcattctggtgatggtatttaacactttaaataaaacattttctttacaggcgtctgcagtgctttctctgacttttctccccacacagccctgagcctgctgcagctcattccctgaactcatgtgtcatttaaagaatgaaatcaccgtctcctacttctcgataacataagtggactgctggtcttagcagcggccctcagtagagcatttctttaaaacgccaaaggatttctgctcacactatgaaaaggtgctgttttttaaaaggttgttattttggattgagtttctttctgattaaatgactcagcaactcacagattttttgagtgaaatttttaatttagtcatggccttcactgacagcatagtcacaaatactcaggcacaggctctgctagcccctgggtgaagatggcgaaggcataactggctttatgcagcatatgtgtttctgctaaagtgtcagttttgctttgtggggagtggagggtgtgttttcgggatggggagacgtggtaacctgacatgtaacaacctgtccggagactagcttctacgtgtggatatgaatgggtgagaggatttctccatatccttctggggcgattcctcaactgggagaaggaaaccctgcagagttctcatgggagtctgcttcaggtttgaaatttaagagctagtttggattcatgtttagtaatcgaactgaaatctaagtctagctgtctctctattcttggaaacaaccatttcctccatttccaaagactcaactcgagtccaattccccctatctgtcccatatatttttcctttatcccatatataccccctactctagtgaattgtttctttgttgttcattcctgttctttgttgttcatatacattcctgtatatgaacaacattttcctttatcccatatataccccctactctagtgaattgtttctttgttgttcattcctgtttctttgttgttcatatacattcctgttataaaaattcccttccctttcttatgtgccctctcctgaaaagcccttctacttttctcaataatgatccatgcgagtcccttcttgcaactcccagctcacgaatgagctctttcggcaactcctgactaaaccctaacaacatggctgccattgatgccaacaccttcactttcccagggaccccagatgccaaggctccataggcaacaataaaggatatgatggtcctgtagtgggtatgatagaattaggcaagagatcaccaaagctgtctgcctactactgatgtaaaccttgacattctgtgcacgtaaaaatcatgtgctcaatgtgtttgtctcaactccctcagctcgtgatgccctcagggttctgtgggcatttatgcactgaagaaacaggagttcacacatccacctctggactgtgaaatgtgtattgagaaatactttgcaagagagaatttttttaagtgaacaaacaacaagtctgtgccacacacatcttccatatgccctgactcaggtcacttaatctccaggatttcatttcctcacctggaaaatatggagtttgaggtagattctcatctatcattaaatcaacactttaactaaaacgtaagctccttcagggcagagaccgtatcttcagtatcaaaaacaatgtttgacacatagctgctcaataaacatctgtccaatgaa

SEQ ID NO: 5 - protein, 486 aa, Homo sapiens
MYDNLYLHGIEDSEAGSADSYTSRPSDSDVSLEEDREAIRQEREQQAAIQLERAKSKPVAFAVKTNVSYCGALDEDVPVPSTAISFDAKDFLHIKEKYNNDWWIGRLVKEGCEIGFIPSPLRLENIRIQQEQKRGRFHGGKSSGNSSSSLGEMVSGTFRATPTSTAKQKQKVTEHIPPYDVVPSMRPVVLVGPSLKGYEVTDMMQKALFDFLKHRFDGRISITRVTADISLAKRSVLNNPSKRAIIERSNTRSSLAEVQSEIERIFELARSLQLVVLDADTINHPAQLIKTSLAPIIVHVKVSSPKVLQRLIKSRGKSQSKHLNVQLVAADKLAQCPPEMFDVILDENQLEDACEHLGEYLEAYWRATHTTSSTPMTPLLGRNLGSTALSPYPTAISGLQSQRMRHSNHSTENSPIERRSLMTSDENYHNERARKSRNRLSSSSQHSRDHYPLVEEDYPDSYQDTYKPHRNRGSPGGYSHDSRHRL

SEQ ID NO: 6 - protein, 484 aa, Homo sapiens
MYDDSYVPGFEDSEAGSADSYTSRPSLDSDVSLEEDRESARREVESQAQQQLERAKHKPVAFAVRTNVSYCGVLDEECPVQGSGVNFEAKDFLHIKEKYSNDWWIGRLVKEGGDIAFIPSPQRLESIRLKQEQKARRSGNPSSLSDIGNRRSPPPSLAKQKQKQAEHVPPYDVVPSMRPVVLVGPSLKGYEVTDMMQKALFDFLKHRFDGRISITRVTADLSLAKRSVLNNPGKRTIIERSSARSSIAEVQSEIERIFELAKSLQLVVLDADTINHPAQLAKTSLAPIIVFVKVSSPKVLQRLIRSRGKSQMKHLTVQMMAYDKLVQCPPESFDVILDENQLEDACEHLAEYLEVYWRATHHPAPGPGLLGPPSAIPGLQNQQLLGERGEEHSPLERDSLMPSDEASESSRQAWTGSSQRSSRHLEEDYADAYQDLYQPHRQHTSGLPSANGHDPQDRLLAQDSEHNHSDRNWQRNRPWPKDSY

SEQ ID NO: 7 - protein, 605 aa, Homo sapiens
MQCCGLVHRRRVRVSYGSADSYTSRPSDSDVSLEEDREAVRREAERQAQAQLEKAKTKPVAFAVRTNVSYSAAHEDDVPVPGMAISFEAKDFLHVKEKFNNDWWIGRLVKEGCEIGFIPSPVKLENMRLQHEQRAKQGKFYSSKSGGNSSSSLGDIVPSSRKSTPPSSAIDIDATGLDAEENDIPANHRSPKPSANSVTSPHSKEKRMPFFKKTEHTPPYDVVPSMRPVVLVGPSLKGYEVTDMMQKALFDFLKHRFEGRISITRVTADISLAKRSVLNNPSKHAIIERSNTRSSLAEVQSEIERIFELARTLQLVVLDADTINHPAQLSKTSLAPIIVYVKISSPKVLQRLIKSRGKSQAKHLNVQMVAADKLAQCPPELFDVILDENQLEDACEHLADYLEAYWKATHPPSSSLPNPLLSRTLATSSLPLSPTLASNSQGSQGDQRTDRSAPIRSASQAEEEPSVEPVKKSQHRSSSSAPHHNHRSGTSRGLSRQETFDSETQESRDSAYVEPKEDYSHDHVDHYASHRDHNHRDETHGSSDHRHRESRHRSRDVDREQDHNECNKQRSRHKSKDRYCEKDGEVISKKRNEAGEWNRDVYIRQ

SEQ ID NO: 8 - protein, 598 aa, Homo sapiens
MVQKTSMSRGPYPPSQEIPMEVFDPSPQGKYSKRKGRFKRSDGSTSSDTTSNSFVRQGSAESYTSRPSDSDVSLEEDREALRKEAERQALAQLEKAKTKPVAFAVRTNVGYNPSPGDEVPVQGVAITFEPKDFLHIKEKYNNDWWIGRLVKEGCEVGFIPSPVKLDSLRLLQEQKLRQNRLGSSKSGDNSSSSLGDVVTGTRRPTPPASAKQKQKSTEHVPPYDVVPSMRPIILVGPSLKGYEVTDMMQKALFDFLKHRFDGRISITRVTADISLAKRSVLNNPSKHIIIERSNTRSSLAEVQSEIERIFELARTLQLVALDADTINHPAQLSKTSLAPIIVYIKITSPKVLQRLIKSRGKSQSKHLNVQIAASEKLAQCPPEMFDIILDENQLEDACEHLAEYLEAYWKATHPPSSTPPNPLLNRTMATAALAASPAPVSNLQGPYLASGDQPLERATGEHASMHEYPGELGQPPGLYPSSHPPGRAGTLRALSRQDTFDADTPGSRNSAYTELGDSCVDMETDPSEGPGLGDPAGGGTPPARQGSWEDEEEDYEEELTDNRNRGRNKARYCAEGGGPVLGRNKNELEGWGRGVYIR

SEQ ID NO: 9 - DNA, 8074 bp, Homo sapiens
  source 1..8074: mol_type = unassigned DNA
agtcaccagtctgtggttgaggaagggttttgctttttgtgctctgtattgggaaggcagataaaatgccaggtctggactgcctctggggataacctcaccctgtgggatgtgaatgaatagcatcttgcctggtaaatccacaggaattgataaggcaggcgcagttctcccaaacaggccttttctctttaagctgtagctgtggtttctgcagcaattttgtttttgccttgaaagaggtgctctggattatcacacctccatgtatgacaatttgtacctgcatggaattgaagactcggaggctggttcagcggattcctacacaagcaggccgtctgactccgatgtctctttggaagaggaccgggaagcaattcgacaggagagagaacagcaagcagctatccagcttgagagagcaaagtccaaacctgtagcatttgccgtgaagacaaatgtgagctactgcggcgccctggacgaggatgtgcctgttccaagcacagctatctcctttgatgctaaagactttctacatattaaagagaaatataacaatgattggtggataggaaggctggtgaaagagggctgtgaaattggcttcattccaagtccactcagattggagaacatacggatccagcaagaacaaaaaagaggacgttttcacggagggaaatcaagtggaaattcttcttcaagtcttggagaaatggtatctgggacattccgagcaactcccacatcaacagcaaaacagaagcaaaaagtgacggagcacattcctccttacgatgttgtaccgtcaatgcgtccggtggtgttagtggggccgtcactgaaaggttacgaggtaacagacatgatgcagaaagccctctttgatttcctgaagcacaggtttgatgggaggatttcaataacgagagtgacagctgacatttctcttgctaagaggtctgtcctaaataatcccagcaagagagcaataattgaacgttcgaacacccggtccagcttagcggaagtacaaagtgaaattgaaagaatctttgagttggcaagatctttgcaactggttgttcttgatgcagacaccatcaatcacccagcacaacttataaagacttccttagcaccaattattgttcatgtaaaagtctcatctccaaaggttttacagcggttgattaaatctagaggaaagtcacaaagtaaacacttgaatgttcaactggtggcagctgataaacttgcacaatgccccccagaaatgtttgatgttatattggatgaaaatcagcttgaggatgcatgtgaacatctaggggagtacctggaggcgtactggcgtgccacccacacaaccagtagcacacccatgaccccgctgctgggaaggaatttgggctccacggcactctcaccatatcccacagcaatttctgggttacagagtcagcgaatgaggcacagcaaccactccacagagaactctccaattgaaagacgaagtctaatgacctctgatgaaaattatcacaatgaaagggctcggaagagtaggaaccgcttgtcttccagttctcagcatagccgagatcattaccctcttgtggaagaagattaccctgactcataccaggacacttacaaaccccataggaaccgaggatcacctgggggatatagccatgactcccgacataggctttgagtctaatgaaacaaaaaatattcatctgttgacaatttgccatagcagtgctaggataaaccaatcatcttaacttggctaacatagcacagtatttactgtgctaatgggctgctgtcattttatgctaagtaaggggcaaaaaaaaaaattacattatgcccttgagtctagatggatattagatgcccgatcatatagatatttttaagtgcaacatttacatgataacagtacattttgttttcttcatagatttagacacatcaatttgtaatttagggtacttacaaggcacatataaaataatttcccatgctggaaattccaaatgaccagttccagttggaaccaatttataaaccaattcctgttggaatttgggtgaattgactagaaagtctacttgagcatgttgtaatcaactgaaaaatctggggggaaaaaaaaaaaactaataagaaaatcgaaataccaatagaagccaaatccagctttggtatttatttgctggaagctaaatttacacttaaagttggagtgtataaacattttaatatatgttaatgttgccagaatgacttttcagacttaccaaatgtattgatagtgccaaaaaaaaaatctcagaaatcttaatgcagaaattatatagtctcctcagattttaatatttttatttctttcctggcacagctgttgtattcaaagtgttttgctttttgtttggtcagtactgttagctcatttgaagttggcaaaaacaaatacaaacaagcaaaaaacctcaaatttttttcagcagtgagagagaatagtaaagagaggcccaagcatagctggttatgtgaaatataaaatggggtgttgttctccctttaatcaatctgagcacaactacatacatccaggaatctgaagtcagatagtaaggatgtggtaccaatcctcccgagtgcaatgctaaacacttccaggtgggaattcatctatttcacttaacacaaacgcagggtctgtatttcctgccaacaagattgagtatctttccattgcttctctcagtctctgtctttgaatatctgaatgttctggcccttcaacaaattctttaaatcatgctaaggaatttcgttttatgtggacacactcaactttgatattcaggagatgtttttacaaaatgactaattgaaaaaagttgaaacaaatgattttaaaagcttgcagtaaaaaataaactcatagattcagcaaatatttgttgactaaaattatagcatttttaatgcagaaaattataaagaatttggagattgagctgccttatttctaatgaacactcctcttcatcttattaagtcttcacagtttttccagtggctataattatggaattggtgacctatgttttatattatcaaggaagccttagattcactctgatctaagaaattattgaaattgaccctttttacacaaatgtctttaaaaaaaaaaacttatttctaaatgtccatagtgcatgcattttctaaaaccccaataagtccagccataaaagttcttttctgctagtacatgtcatgaagcctatttttgaaaataatgataattaattttatagtatcttcttttctccccaagcctttcagtattcaggcactcaaagcatgcaccagttcagtaatcttcttgaccatggaaataagttccacattactgatgagttatatatatatcttgaaccccattacaacctcaccacatccccctgcatttatgttcagtgtttgtaccccattatgaacctgtttagctgataatgaggatacctataattctgtaagtcatttcaaatatttgatacaattaaaaaatatgggcttagtaaattttaaaagttaacatgtgatactggcttgtgagattcttctgtcatctagtgaagcttataaaactttcagttaagacattttaaaagagttatatccattaggcactttaataaacatataacctgccattactgaatactttgttagaacacagaagagcatcttccagcaaacaaaaatatatttattacatgtacagcacatacttgcatgagaaagaaaacattagccaaaacatttttctatatgctttactggtttcttgtttatgcgcattaaagtatttaattgcaaagagtgcaccaaggtgtacaaatttagactagacagctgttcagctttttctgtagtagcattagcagtttggtcatttaactactttaagatacttacattttatggcaggtagctgtaacagcatttaaatatatttcctagtcatcaagattaccatcttctttcaactttttaaaagtcacttgtttttaaaggtttttcaaagccttgtttttagtcttccatgtcatgccgataaagagagtctcattcaatatctgtgcacaaaaccaatctatcaattaccatcagatgtccatacctgttttgctttgcattcttattcaaaagctgacaatcttggaaagggtataatgtatattttattgatggatatatttaaatgtttactttaatttggaatattctttccccttctttctgtaataagtttatcttcctcaaataaggggatatacatcttattttttttaaagacttaagagtttattgtgtaaatgatacttaaaatctagatatatactaaatgaaaagttttatttttgccaagcacaccaagcacctttgaaaaatacatactccatactttgtttttaattaatttcatttaaagatagcaaatgattataagcaagagaaacaagtcttttactgttttgaggctgttttaacttacagtataagccacatactagaattctgtaatttcagtcacagacatataataatgtggactcatgtgacaatgtggaaatgtagttctggaaaaaaacgaatgcaatttgtcaatcctgatgttaattctcaagggtataatgtcactctaatattataaccaacaacattatcttgtttaatatttaaagcaacacttagaaatgtttataaatgatgattctctcttatctttatacagttctattggagagaaggttgaaattgttcacaatttagatgatggtctttcctaataagagtcttaattttgtgactatataagtcattgcagttagtctttaatgaaatgaattttattaatgtaaaacaaattctgagacaattatttgtttcctttaaataatctgctatttaattattattgtcgttgtttgcaaaatgtatttttggaatgttaacctcttttcatacctgcatacttgaacttgtcttttgtgtgggggccttaaaattcataataggaagtggaggcagtgaaaggaagtaagaaaaggaaattatgctaaaagatggtttgcaaatttaaattctttaaatgaacatatcacaaaatgttgttaagacccaggatgtccgttaagaatgtgtcttggcaaccttaatttgcatgttagtatatattttcttagttacacagttttattttggtatttgcttaaaaaatgtaataccattgtagaaattaccctatgtactgatatatttactacctcttctctagagaagacaatctgtctttaaagggaaaaacactggtcaataagtagtgatcaaaattatcactattctggctaaccttgctgcaaagctaaatacatagattattaaaatatgccttccctttagattttaaactgtttccaaaatgctgtttggcaccctttcatttcttccacgttagtgaagttttctttgaattggttcacagtttatcatgaggcaaggttaatttaacaaataagtcatcatagggaacaaaacaaactgcaagggaattgcctttgtgtgcaacttgttataatatctagttctaagtctaaaggagtaaaagtccaaagactttatgtttaagagcagctggtttgcaaattcacattttgttggtccttgacttccacagagcatgacttagccatttactgggtaaggatatgcataatcttggactcccatgtaattggaataaacttcatatcaatgtttcaaataaatgctatgtaccccttattccttttacatttctgcctttgtcttttatgacaatgttgcatgttgtgtttttttaaattatataaaactcatccttgaatgactgtatgaatgaaaaatttataggaaattatgtaataaaatctgttttattagtttctcatacctttaaatactgtcaatactataactgttagtaagttacttactcaagctgtgtctatggggcagagaacagtcctcttctttaaaataatcatgatcaaaccagtcaagtcaatccaattagctgttagacagacaataaggataaccttctttttgacttaagcagtcaagttagtcaaaatagaaaaattggcttatatgggcacccagaactacagcattcaaaaattacttttaaaagtttcttcttttattaaaatggactttcttaaataaaatacatcctctgatagcagctatctctttttacttgtaatgattaattctaaatgcttcatgtaattgagggacattatgagaagactagaaagtatctcatcagataatcctaagggctgtaacttctacttgccataaacaaagtataattaagagtgagtagcaattggtttggggagtcgtagttatatatgaaactttcttttgtttctcatgatgtatgtaaaatgtttgcagaaggatggtagaaaatgttactaatgaatttagagtggtttgggagaatacactgatacttcctcatttgaaaattataaggagttacaaggggaactaagaaccagaagtggctggcctctagcttttcaatacctgataagtgttcttggttatgttttgaacacttaaatttctcaacaaatggctatgtctatatttatgtaggggccacccttgccctacttgcatgccactttgacttttaactgaaatcagtgtgaattctgtatgcataagggatgtaataaagaggcgggatcattaacgctatatcgttgcatactatttttcatatgccttacaatattttctgggatgttattaaaatgaaagcattttattgccaaaataaatctatgttgttacttataatctgcattagcagtagaaaaataatgtagcaaaaacatattgtacagtacaaaattataaaatatatattttgccaataatataaaaatagattgaggagaaagaagcacatttgttatagcacaaacttataacaaaatccaccgttaaatttggcagtcatattttttaaaagtatgtcgatttagttctaagttttgtcagatttacttcatctaccatctttttaacttgctgcttgatacttttccagcaatttatttcatagccttgcaatctgtggatgtaatttcttttgttttattcccttccataaaatttattaaattttattttgtgataacactcataccatattttctcctcttatttcttctctttcaataacaacagcagaaagtcacagcaagtgagctcatagggctcctctgaattttggacatgtgtgagatttttgtatttttcacagcctcgagtcagaatgatcaataaagatgtcccaaccacaaaaaattatattagttagggattctttaatttgttttgtactgaatactactttgaaacataagttaaagccattagctgtgtttttgtagggatggtgggaaggaataaaaaataaatgtattaacataaaacatactaaaacagaaatatggcaaaatgatgtaaaaagatttttcctactattctgagggctgttgaaatcccaggctattgctatatctccattgacaaagtgggataaaaaatgcctttgtcaaataattttttttctttctttaaattttagttatttattttaataatttgtccccttttatttttcttatctaggtatgtgtagcattctacaaggcatgctgggtaaaatatactatgcaaatgtgtacagtgctgcatgttttgttctccctctgctttagtggctttaaagaaaaaaacgtggtgttatttttgtgaataaaaacattaacaaagaaaatgcagagtgcatgcatagagtatcctcatttctatggatttgttttatggaatttaaatgtgtacaaaacaactgcattactttttttcttttatataataaatgaaaaaaacactgaaa

SEQ ID NO: 10 - DNA, 2931 bp, Homo sapiens
  source 1..2931: mol_type = unassigned DNA
ccccttcctccccgccgccacggccccgtaggtgctcggggacccaccttccacctagcacgggttcgttcccctctcccggcctggcccgggctccccggtggccgccgccccctcgccgcccccgccttctcccggggagggggtcaggtgggcgggcactattgttgtaggagccggcgccagattcctcagccgcgctcggggtgggaccggctgggtttgggggggtggggtggggggagcggtgatctgagctccgagcagctggtcttcgcggctcgctccctccttcgcgctctctcgctccctgccgccgcccgcagggctgcggggctcggtggcatctcccgggcgcggcccgcagtccttgcccctgcctccgggccgctcccgcccccggcgccgctcgctcccccgacccggactcccccatgtatgacgactcctacgtgcccgggtttgaggactcggaggcgggttcagccgactcctacaccagccgcccatctctggactcagacgtctccctggaggaggaccgggagagtgcccggcgtgaagtagagagccaggctcagcagcagctcgaaagggccaagcacaaacctgtggcatttgcggtgaggaccaatgtcagctactgtggcgtactggatgaggagtgcccagtccagggctctggagtcaactttgaggccaaagattttctgcacattaaagagaagtacagcaatgactggtggatcgggcggctagtgaaagagggcggggacatcgccttcatccccagcccccagcgcctggagagcatccggctcaaacaggagcagaaggccaggagatctgggaacccttccagcctgagtgacattggcaaccgacgctcccctccgccatctctagccaagcagaagcaaaagcaggcggaacatgttcccccatatgacgtggtgccctccatgcggcctgtggtgctggtgggaccctctctgaaaggttatgaggtcacagacatgatgcagaaggctctcttcgacttcctcaaacacagatttgatggcaggatctccatcacccgagtcacagccgacctctccctggcaaagcgatctgtgctcaacaatccgggcaagaggaccatcattgagcgctcctctgcccgctccagcattgcggaagtgcagagtgagatcgagcgcatatttgagctggccaaatccctgcagctagtagtgttggacgctgacaccatcaaccacccagcacagctggccaagacctcgctggcccccatcatcgtctttgtcaaagtgtcctcaccaaaggtactccagcgtctcattcgctcccgggggaagtcacagatgaagcacctgaccgtacagatgatggcatatgataagctggttcagtgcccaccggagtcatttgatgtgattctggatgagaaccagctggaggatgcctgtgagcacctggctgagtacctggaggtttactggcgggccacgcaccacccagcccctggccccggacttctgggtcctcccagtgccatccccggacttcagaaccagcagctgctgggggagcgtggcgaggagcactccccccttgagcgggacagcttgatgccctctgatgaggccagcgagagctcccgccaagcctggacaggatcttcacagcgtagctcccgccacctggaggaggactatgcagatgcctaccaggacctgtaccagcctcaccgccaacacacctcggggctgcctagtgctaacgggcatgacccccaagaccggcttctagcccaggactcagagcacaaccacagtgaccggaactggcagcgcaaccggccttggcccaaggatagctactgacagcctcctgctgccctaccctggcaggcacaggcgcagctggctggggggcccactccaggcagggtggcgttagactggcatcaggctggcactaggctcagcccccaaaaccccctgcccagccccagcttcagggctgcctgtggtcccaaggttctgggagaaacaggggaccccctcacctcctgggcagtgacccctactaggctcccattccaggtactagctgtgtgttctgcacccctggcaccttcctctcctcccacacaggaagctgccccactgggcagtgccctcaggccaggatccccttagcagggtccttcccaccagactcagggaagggatgccccattaaagtgacaaaagggtggggtgtgggcaccatggcatgaggaagaaacaaggtccctgagcaggcacaagtcctgacagtcaagggactgctttggcatccagggcctccagtcacctcactgccatacattagaaatgagacaatcaaagcccccccagggtggcacacccatctgtttgctggggtgtggcagccacatccaagactggagcagcaggctggccacgctcgggccagagagagctcacagctgaagctcttggagggaagggctctcctcaccctgccaggaagcttcttaacatgtgacaggaccagggaccaggagcatggtgaagccaagtggcagatgggagccaacctggatgggggtttggggaaggagggcatgtgtagcagagaacttaggggggcctccttgcctttctcattcttttgccctgcatcctgtcatttctgttcttgtccctcatacatctttggagaaccgggctccagactttgttccctgactcatagctgccgcttgttaggttagggttagatggggagagacagggcacagaggacctgtctccccggctactcttgccttatggctctagtgtgtgacctacagagcatgctccacaagcccctgcctcacctcactgtcatcactaataaacatcatgcacagtc

SEQ ID NO: 11 - DNA, 6199 bp, Homo sapiens
  source 1..6199: mol_type = unassigned DNA
gcattgtgaagaaggcaatcataggcgagcagccgcgggagcaggaacagcagcgtgctaagaagcagtcacataaacagcagcaggagtaggcctcctgcttttcaaaagcagagtactgcagggtcgcgaaatgcaagacactcagatgtttgaaaatctcccgagttgagaatggctactgtaaaagcgtcaccaagaaactctgacgatctggacagtcctaactctgtgttagcaatacttacttccggaaaattaatgctacttcttgtagatttttgcaaataggaaacccccttgaagaagatctcaaattacgccccccacccccaaaaaaagacaaacaggggagaacaaagttttggcatgcctgcaggaacggtggcttttttagaaactacctaggaggcagaagctaagtgatttgctcatgcctcttacctgggagtagaaggtgggaagaaatggaccgaggctgtgacgagaagacaaggcacagtgcagcttggtgaagccacacgctgactgcgttctgccccctcttcatgcagtgctgcgggctggtgcatcgccggcgagtacgggtgtcctatggttcggcagactcctacactagccgtccatccgattccgatgtatctctggaggaggaccgggaggcagtgcgcagagaagcggagcggcaggcccaggcacagttggaaaaagcaaagacaaagcccgttgcatttgcggttcggacaaatgtcagctacagtgcggcccatgaagatgatgttccagtgcctggcatggccatctcattcgaagcaaaagattttctgcatgttaaggaaaaatttaacaatgactggtggatagggcgattggtaaaagaaggctgtgaaatcggattcattccaagcccagtcaaactagaaaacatgaggctgcagcatgaacagagagccaagcaagggaaattctactccagtaaatcaggaggaaattcatcatccagtttgggtgacatagtacctagttccagaaaatcaacacctccatcatctgctatagacatagatgctactggcttagatgcagaagaaaatgatattccagcaaaccaccgctcccctaaacccagtgcaaacagtgtaacgtcaccccactccaaagagaaaagaatgcccttctttaagaagacagagcacactcctccgtatgatgtggtaccttccatgcgaccagtggtcctagtgggcccttctctgaagggctacgaggtcacagatatgatgcaaaaagcgctgtttgattttttaaaacacagatttgaagggcggatatccatcacaagggtcaccgctgacatctcgcttgccaaacgctcggtattaaacaatcccagtaagcacgcaataatagaaagatccaacacaaggtcaagcttagcggaagttcagagtgaaatcgaaaggatttttgaacttgcaagaacattgcagttggtggtccttgacgcggatacaattaatcatccagctcaactcagtaaaacctccttggcccctattatagtatatgtaaagatttcttctcctaaggttttacaaaggttaataaaatctcgagggaaatctcaagctaaacacctcaacgtccagatggtagcagctgataaactggctcagtgtcctccagagctgttcgatgtgatcttggatgagaaccagcttgaggatgcctgtgagcaccttgccgactatctggaggcctactggaaggccacccatcctcccagcagtagcctccccaaccctctccttagccgtacattagccacttcaagtctgcctcttagccccaccctagcctctaattcacagggttctcaaggtgatcagaggactgatcgctccgctcctatccgttctgcttcccaagctgaagaagaacctagtgtggaaccagtcaagaaatcccagcaccgctcttcctcctcagccccacaccacaaccatcgcagtgggacaagtcgcggcctctccaggcaagagacatttgactcggaaacccaggagagtcgagactctgcctacgtagagccaaaggaagattattcccatgaccacgtggaccactatgcctcacaccgtgaccacaaccacagagacgagacccacgggagcagtgaccacagacacagggagtcccggcaccgttcccgggacgtggatcgagagcaggaccacaacgagtgcaacaagcagcgcagccgtcataaatccaaggatcgctactgtgaaaaggatggagaagtgatatcaaaaaaacggaatgaggctggggagtggaacagggatgtttacatccgccaatgagttttgcccgtttgtgtttttttttttttttttttgaagtcttgtataactaacagcatccccaaaacaaagtctttggggtctacactgcaatcatatgtgatctgtcttgtaatattttgtattattgctgttgcttgaatagcaatagcatggatagagtattgagatactttttcttttgtaagtgctacataaattggcctggtatggctgcagtcctccggttgcatactggactcttcaaaaactgttttgggtagctgccacttgaacaaaatctgttgccacccaggtgatgttagtgttttaagaaatgtagttgatgtatccaacaagccagaatcagcacagataaaaagtggaatttcttgtttctccagatttttaatacgttaatacgcaggcatctgatttgcatattcattcatggaccactgtttcttgcttgtacctctggctgactaaatttggggacagattcagtcttgccttacacaaaggggatcataaagttagaatctattttctatgtactagtactgtgtactgtatagacagtttgtaaatgttatttctgcaaacaaacaccttcttattatatatataatatatatatatatcagtttgatcacactattttagagtcttaatgccaagtcagcagatttgctttatgaattacagggactagaaatgcccacattcaggaaatttgtaataacattgtctagacacctatcctcattctagtagaaagtgtgtacatactgtaaatatgtgtgattgcttgacttgaaaaggtttgaattctgaatgttataccatccttgtaagtaagtttgtaatttccaccataaattatggtaaatataaaactccagaggttgttctactccatacagttcacactgattgtgacacattcttagtagctagtgtctgttctagtcactgcactggagtctacgagccggaactcgctatatgcacgtgtgtgtgtccgtatgtaagaaagtgtgcaccgagtgactgaatggttgagatgaattggaatgctgaagactaacgaagaaactagagactgatatcgagcattctgcccacctcgctctgtatttaattaattgtgctatatgttgctttaacaacccattgagcagtcagggaatgtgagtaagcttgctgccaaaggtaactaggaaagcattcatctgctgcctccttgtttttgctcctagagagtgaaaatacaggcaattttactgtgagtgtttcactggaaatgtacaatctttgtgtgttagagtatttgttttagtaagaaatgtttacacagcttgtggaattatttcgtgggaaaataaatttttataacttctcccacttcaatttctaaccttgcctattgttcctgttgtttgtttacctcccagttacctaccattcctccccaccaccgactccagcaggttcactgtctgtcagaacccagaagtgcttcttataaccaaagtttctgttcttcagaagaaatcagggcaaaatggggtgacttgaagtgaataaaatgttaagaatattatcctacataagacatgtacacagaaggggaaccttgaagacattatctccatgcctcaattacactgctgtaagaagcttacaatgtcatcatgtttaaggctaagaccttttccgtgtctcaagtgtatattttgtccagttataactggatggtaagacagtattaagagtgcaagtacctggcacttgaagtttgtcccaggaaaatgcctgtgtataattacctaacttcagatctgcacattaacttatttaacaaaataaatcagccgggcgcagtggctcatgcctgtaatcccagcaatttgggaggctgaggagggtggatcacctgaggtcaggagttcaagaccagcctggccaacgtggcgaaaccccgtctctactaaaaagataaaaaattagccaggtgtggtggtgcacacctgtaatcccagctacttgggaggctgaggcaggagaattgcttgaacccaggaggtggagcctgcagtaagccaagatcacaccactgtactccagcctgggtgacagagtgagactccatctccaaaaaagaaaaaaaaacaaaaaacaaaaaaaactatccagcaaaattataaaatctactttgttttgcttccttgattattccaagttcttaccaaatattcttagccttttataagtaaaactgttttacatttaactccttatttcccttacccccaagaaaacgcaatattaaaaatgattaagctggggtggtagagtatacctgtagacccagctacttgggaggctgagataggaggatcccttgagcccaggaggtggaggctgcagtgaaccgtgactgtaccaatgcactccagcctggtgacaacctggtgtttaaaaaaagaaaataaaaattagttacatatataatgttttatcagcttatatatgaaaaaatttccctcagttcgttaattagccctacactgtttacataaatttatttactgagtataaaatcagtagtcataataaacttgacaattcttacattggtaggaaaccatattctaattaaaattgaatttataaaatatttctgataaaacttttgtcatggtaagagattcacaattattagttcaatcctttattatttcatcccaattaatgattttagaatatttaaattcttgaaacatagctagtatttatcttacttgctgctatcattaatccctttcaaaaagtagtggtgagagtttccatcttttttattttgtatctagattcttagcttgcactttactgtagaacatattagtgcaaatcagaatattcctcaaagaactagcttgaaggatttgacataagagccgctatatgtgaaaaactgtatagtggacatatgtgtaaactgtgtagtaaatctgtaaatgaggaaataatctcaagggcaatgggatatttactgacctgcggaatgtaaagttacagtcttttcacacaacaaaggcttaaccttaacctactcagttgtagagccatttttgtagtcaacctagaaaatgctggaaatgtatttaatagttttttttttttttttttttggtcatatccatttcagtctttcctatgctctttctctactgctcatttaagttactgttacaaaaaggtgctgctaaatgtaggatgtcttagtcatgttgtactttgggacaatgccacatttttaacatggtctgctatgcattcctgttaaacattcactgtcagctacactgaactgtctaacaaacctggtttaaagtaattcatataaaacaaataaagagctggcttctgcactgtttattagtagtagtattaattgccatgttagaaatgattcatgtagttgagactatattaaatattcaacacttcccagctgcagggcatctttcttggggcaatgctaaatactttatgttcctttagtaatcttcctattatgtctgattaaaaaaaaagatacctccacgtagataaaatattttattcaacgtgagctctagcagaataatctgggtcagttatgagttctggatcaagagtttaatttctgctgcgctgtacttttaactatttgtactttgcttcattatttaaaacatgacacagggttttaaagtaaatgtactcactgttgaacttaagtacttcttctgtttcatagaaatgttaatattaaatgttaaaatatatattaattttccctcaca

SEQ ID NO: 12 - DNA, 3711 bp, Homo sapiens
  source 1..3711: mol_type = unassigned DNA
agtggagagagcgggaggagcgagggaaggcaggaaggaggcagccgaaggccgagctgggtggctggaccgggtgctggctgcgccgcgctgctttcggctcccacggcctctcccatgcgctgagggagcccggctgggccgggccggcggcgggaggggaggctcctctccatggtccagaagaccagcatgtcccggggcccttacccaccctcccaggagatccccatggaggtcttcgaccccagcccgcagggcaaatacagcaagaggaaagggcgattcaaacggtcagatgggagcacgtcctcggataccacatccaacagctttgtccgccagggctcagcggagtcctacaccagccgtccatcagactctgatgtatctctggaggaggaccgggaagccttaaggaaggaagcagagcgccaggcattagcgcagctcgagaaggccaagaccaagccagtggcatttgctgtgcggacaaatgttggctacaatccgtctccaggggatgaggtgcctgtgcagggagtggccatcaccttcgagcccaaagacttcctgcacatcaaggagaaatacaataatgactggtggatcgggcggctggtgaaggagggctgtgaggttggcttcattcccagccccgtcaaactggacagccttcgcctgctgcaggaacagaagctgcgccagaaccgcctcggctccagcaaatcaggcgataactccagttccagtctgggagatgtggtgactggcacccgccgccccacaccccctgccagtgccaaacagaagcagaagtcgacagagcatgtgcccccctatgacgtggtgccttccatgaggcccatcatcctggtgggaccgtcgctcaagggctacgaggttacagacatgatgcagaaagctttatttgacttcttgaagcatcggtttgatggcaggatctccatcactcgtgtgacggcagatatttccctggctaagcgctcagttctcaacaaccccagcaaacacatcatcattgagcgctccaacacacgctccagcctggctgaggtgcagagtgaaatcgagcgaatcttcgagctggcccggacccttcagttggtcgctctggatgctgacaccatcaatcacccagcccagctgtccaagacctcgctggcccccatcattgtttacatcaagatcacctctcccaaggtacttcaaaggctcatcaagtcccgaggaaagtctcagtccaaacacctcaatgtccaaatagcggcctcggaaaagctggcacagtgcccccctgaaatgtttgacatcatcctggatgagaaccaattggaggatgcctgcgagcatctggcggagtacttggaagcctattggaaggccacacacccgcccagcagcacgccacccaatccgctgctgaaccgcaccatggctaccgcagccctggctgccagccctgcccctgtctccaacctccagggaccctaccttgcttccggggaccagccactggaacgggccaccggggagcacgccagcatgcacgagtacccaggggagctgggccagcccccaggcctttaccccagcagccacccaccaggccgggcaggcacgctacgggcactgtcccgccaagacacttttgatgccgacacccccggcagccgaaactctgcctacacggagctgggagactcatgtgtggacatggagactgacccctcagaggggccagggcttggagaccctgcagggggcggcacgcccccagcccgacagggatcctgggaggacgaggaagaagactatgaggaagagctgaccgacaaccggaaccggggccggaataaggcccgctactgcgctgagggtgggggtccagttttggggcgcaacaagaatgagctggagggctggggacgaggcgtctacattcgctgagaggcaggggccacacggcgggaggaagggctctgagcccaggggaggggagggagcgaggggctcacacctgacatgtattcgcctccagggggcgctgtctccctcctttcagatgcctttgctcaaagcttggggtttctttggtgttaccatcccagctcccgggaggcccttaagccccagctgtcggtttttacctgcctgttgtggatggatgggggatacccacctttctgaagtgtcccctttctcccatcttaaggggctctcctccctcaccctcctagagaaaaggtgcacttccttaactctttctactcggggccctaagtgacggtcctaagtgggatggctctccttctcccaagctgcagtactggggaagggctgggcgcttttcctggaaagggaggccacagattctttcccatgggggctctcttccccagaccccagatccaaggtccctcaccctgcctgccccttcctcccagcttcctggcagcatcgtctggtcggtgaaagccatagcatggacaccccatggggagcttgtcttggggagggttctgggtggaagctggcaggcatacagcaccctctaccctccgtggccatggcaacgtccagggcccagaaccctgaggagtgagcggccgagacgctgctccccaccccccacctccatgcctcagcctttgcctaccccaggaatgagcttggcctccaacatcccttgcctgctgccattagtagagggggcccctctgcatctgagccccccatccctgtgccacctgggtgtggagcccatggaacactctggtccgcctcattttaaaccaaaaaactgctccttcaccctcaccctgaggccccaggggagaggacccgtgggatggtgcccaggggttgccttgggacctcggatctcctctggggggcttggctgctgctgttgctgctctgtatttgcctctcgtgattctgtttgttacccatgttcacttcccccaggagaggccttggtaccccctctcccctggggcatccctttgccttggcatccctgtagcccagcaaccctgcccctccccagcatcccagctgggccagagagagccgagtgtgccaacaaggactggggcctgcccggctgcccgcctcagggatgggcacctcatgcctgtctcgccacctcctgtgccaatgtcccaccctccacctgggggtggggtgcagcttccacttactgattagaagacaccactgccctcccttcccccctccctgtctggtgtcctgtgcccccatctgtctgtctatatttgtctgtactcccctaggagaagtattttgccatatataaaaccactgtcctgtcctttgtggctgcctcccaagcctgcttctttgtcctcgccacatagtcgtcagcgtaggcacctgggagctgctgatatgcacggggagttgaaagggtgggtgcctgaagatgttgtgccctgagtcattgactcaaaagaaaagatgatcctttgattttggccctctgatgtattgtgcccaagccaggagctgcttgggcagtcccagctccacactggccctgagccccttcacttacctgtctctccacaagtagagccaaaggcaatgggaagctcaatgttgctcagtgggtgagatccagacccactggtgcaatgtcttaaatacacatgactgtttttctgc

SEQ ID NO: 13 - protein, 19 aa, synthetic construct
TFFYGGSRGKRNNFKTEEY

SEQ ID NO: 14 - protein, 19 aa, synthetic construct
YEETKFNNRKGRSGGYFFT

SEQ ID NO: 15 - protein, 10 aa, synthetic construct
LRKLRKRLLR

SEQ ID NO: 16 - protein, 10 aa, synthetic construct
CGHKAKGPRK

SEQ ID NO: 17 - protein, 5 aa, synthetic construct
  SITE 1..5: note = cyclic peptide
RGDFK

SEQ ID NO: 18 - protein, 16 aa, synthetic construct
FKESWREARGTRIERG

SEQ ID NO: 19 - protein, 16 aa, synthetic construct
GREIRTGRAERWSEKF

SEQ ID NO: 20 - protein, 13 aa, synthetic construct
  SITE 13: note = Attached to an O-beta-glucoside
YGGFLGGYTGFLS

SEQ ID NO: 21 - protein, 7 aa, synthetic construct
  SITE 7: note = Attached to a monosaccharide
GFTGFLS

SEQ ID NO: 22 - protein, 23 aa, synthetic construct
HGLASTLTRWAHYNALIRAFGGG

SEQ ID NO: 23 - skipped

SEQ ID NO: 24 - protein, 5 aa, synthetic construct
LPFFD

SEQ ID NO: 25 - protein, 8 aa, synthetic construct
  SITE 1..8: note = Cyclized peptide
  SITE 1: note = Attached to diaminopropionic acid
KAPETALD

SEQ ID NO: 26 - protein, 16 aa, synthetic construct
RQIKIWFQNRRMKWKK

SEQ ID NO: 27 - protein, 39 aa, synthetic construct
KSVRTWNEIIPSKGCLRVGGRCHPHVNGGGRRRRRRRRR

SEQ ID NO: 28 - protein, 34 aa, synthetic construct
YTWMPENPRPGTPCDIFTNSRGKRASNGGGGGGC

SEQ ID NO: 29 - protein, 36 aa, synthetic construct
MCMPCFTTDHQMARKCDDCCGGKGRGKCYGPQCLCR

SEQ ID NO: 30 - protein, 7 aa, synthetic construct
HAIYPRH

SEQ ID NO: 31 - protein, 16 aa, synthetic construct
GRKKRRQRRRPPQGWC

SEQ ID NO: 32 - protein, 11 aa, synthetic construct
YGRKKRRQRRR

SEQ ID NO: 33 - protein, 11 aa, synthetic construct
RRRQRRKKRGY

SEQ ID NO: 34 - protein, 12 aa, synthetic construct
TGNYKALHPHNG

SEQ ID NO: 35 - protein, 12 aa, synthetic construct
THRPPMWSPVWP

SEQ ID NO: 36 - protein, 12 aa, synthetic construct
PWVPSWMPPRHT

SEQ ID NO: 37 - protein, 8 aa, synthetic construct
  SITE 2..8: note = Cyclized peptide
CMPRLRGC

SEQ ID NO: 38 - DNA, 4842 bp, Rattus norvegicus
  source 1..4842: mol_type = unassigned DNA
atgtcctcggccgtggggccccgaggtcctcgcccacccacggtgcctccccctatgcaagaactgcccgacctgagccacctgaccgaggaggagaggaacattatcatggcagtgatggaccggcagaaggaagaggaggaaaaagaagaggccatgctcaagtgtgttgtcagggacatggcgaagcctgctgcctgcaaaacaccaagaaatgctgaaagccagccccatcaaccaccactgaacattttcagatgtgtctgtgttcccagaaagccaagcagcgaagagggaggcccagaaagagactggagattgcatcaacagtttgaaagctacaaggagcaagtgagaaaaatcggagaggaagcgaggcgttaccagggcgagcacaaggatgatgccccgacgtgtggaatctgtcataagacaaagtttgctgatggatgtggccatctctgctcctattgtcgcaccaagttctgtgcacgctgcggaggccgtgtgtctctgcgatcgaacaatgaggacaaagtggttatgtgggtatgcaatttatgtcgaaagcaacaagaaatcttaacgaaatctggagcgtggttctttggaagtggccctcagcagcctagtcaagatgggactctgagtgacacggccacaggtgctggatctgaggtgccaagagaaaagaaagcaaggctccaagagcgatcaaggtctcagacgcccttgagtacagcagctgtctcttcccaagacactgctacccccggtgcaccgttggacaggaacaaaggggctgagccctcacagcaagccttgggtcctgaacagaagcaggcatcaagatcaagaagcgagccaccgagggaaaggaagaaggctccagggctttcagagcagaatggcaagggaggccagaagagcgagcgcaaacgtgtccccaagtctgtggtgcaacccggggaagggatcgcggatgagagggagaggaaagagaggcgggaaacccgcaggttggagaaagggcgctcccaggactactcagaccggcctgagaaacgcgacaatggcagggtggcggaagaccagaagcagaggaaggaggaggagtaccagactaggtaccgcagcgaccctaacctggctcgctacccggtgaaggcgccgccagaggagcagcagatgcgcatgcacgcccgggtgtcccgagcgaggcacgagcggcgccacagcgacgtggcgctcccgcacaccgaggcagctgccgccgcgccggctgaggccacggcgggcaagcgcgcgccggccaccgccagggtctctcccccggagtccccgcgcgcacgcgcgcccgccgcccagcctcccaccgagcacgggccaccgccgccgcggccagccccgggtcccgcagagccacccgagccgcgcgtccccgagccgctccgtaagcagggccgcctggacccgggctcggccgtgcttctgcgcaaggccaagcgcgagaaggcggagagcatgctgcggaacgactcgctgagctccgatcagtccgagtccgtgcggccatccccgcccaagcctcaccggcccaagcggggaggcaagagacgtcagatgtcggtgagcagctcggaggaggagggcgtgtccacaccggagtacacgagctgcgaggacgtggagctggagagcgagagcgtgagcgagaaaggtgacttggattactactggttggatcccgccacgtggcacagcagggaaacgtcgcctatcagttcgcatcctgtaacgtggcagccgtctaaagagggagatcgactaatcggccgtgttattcttaacaaaagaacaaccatgcccaaagaatcaggtgcattattgggtctgaaggtggttggaggaaaaatgacggacttagggcgccttggtgctttcatcaccaaagtaaagaagggcagcctggcagacgtcgtcggacacctaagagcaggggacgaagtcctagagtggaatggtaaacccctgccgggagcaacaaacgaagaagtttacaacattatcttagaatcaaaatcagaacctcaagttgagattattgtttcaaggcctattggtgacatccccaggatccctgagagttcccatcctcccctggagtccagttcaagttcctttgaatctcagaaaatggaaaggccttctatttctgttatttctccaaccagccctggagctctgaaagatgccccacaagtcttaccagggcaactctcagtgaagctatggtatgataaagtggggcaccagctgattgtaaatgttctacaagcaacagatctaccccctagagtagatggccgtcccaggaatccctatgtaaaaatgtattttcttccagatagaagcgacaaaagtaaaaggagaaccaaaacagtaaagaaacttctagagccaaaatggaaccagacatttgtctactcacacgtacatcgtagagattttcgagagcgaatgttagagattaccgtgtgggaccagccgagagtacaggacgaagagagtgaatttcttggagagatcctcatagagttggaaacagcgcttttagatgatgagccccattggtataaactccagacacatgacgaatcttcactacctctgcctcagccatcaccgttcatgcccaggcggcatattcatggagagagctccagcaaaaagctacaaagatctcagcgaatcagtgatagtgacatctcagattatgaggttgatgatggtattggagtagtgcctccagtgggttatagagctagtgctagagagagtaaagcaaccacgttaacagtgccagagcaacaaagaactacacatcaccgctcacgttccgtgtctcctcatcgcggcgatgatcagggaaggcctcgttcacgtttaccaaatgtgccattacagaggagcttagatgaaattcatccaacacgaaggtcacgttctccaacccgacaccatgatgcctcccgaagcccggccgatcacagatccagacatgtggaaagtcaatattcgtcagagccagacagtgagcttctcatgctgcccagagcaaaacgaggacgaagtgcagaaagcctacacatgaccagtgaactgcagccctctcttgacagggctaggagtgctagtaccaactgcttgagaccagatactagtttgcattcaccagaacgagaaaggcactccagaaagtctgaaagatgtagcatccaaaaacagtctaggaaaggcacagcctctgatgcagacagggttctcccaccatgcctttctagaaggggatacgcaaccccaagagcaaccgatcaaccggtcgttaggggaaagcatcccactcgttcacggtcgagcgagcactctagtgtcagaaccctgtgttctatgcaccaccttgcccccggagggtcggcgccaccttctccacttctgacaagaacgcaccgacaaggaagcccaacccagtctcctccagcagacacatccttcggcagtcgccgtggaagacagctcccacaggtgccagttcgaagcggcagtatagaacaagcaagcttagtagtggaggagcgaacgagacagatgaaaatgaaagttcaccgatttaagcagacaacagggtctgggtctagtcaagaacttgaccacgagcaatactccaagtacaacatacataaagatcagtacagaagctgtgataacgcgtctgccaagtcttcagatagtgatgtcagtgatgtgtccgccatttccagagccagcagtacctcacgcctcagcagcacaagctttatgtcagagcagtctgagcgccccaggggtaggatcagttcatttacccccaaaatgcaaggcagacggatggggacttcaggaagagccatcatcaagagcaccagtgtaagtggagagatatatacactggaacgtaatgacggtagccagtcggacacggccgtgggtaccgtcggagccggtggaaagaaacgaagatccagcctgagcgccaaagtggtagccattgtgtctcgaagaagcaggagcacgtcacagctcagccagacagagtcgggccacaagaagttgaaaagcaccatccagaggagtacggaaacaggaatggcagctgaaatgcggaagatggtgagacagccgagccgggagtccacggatggcagcatcaacagttatagctcggaaggaaacttgatatttcctggagttcgagtaggacccgacagtcagttcagtgatttccttgatgggttgggaccagcgcagctcgttggccgtcagacgctcgccaccccggccatgggcgatatccaaatcgggatggaggataagaagggtcagttggaggttgaggttatcagagcccggagccttacacaaaaacctggttccaaatctacacccgctccctatgtgaaagtatatcttttggaaaatggagcctgtattgccaaaaagaagacaagaattgcacggaaaactctcgatcctttgtatcagcagtccctggtttttgatgaaagtccacagggtaaagttcttcaggtgattgtctggggtgactatggaagaatggaccacaaatgctttatgggtgtggctcaaatcttgttggaagaacttgatctatccagcatggtgattggatggtataaattgttccctccgtcctcactggtggatcccactctcgctcccctgacccgccgggcttcccaatcatctctggaaagttcgtccgggcctccctgcatccggtca

SEQ ID NO: 39 - DNA, 570 bp, Rattus norvegicus
  source 1..570: mol_type = unassigned DNA
atgtcctcggccgtggggccccgaggtcctcgcccacccacggtgcctccccctatgcaagaactgcccgacctgagccacctgaccgaggaggagaggaacattatcatggcagtgatggaccggcagaaggaagaggaggaaaaagaagaggccatgctcaagtgtgttgtcagggacatggcgaagcctgctgcctgcaaaacaccaagaaatgctgaaagccagccccatcaaccaccactgagattgcatcaacagtttgaaagctacaaggagcaagtgagaaaaatcggagaggaagcgaggcgttaccagggcgagcacaaggatgatgccccgacgtgtggaatctgtcataagacaaagtttgctgatggatgtggccatctctgctcctattgtcgcaccaagttctgtgcacgctgcggaggccgtgtgtctctgcgatcgaacaatgaggacaaagtggttatgtgggtatgcaatttatgtcgaaagcaacaagaaatcttaacgaaatctggagcgtggttctttggaagtggccctcagcagcctagtcaagat

SEQ ID NO: 40 - protein, 1614 aa, Rattus norvegicus
MSSAVGPRGPRPPTVPPPMQELPDLSHLTEEERNIIMAVMDRQKEEEEKEEAMLKCVVRDMAKPAACKTPRNAESQPHQPPLNIFRCVCVPRKPSSEEGGPERDWRLHQQFESYKEQVRKIGEEARRYQGEHKDDAPTCGICHKTKFADGCGHLCSYCRTKFCARCGGRVSLRSNNEDKVVMWVCNLCRKQQEILTKSGAWFFGSGPQQPSQDGTLSDTATGAGSEVPREKKARLQERSRSQTPLSTAAVSSQDTATPGAPLDRNKGAEPSQQALGPEQKQASRSRSEPPRERKKAPGLSEQNGKGGQKSERKRVPKSVVQPGEGIADERERKERRETRRLEKGRSQDYSDRPEKRDNGRVAEDQKQRKEEEYQTRYRSDPNLARYPVKAPPEEQQMRMHARVSRARHERRHSDVALPHTEAAAAAPAEATAGKRAPATARVSPPESPRARAPAAQPPTEHGPPPPRPAPGPAEPPEPRVPEPLRKQGRLDPGSAVLLRKAKREKAESMLRNDSLSSDQSESVRPSPPKPHRPKRGGKRRQMSVSSSEEEGVSTPEYTSCEDVELESESVSEKGDLDYYWLDPATWHSRETSPISSHPVTWQPSKEGDRLIGRVILNKRTTMPKESGALLGLKVVGGKMTDLGRLGAFITKVKKGSLADVVGHLRAGDEVLEWNGKPLPGATNEEVYNIILESKSEPQVEIIVSRPIGDIPRIPESSHPPLESSSSSFESQKMERPSISVISPTSPGALKDAPQVLPGQLSVKLWYDKVGHQLIVNVLQATDLPPRVDGRPRNPYVKMYFLPDRSDKSKRRTKTVKKLLEPKWNQTFVYSHVHRRDFRERMLEITVWDQPRVQDEESEFLGEILIELETALLDDEPHWYKLQTHDESSLPLPQPSPFMPRRHIHGESSSKKLQRSQRISDSDISDYEVDDGIGVVPPVGYRASARESKATTLTVPEQQRTTHHRSRSVSPHRGDDQGRPRSRLPNVPLQRSLDEIHPTRRSRSPTRHHDASRSPADHRSRHVESQYSSEPDSELLMLPRAKRGRSAESLHMTSELQPSLDRARSASTNCLRPDTSLHSPERERHSRKSERCSIQKQSRKGTASDADRVLPPCLSRRGYATPRATDQPVVRGKHPTRSRSSEHSSVRTLCSMHHLAPGGSAPPSPLLTRTHRQGSPTQSPPADTSFGSRRGRQLPQVPVRSGSIEQASLVVEERTRQMKMKVHRFKQTTGSGSSQELDHEQYSKYNIHKDQYRSCDNASAKSSDSDVSDVSAISRASSTSRLSSTSFMSEQSERPRGRISSFTPKMQGRRMGTSGRAIIKSTSVSGEIYTLERNDGSQSDTAVGTVGAGGKKRRSSLSAKVVAIVSRRSRSTSQLSQTESGHKKLKSTIQRSTETGMAAEMRKMVRQPSRESTDGSINSYSSEGNLIFPGVRVGPDSQFSDFLDGLGPAQLVGRQTLATPAMGDIQIGMEDKKGQLEVEVIRARSLTQKPGSKSTPAPYVKVYLLENGACIAKKKTRIARKTLDPLYQQSLVFDESPQGKVLQVIVWGDYGRMDHKCFMGVAQILLEELDLSSMVIGWYKLFPPSSLVDPTLAPLTRRASQSSLESSSGPPCIRS

SEQ ID NO: 41 - protein, 190 aa, Rattus norvegicus
MSSAVGPRGPRPPTVPPPMQELPDLSHLTEEERNIIMAVMDRQKEEEEKEEAMLKCVVRDMAKPAACKTPRNAESQPHQPPLRLHQQFESYKEQVRKIGEEARRYQGEHKDDAPTCGICHKTKFADGCGHLCSYCRTKFCARCGGRVSLRSNNEDKVVMWVCNLCRKQQEILTKSGAWFFGSGPQQPSQD

SEQ ID NO: 42 - DNA, 1560 bp, Mus musculus
  source 1..1560: mol_type = unassigned DNA
atgtcgtcctcgtacggcaagaacggggcggcggacgggccgcactcccccagctcacaggtggcccgaggcaccacgacccggaggagcaggttgaaaagatcggatggcagcaccacttctaccagcttcatcctcagacaggggtcagcagattcctatacaagcaggccatcggactccgatgtctccttggaagaggaccgggaagcgatccggcaagagcgagaacagcaagcagctatccagcttgagagagcgaagtccaaacctgtagcttttgcggtgaaaacgaacgtgagctactgtggtgccctggacgaagatgtgcccgttcccagcacagccatctcctttgacgccaaggactttcttcacattaaagagaaatataacaatgattggtggataggaagactggtaaaagagggctgtgagattggcttcatcccaagtccactgcgcttggagaatattcggattcaacaggaacagaaaagaggccgttttcatggcgggaaatcgagtggaaactcttcctccagtctgggagaaatggtatcaggaacattccgagcaactcccacaacaacagcaaaacagaagcagaaagtgacggagcacattcctccgtatgacgtcgtgccgtcaatgcgtcctgtggtgttagtggggccatcactgaaaggttatgaggtaacagacatgatgcagaaagccctctttgatttcctgaagcacaggtttgatgggaggatatcaataacaagagtgacagctgacatttctcttgctaagagatctgtcctcaacaatcctagcaagagagcaataattgaacgttccaacaccagatccagcttagcggaagtacaaagtgaaattgaaagaatttttgagttggcaagatctttgcaattggttgttcttgatgcagacaccatcaaccacccagcacagctgataaagacatccttagcacccatcatcgtccacgtgaaggtctcgtccccaaaggttttacagcggctgattaagtccagaggaaagtcccaaagcaaacacttgaatgttcaactggtggcggccgataaactggcccagtgcccgcctgaaatgtttgatgttatattagatgagaatcaacttgaggatgcctgtgaacatctgggagagtacctggaggcatactggcgtgccacccacacgagcagtagcacccctatgaccccattactggggcggaacgtgggctccacagccctctcaccatatcccacagcaatctctggattacagagtcagcgaatgagacacagcaaccattctacagagaattctccaattgaaagacgaagcctaatgacctcggatgaaaattaccacaatgagagggcccgcaagagtaggaaccgcttgtcttccagctcccagcacagccgagaccactaccctctggtggaagaagattacccggactcgtaccaggacacttataagccccataggaaccgaggatcgcccggggggtgcagccatgactcccgacataggctttga

SEQ ID NO: 43 - DNA, 1560 bp, Mus musculus
  source 1..1560: mol_type = unassigned DNA
atgtcgtcctcctacgccaagaacggggcggcggacgggccgcactcccccagctcgcaggtggcccgaggcaccacgacccggaggagcaggttgaaaagatccgatggcagcaccacttccaccagcttcatcctcagacaggggtcagcagattcctatacaagcaggccatcggactccgatgtctctttggaagaggaccgggaagcgattcggcaagagagagaacagcaagcagctatccagcttgagagagcgaagtccaaacctgtagcttttgcggtgaaaacgaatgtgagctactgtggtgccctggatgaggatgtgcccgttcccagcacagccatctcctttgacgccaaggactttcttcacattaaagagaaatataacaatgattggtggataggaaggctggtgaaggagggctgtgagatcggcttcatcccaagtccactgcgtttggagaacatacggattcaacaggaacagaaaagaggccgttttcatggcgggaaatcgagtggaaactcttcctccagtctgggagaaatggtgtcaggaacatttcgagcaactcccacaacaacagcaaaacagaagcagaaagtgacggagcacattcctccgtatgatgttgtgccatcaatgcgtcctgtggtgttagtagggccatcactgaaaggttatgaggtaacagacatgatgcagaaagccctctttgatttcctgaagcacaggtttgatgggaggatatcaataaccagagtgacagctgacatttctcttgctaagaggtctgtcctcaacaatcctagcaagagagcaataattgaacgttcaaacaccagatccagcctagcggaagtacagagtgaaattgaaagaattttcgagttggcaagatctttgcaattggttgttctcgatgcagacaccatcaatcacccagcacagctgataaagacatcgttagcgcccatcatcgtccacgtgaaggtctcgtccccaaaggttttacagcggctgattaagtccagaggaaagtcccagagtaaacacttgaatgttcaactggtggcagctgataaactggcccagtgtccccctgaaatgtttgatgttatattagatgagaatcaacttgaggatgcctgtgaacatctgggagagtacctggaggcatattggcgtgccacccacacaagcagtagcacccctatgaccccattgctggggaggaacgtgggctccacagctctctcaccgtatcccacagcaatctctggactacagagtcagcggatgaggcatagcaaccactctacagagaattctccaattgaaagacgaagcctaatgacctctgatgaaaattaccacaatgagagggcccgcaagagtaggaaccgcttgtcctccagctcccagcacagccgagatcactaccctctggtggaagaagattatcctgactcataccaggacacttacaaaccccataggaaccgaggctcacctgggggatgcagccatgactcccgacataggctttga

SEQ ID NO: 44 - DNA, 1794 bp, synthetic construct
  source 1..1794: mol_type = other DNA
atggtccagaagagcggcatgtcccggggcccttacccaccttcccaagagatccctatggaggtcttcgaccccagcccacagggcaaatacagcaagaggaaagggcggttcaaaaggtcagatgggagcacctcctcagataccacatccaacagctttgtccgtcagggctcagcagagtcctacacgagccggccgtcagactccgacgtgtccctggaggaggaccgggaagccttaaggaaggaggcagagcgccaggccttagcccagctcgagaaagccaagaccaaaccagtggcctttgctgttcggacaaatgttggctacaatccgtctccaggagatgaggtgccagtgcagggagtggccatcacctttgagcccaaggacttcctgcacatcaaggagaaatacaataatgactggtggatcgggaggctggtgaaggaaggctgtgaggttggtttcatccccagccctgtcaaactggacagccttcgcctgctgcaggaacagacgctgcgccaaaaccgcctcagctccagcaagtcaggtgacaactccagttccagtctgggagatgtggtgactggcacccgccgccccacaccccccgccagtgccaaacagaagcagaagtcgacagagcacgtgcccccctatgacgtggtgccttccatgaggcccatcatcctggtgggaccatcgctcaagggctatgaggtaactgacatgatgcagaaggcgttgtttgacttcttgaagcatcggtttgatggcaggatttccatcacccgggtaacggctgacatttccctggccaagcgctcggtcctcaacaaccccagcaaacacattatcatcgagcgctccaacactcgctccagcctggctgaggtacagagtgaaatcgagcggatcttcgagctggcccggaccttgcagctggttgccctggacgccgacaccatcaaccacccagcccagctctctaaaacctcgctggcccccatcattgtttacatcaagatcacatcccccaaggtactgcagaggctcatcaaatcccgagggaagtctcaatccaaacacctcaatgtccaaatagcagcctcggagaagctggcacagtgtccccccgaaatgtttgacataatcctggacgagaaccaattggaagatgcctgcgagcacctggcggagtacttggaagcctactggaaggccacacacccgcctagcagcacgccacccaatccgctgctgaaccgcaccatggctaccgccgctctggctgccagccctgcccccgtctccaacctccagggaccctaccttgcttccggggaccagccgctggaccgggccactggggagcatgccagtgtgcacgagtaccccggggagctgggccagcccccaggcctttaccccagcaaccacccacctggccgggcaggcaccctgcgggcgctatcccgccaagacacctttgatgctgacacccccggcagccgaaactctgcctacacggagccaggagactcgtgtgtggacatggagacagacccctcagagggcccagggcctggagaccctgcagggggaggtacaccaccagctcggcagggctcctgggaagaggaggaagattatgaggaggagatgaccgacaacaggaaccggggccggaataaggcccgctactgtgcggagggtggtgggccggttctggggcgcaataagaatgagctggagggctggggacaaggcgtctacatccgctga

SEQ ID NO: 45 - protein, 519 aa, Mus musculus [sequence omitted]

SEQ ID NO: 46 - protein, 519 aa, synthetic construct
MSSSYAKNGAADGPHSPSSQVARGTTTRRSRLKRSDGSTTSTSFILRQGSADSYTSRPSDSDVSLEEDREAIRQEREQQAAIQLERAKSKPVAFAVKTNVSYCGALDEDVPVPSTAISFDAKDFLHIKEKYNNDWWIGRLVKEGCEIGFIPSPLRLENIRIQQEQKRGRFHGGKSSGNSSSSLGEMVSGTFRATPTTTAKQKQKVTEHIPPYDVVPSMRPVVLVGPSLKGYEVTDMMQKALFDFLKHRFDGRISITRVTADISLAKRSVLNNPSKRAIIERSNTRSSLAEVQSEIERIFELARSLQLVVLDADTINHPAQLIKTSLAPIIVHVKVSSPKVLQRLIKSRGKSQSKHLNVQLVAADKLAQCPPEMFDVILDENQLEDACEHLGEYLEAYWRATHTSSSTPMTPLLGRNVGSTALSPYPTAISGLQSQRMRHSNHSTENSPIERRSLMTSDENYHNERARKSRNRLSSSSQHSRDHYPLVEEDYPDSYQDTYKPHRNRGSPGGCSHDSRHRL

SEQ ID NO: 47 - protein, 597 aa, synthetic construct
MVQKSGMSRGPYPPSQEIPMEVFDPSPQGKYSKRKGRFKRSDGSTSSDTTSNSFVRQGSAESYTSRPSDSDVSLEEDREALRKEAERQALAQLEKAKTKPVAFAVRTNVGYNPSPGDEVPVQGVAITFEPKDFLHIKEKYNNDWWIGRLVKEGCEVGFIPSPVKLDSLRLLQEQTLRQNRLSSSKSGDNSSSSLGDVVTGTRRPTPPASAKQKQKSTEHVPPYDVVPSMRPIILVGPSLKGYEVTDMMQKALFDFLKHRFDGRISITRVTADISLAKRSVLNNPSKHIIIERSNTRSSLAEVQSEIERIFELARTLQLVALDADTINHPAQLSKTSLAPIIVYIKITSPKVLQRLIKSRGKSQSKHLNVQIAASEKLAQCPPEMFDIILDENQLEDACEHLAEYLEAYWKATHPPSSTPPNPLLNRTMATAALAASPAPVSNLQGPYLASGDQPLDRATGEHASVHEYPGELGQPPGLYPSNHPPGRAGTLRALSRQDTFDADTPGSRNSAYTEPGDSCVDMETDPSEGPGPGDPAGGGTPPARQGSWEEEEDYEEEMTDNRNRGRNKARYCAEGGGPVLGRNKNELEGWGQGVYIR